(19) United States
(12) Patent Application Publication
(10) Pub. No.: US 2025/0205335 A1
(43) Pub. Date: Jun. 26, 2025

(54) SYNTHETIC TUMOR-INFILTRATING LYMPHOCYTES (TILS)

(71) Applicants: HANGZHOU CHENGYUAN BIOTECHNOLOGY CO., LTD., Zhejiang (CN); KESHIHUA (NANJING) BIOTECHNOLOGY CO., LTD., Jiangsu (CN)

(72) Inventors: Yue ZHAO, Guangdong (CN); Jiekun XUAN, Zhejiang (CN); Shiping JIAO, Jiangsu (CN); Qing XIONG, Jiangsu (CN)

(73) Assignees: HANGZHOU CHENGYUAN BIOTECHNOLOGY CO., LTD., Zhejiang (CN); KESHIHUA (NANJING) BIOTECHNOLOGY CO., LTD., Jiangsu (CN)

(21) Appl. No.: 18/847,272

(22) PCT Filed: Mar. 17, 2023
(86) PCT No.: PCT/CN2023/082120

§ 371 Date: Sep. 16, 2024

(30) Foreign Application Priority Data
Mar. 17, 2022 (WO) PCT/CN2022/081501

(51) Int. Cl. A61K 40/11; A61K 40/32; A61K 40/42; A61K 40/50; A61P 35/00; C07K 14/725

(52) U.S. Cl. CPC A61K 40/11; A61K 40/32; A61K 40/428; A61K 40/50; A61P 35/00; C07K 14/7051

(57) ABSTRACT

The present disclosure provides compositions and methods for producing modified cells (e.g., synthetic tumor-infiltrating lymphocytes or TILs). The modified cells can be used to prepare a pharmaceutical composition to be administered into a subject in need thereof. The modified cells can be polyclonal TCR-T cells expressing two or more different subject-specific T-cell receptors (TCRs).

### CROSS-REFERENCE TO RELATED APPLICATIONS

This application claims the benefit of International Application No. PCT/CN2022/081501, filed on Mar. 17, 2022, the entire content of which is incorporated herein by reference in its entirety.

### BACKGROUND OF THE INVENTION

Novel therapies are constantly developed for cancer treatment. Adoptive cell therapies (ACTs) are a potent approach for treating cancer but also for treating other diseases such as infections and graft versus host disease. Adoptive cell transfer is the passive transfer of ex vivo grown cells, most commonly immune-derived cells, into a host with the goal of transferring the immunologic functionality and characteristics of the transplant. Adoptive cell transfer can be autologous, as is common in adoptive T-cell therapies, or allogeneic as typical for treatment of infections or graft-versus-host disease. Clinically, common embodiments of this approach can include transfer of either immune-promoting or tolerogenic cells such as lymphocytes to patients to either enhance immunity against viruses and cancer or to promote tolerance in the setting of autoimmune disease, such as type I diabetes or rheumatoid arthritis.

The adoptive transfer of tumor infiltrating lymphocytes (TILs), especially autologous TILs, or genetically re-directed peripheral blood mononuclear cells has been used to treat patients with advanced solid tumors such as melanoma as well as patients with CD19-expressing hematologic malignancies. In ACT, the most commonly used cell types are the T-cells, sometimes sorted for CD8+, but other variations include CD4+ cells, NK-cells, delta-gamma T-cells, regulatory T-cells and peripheral blood mononuclear cells. Cells can be unmodified such as in TIL therapy or genetically modified.

### SUMMARY OF THE INVENTION

However, the efficacy of conventional TIL therapy in moderately immunogenic tumor (e.g., most epithelial tumors) is still inconsistent. There is a clear need to develop improved TIL therapy with improved efficacy to act against various tumors (e.g., solid tumors). Provided herein are novel compositions and methods to produced modified cells expressing polyclonal subject-specific TCRs for treatment of cancers. The compositions and methods described herein can offer fast turnaround time with low cost for personalized T cell therapy to a subject in need thereof having a solid tumor.

In an aspect, the present disclosure provides a method of producing a plurality of synthetic tumor-infiltrating lymphocytes (TILs), the method comprising: (a) obtaining a resection or biopsy sample comprising a plurality of T cells from a solid tumor lesion of a subject; (b) sequencing the plurality of T cells of the resection or biopsy sample to obtain natively paired T-cell receptor (TCR) alpha chain and beta chain sequences of TCRs of the plurality of T cells; (c) synthesizing a plurality of polynucleotide sequences encoding the TCRs or a subset thereof from the natively paired TCR alpha chain and beta chain sequences obtained in (b) comprising a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence; and (d) delivering the plurality of polynucleotide sequences or derivative thereof into a plurality of recipient cells by contacting a mixture comprising the plurality of polynucleotide sequences or derivative thereof with the plurality of recipient cells, thereby producing the plurality of synthetic TILs, wherein a first fraction of the plurality of synthetic TILs exogenously expresses the first polynucleotide sequence encoding the first natively paired TCR and a second fraction of the plurality of synthetic TILs exogenously expresses the second polynucleotide sequence encoding the second natively paired TCR, and wherein a third fraction of the plurality of synthetic TILs exogenously expressing both the first and the second polynucleotide sequence is less than the product of the first fraction and the second fraction.

In some embodiments, a cell of the plurality of synthetic TILs (i) exogenously expresses only one polynucleotide sequence of the plurality of polynucleotide sequences or (ii) does not exogenously express any of the plurality of polynucleotide sequences or derivative thereof. In some embodiments, none of the plurality of synthetic TILs exogenously expresses both the first and the second polynucleotide sequence. In some embodiments, a cell of the plurality of synthetic TILs that exogenously expresses the first polynucleotide sequence does not exogenously express the second polynucleotide sequence. In some embodiments, the method further comprises obtaining expression level of at least 10 genes in (b).

In another aspect, the present disclosure provides a method of producing a plurality of synthetic tumor-infiltrating lymphocytes (TILs), the method comprising: (a) obtaining a resection or biopsy sample comprising a plurality of T cells from a solid tumor lesion of a subject; (b) sequencing the plurality of T cells of the resection or biopsy sample to obtain natively paired T-cell receptor (TCR) alpha chain and beta chain sequences of TCRs of the plurality of T cells; (c) synthesizing a plurality of polynucleotide sequences encoding the TCRs or a subset thereof from the natively paired T-cell receptor (TCR) alpha chain and beta chain sequences obtained in (b) comprising a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence; and (d) delivering the plurality of polynucleotide sequences or derivative thereof into a plurality of recipient cells by contacting a mixture comprising the plurality of polynucleotide sequences or derivative thereof with the plurality of recipient cells, thereby producing the plurality of synthetic TILs, wherein a cell of the plurality of synthetic TILs that exogenously expresses the first polynucleotide sequence does not exogenously express the second polynucleotide sequence.

In some embodiments, a first fraction of the plurality of synthetic TILs exogenously expresses the first polynucleotide sequence and a second fraction of the plurality of synthetic TILs exogenously expresses the second polynucleotide sequence, and wherein a third fraction of the plurality of synthetic TILs exogenously expressing both the first and the second polynucleotide sequence is less than the product of the first fraction and the second fraction. In some embodiments, the method further comprises obtaining expression level of at least 10 genes in (b).

In another aspect, the present disclosure provides a method of producing a plurality of synthetic tumor-infiltrating lymphocytes (TILs), the method comprising: (a) obtaining a resection or biopsy sample comprising a plurality of T cells from a solid tumor lesion of a subject; (b) sequencing the plurality of T cells of the resection or biopsy sample to obtain (i) expression level of at least 10 genes and (ii) natively paired T-cell receptor (TCR) alpha chain and beta chain sequences of TCRs of the plurality of T cells; (c) synthesizing a plurality of polynucleotide sequences encoding the TCRs or a subset thereof from the natively paired TCR alpha chain and beta chain sequences obtained in (b) comprising a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence; and (d) delivering the plurality of polynucleotide sequences or derivative thereof into a plurality of recipient cells by contacting a mixture comprising the plurality of polynucleotide sequences or derivative thereof with a plurality of recipient cells, thereby producing the plurality of synthetic TILs.

In some embodiments, a first fraction of the plurality of synthetic TILs exogenously expresses the first polynucleotide sequence and a second fraction of the plurality of synthetic TILs exogenously expresses the second polynucleotide sequence, and wherein a third fraction of the plurality of synthetic TILs exogenously expressing both the first and the second polynucleotide sequence is less than the product of the first fraction and the second fraction.

In some embodiments, a cell of the plurality of synthetic TILs (i) exogenously expresses only one polynucleotide sequence of the plurality of polynucleotide sequences or (ii) does not exogenously express any of the plurality of polynucleotide sequences or derivative thereof. In some embodiments, none of the plurality of synthetic TILs exogenously expresses both the first and the second polynucleotide sequence.

In some embodiments, a cell of the plurality of synthetic TILs that exogenously expresses the first polynucleotide sequence does not exogenously express the second polynucleotide sequence.

In some embodiments, the first fraction, the second fraction, or the third fraction is determined by sequencing or pMHC multimer staining. In some embodiments, the sequencing comprises single cell sequencing, and wherein the single cell sequencing comprises barcoding each cell in an individual compartment. In some embodiments, the pMHC multimer staining comprises using a plurality of pMHC multimers, each specific for binding to a TCR encoded by a polynucleotide sequence of the plurality of polynucleotide sequences or derivative thereof in (d).

In some embodiments, delivering the plurality of polynucleotide sequences or derivative thereof into the plurality of recipient cells in (d) comprises site-specific knocking in a polynucleotide sequence into a genomic locus of a recipient cell of the plurality of recipient cells. In some embodiments, the genomic locus of the recipient cell leads to monoallelic expression of the polynucleotide sequence. In some embodiments, the polynucleotide sequence is site-specifically knocked in in-frame into the genomic locus of the recipient cell of the plurality of recipient cells. In some embodiments, the genomic locus is located on a sex chromosome. In some embodiments, the genomic locus is a T-cell receptor alpha constant (TRAC) locus. In some embodiments, the site-specific knocking in the polynucleotide sequence into the genomic locus comprises using a gene editing method selected from the group consisting of clustered regularly interspaced short palindromic repeats (CRISPR), transcription activator-like effector (TALE) nucleases (TALENs), meganucleases, zinc finger nuclease (ZFN), or megaTAL nucleases method.

In some embodiments, the site-specific knocking in the polynucleotide sequence into the genomic locus comprises using CRISPR method. In some embodiments, the CRISPR method comprises delivering a ribonucleoprotein (RNP) complex comprising a Cas9 protein and a single guide RNA (sgRNA) into the recipient cell. In some embodiments, the sgRNA targets the TRAC locus. In some embodiments, the site-specific knocking in the polynucleotide sequence into the genomic locus comprises cutting the genomic locus using the gene editing method, and inserting the polynucleotide sequence into the genomic locus via homologous recombination.

In some embodiments, an endogenous TCR is inactivated in a recipient cell of the plurality of recipient cells. In some embodiments, the endogenous TCR is knocked down or knocked out in the recipient cell of the plurality of recipient cells. In some embodiments, an endogenous T-cell receptor beta constant (TRBC) locus is inactivated in a recipient cell of the plurality of recipient cells. In some embodiments, the endogenous TRBC locus is knocked down or knocked out in the recipient cell of the plurality of recipient cells. In some embodiments, an endogenous TCR is inactivated in at least about 80% of the plurality of recipient cells. In some embodiments, the endogenous TCR is knocked down or knocked out in at least about 80% of the plurality of recipient cells. In some embodiments, the endogenous TCR is inactivated using a gene editing method selected from the group consisting of clustered regularly interspaced short palindromic repeats (CRISPR), transcription activator-like effector (TALE) nucleases (TALENs), meganucleases, zinc finger nuclease (ZFN), or megaTAL nucleases method.

In some embodiments, the endogenous TCR is inactivated prior to, concurrently with, or after delivering the plurality of polynucleotide sequences or derivative thereof into the plurality of recipient cells in (d). In some embodiments, the endogenous TCR is inactivated concurrently with delivering the plurality of polynucleotide sequences or derivative thereof into the plurality of recipient cells in (d).

In some embodiments, the endogenous TCR is inactivated using CRISPR method. In some embodiments, the CRISPR method comprises delivering a ribonucleoprotein (RNP) complex comprising a Cas9 protein and a single guide RNA (sgRNA) into the recipient cell. In some embodiments, the sgRNA comprises a sgRNA targeting the TRBC locus. In some embodiments, the endogenous TCR is inactivated concurrently with the site-specific knocking in the polynucleotide sequence into the genomic locus by delivering the RNP comprising the Cas9 protein and sgRNAs that comprises a sgRNA targeting the TRBC locus and a sgRNA targeting the TRAC locus.

In some embodiments, at least about 50% of the plurality of synthetic TILs exogenously express only a single copy of polynucleotide sequence encoding a natively paired TCR of the TCRs of the plurality of T cells.

In some embodiments, the method further comprises, subsequent to (d), expanding the plurality of synthetic TILs. In some embodiments, the mixture comprises polynucleotide sequences encoding at least 5 different natively paired TCRs, and wherein a unique TCR of the at least 5 different natively paired TCRs has a first frequency in the mixture. In some embodiments, after expansion the unique TCR has a second frequency in the plurality of synthetic TILs. In some embodiments, the second frequency is within 0.1-fold to 10-fold of the first frequency. In some embodiments, the polynucleotide sequences encoding the at least 5 different natively paired TCRs are mixed at equal molar ratio. In some embodiments, after expansion each unique TCR maintains an equal proportion within the plurality of synthetic TILs or has a proportion that does not go below 0.1-fold or exceed 10-fold of a proportion of any of the other unique TCRs within the plurality of synthetic TILs.

In some embodiments, the method further comprises administering at least one cell or progeny thereof of the plurality of synthetic TILs into the subject having the solid tumor lesion. In some embodiments, prior to administering, the plurality of synthetic TILs have not been subjected to any selection or screening for determining tumor reactivity of the plurality of synthetic TILs or subset thereof.

In some embodiments, the method further comprises determining a tumor reactivity of the plurality of synthetic TILs. In some embodiments, determining the tumor reactivity comprises: (i) providing a tumor cell or derivative thereof from the subject; (ii) contacting the plurality of synthetic TILs with the tumor cell or derivative thereof; (iii) determining the tumor reactivity of the plurality of synthetic TILs against the tumor cell or derivative thereof; and (iv) determining whether to administer the plurality of synthetic TILs or subset thereof into the subject. In some embodiments, the method further comprises administering at least one cell or progeny thereof of the plurality of synthetic TILs into the subject having the solid tumor lesion.

In some embodiments, the method further comprises, prior to synthesizing in (c), calculating a score using the expression level of at least 3 genes of the at least 10 genes obtained in (b); ranking the plurality of T cells based on the score; and selecting at least two TCRs from the top 50% of the plurality of T cells according to the score. In some embodiments, the plurality of polynucleotide sequences encoding the TCRs or a subset thereof synthesized in (c) comprises polynucleotide sequences encoding the at least two TCRs selected.

In some embodiments, a time interval between obtaining the resection or biopsy sample and administering the at least one cell of the plurality of synthetic TILs into the subject is less than eight weeks. In some embodiments, a time interval between obtaining the resection or biopsy sample in (a) and the delivering in (d) is no more than 7 weeks.

In some embodiments, the plurality of synthetic TILs exogenously express at least about 2, 3, 4, 5, 10, 20, 30, 40, 50, 100, 500, 1,000 or more different TCRs. In some embodiments, two or more cells or progenies thereof of the plurality of synthetic TILs are administered into the subject, and the two or more cells administered exogenously express at least about 2, 3, 4, 5, 10, 20, 30, 40, 50, 100, 500, 1,000 or more different TCRs.

In some embodiments, the plurality of synthetic TILs comprises a subpopulation of cells expressing TCRs that recognize tumor antigens and a subpopulation of cells expressing TCRs that do not recognize tumor antigens.

In some embodiments, a volume of the resection or biopsy sample is less than about 100 mm³ (μL), or a weight of the resection or biopsy sample is less than about 0.1 g.

In some embodiments, the plurality of polynucleotide sequences or derivative thereof is delivered into the plurality of recipient cells by a vector. In some embodiments, the vector is a non-viral vector. In some embodiments, the non-viral vector is a plasmid, a transposon, a self-amplifying RNA, or any combination thereof. In some embodiments, the vector is a viral vector. In some embodiments, the viral vector is an adenoviral vector, an adeno-associated viral (AAV) vector, a herpes simplex viral vector, lentiviral vector, a retroviral vector, or any combination thereof. In some embodiments, the viral vector is the AAV vector. In some embodiments, the vector does not comprise a promoter. In some embodiments, the plurality of recipient cells are a plurality of T cells.

In some embodiments, the plurality of recipient cells are isolated from a subject. In some embodiments, the subject is the same subject where the plurality of T cells in (a) are obtained. In some embodiments, the plurality of recipient cells are primary T cells. In some embodiments, the plurality of recipient cells are peripheral T cells.

In some embodiments, the mixture comprises at least about 2, 3, 4, 5, 10, 20, 30, 40, 50, 100, 500, 1,000 or more different polynucleotide sequences encoding at least about 2, 3, 4, 5, 10, 20, 30, 40, 50, 100, 500, 1,000 or more different natively paired TCRs.

In some embodiments, the subject has failed a prior therapy. In some embodiments, the prior therapy is a checkpoint blockade therapy.

In another aspect, the present disclosure provides a method of producing a plurality of synthetic tumor-infiltrating lymphocytes (TILs), the method comprising: (a) obtaining a resection or biopsy sample comprising a plurality of T cells from a solid tumor lesion of a subject; (b) synthesizing a plurality of polynucleotide sequences encoding natively paired TCRs or a subset thereof of the plurality of T cells obtained in (a) comprising a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence; and (c) delivering the plurality of polynucleotide sequences or derivative thereof into a plurality of recipient cells by contacting a mixture comprising the plurality of polynucleotide sequences or derivative thereof with a plurality of recipient cells, thereby producing the plurality of synthetic TILs.

In some embodiments, the method further comprises, prior to (b), sequencing the plurality of T cells of the resection or biopsy sample to obtain natively paired T-cell receptor (TCR) alpha chain and beta chain sequences of TCRs of the plurality of T cells. In some embodiments, the method further comprises obtaining expression level of at least 10 genes from the sequencing.

In another aspect, the present disclosure provides a method of producing a plurality of synthetic tumor-infiltrating lymphocytes (TILs), the method comprising: (a) delivering a plurality of polynucleotide sequences or derivative thereof into a plurality of recipient cells by contacting a mixture comprising the plurality of polynucleotide sequences or derivative thereof with the plurality of recipient cells, wherein each polynucleotide sequence of the plurality encodes a cognate pair of TCR alpha chain and a TCR beta chain, and wherein each polynucleotide sequence is knocked into a genomic locus for monoallelic expression of the polynucleotide sequence; and (b) inactivating an endogenous TCR gene of each recipient cell of the plurality of recipient cells or an endogenous TCR gene of each recipient cell of the plurality of recipient cells has been inactivated.

In some embodiments, at least about 50% of the plurality of synthetic TILs exogenously express only a single copy of polynucleotide sequence encoding a natively paired TCR of the TCRs of the plurality of T cells. In some embodiments, the genomic locus is TRAC locus. In some embodiments, the endogenous TCR gene is disrupted by knocking down or knocking out TRBC locus. In some embodiments, the mixture comprises at least about 2, 3, 4, 5, 10, 20, 30, 40, 50, 100, 500, 1,000 or more different polynucleotide sequences encoding at least about 2, 3, 4, 5, 10, 20, 30, 40, 50, 100, 500, 1,000 or more different natively paired TCRs.

In some embodiments, the method further comprises, prior to (a), synthesizing the plurality of polynucleotide sequences encoding different cognate pairs of TCR alpha chains and TCR beta chains. In some embodiments, synthesizing comprises assembling the plurality of polynucleotide sequences encoding different cognate pairs of TCR alpha chains and TCR beta chains in a same mixture. In some embodiments, synthesizing comprises chemically synthesizing the plurality of polynucleotide sequences individually.

In some embodiments, an endogenous TCR gene of each recipient cell of the plurality of recipient cells has been inactivated prior to delivering in (a). In some embodiments, inactivating an endogenous TCR gene of each recipient cell of the plurality of recipient cells in (b) is done prior to, concurrently, or after delivering in (a).

In some embodiments, the method further comprises, prior to (a), obtaining a resection or biopsy sample comprising a plurality of T cells from a solid tumor lesion of a subject, and sequencing the plurality of T cells of the resection or biopsy sample to obtain natively paired T-cell receptor (TCR) alpha chain and beta chain sequences of TCRs of the plurality of T cells.

In some embodiments, the method further comprises selecting a plurality of sequences encoding a plurality of natively paired TCRs from the sequencing, wherein the plurality of sequences selected or a subset thereof is the plurality of polynucleotide sequences delivered in (a).

In some embodiments, inactivating in (b) is done concurrently, prior to or after delivering in (a).

In some embodiments, the method further comprises expanding the plurality of synthetic TILs. In some embodiments, a frequency of a unique TCR in the mixture prior to delivering is within 0.1-fold to 10-fold of a frequency of the unique TCR in the plurality of synthetic TILs after expansion.

In some embodiments, the method further comprising administering at least one cell or progeny thereof of the plurality of synthetic TIL to the subject. In some embodiments, after administering, the synthetic TILs persist in the subject in need thereof for at least about 30 days. In some embodiments, after administering, the percentage of the synthetic TILs or progeny thereof among the subject's peripheral T cells is at least about 20%. In some embodiments, the percentage of CD62L⁺ cells of the synthetic TILs or progeny thereof among the subject's peripheral T cells is at least about 50%. In some embodiments, the percentage persists for at least about 30 days. In some embodiments, a concentration of the synthetic TILs in a peripheral blood sample of the subject is at least about 0.2×10⁶ cells per mL of peripheral blood. In some embodiments, the concentration persists for at least about 30 days with or without IL-2 infusion.

In another aspect, the present disclosure provides a pharmaceutical composition comprising one or more synthetic TILs described herein, and a pharmaceutically acceptable carrier.

In another aspect, the present disclosure provides a mixture comprising at least 2, 5, 10, 15, 20, 25, 30, 50, 100, 200, 500, or 1000 modified cells, each modified cell of the mixture exogenously expressing a polynucleotide sequence encoding a distinct cognate pair of a TCR alpha chain and a TCR beta chain, wherein the polynucleotide sequence is knocked into a genomic locus for monoallelic expression of the polynucleotide sequence, and wherein an endogenous TCR gene of the modified cell is inactivated (e.g., knocked out).

In another aspect, the present disclosure provides a mixture of cells comprising a plurality of modified cells, each modified cell of the plurality exogenously expressing a polynucleotide sequence encoding a distinct cognate pair of a TCR alpha chain and a TCR beta chain, wherein the polynucleotide sequence is knocked into a genomic locus for monoallelic expression of the polynucleotide sequence. In some embodiments, an endogenous TCR gene of the modified cell is inactivated.

In some embodiments, each distinct cognate pair of a TCR alpha chain and a TCR beta chain is from a subject having a solid tumor lesion or is expressed endogenously in a T cell from a subject having a solid tumor lesion, and wherein the subject is intended to be administered with the mixture of modified cells or a pharmaceutical composition comprising the mixture of modified cells.

In some embodiments, the mixture comprises at least 10 modified cells, each modified cell of the mixture exogenously expressing a polynucleotide sequence encoding a distinct cognate pair of a TCR alpha chain and a TCR beta chain.

In some embodiments, the genomic locus is TRAC locus. In some embodiments, the endogenous TCR gene of the modified cell is knocked out or knocked down. In some embodiments, the endogenous TCR gene comprises a TRBC locus. In some embodiments, the modified cell is a T cell. In some embodiments, the T cell is an allogeneic T cell or an autologous T cell.

In some embodiments, the distinct cognate pair of a TCR alpha chain and a TCR beta chain of each modified cell is selected by sequencing TCRs from a plurality of T cells from a subject. In some embodiments, the distinct cognate pair of a TCR alpha chain and a TCR beta chain of each modified cell is selected by a pre-designed scoring method based on sequencing data of TCRs from a plurality of T cells from a subject. In some embodiments, the distinct cognate pair of a TCR alpha chain and a TCR beta chain of each modified cell has been validated to be functional. In some embodiments, the distinct cognate pair of a TCR alpha chain and a TCR beta chain of each modified cell is tumor-reactive. In some embodiments, the mixture comprises both functional and non-functional TCRs. In some embodiments, the mixture comprises both tumor-reactive and tumor-nonreactive TCRs. In some embodiments, the mixture of modified cells have been expanded in vitro.

In another aspect, the present disclosure provides a mixture of cells comprising a plurality of modified cells, each modified cell of the plurality exogenously expressing a cognate pair of a TCR from a subject, wherein the mixture of cells expresses at least 10 different cognate pairs of TCRs, wherein each TCR of the at least 10 different cognate pairs of TCRs is expressed in at least 2% of the mixture of cells, and wherein at least 50% of the mixture of cells each exogenously expresses only one cognate pair of TCR. In some embodiments, the subject is intended to be administered with the mixture or a pharmaceutical composition comprising the mixture. In some embodiments, a polynucleotide sequence encoding a cognate pair of TCR is knocked into a genomic locus for monoallelic expression of the polynucleotide sequence in each modified cell of the at least 50% of the mixture exogenously express only one cognate pair of TCR. In some embodiments, the modified cells persist for at least about 30 days in the subject when administered into the subject.

In another aspect, the present disclosure provides a pharmaceutical composition comprising the mixture of cells comprising modified cells described herein and a pharmaceutically acceptable carrier.

In another aspect, the present disclosure provides a composition for use in the manufacture of a medicament for treating a subject having a cancer, comprising the mixture of cells comprising modified cells described herein.

In another aspect, the present disclosure a method of treating cancer in a subject in need thereof, the method comprising administering to the subject a therapeutically effective amount of the pharmaceutical composition of described herein or a pharmaceutical composition comprising the mixture described herein.

In another aspect, the present disclosure a method of treating cancer in a subject in need thereof, the method comprising administering to the subject a pharmaceutical composition comprising (a) a synthetic TIL produced according to the method described herein or a mixture of two or more synthetic TILs produced according to the method described herein; and (b) a pharmaceutically acceptable carrier.

In another aspect, the present disclosure provides a method of treating a cancer in a subject in need thereof, the method comprising administering a pharmaceutical composition comprising a mixture of cells into the subject in need thereof, wherein the mixture of cells exogenously expresses at least 10 different cognate pairs of TCRs from a subject, and wherein at least 50% of the mixture of cells each exogenously expresses only one cognate pair of TCR.

In some embodiments, the mixture of cells are a mixture of modified cells. In some embodiments, the mixture of cells comprise a plurality of modified cells and a plurality of unmodified cells. In some embodiments, each TCR of the at least 10 different cognate pairs of TCRs is expressed in at least 2% of the mixture of modified cells. In some embodiments, the polynucleotide sequence is knocked into a genomic locus for monoallelic expression of the polynucleotide sequence. In some embodiments, the subject from whom the TCRs are obtained is the same as the subject to be treated. In some embodiments, an endogenous TCR gene of the modified cell is inactivated.

In some embodiments, the modified cell is a T cell. In some embodiments, the T cell is a CD4⁺ T cell, a CD8⁺ T cell, a CD4⁺/CD8⁺ T cell, a helper T cell, a cytotoxic T cell, a memory T cell, a regulatory T cell, a natural killer T cell, an alpha beta T cell, or a gamma delta T cell. In some embodiments, the T cell is an allogenic T cell or an autologous T cell. In some embodiments, the modified cell is isolated from the subject being administered with the composition. In some embodiments, the distinct cognate pair of each modified cell is from the subject in need thereof or is expressed endogenously by a T cell from the subject in need thereof.

In some embodiments, the mixture comprises at least 10 modified cells. In some embodiments, the genomic locus is TRAC locus. In some embodiments, the endogenous TCR gene of the modified cell is knocked out or knocked down. In some embodiments, the endogenous TCR gene comprises a TRBC locus. In some embodiments, the subject in need thereof has been subjected to one or more lines of prior therapy or has failed one or more lines of prior therapy. In some embodiments, the cancer is metastatic or refractory. In some embodiments, the cancer is a solid tumor.

In some embodiments, the method further comprises administering an IL-2 into the subject after administering the pharmaceutical composition. In some embodiments, prior to administering, detecting tumor reactivity of the mixture of cells.

In some embodiments, the mixture of modified cells persist in the subject for at least about 30 days. In some embodiments, after administering, the percentage of the modified cells or progeny thereof among the subject's peripheral T cells is at least about 20%. In some embodiments, the percentage of CD62L⁺ cells of the synthetic TILs or progeny thereof among the subject's peripheral T cells is at least about 50%. In some embodiments, the percentage persists for at least about 30 days. In some embodiments, a concentration of the synthetic TILs in a peripheral blood sample of the subject is at least about 0.2×10⁶ cells per mL of peripheral blood. In some embodiments, the concentration persists for at least about 30 days with or without IL-2 infusion.

In another aspect, the present disclosure provides use of the modified cells (e.g., synthetic TILs) described herein in the preparation of a medicament for use in treating cancer (e.g., solid tumor).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “Fig.”, and “FIGURE” herein) of which:

FIG. 1 depicts experimental data showing quality of 48 individually amplified TCR genes assessed by fragment analyzer.

FIG. 2 depicts example tetramer staining showing single-copy insertion of TCRs.

FIG. 3 depicts example flowcytometry analysis of the polyclonal synthetic TILs.

FIG. 4 depicts an example scheme to sequence the mRNA transcript carrying the exogenous TCRs.

FIGS. 5A-5C depict an example of processing of needle biopsy sample of a tumor. FIG. 5A depicts a needle biopsy sample in tissue storage solution. FIG. 5B depicts quality control data of the resultant single cell suspension using Bio-Rad TC20 Automated Cell Counter. FIG. 5C depicts phase-contrast microscopic image of the resultant single cell suspension.

FIG. 6 depicts experimental quality control data for the single-cell RNA-Seq of the needle biopsy sample. Gene expression profile of 7,983 cells (including tumor cells and non-tumor cells in the sample) were obtained.

FIG. 7 depicts experimental quality control data for the single-cell RNA-Seq of the needle biopsy sample. TCR sequences of 4,528 cells were detected, out of which 3,462 cells contain productive V-J spanning pair.

FIG. 8 depicts experimental quality control data for the single-cell RNA-Seq (gene expression profile). The distribution of feature count (e.g., gene count), transcript count (e.g., UMI count), percentage of mitochondria-derived transcripts, and percentage of ribosome-derived transcripts are shown from left to right.

FIG. 9 depicts experimental data showing Uniform Manifold Approximation and Projection (UMAP) showing the gene expression profile of all T cells in the needle biopsy sample. The grayscale shows the signature score of each T cell.

FIG. 10 depicts experimental data showing the UMAP coordinates of all T cells containing TCR2 (the ‘CAVI’ TCR).

FIG. 11 depicts experimental data showing the UMAP coordinates of all T cells containing TCR1 (the ‘CAVH’ TCR).

FIG. 12 depicts experimental data showing the UMAP coordinates of all T cells containing TCR3 (the ‘CADS’ TCR).

FIG. 13 depicts experimental data showing tumor-reactivity of TCR1, TCR2 and TCR3 measured by IFNg secretion when the T cells carrying the TCR is co-cultured with primary tumor.

FIG. 14 depicts experimental data showing that tumor size reduction was observed for the lung lesion of the hepatocellular carcinoma patient (Patient 1) after synthetic TIL infusion.

FIG. 15 depicts Real-Time Cell Analysis (RTCA) assay for the 3 TCRs discovered for Patient 2.

FIG. 16 depicts experimental data showing that tumor size reduction was observed for the urothelial cancer patient (Patient 3) after synthetic TIL infusion. The cross bars show the size the tumor lesion.

FIGS. 17A-17C depict an example scheme of generating a nucleic acid construct encoding a T-cell receptor.

FIGS. 18A-18C depict schematics of TCR genes. FIG. 18A depicts a schematic of germline genomic DNA of a TCR V gene. FIG. 18B depicts a schematic of rearranged genomic DNA of a TCR V-J gene. FIG. 18C depicts a schematic of rearranged genomic DNA of a TCR V-D-J gene.

FIG. 19 depicts a scheme of potential challenge associated with linking a CDR3-J polynucleotide to the correct V gene germline polynucleotide. The dashed arrows depict linking can happen between the CDR3-J polynucleotide and the incorrect V gene germline polynucleotide.

FIG. 20 depicts a scheme of linking a CDR3-J polynucleotide (the gray box connected to the white box) to the designated, pre-synthesized V gene germline polynucleotide (the black box connected to the gray box pointed by the thin arrow), by overlapping primer extension. The top thick arrow (603) depicts hybridization between the connector sequence on the pre-synthesized V gene germline polynucleotide (601) and the connector sequence on the CDR3-J polynucleotide (602). The bottom thick arrow (604) depicts primer extension. 601 may be referred to as a connector sequence and 602 may be referred to as an anti-connector sequence (or vice versa).

FIG. 21 depicts linking a CDR3-J polynucleotide and the designated V gene germline polynucleotide using arbitrary connector (701) and anti-connector (702) sequences.

FIG. 22 depicts a general principle of TCR gene self-assembly. 801: a pre-synthesized V gene germline polynucleotide. 802: a polynucleotide comprising a CDR3-J sequence (e.g., a CDR3-J polynucleotide). 803: a nucleic acid sequence comprising a V gene germline polynucleotide sequence and a CDR3-J sequence. X is the number of polynucleotides each being a portion of a different V gene germline polynucleotide. Y is the number of CDR3-J polynucleotides. Y may be much larger than X. The arrow indicates a bulk reaction where each CDR3-J polynucleotide is linked to the designated, pre-synthesized V gene germline polynucleotide.

FIG. 23A and FIG. 23B depict an example scheme to produce TCR gene pool from which individual TCR gene can be selectively amplified by PCR using a primer corresponding to the Zip sequence of the TCR gene.

FIG. 24A and FIG. 24B depict flowcytometry analysis of modified cells infected with lentiviral particles containing exogenous TCRs.

FIGS. 25A-25D depict single cell transcriptomics data of the fine needle biopsy of patient CMI-0102. FIG. 25A depicts module E score, calculated by gene expression levels of about 30 genes implicated in T cell exhaustion. FIG. 25B depicts automatic cell clustering. FIG. 25C depicts module T score, calculated by gene expression levels of about 30 genes implicated by secondary lymphoid structure and T cell activation. FIG. 25D depicts clonality.

FIGS. 26A-26D depict single cell transcriptomics data of the fine needle biopsy of patient CMI-0102. Expression levels of CD3D, CD8A, CD4, and FOXP3 in each cell are shown in FIGS. 26A, 26B, 26C and 26D, respectively.

FIG. 27 depicts single cell transcriptomics data of the fine needle biopsy of patient CMI-0102, showing location of the T cells carrying the TCRs chosen for the manufacturing synthetic TIL cells, as displayed on UMAP.

FIG. 28 depicts abundance of each TCR in the R&D grade plasmid mixture (panel A), GMP grade plasmid mixture (panel B), GMP grade AAV mixture (panel C) and synthetic TIL cell mixture (panel D) for patient CMI-0102. The unit of the values shown is %. The dashed line shows the position 1/N*100, where N is the number of TCRs mixed in equal proportion.

FIG. 29A depicts level of tumor biomarker CEA of patient CMI-0102 on different days after infusion of T cell product. FIG. 29B depicts body temperature. For example, D10 means day 10 after infusion. Baseline means the day before infusion. LD-0 and LD-5 mean the day of lymphodepletion and the 5th day after lymphodepletion.

FIG. 30 depicts serum level of IL-6, CRP and Ferritin of patient CMI-0102 on different days after infusion of T cell product. The upper limit of normal range is shown as dashed line.

FIGS. 31A-31D depict blood cell count (FIGS. 31A and 31B) and pharmacokinetics (FIGS. 31C and 31D) of the synthetic TIL cells for patient CMI-0102. WBC: white blood cell count. LYMPH: lymphocyte count. In FIG. 31A and FIG. 31B, lower bound of normal range is shown as dash lines. In FIG. 31C and FIG. 31D, the word “disTIL” is used to describe synthetic TIL cell product. FIG. 31C shows the abundance of synthetic TIL cells as percentage of all CD3⁺ T cells. FIG. 31D shows the absolute number of synthetic TIL cells in log₁₀ scale.

FIG. 32 depicts single-cell transcriptomic analysis of peripheral T cells of patient CMI-0102 at days 10, 14 and 21 after infusion of synthetic TIL cells. Expression levels of the several markers are highlighted.

FIGS. 33A-33D depict Single-cell transcriptomic and clonotype analysis of synthetic TIL cells in the peripheral T cells of patient CMI-0102 at days 10, 14 and 21 after infusion of synthetic TIL cells. FIG. 33A depicts exhaustion score calculated by the expression levels of about 30 genes implicated in T cell exhaustion. FIG. 33B depicts Relative abundance of different exogenous TCRs (used to manufacture the synthetic TIL cells). The T cells carrying the exogenous TCRs are highlighted on the UMAP of FIG. 33C (day 10), FIG. 33D (day 14), and FIG. 33E (day 21).

FIGS. 34A-34D depict single cell transcriptomics data of the fine needle biopsy of patient CMI-0104. FIG. 34A depicts module E score, calculated by gene expression levels of about 30 genes implicated in T cell exhaustion. FIG. 33B depicts automatic cell clustering. FIG. 33C depicts module T score, calculated by gene expression levels of about 30 genes implicated by secondary lymphoid structure and T cell activation. FIG. 33D depicts clonality.

FIGS. 35A-35C depicts single cell transcriptomics data of the fine needle biopsy of patient CMI-0104. Expression levels of CD8A, CD4, and FOXP3 in each cell are shown in FIGS. 35A, 35B and 35C, respectively.

FIG. 36 depicts single cell transcriptomics data of the fine needle biopsy of patient CMI-0104, showing location of the T cells carrying the TCRs chosen for the manufacturing synthetic TIL cells, as displayed on UMAP.

FIG. 37 depicts abundance of each TCR in the R&D grade plasmid mixture (panel A), GMP grade plasmid mixture (panel B), GMP grade AAV mixture (panel C) and synthetic TIL cell mixture (panel D) for patient CMI-0104. The unit of the values shown is %. The dashed line shows the position 1/N*100, where N is the number of TCRs mixed in equal proportion.

FIG. 38 depicts body temperature of patient CMI-0104 on different days after infusion of T cell product.

FIG. 39 depicts serum level of IL-6, CRP and Ferritin of patient CMI-0104 on different days after infusion of T cell product. The upper limit of normal range is shown as dashed line.

FIGS. 40A-40D depict blood cell count (FIGS. 40A and 40B) and pharmacokinetics (FIGS. 40C and 40D) of the synthetic TIL cells for patient CMI-0104. WBC: white blood cell count. LYMPH: lymphocyte count. In A and B, lower bound of normal range is shown as dash lines. In FIG. 40C and FIG. 40D, the word “disTIL” is used to describe synthetic TIL cell product. FIG. 40C shows the abundance of synthetic TIL cells as percentage of all CD3+ T cells. FIG. 40D shows the absolute number of synthetic TIL cells. The duration of IL-2 infusion is indicated on each plot.

### DETAILED DESCRIPTION OF THE INVENTION

In this disclosure, the use of the singular includes the plural unless specifically stated otherwise. Also, the use of “or” means “and/or” unless stated otherwise. Similarly, “comprise,” “comprises,” “comprising,” “include,” “includes,” and “including” are not intended to be limiting.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, or within 2-fold, of a value. Where particular values are described in the application and claims, unless otherwise stated the term “about” meaning within an acceptable error range for the particular value should be assumed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “polynucleotide”, “nucleic acid” and “oligonucleotide” are used interchangeably in the present disclosure. They can refer to a polymeric form of nucleotides of various length. They may comprise deoxyribonucleotides and/or ribonucleotides, or analogs thereof. A polynucleotide may include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. A polynucleotide may have any three-dimensional structure and may perform various functions. A polynucleotide can have various configurations, such as linear, circular, stem-loop, and branched. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), circular RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotides may include one or more nucleotide variants, including nonstandard nucleotide(s), non-natural nucleotide(s), nucleotide analog(s) and/or modified nucleotides.

The term “sequence,” as used herein, refers to the order of nucleotides in a nucleic acid molecule, or the order of amino acid residues of a peptide. A nucleic acid sequence can be a deoxyribonucleic acid (DNA) sequence or ribonucleic acid (RNA) sequence; can be linear, circular or branched; and can be either single-stranded or double-stranded. A sequence can be mutated such that it is different from a reference sequence (e.g., wildtype sequence). A sequence can be of any length, for example, between 2 and 1,000,000 or more amino acids or nucleotides in length (or any integer value there between or there above), e.g., between about 100 and about 10,000 nucleotides or between about 200 and about 500 amino acids or nucleotides. In some cases, a given nucleic acid sequence can encompass the sequence information of the given nucleic acid sequence and a reverse complement sequence of the given nucleic acid sequence. In some cases, a DNA sequence can encompass the sequence information of the corresponding RNA sequence that is transcribed from the DNA. The sequence can be alphabetical representation of a polynucleotide or polypeptide molecule. The sequence can be a piece of information that can be used by a computer processor. In some cases, the nucleic acid sequence may be used to refer to the physical nucleic acid molecule itself.

The term “blunt end,” as used herein, refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion having at least one nucleotide in length, referred to herein as an “overhang” or “sticky end.”

The term “TCR V gene,” as used herein, refers to a genomic nucleic acid sequence of a T-cell receptor variable (V) gene, in germline configuration, that comprises the sequence encoding the first part of the leader peptide (e.g., L-PART1 as defined in IMGT), an intron (e.g., V-INTRON as defined in IMGT) and an exon (e.g., V-EXON as defined in IMGT), with a 5′UTR and a 3′UTR (including recombination signal sequence). The recombination signal sequence can comprise a heptamer (e.g., V-HEPTAMER as defined in IMGT) and a nonamer (e.g., V-NONAMER as defined by IMGT), separated by a spacer element (e.g., V-SPACER as defined by IMGT). V-EXON encompasses the sequence encoding the second part of the leader peptide (L-PART2) and V-REGION. Examples of TCR V gene include TCR alpha variable (TRAV) gene, TCR beta variable (TRBV) gene, TCR gamma variable (TRGV) gene, and TCR delta variable (TRDV) gene. A nucleic acid described herein can comprise a sequence derived from the TCR V gene. By “derived from,” it means a sequence having a sequence identity of at least about 40%, at least about 50%, at least about 60%, at least about 70%, at least about 80%, at least about 90%, at least about 95%, or 100% with a reference sequence. A sequence derived from a TCR V gene can be a full length sequence of the genomic nucleic acid sequence of a TCR V gene as described above. A sequence derived from a TCR V gene can be a portion of the TCR V gene comprising at least 10, 20, 30, 40, 50, 60, 70, 80, 90, 100, 200, 300, 400, 500, or more nucleotides of the TCR V gene. A sequence derived from a TCR V gene can be a codon-optimized (or codon-diversified) nucleic acid sequence. A codon-optimized sequence of a given nucleic acid sequence refers to a modified nucleic acid sequence whose protein-coding region encodes the same amino acid sequence as the protein-coding region of the given nucleic acid. The modified nucleic acid sequence may have a sequence different from the given nucleic acid sequence or can be derived from the given nucleic acid. Codon optimization may be implemented to remove restriction site, to remove unwanted secondary structure in the polynucleotide sequence, to promote correct linking of a CDR3-J polynucleotide and the designated pre-synthesized portion of a TCR V gene, or for other purposes. Codon optimization or codon diversification can be achieved by altering one or more nucleotides of a given nucleic acid sequence. For example, codon optimization or codon diversification can be achieved by computational methods. Codon optimization and codon diversification may be used interchangeably in the present disclosure.

The term “V-REGION,” as used herein, refers to coding region of a TCR V gene (includes 1 or 2 nucleotides before the V-HEPTAMER, if present) in germline genomic DNA or cDNA, or variable (V) region usually trimmed in 3′ by the V-(D)-J rearrangement in rearranged genomic DNA or cDNA.

The term “D-REGION,” as used herein, refers to coding region of a TCR D gene (includes 1 or 2 nucleotide(s) after the 5′ D-HEPTAMER and/or before the 3′ D-HEPTAMER, if present) in germline genomic DNA or cDNA, or diversity (D) region usually trimmed in 5′ and/or 3′ by the D-J or V-D-J rearrangement in partially-rearranged or in rearranged genomic DNA or in cDNA.

The term “J-REGION,” as used herein, refers to coding region of a TCR J gene (includes 1 or 2 nucleotide(s) after J-HEPTAMER, if present) in germline genomic DNA or cDNA, or joining (J) region usually trimmed in 5′ by the V-(D)-J rearrangement in rearranged genomic DNA or cDNA.

The term “V-J-REGION,” as used herein, refers to coding region of a TCR chain that comprises V-REGION and J-REGION, in rearranged genomic DNA or cDNA.

The term “V-D-J-REGION,” as used herein, refers to coding region of a TCR chain that comprises V-REGION, D-REGION, and J-REGION, in rearranged genomic DNA or cDNA.

The terms “link” or “connect” are used interchangeably in the present disclosure. They refer to physically linking two or more nucleic acid molecules. The two or more nucleic acid molecules may be linked such that the two or more nucleic acid molecules form a continuous nucleic acid molecule. The two or more nucleic acid molecules can be covalently linked or non-covalently linked. Linking may be accomplished in a variety of manners, including formation of hydrogen bonds, ionic bonds, covalent bonds, or van der Wals forces. In some cases, two or more nucleic acid molecules can be linked to form a continuous nucleic acid molecule through phosphodiester bonds. In some cases, two or more nucleic acid molecules can be operatively linked to form a continuous nucleic acid molecule. In some cases, two or more nucleic acid molecules can be operatively linked to form a continuous nucleic acid molecule such that the resulting molecule can be expressed in a cell.

Percent (%) sequence identity with respect to a reference nucleic acid sequence (or peptide sequence) is the percentage of nucleotides (or amino acid residues in case of peptide sequence) in a candidate sequence that are identical with the nucleotides (or amino acid residues) in the reference nucleic acid sequence (or peptide sequence), after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTALW, ALIGN or Megalign (DNASTAR) software. Those skilled in the art can determine appropriate parameters for aligning sequences, including any algorithms needed to achieve maximal alignment over the full length of the sequences being compared.

The term “endogenous,” as used herein, refers to refers to a substance that is native to an organism, cell, tissue or system.

The term “exogenous,” as used herein, refers to a substance present in an organism, cell, tissue or system other than its own native source. The substance can be introduced from other sources into the organism, cell, tissue or system.

The term “expression,” as used herein, is defined as the transcription and/or translation of a particular nucleic acid sequence driven by its promoter.

The term “exogenously expressing” or “exogenously expressed” refers to an expression of a polypeptide from an exogenous polynucleotide sequence (e.g., a polynucleotide sequence not derived or originated from the host cell) introduced to the host cell. An exogenous protein can be a protein expressed by an exogenous polynucleotide sequence that is not derived or originated from the host cell.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include, but are not limited to, cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “cognate pair,” as used herein, refers to an original or native pair of two nucleic acid molecules or proteins encoded by the two nucleic acid molecules that are contained within or derived from an individual cell. The cognate pair can be natively paired chains within the individual cell. For example, a cognate pair of T-cell receptor (TCR) can be a natively paired TCR alpha and beta chains within or derived from an individual cell. For another example, a cognate pair of T-cell receptor (TCR) can be a natively paired TCR gamma and delta chains within or derived from an individual cell.

The term “knockdown,” as used herein, refers to a decrease in gene expression of one or more genes.

The term “knockout,” as used herein, refers to the ablation of gene expression of one or more genes.

The term “modified,” as used herein, refers to a changed state or structure of a molecule or cell. The term “modified” can be used interchangeably with “engineered,” “synthetic” or “artificial.” Molecules may be modified in many ways, including chemically, structurally, and functionally. Cells may be modified through the introduction of exogenous nucleic acids or polypeptides. In some cases, the modified cell described herein expresses an exogenous T-cell receptor (TCR). The modified cell that exogenously expresses an TCR, which TCR is endogenous to a tumor-infiltrating lymphocyte (TIL) isolated from a tumor sample from a subject, can be referred to as “synthetic TIL” in the present disclosure.

The term “polyclonal T-cell receptors (TCRs)” describes a population of different TCR molecules which is capable of binding to or reacting with different specific antigenic determinants from the same or from different antigens. The term “polyclonal TCR-T cells” refers to a population of cells expressing polyclonal TCRs.

The term “productively express,” as used herein, means that a sequence can be translated into a protein, and if the protein is a TCR chain, it can form a cognate pair with the other TCR chain. The protein can be a functional protein. The protein can be expressed on a cell surface. In some cases, the productively expressed TCR can form a TCR complex with CD3 subunits (e.g., CD3 epsilon, CD3 gamma, CD3 delta, and CD3 zeta).

The term “line of therapy,” as used herein, refers to a treatment that consists of one or more complete treatment cycles with a single agent, surgery, or ration therapy, a regimen consisting of a combination of several drugs, surgery, or radiation therapy, or a planned sequential therapy of various regimens. A treatment is considered a new line of therapy if any one of the following two conditions is met:
(i) Start of a new line of treatment after discontinuation of a previous line of treatment: If a treatment regimen is discontinued for any reason and a different regimen is started, it should be considered a new line of therapy. A regimen is considered to have been discontinued if all the drugs, radiation therapy or surgery in that given regimen have been stopped. A regimen is not considered to have been discontinued if some of the drugs, radiation therapy, or surgery of the regimen, but not all, have been discontinued.
(ii) The unplanned addition or substitution of one or more drugs, radiation therapy, or surgery in an existing regimen: Unplanned addition of a new drug, a new radiation therapy, or a new surgery or unplanned switching to a different drug (or combination of drugs), a different radiation therapy, or a different surgery for any reason is considered a new line of therapy.

In the present disclosure, the polynucleotide sequence may be described at domain level. Each domain name can correspond to a specific polynucleotide sequence. For example, domain ‘A’ may have a sequence of 5′-TATTCCC-3′, domain ‘B’ may have a sequence of 5′-AGGGAC-3′, and domain ‘C’ may have a sequence of 5′-GGGAAGA-3′. In this case the polynucleotide having a sequence that is the concatenation of domains A, B, and C, can be written as [A|B|C}. The symbol ‘[’ denotes the 5′ end, the symbol ‘}’ denotes the 3′ end, and the symbol ‘I’ separates domain names. An ssDNA or a section of ssDNA having sequence ‘X’ can be referred to as [X}. An asterisk sign shows sequence complementarity. For example domain [X*} is the reverse complement of domain [X}. The notation ds[X} can be used to describe a double-stranded DNA formed by [X} and [X*}. In some cases, especially in situation where it is not necessary to distinguish dsDNA and ssDNA, a dsDNA whose one strand has the sequence [X} may also be loosely referred to as [X}. A single-stranded RNA molecule or segment with the sequence identical to [X} (except replacing T with U) may also be referred to as [X}. Depending on the context, the domain name may refer to an exact sequence or describe a general function of a DNA or domain. For example, [RBS} may be used to describe a ribosome binding site, although the exact sequence for [RBS} may vary. Parentheses can be used to group a concatenation of domains, and the reverse-complement operation (denoted by ‘*’) can be applied to the concatenation by adding the ‘*’ following the closing parenthesis. For example, [(X|Y)*} is the same as [Y*|X*}. A double-stranded DNA formed by two strands [X} and [X*} can be written as [X}:[X*}. A double-stranded segment of a double-stranded DNA can be written in similar manner. For example, a dsDNA formed by [X|Y} and [Y*|X*} can be said to have double-stranded segments [X}:[X*} and [Y}:[Y*}. A double-stranded segment [X}:[X*} can also be called “double-stranded DNA [X}” or “dsDNA [X}” without creating ambiguity.

### Overview

Adoptive cell therapy (ACT) is a promising approach to treat cancers (e.g., solid tumors). In particular, tumor-infiltration lymphocyte (TIL) therapy, where tumor-infiltrating T cells (or TITs) can be harvested from a surgical resection, expanded ex vivo, and infused back to the patient, is on the verge of FDA approval (as of January 2022) for the treatment of melanoma. However, the efficacy of conventional TIL therapy in moderately immunogenic tumor (e.g., most epithelial tumors) is still inconsistent. A key limitation of TIL therapy can be that, within the TIT population, only a small fraction may be tumor-reactive, and these T cells can be in the terminally differentiated and/or dysfunctional state, while the vast majority of the T cells are tumor-nonreactive cells with a proliferative phenotype (e.g., naïve, central memory or stem-cell memory). Thus, when these T cells are expanded ex vivo, the tumor-nonreactive, more proliferative T cells can grow much faster and dominate the cell population for infusion. Moreover, initiation of the ex vivo TIL expansion may need a large amount of TIL cells which in turn can lead to the need of a large quantity of tumor material often only achievable from a surgical sample. However, since surgery can be highly invasive and may not result in better patient outcome, it may not be recommended for late-stage patients.

To solve these problems, the present disclosure provides compositions and methods to produce a synthetic version of TIL (or TIT) cells where the fraction of tumor-reactive T cells can be enriched, and only a small tumor sample (e.g., available from biopsy) can be sufficient. The novel aspects of the methods provided herein can include: (1) novel minimally invasive sampling method to obtain both the T cell receptor (TCR) sequence and transcriptome information of each TIL cells, (2) novel algorithm to select TCRs that are likely to be tumor-reactive, (3) a novel method to produce hundreds of or thousands of or more therapeutic-grade TCR genes at low cost and fast turn-around time, and (4) a novel method to introduce a mixture of hundreds of, thousands of or more TCR genes to primary T cells where the majority of T cells only productively express one copy of exogenous TCR. In various embodiments, the methods described herein comprises delivering a mixture of sequences encoding different exogenous TCRs into a population of cells. In such cases, if the cell acquires more than one copy of sequence, it may result in unwanted consequences such as mispairing of the TCR chains, losing intended functions of the TCR, and showing unintended functions (e.g., activity against self-antigens). The compositions and methods provided herein can ensure each cell only receives a single copy of the sequence encoding an exogenous TCR when contacting a mixture of sequences encoding the TCRs and the cells.

### T-Cell Receptors (TCRs)

The present disclosure provides compositions and methods for producing modified cells (e.g., modified T cells) comprising exogenous TCRs. The exogenous TCRs can be from a subject in need thereof that will be administered with the compositions described herein. In some cases, these exogenous TCRs can be referred to as autologous TCRs.

The TCR can be used to confer the ability of T cells to recognize antigens associated with various cancers or infectious organisms. The TCR is made up of two chains, e.g., an alpha (a) chain and a beta (B) chain or a gamma (Y) and a delta (8) chain. The proteins which make up these chains are encoded by DNA, which employs a unique mechanism for generating the tremendous diversity of the TCR. This multi-subunit immune recognition receptor associates with the CD3 complex and binds peptides presented by the MHC class I and II proteins on the surface of antigen-presenting cells (APCs). Binding of a TCR to the antigenic peptide on the APC can be a central event in T-cell activation, which occurs at an immunological synapse at the point of contact between the T cell and the APC.

The TCR may recognize the T cell epitope in the context of an MHC class I molecule. MHC class I proteins can be expressed in all nucleated cells of higher vertebrates. The MHC class I molecule is a heterodimer composed of a 46-kDa heavy chain which is non-covalently associated with the 12-kDa light chain β-2 microglobulin. In humans, there are several MHC alleles, such as, for example, HLA-A2, HLA-A1, HLA-A3, HLA-A24, HLA-A28, HLA-A31, HLA-A33, HLA-A34, HLA-B7, HLA-B45 and HLA-Cw8. In some embodiments, the MHC class I allele is an HLA-A2 allele, which in some populations is expressed by approximately 50% of the population. In some embodiments, the HLA-A2 allele can be an HLA-A*0201, *0202, *0203, *0206, or *0207 gene product. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203.

In some embodiments, the TCR may recognize the T cell epitope in the context of an MHC class II molecule. MHC class II proteins can be expressed in a subset of APCs. In humans, there are several MHC class II alleles, such as, for example, DR1, DR3, DR4, DR7, DR52, DQ1, DQ2, DQ4, DQ8 and DPI. In some embodiments, the MHC class II allele is an HLA-DRB1*0101, an HLA-DRB*0301, an HLA-DRB*0701, an HLA-DRB*0401 or an HLA-DQB1*0201 gene product.

The TCR chain can comprise a variable domain (or variable region) and a constant domain (or constant region). The variable domain can be a V-DOMAIN as defined by IMGT unique numbering system. The variable domain can correspond to V-J-REGION or V-D-J-REGION of a TCR chain. The constant domain can be C-DOMAIN as defined by IMGT unique numbering system. In some cases, the constant domain can be a portion of the constant region. For example, a full-length constant region can comprise the constant domain (an extracellular region), a connecting region, a transmembrane region, and a cytoplasmic region.

The variable domain of TCRα or TCRδ chain can be encoded by a number of variable (V) and joining (J) gene segments in the germline, while variable domain of TCRβ or TCRγ chain is additionally encoded by diversity (D) gene segments. Each gene segment can be flanked by recombination signal sequences. The recombination signals can comprise a heptamer and a nonamer, separated by a spacer element. The spacer element can be 12 or 23 bp long. During V(D)J recombination, one random allele of each gene segment is recombined with the others to form a functional variable domain. Recombination of the variable domain with a constant (C) gene segment can result in a functional TCR chain transcript. Additionally, random nucleotides may be added and/or deleted at the junction sites between the gene segments. This process can lead to strong combinatorial (depending on which gene regions will recombine) and junctional diversity (depending on which and how many nucleotides will be added/deleted), resulting in a large and highly variable TCR repertoire, which can ensure the identification of a plethora of antigens. Additional diversity can be achieved by the pairing (also referred to as “assembly”) of α and β or γ and β chains to form a functional TCR. By recombination, random insertion, deletion and substitution, the small set of genes that encode the T cell receptor has the potential to create between 1015 and 1020 TCR clonotypes. As used herein, a “clonotype” refers to a population of immune cells that carry an identical immunoreceptor. For example, a clonotype refers to a population of T cells that carry an identical TCR, or a population of B-cells that carry an identical BCR (or antibody). “Diversity” in the context of immunoreceptor diversity refers to the number of immunoreceptor (e.g., TCR, BCR and antibody) clonotypes in a population. As used herein, a “cognate pair combination” refers to the native combination of the two chains (e.g., TCRα and TCRβ, or TCRγ and TCRδ) of a TCR from a T cell. The same cognate pair combination of the two chains can result in the same TCR. For example, the T cells having the same clonotype have the same cognate pair combinations of TCRα and TCRβ chains. The higher diversity in clonotype may indicate higher diversity in cognate pair combination.

Each TCR chain can contain three hypervariable loops in its structure, termed complementarity determining regions (CDR1-3). CDR1 and CDR2 can be encoded by V genes and may be required for interaction of the TCR with the MHC complex. CDR3, however, is encoded in part by the (1) junctional region between the V and J genes (in the case of TCRα or TCRγ), or (2) the junctional region between the V and D genes and the junctional region between the D and J genes (in the case of TCRβ or TCRδ), and therefore can be highly variable. CDR3 may be the region of the TCR in direct contact with the peptide antigen. CDR3 can be used as the region of interest to determine T cell clonotypes. The sum of all TCRs by the T cells of one individual is termed the TCR repertoire or TCR profile. The TCR repertoire can change with the onset and progression of diseases. Therefore, determining the immune repertoire status under different disease conditions, such as cancer, autoimmune, inflammatory and infectious diseases may be useful for disease diagnosis and prognosis.

TCR should be understood to encompass full-length TCRs as well as antigen-binding portions or antigen-binding fragments (also called MHC-peptide binding fragments) thereof. In some embodiments, the TCR is an intact or full-length TCR. In some embodiments, the TCR is an antigen-binding portion that is less than a full-length TCR but that binds to a specific antigenic peptide bound to an MHC molecule, e.g., an MHC-peptide complex. In some cases, an antigen-binding portion or fragment of a TCR can contain only a portion of the structural domains of a full-length or intact TCR, but yet is able to bind the epitope (e.g., MHC-peptide complex) to which the full TCR binds. In some cases, an antigen-binding portion or fragment of a TCR contains the variable domains of a TCR, such as variable α chain and variable β chain of a TCR, sufficient to form a binding site for binding to a specific MHC-peptide complex, such as generally where each chain contains three complementarity determining regions. Polypeptides or proteins having a binding domain which is an antigen-binding domain or is homologous to an antigen-binding domain are included.

A TCR molecule can be formed by an alpha chain (a chain or TCRα chain, encoded by TRA gene/sequence) and a beta chain (β chain or TCRβ chain, encoded by TRB gene/sequence), or a gamma chain (γ chain or TCRγ chain, encoded by TRG gene/sequence) and a delta chain (δ chain or TCRδ chain, encoded by TRD gene/sequence). These immunoreceptor chains can have variable domains (e.g., encoded by the rearranged VDJ or VJ regions). Parts of the variable domains can be hypervariable. The hypervariable regions can include complementarity determining regions (CDRs), for example, CDR1, CDR2 and CDR3. In some cases, within one T cell, only one functional α chain sequence and one functional β chain sequence may be expressed. In some cases, within one T cell, only one functional γ chain sequence and one functional δ chain sequence may be expressed.

### Methods for Producing Modified Cells Expressing Exogenous TCRs

The present disclosure provides a method of producing a plurality of modified cells called synthetic tumor-infiltrating lymphocytes (TILs). In various cases, the method described herein can produce a mixture containing the plurality of the synthetic TILs. The method can comprise obtaining a sample (e.g., a resection or biopsy sample) comprising a plurality of T cells from a subject. The sample can be obtained from a solid tumor lesion of the subject. The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline. In some cases, the subject is a cancer patient. In some cases, the plurality of T cells of the sample can be sequenced to obtain natively paired T-cell receptor (TCR) information. For example, the plurality of T cells can be sequenced to obtain native pairs of TCR alpha chain and TCR beta chain sequences of TCRs of the plurality of T cells. The sequencing can be done by various sequencing methods, including but not limited to, Sanger sequencing, high-throughput sequencing, sequencing-by-synthesis, single-molecule sequencing, single cell sequencing, sequencing-by-ligation, RNA-Seq, Next generation sequencing (NGS), Digital Gene Expression, Clonal Single MicroArray, shotgun sequencing, Maxim-Gilbert sequencing, and massively-parallel sequencing. In some cases, the sequencing comprises single-cell sequencing.

After obtaining the natively paired TCR information, the sequences encoding different cognate pairs of TCRs can be synthesized. In some cases, a plurality of polynucleotide sequences encoding the TCRs or a subset thereof from the natively paired TCR alpha chain and beta chain sequences obtained in sequencing can be synthesized. In some cases, all TCRs obtained in sequencing can be synthesized. In some cases, a subset of the TCRs obtained in sequencing can be synthesized. The plurality of polynucleotide sequences encoding the TCRs or a subset thereof can comprise a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence. The plurality of polynucleotide sequences encoding the TCRs or a subset thereof can comprise at least 20, 30, 40, 50, 60, 70, 80, 90, 100, 200, 300, 400, 500, 600, 700, 800, 900, 1,000, 1200, 1,500, 1,800, 2,000, 5,000, 10,000, 20,000, 50,000, 100,000 or more different sequences, each encoding a different cognate pair of TCR. The plurality of polynucleotide sequences encoding the TCRs or a subset thereof can encode at least 20, 30, 40, 50, 60, 70, 80, 90, 100, 200, 300, 400, 500, 600, 700, 800, 900, 1,000, 1200, 1,500, 1,800, 2,000, 5,000, 10,000, 20,000, 50,000, 100,000 or more different cognate pairs of TCRs.

The plurality of polynucleotide sequences or derivative thereof (e.g., a copy thereof) can be delivered into a plurality of recipient cells to produce a plurality of synthetic TILs. In some cases, the delivering is conducted by contacting a mixture (e.g., a pool) comprising the plurality of polynucleotide sequences or derivative thereof with the plurality of recipient cells. In some cases, each polynucleotide of the plurality of polynucleotide sequences or derivative thereof is not delivered into a recipient cell in an individual reaction. For example, a sequence encoding a TCR may not be separated from other different sequences encoding different TCRs prior to delivering into a recipient cell. The delivering can be done using various gene delivery methods described herein. In some cases, a recipient cell only acquires and/or expresses a single copy of the sequence encoding a TCR. For example, in some cases, after delivering, a first fraction of the plurality of synthetic TILs exogenously expresses the first polynucleotide sequence encoding the first natively paired TCR and a second fraction of the plurality of synthetic TILs exogenously expresses the second polynucleotide sequence encoding the second natively paired TCR, and wherein a third fraction of the plurality of synthetic TILs exogenously expressing both the first and the second polynucleotide sequence is less than the product of the first fraction and the second fraction. For example, in the plurality of synthetic TILs having a total number of at least about 1×10⁴, the number of cells exogenously expressing the first polynucleotide sequence encoding the first natively paired TCR (regardless if expressing the second polynucleotide) can be at least about 1×10³, the number of cell exogenously expressing the second polynucleotide sequence encoding the second natively paired TCR (regardless if expressing the first polynucleotide) can be at least about 1×10³, and the number of cells expressing both can be less than about 1×10². In some cases, a cell of the plurality of synthetic TILs (i) exogenously expresses only one polynucleotide sequence of the plurality of polynucleotide sequences or (ii) does not exogenously express any of the plurality of polynucleotide sequences or derivative thereof. In some cases, none of the plurality of synthetic TILs exogenously expresses both the first and the second polynucleotide sequence. In some cases, a cell of the plurality of synthetic TILs that exogenously expresses the first polynucleotide sequence does not exogenously express the second polynucleotide sequence.

In some cases, expression level of one or more genes in the plurality of T cells may also be obtained by sequencing. The one or more genes can be used to determine whether a cell is a potential tumor-reactive T cell and carries tumor-reactive TCR. The one or more genes can comprise at least about 3, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more genes. The one or more genes can comprise a gene selected from the group consisting of CXCL13, CTLA4, DUSP4, ENTPD1, RBPJ, TNFSF4, GK, PDCD1, LAG3, FKBPIA, MYO7A, FABP5, GAPDH, TIGIT, CDCA7, ZBED2, RGS1, HAVCR2, MKI67, GZMB, SPRY1, PHLDA1, ASPM, MYO1E, TOX, LSP1, MCM10, MAD2L1, OGN, PCLAF, UBE2T, GNG4, LY6E, TPI1, TNFRSF18, RGS2, CLEC2D, SAMSN1, TSHZ2, HMGN2, and any combination thereof. In some cases, the plurality of T cells of the sample from the subject can be sequenced to obtain (i) expression level of one or more genes (e.g., at least 10 genes) and (ii) natively paired TCR alpha chain and beta chain sequences of TCRs of the plurality of T cells. In some cases, prior to synthesizing the plurality of polynucleotide sequences encoding the cognate pairs of TCRs, a score can be calculated using the expression level of the one or more genes obtained from the plurality of T cells from a subject. For example, the score can be calculated using the expression level of at least 3 genes obtained. The score can be calculated for each individual cell of the plurality of T cells. Next, the plurality of T cells can be ranked based on the score. At least 2, 3, 4, 5, 6, 7, 8, 9, 10, 11, 12, 13, 14, 15, 16, 17, 18, 19, 20, 30, 40, 50, 60, 70, 80, 90, 100, 120, 150, 200, 300, 400, 500 or more TCRs can be selected from the top 50% of the plurality of T cells according to the score. In some cases, at least 2, 3, 4, 5, 6, 7, 8, 9, 10, 11, 12, 13, 14, 15, 16, 17, 18, 19, 20, 30, 40, 50, 60, 70, 80, 90, 100, 120, 150, 200, 300, 400, 500 or more TCRs can be selected from the top 20%, 30%, 40%, 50%, 60%, 70%, 80%, or 90% of the plurality of T cells according to the score. The selected TCRs can then be synthesized and delivered into the recipient cells as a mixture or a pool. The selected TCRs can be functional TCRs. The selected TCRs can be tumor-reactive TCRs. The selected TCR can exhibit better tumor reactivity or cytotoxicity than those unselected TCRs. The selected TCRs can be synthesized and pooled in a mixture for delivering into recipient cells. The plurality of polynucleotide sequences encoding the TCRs or a subset thereof synthesized can comprise polynucleotide sequences encoding the at least 2, 3, 4, 5, 6, 7, 8, 9, 10, 11, 12, 13, 14, 15, 16, 17, 18, 19, 20, 30, 40, 50, 60, 70, 80, 90, 100, 120, 150, 200, 300, 400, 500 or more TCRs selected.

In some cases, a first fraction of the plurality of synthetic TILs exogenously expresses the first polynucleotide sequence encoding the first natively paired TCR and a second fraction of the plurality of synthetic TILs exogenously expresses the second polynucleotide sequence encoding the second natively paired TCR, and wherein a third fraction of the plurality of synthetic TILs exogenously expressing both the first and the second polynucleotide sequence is less than the product of the first fraction and the second fraction. The first fraction, the second fraction, or the third fraction can be determined by various methods. For example, the fraction of cells can be determined by sequencing such as single-cell sequencing. The single cell sequencing may comprise barcoding each cell in an individual compartment. In some cases, the CDR3 beta of the exogenous TCRs can be amplified and subject to sequencing. The read number corresponding to a first TCR divided by the total read number can be a measurement of the first fraction. The read number corresponding to a second TCR divided by the total read number can be a measurement of the second fraction. The read numbers can be unique molecular identifier (UMI)-corrected. For another example, the fraction of cells can be determined by pMHC multimer staining. The pMHC multimer staining can comprises using a plurality of pMHC multimers, each specific for binding to a TCR encoded by a polynucleotide sequence.

The recipient cell can acquire and/or express a single copy of the sequence encoding an exogenous TCR (e.g., the cognate TCR identified by sequencing from the sample obtained from the subject). In some cases, the recipient cell may acquire two or more sequences, each encoding a different TCR, but only one sequence is expressed. For example, only one sequence can be knocked into a genomic locus for expression. The expression can be controlled through the endogenous promoter. Other sequences that are not knocked into the genomic locus may not be expressed. Other sequences may be degraded inside the cell. For example, the vector containing the sequences may not comprise a promoter. In some cases, the delivering the plurality of polynucleotide sequences or derivative thereof into the plurality of recipient cells can comprise site-specifically knocking in a polynucleotide sequence into a genomic locus of a recipient cell of the plurality of recipient cells. The genomic locus of the recipient cell can lead to monoallelic expression of the polynucleotide sequence. The monoallelic expression can comprise the expression of a gene from a single allele against the background of a diploid heterozygous genome. The monoallelic expression can comprise the expression of a gene from a single locus. In some cases, the monoallelic expression of the cognate TCR pair having a TCR alpha chain and a TCR beta chain is controlled by a single promoter at the knock-in locus. The sequences encoding the two chains of a cognate TCR can be expressed contiguously in frame from a single promoter. The sequences encoding the two chains of a cognate TCR can be linked by a sequence encoding a self-cleaving peptide. In some cases, the monoallelic expression of the cognate TCR pair having a TCR alpha chain and a TCR beta chain may be controlled by two promoters at the knock-in locus. For example, the two promoters can comprise a first promoter for the TCR alpha chain, and a second promoter for the TCR beta chain. The polynucleotide sequence can be site-specifically knocked in in-frame into the genomic locus of the recipient cell of the plurality of recipient cells. The genomic locus can be located on a sex chromosome. The genomic locus can be a T-cell receptor alpha constant (TRAC) locus.

The site-specific knock-in of the polynucleotide sequence into the genomic locus can be done using a gene editing method described herein. In some cases, the gene editing method can comprise clustered regularly interspaced short palindromic repeats (CRISPR), transcription activator-like effector (TALE) nucleases (TALENs), meganucleases, zinc finger nuclease (ZFN), megaTAL nucleases method, or any combination thereof. In some cases, the site-specific knock-in of the polynucleotide sequence into the genomic locus is done by using CRISPR method. The CRISPR method can comprise delivering a ribonucleoprotein (RNP) complex comprising a Cas9 protein and a single guide RNA (sgRNA) into the recipient cell. The sgRNA can target a locus on a sex chromosome for monoallelic expression. In some cases, the sgRNA can target the TRAC locus. The site-specific knock-in of the polynucleotide sequence into the genomic locus can comprise cutting the genomic locus using the gene editing method, and inserting the polynucleotide sequence into the genomic locus via homologous recombination.

In various cases, an endogenous TCR can be inactivated in the recipient cell. The endogenous TCR can be knocked down (e.g., by RNA interference) or knocked out (e.g., by a gene editing method described herein) in the recipient cell. In some cases, the endogenous T-cell receptor beta constant (TRBC) locus is inactivated in the recipient cell. The endogenous TRBC locus can be knocked down or knocked out in the recipient cell.

In some cases, an endogenous TCR is inactivated in at least about 40%, 50%, 60%, 70%, 80%, 90%, 99% or 100% of the plurality of recipient cells.

The endogenous TCR can be knocked down or knocked out in at least about 40%, 50%, 60%, 70%, 80%, 90%, 99% or 100% of the plurality of recipient cells. The endogenous TCR can be inactivated using a gene editing method described herein. For example, the endogenous TCR can be selected from the group consisting of clustered regularly interspaced short palindromic repeats (CRISPR), transcription activator-like effector (TALE) nucleases (TALENs), meganucleases, zinc finger nuclease (ZFN), megaTAL nucleases method, and any combination thereof.

The endogenous TCR can be inactivated prior to, concurrently with, or after delivering the plurality of polynucleotide sequences or derivative thereof into the plurality of recipient cells in (d). In some cases, the endogenous TCR can be inactivated concurrently with delivering the plurality of polynucleotide sequences or derivative thereof into the plurality of recipient cells in (d). For example, the vector containing the sequence encoding the exogenous TCR and the agents (e.g., nuclease or RNA) for gene editing can be delivered concurrently into the recipient cell. The endogenous TCR can be inactivated using CRISPR method. The CRISPR method can comprise delivering a ribonucleoprotein (RNP) complex comprising a Cas9 protein and a single guide RNA (sgRNA) into the recipient cell. The sgRNA can comprise a sgRNA targeting the TRBC locus. The endogenous TCR can be inactivated concurrently with the site-specific knock-in of the polynucleotide sequence into the genomic locus by delivering the RNP comprising the Cas9 protein and sgRNAs that comprises a sgRNA targeting the TRBC locus and a sgRNA targeting the TRAC locus.

Each cell of the plurality of synthetic TILs can exogenously express only a single type of TCR. Each cell of the plurality of synthetic TILs can exogenously express a single copy of the polynucleotide sequence encoding a TCR. In some cases, at least about 40%, 50%, 60%, 70%, 80%, 90%, 99% or 100% of the plurality of synthetic TILs exogenously express only a single copy of polynucleotide sequence encoding a natively paired TCR of the TCRs of the plurality of T cells. In other words, each cell of the at least about 40%, 50%, 60%, 70%, 80%, 90%, 99% or 100% of the plurality of synthetic TILs exogenously expresses only a single copy of polynucleotide sequence encoding a natively paired TCR.

The synthetic TILs provided herein can be expanded prior to being administered into a subject from who the cognate pairs of TCRs are obtained. The “activation and expansion” methods described in the present disclosure can be applicable to expand the synthetic TILs.

A unique cognate pair of TCR (e.g., a clonotype) can have an initial frequency in the mixture comprising a plurality of polynucleotide sequences encoding a plurality of different cognate pairs of TCRs (e.g., the TCRs selected from the sequencing data) before delivering into the recipient cells for expression. During the process of producing the synthetic TILs, the frequency of the unique TCR can be maintained within 0.1-fold to 10-fold (e.g., within 0.2-fold to 5-fold, or within 0.5-fold to 2-fold) of the initial frequency in the mixture. In some cases, the frequency of the unique TCR can be maintained within 10-fold, 9-fold, 8-fold, 7-fold, 6-fold, 5-fold, 4-fold, 3-fold, 2-fold or less of the initial frequency. In some cases, after delivering the sequences into recipient cells for expression and expansion of the synthetic TILs, the unique cognate pair of TCR can have a frequency that is close to or within 10-fold, 9-fold, 8-fold, 7-fold, 6-fold, 5-fold, 4-fold, 3-fold, 2-fold or less of the initial frequency. In other words, the methods described herein or the synthetic TILs provided herein can be resistant to growth bias. During the manufacturing process described herein, cells expressing one or more unique TCRs may not outgrow or competitively grow better than other unique TCRs in a mixture.

The frequency described herein refers to the relative abundance, a proportion, or copy number of a unique TCR in the mixture of TCRs. For example, if a mixture of 10 TCRs are selected for producing the synthetic TILs, and each sequence encoding a unique TCR is mixed at equimolar proportion with other sequences, the frequency of this unique TCR is 1/10 or 10% in the mixture (see Example 9). After expansion of the synthetic TILs, the frequency can be determined by various methods including sequencing. Using sequencing as an example, the frequency can be calculated by sequencing reads of a particular TCR divided by total sequencing reads of the TCR repertoire. In some cases, the frequency can be determined by unique molecule index (UMI) count of a particular TCR divided by total UMI count of the TCR repertoire. The UMI can be added to the nucleic acid molecules to be sequenced when preparing the sequencing library using standard protocols.

In various embodiments, the synthetic TIL prepared herein can be administered into a subject. The subject can be the same subject from whom the plurality of source T cells are obtained. The subject can be a cancer patient. The subject can have a solid tumor lesion. The subject can have an advanced or metastatic solid tumor. Examples of the cancer include, but not limited to, a melanoma, renal cell carcinoma, lung cancer, bladder cancer, breast cancer, cervical cancer, colorectal cancer, head and neck cancer, colon cancer, gall bladder cancer, laryngeal cancer, liver cancer (e.g., hepatocellular carcinoma), thyroid cancer, stomach cancer, salivary gland cancer, prostate cancer, pancreatic cancer, urothelial cancer, or Merkel cell carcinoma. In some cases, the cancer can be adrenocortical carcinoma, adrenal cortex cancer, AIDS-related cancers, anal cancer, appendix cancer, astrocytoma, atypical teratoid/rhabdoid tumor, basal cell carcinoma, bile duct cancer, extrahepatic, bladder cancer, bone cancer (includes Ewing sarcoma and osteosarcoma and malignant fibrous histiocytoma), brain tumors, breast cancer, carcinoid tumor (gastrointestinal), carcinoma of unknown primary, cervical cancer, cholangiocarcinoma, chronic myeloproliferative neoplasms, colorectal cancer, cutaneous t-cell lymphoma, ductal carcinoma in situ (dcis), endometrial cancer, esophageal, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastrointestinal carcinoid tumor, gastrointestinal stromal tumors (GIST), germ cell tumors, extragonadal, ovarian, testicular, gestational trophoblastic disease, gliomas, hairy cell leukemia, head and neck cancer, hepatocellular (liver) cancer, Langerhans cell histiocytosis, Hodgkin lymphoma, hypopharyngeal cancer, intraocular melanoma, islet cell tumors, pancreatic neuroendocrine tumors, laryngeal cancer, lip and oral cavity cancer, liver cancer (primary), lung cancer, lymphoma, macroglobulinemia, male breast cancer, malignant fibrous histiocytoma of bone and osteosarcoma, melanoma, intraocular (eye), Merkel cell carcinoma, mesothelioma, malignant, metastatic squamous neck cancer with occult primary, mouth cancer, multiple myeloma/plasma cell neoplasms, mycosis fungoides, myelodysplastic syndromes, nasal cavity and paranasal sinus cancer, nasopharyngeal cancer, neuroblastoma, non-Hodgkin lymphoma, non-small cell lung cancer, oral cancer, oropharyngeal cancer, osteosarcoma and malignant fibrous histiocytoma of bone, ovarian cancer, pancreatic, paraganglioma, paranasal sinus and nasal cavity cancer, parathyroid cancer, penile cancer, pharyngeal cancer, pheochromocytoma, pituitary tumor, plasma cell neoplasm/multiple myeloma, pregnancy and breast cancer, primary central nervous system (CNS) lymphoma, primary peritoneal cancer, prostate cancer, rectal cancer, renal cell (kidney) cancer, retinoblastoma, salivary gland cancer, sarcoma, Ewing sarcoma, Kaposi sarcoma, osteosarcoma, rhabdomyosarcoma, uterine sarcoma, skin cancer, small cell lung cancer, small intestine cancer, soft tissue sarcoma, squamous cell carcinoma, squamous neck cancer with occult primary, metastatic, stomach (gastric) cancer, testicular cancer, throat cancer, thymoma and thymic carcinoma, thyroid cancer, transitional cell cancer of the renal pelvis and ureter, ureter and renal pelvis, transitional cell cancer, urethral cancer, uterine cancer, endometrial and uterine sarcoma, vaginal cancer, vulvar cancer, Waldenströn macroglobulinemia, or Wilms' tumor. In some cases, the method can further comprise administering at least one cell of the plurality of synthetic TILs into the subject having the solid tumor lesion.

In some cases, prior to administering, the plurality of synthetic TILs have not been subjected to any selection or screening for determining tumor reactivity of the plurality of synthetic TILs or subset thereof. For example, the plurality of synthetic TILs, each exogenously expressing a different TCR, may not have been contacted with a tumor antigen, and any subset thereof that can bind to the tumor antigen may not have been selected for administration. In some cases, the plurality of synthetic TILs can comprise both tumor-reactive cells and tumor non-reactive cells.

In some cases, a tumor reactivity of the plurality of synthetic TILs may be determined prior to administration. The tumor reactivity of the plurality of synthetic TILs may be determined after expansion and prior to administration. The plurality of synthetic TILs can be in a mixture and the tumor reactivity of the plurality of synthetic TILs can be determined together. In such cases, the plurality of synthetic TILs can comprise both tumor-reactive cells and tumor non-reactive cells. In some cases, determining the tumor reactivity can comprise: (i) providing a tumor cell or derivative thereof (e.g., an offspring or cell constituents of the tumor cell) from the subject; (ii) contacting the plurality of synthetic TILs with the tumor cell or derivative thereof; (iii) determining the tumor reactivity of the plurality of synthetic TILs against the tumor cell or derivative thereof; and (iv) determining whether to administer the plurality of synthetic TILs or subset thereof into the subject. Determining tumor reactivity can be done by various methods. For example, a mixture of the plurality of synthetic TILs can be co-cultured with tumor cell or derivative thereof and tested for cytokine release using methods such as ELISA and ELISPOT. In some cases, after determining the tumor reactivity of the mixture, at least one cell of the plurality of synthetic TILs or the mixture can be administered into the subject having the solid tumor lesion.

The methods described herein can offer fast turnaround time from sample isolation to the preparation of a pharmaceutical composition for administration. In some cases, a time interval between obtaining the sample (e.g., a resection or biopsy sample) and administering the at least one cell of the plurality of synthetic TILs into the subject can be less than 2, 3, 4, 5, 6, 7, 8, or more weeks. In some cases, a time interval between obtaining the sample (e.g., resection or biopsy sample) and administering the at least one cell of the plurality of synthetic TILs into the subject can be no more than 10, 9, 8, 7, 6, 5, 4, 3 or less weeks. In some cases, a time interval between obtaining the sample and administering the at least one cell of the plurality of synthetic TILs into the subject can be less than 8 weeks. In some cases, a time interval between obtaining the sample and administering the at least one cell of the plurality of synthetic TILs into the subject can be less than 4 weeks. In some cases, a time interval between obtaining the sample and administering the plurality of synthetic TILs comprising at least about 10, 20, 30, 40, 50, 60, 70, 80, 90, 100 or more different TCRs into the subject can be less than 2, 3, 4, 5, 6, 7, 8, or more weeks. In some cases, a time interval between obtaining the sample and administering the plurality of synthetic TILs comprising at least about 10, 20, 30, 40, 50, 60, 70, 80, 90, 100 or more different TCRs into the subject can be less than 8 weeks. In some cases, a time interval between obtaining the sample and administering the plurality of synthetic TILs comprising at least about 10, 20, 30, 40, 50, 60, 70, 80, 90, 100 or more different TCRs into the subject can be less than 4 weeks.

In some cases, a time interval between obtaining the sample and the delivering the synthetic sequences encoding the TCRs into the recipient cells can be no more than 8, 7, 6, 5, 4, 3 or less weeks. In some cases, a time interval between obtaining the sample and the delivering the synthetic sequences encoding the TCRs into the recipient cells can be no more than 7 weeks. In some cases, a time interval between obtaining the sample and the delivering the synthetic sequences encoding the TCRs into the recipient cells can be no more than 3 weeks. In some cases, a time interval between obtaining the sample and the delivering the synthetic sequences encoding at least about 10, 20, 30, 40, 50, 60, 70, 80, 90, 100 or more different TCRs into the recipient cells can be no more than 8, 7, 6, 5, 4, 3 or less weeks. In some cases, a time interval between obtaining the sample and the delivering the synthetic sequences encoding at least about 10, 20, 30, 40, 50, 60, 70, 80, 90, 100 or more different TCRs into the recipient cells can be no more than 7 weeks. In some cases, a time interval between obtaining the sample and the delivering the synthetic sequences encoding at least about 10, 20, 30, 40, 50, 60, 70, 80, 90, 100 or more different TCRs into the recipient cells can be no more than 3 weeks.

The methods described herein can produce a pool of synthetic TILs expressing different TCRs. The pool of synthetic TILs can be polyclonal TCR-T cells. The plurality of synthetic TILs can exogenously express at least about 2, 3, 4, 5, 10, 20, 30, 40, 50, 100, 500, 1,000, 10,000, 100,000 or more different TCRs.

The two or more cells of the plurality of synthetic TILs can be administered into the subject, and the two or more cells administered can exogenously express at least about 2, 3, 4, 5, 10, 20, 30, 40, 50, 100, 500, 1,000, 10,000, 100,000 or more different TCRs. The plurality of synthetic TILs can comprise a subpopulation of cells expressing TCRs that recognize tumor antigens and a subpopulation of cells expressing TCRs that do not recognize tumor antigens. In various cases, IL-2 can be administered (e.g., intravenously via infusion) prior to, concurrently, or after administering the synthetic TILs described herein.

The sample can be a tumor sample. The tumor sample can be a biopsy sample such as core biopsy or fine needle biopsy sample. These samples may have a small volume (e.g., <1000 mm³ (μL), <500 mm³, <100 mm³, <50 mm³) because even a small volume of tumor sample may contain sufficient cells for sequencing. In some cases, the volume of a tumor sample can be equal to or at most about 2000 mm³, 1000 mm³, 800 mm³, 500 mm³, 100 mm³, 50 mm³, 20 mm³ or less. Thus, this method can be applicable to situations where large surgical tumor sample is difficult to obtain. For example, a volume of the resection or biopsy sample can be less than about 200, 150, 100, 50, 20 or less mm³. In some cases, the volume of the resection or biopsy sample can be less than about 100 mm³. A weight of the resection or biopsy sample can be less than about 1 gram (g), 0.9 g, 0.8 g, 0.7 g, 0.6 g, 0.5 g, 0.4 g, 0.3 g, 0.2 g, 0.1 g, 0.08 g, 0.07 g, 0.06 g, 0.05 g, 0.04 g, 0.03 g, 0.02 g, 0.01 g or less. In some cases, a weight of the resection or biopsy sample can be less than about 0.1 g. In some cases, a weight of the resection or biopsy sample can be less than about 0.03 g. In some cases, a weight of the resection or biopsy sample can be no more than 0.1 g, 0.08 g, 0.05 g, 0.03 g or less.

The sequences encoding exogenous TCRs can be delivered into recipient cells for expression. The sequences encoding exogenous TCRs can be delivered as a mixture. The mixture can comprise at least about 2, 3, 4, 5, 10, 20, 30, 40, 50, 100, 500, 1,000, 10,000, 100,000 or more different polynucleotide sequences encoding at least about 2, 3, 4, 5, 10, 20, 30, 40, 50, 100, 500, 1,000, 10,000, 100,000 or more different natively paired TCRs. Delivering can be done by various methods described herein. For example, the plurality of polynucleotide sequences or derivative thereof can be delivered into the plurality of recipient cells by a vector. The vector can be a non-viral vector. The non-viral vector can be a plasmid, a transposon, a self-amplifying RNA, or any combination thereof. The vector can be a viral vector. The viral vector can be an adenoviral vector, an adeno-associated viral (AAV) vector, a herpes simplex viral vector, lentiviral vector, a retroviral vector, or any combination thereof. The viral vector can be the AAV vector. The viral vector can be the lentiviral vector. The vector may not comprise a promoter.

The recipient cells can be obtained from various sources. The recipient cells can be cell line cells or primary cells isolated from a subject. The plurality of recipient cells can be a plurality of T cells. The plurality of recipient cells can be isolated from the same subject where the plurality of source T cells (e.g., those cells from which the natively paired TCRs are obtained) are obtained. The plurality of recipient cells can be primary T cells. The plurality of recipient cells can be peripheral T cells.

The subject described herein may have failed a prior therapy. The prior therapy can be a checkpoint blockade therapy.

The sequencing described herein may be optional, in some cases, prior to synthesizing the sequences encoding the TCRs. For example, the method of producing a plurality of synthetic tumor-infiltrating lymphocytes (TILs) can comprise obtaining a resection or biopsy sample comprising a plurality of T cells from a solid tumor lesion of a subject. Next, a plurality of polynucleotide sequences encoding natively paired TCRs or a subset thereof of the plurality of T cells obtained can be synthesized. The plurality of polynucleotide sequences can comprise a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence. Next, the plurality of polynucleotide sequences or derivative thereof can be delivered into a plurality of recipient cells by contacting a mixture comprising the plurality of polynucleotide sequences or derivative thereof with a plurality of recipient cells, thereby producing the plurality of synthetic TILs. In some cases, sequencing can be performed to obtain natively paired T-cell receptor (TCR) alpha chain and beta chain sequences of TCRs of the plurality of T cells. In some cases, expression level of one or more genes (e.g., at least 10 genes) from the sequencing can be obtained.

In some aspects, the present disclosure provides a method of producing a plurality of synthetic tumor-infiltrating lymphocytes (TILs), the method comprising delivering a plurality of polynucleotide sequences or derivative thereof into a plurality of recipient cells by contacting a mixture comprising the plurality of polynucleotide sequences or derivative thereof with the plurality of recipient cells. Each polynucleotide sequence of the plurality can encode a cognate pair of TCR alpha chain and a TCR beta chain. Each polynucleotide sequence can be knocked into a genomic locus for monoallelic expression of the polynucleotide sequence. In some cases, an endogenous TCR gene of each recipient cell of the plurality of recipient cells can be inactivated. In some cases, an endogenous TCR gene of each recipient cell of the plurality of recipient cells can be inactivated prior to, concurrently, or after delivering the sequence encoding the cognate pair of TCR for expression. In some cases, an endogenous TCR gene of each recipient cell of the plurality of recipient cells may have been inactivated prior to delivering the sequence encoding the cognate pair of TCR for expression. In some cases, at least about 40%, 50%, 60%, 70%, 80%, 90%, 99% or 100% of the plurality of synthetic TILs exogenously express only a single copy of polynucleotide sequence encoding a natively paired TCR of the TCRs of the plurality of T cells. The genomic locus can be TRAC locus. The endogenous TCR gene can be disrupted by knocking down or knocking out TRBC locus. The mixture can comprise at least about 2, 3, 4, 5, 10, 20, 30, 40, 50, 100, 500, 1,000, 10,000, 100,000 or more different polynucleotide sequences encoding at least about 2, 3, 4, 5, 10, 20, 30, 40, 50, 100, 500, 1,000, 10,000, 100,000 or more different natively paired TCRs.

The polyclonal synthetic TILs produced herein can be used to treat a subject in need thereof, or can be used to prepare a medicament for treating a disease. The disease can be cancer. The cancer can be a solid tumor. In some cases, the cancer is esophageal cancer, gastric cancer, colorectal cancer, head and neck cancer, lung cancer, liver cancer, ovarian cancer, or cervical cancer. In some cases, the cancer is a melanoma, renal cell carcinoma, lung cancer, bladder cancer, breast cancer, cervical cancer, colorectal cancer, head and neck cancer, colon cancer, gall bladder cancer, laryngeal cancer, liver cancer (e.g., hepatocellular carcinoma), thyroid cancer, stomach cancer, salivary gland cancer, prostate cancer, pancreatic cancer, urothelial cancer, or Merkel cell carcinoma. The subject in need thereof can be the subject from whom the natively paired TCRs are obtained. The methods or pharmaceutical compositions provided herein can be used for personalized treatment of the subject in need thereof.

### Source T Cells or T-Cell Receptors

In various aspects, source T cells can be isolated from a sample from a subject from which the natively paired TCRs (or cognate pairs of TCRs) can be obtained and sequenced. The T cells can be used as a source for providing TCRs in the methods described herein for producing synthetic TILs. For example, a plurality of T cells can be isolated from a tissue sample (e.g., a tumor sample or a solid tumor sample) from a subject. The TCRs of the T cells can be sequenced to determine the cognate pairs of TCR alpha chains and beta chains or TCR gamma chains and delta chains.

The T cells can be T cells obtained from a subject expressing subject-derived or subject-specific TCRs. The subject-derived or subject-specific TCRs can be specific to the subject or the tumor of the subject, which subject is intended to be treated with a pharmaceutical composition described herein.

In some cases, the T cells can be CD4+ T cells, CD8+ T cells, or CD4+/CD8+ T cells. In some cases, the T cells can be helper T cells, cytotoxic T cells, memory T cells, regulatory T cells, natural killer T cells, alpha beta T cells, or gamma delta T cells.

The T cell can be obtained from a tissue sample comprising a solid tissue, with non-limiting examples including a tissue from brain, liver, lung, kidney, prostate, ovary, spleen, lymph node (e.g., tonsil), thyroid, thymus, pancreas, heart, skeletal muscle, intestine, larynx, esophagus, and stomach. Additional non-limiting sources include bone marrow, cord blood, tissue from a site of infection, ascites, pleural effusion, spleen tissue, and tumors. In some cases, the T cells can be obtained from a solid tumor lesion from a subject. The T cell can be derived or obtained from a healthy donor, from a patient diagnosed with cancer or from a patient diagnosed with an infection.

The T cells can be isolated from a sample and selected with certain properties by various methods. When isolating T cells from tissues (e.g., isolating tumor-infiltrating T cells from tumor tissues), the tissues made be minced or fragmented to dissociate cells before lysing the red blood cells or depleting the monocytes. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least or equal to about 1, 2, 3, 4, 5, or 6 hours. In yet another aspect, the time period is 10 to 24 hours. In an aspect, the incubation time period is about 24 hours. Longer incubation times may be used to isolate T cells in any situation where there are few T cells as compared to other cell types, such as in isolating tumor infiltrating lymphocytes (TILs) from tumor tissue or from immunocompromised individuals. Further, use of longer incubation times can increase the efficiency of capture of CD8+ T cells. Thus, by simply shortening or lengthening the time T cells are allowed to bind to the anti-CD3/anti-CD28 beads and/or by increasing or decreasing the ratio of beads to T cells, subpopulations of T cells can be selected for or against at culture initiation or at other time points during the process. Additionally, by increasing or decreasing the ratio of anti-CD3 and/or anti-CD28 antibodies on the beads or other surface, subpopulations of T cells can be selected for or against at culture initiation or at other desired time points. In some cases, multiple rounds of selection can be used. In certain aspects, the selection procedure can be performed and the “unselected” cells (cells that may not bind to the anti-CD3/anti-CD28 beads) can be used in the activation and expansion process. “Unselected” cells can also be subjected to further rounds of selection.

Enrichment of a T cell population by negative selection can be accomplished with a combination of antibodies directed to surface markers unique to the negatively selected cells. An example method can be cell sorting and/or selection via negative magnetic immune adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be useful to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

A T cell population can be selected that expresses one or more of IFN-γ, TNF-alpha, IL-17A, IL-2, IL-3, IL-4, GM-CSF, IL-10, IL-13, granzyme B, and perforin, or other molecules, e.g., other cytokines and transcription factors such as T-bet, Eomes, Tef1 (TCF7 in human). Methods for screening for cell expression can be determined, e.g., by the methods described in PCT Publication No.: WO 2013/126712.

For isolation of a population of cells by positive or negative selection, the concentration of cells and surface (e.g., particles such as beads) can be varied. In certain aspects, the volume in which beads and cells are mixed together may be decreased (e.g., increase the concentration of cells) to ensure maximum contact of cells and beads. For example, in an aspect, a concentration of 2 billion cells/mL is used. In another aspect, a concentration of 1 billion cells/mL is used. In a further aspect, greater than 100 million cells/mL is used. In a further aspect, a concentration of cells of at least about 10, 15, 20, 25, 30, 35, 40, 45, or 50 million cells/mL is used. In some aspects, a concentration of cells of at least about 75, 80, 85, 90, 95, or 100 million cells/mL is used. In some aspects, a concentration of cells of at least about 125 or 150 million cells/mL can be used. Using high concentrations can result in increased cell yield, cell activation, and cell expansion. Further, use of high cell concentrations can allow more efficient capture of cells that may weakly express cell surface markers of interest, such as CD28-negative T cells, or from samples where there are many tumor cells present (e.g., leukemic blood, tumor tissue, etc.). Such populations of cells may have therapeutic value. For example, using high concentration of cells can allow more efficient selection of CD8+ T cells that may have weaker CD28 expression.

In some cases, lower concentrations of cells may be used. By significantly diluting the mixture of T cells and surface interactions between the particles and cells can be minimized. This can select for cells that express high amounts of desired antigens to be bound to the particles. For example, CD4+ T cells can express higher levels of CD28 and can be more efficiently captured than CD8+ T cells in dilute concentrations. In some aspects, the concentration of cells used is at least about 5×10⁵/mL, 5×10⁶/mL, or more. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

The source T cells can be tumor-infiltrating lymphocytes (TILs), e.g., tumor-infiltrating T cells (TITs). A TIL can be isolated from an organ afflicted with a cancer. One or more cells can be isolated from an organ with a cancer that can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. One or more TILs can be from a brain, heart, liver, skin, intestine, lung, kidney, eye, small bowel, or pancreas. TILs can be from a pancreas, kidney, eye, liver, small bowel, lung, or heart. The one or more cells can be pancreatic islet cells, for example, pancreatic β cells. In some cases, a TIL can be from a gastrointestinal cancer. A TIL culture can be prepared a number of ways. For example, a tumor can be trimmed from non-cancerous tissue or necrotic areas. A tumor can then be fragmented to about 2-3 mm in length. In some cases, a tumor can be fragmented from about 0.5 mm to about 5 mm in size, from about 1 mm to about 2 mm, from about 2 mm to about 3 mm, from about 3 mm to about 4 mm, or from about 4 mm to about 5 mm. Tumor fragments can then be cultured in vitro utilizing media and a cellular stimulating agent such as a cytokine. In some cases, IL-2 can be utilized to expand TILs from a tumor fragment. A concentration of IL-2 can be about 6000 IU/mL. A concentration of IL-2 can also be about 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL. Once TILs are expanded, they can be subject to in vitro assays to determine tumor reactivity. For example, TILs can be evaluated by FACs for CD3, CD4, CD8, and CD58 expression. TILs can also be subjected to cocultured, cytotoxicity, ELISA, or ELISPOT assays. In some cases, TIL cultures can be cryopreserved or undergo a rapid expansion. A cell, such as a TIL, can be isolated from a donor of a stage of development including, but not limited to, fetal, neonatal, young and adult.

In some cases, the tumor sample can be a surgically removed tumor sample (or a resection sample). The T cells can be TITs isolated from the resection sample. For example, the tumor tissue can be cut up into 3-5 mm² fragments after trimming away fat and connective tissue and disaggregated in cold RPMI 1640 using gentle mechanical pulverization using a Seward Stomacher device (Fisher, Pittsburgh, PA). This process can rapidly produce a single cell suspension without enzymatic digestion. The cell suspension can be filtered through a 75 μm pore-size screen (BD Biosciences, San Jose CA) and washed in culture medium. A portion of the cells used for immediate staining and analysis by flow cytometry can be washed in culture medium and the cell suspension can be layered over a discontinuous 70% followed by a 100% Ficoll Isopaque gradient, and centrifuged to separate the tumor cells (70% interface) from the enriched TITs (100% interface). The enriched TITs can be then washed in D-PBS, 1% BSA and then processed. In some cases, the TITs from tumor samples can be expanded in TIT culture medium (TIL-CM) containing RPMI 1640 with Glutamax (Invitrogen), 10% human AB serum (Sigma, St. Louis, MI), 50 mM 2-mercaptoethanol (Invitrogen), 1 mM pyruvate, 1× Penicillin/Streptomycin (Invitrogen) using 3,000 IU/ml recombinant IL-2.

In some cases, the tumor sample can be a biopsy sample such as core biopsy or fine needle biopsy sample. These samples may have a small volume (e.g., <1000 mm³ (μL), <500 mm³, <100 mm³, <50 mm³) because even a small volume of tumor sample may contain sufficient cells for sequencing. In some cases, the volume of a tumor sample can be equal to or at most about 2000 mm³, 1000 mm³, 800 mm³, 500 mm³, 100 mm³, 50 mm³, 20 mm³ or less. Thus, this method can be applicable to situations where large surgical tumor sample is difficult to obtain. For example, a volume of the resection or biopsy sample can be less than about 200, 150, 100, 50, 20 or less mm³. In some cases, the volume of the resection or biopsy sample can be less than about 100 mm³. A weight of the resection or biopsy sample can be less than about 1 gram (g), 0.9 g, 0.8 g, 0.7 g, 0.6 g, 0.5 g, 0.4 g, 0.3 g, 0.2 g, 0.1 g, 0.08 g, 0.07 g, 0.06 g, 0.05 g, 0.04 g, 0.03 g, 0.02 g, 0.01 g or less. In some cases, a weight of the resection or biopsy sample can be less than about 0.1 g. The T cells can be TIT isolated from the biopsy sample.

In some cases, fresh tumor may not be available, and in such cases, nuclei may be isolated from frozen or fixed tissue. These nuclei may also serve as the input for identifying cognate pairs of TCRs and preparing synthetic TILs described herein. These cells and nuclei can be used without further selection. Alternatively, a specific population of cells or nuclei can be isolated to enrich tumor specific TCRs. For example, expression of cell surface markers CD39, CD69, CD103, CD25, PD-1, TIM-3, OX-40, 4-1BB may be correlated with tumor-reactivity. The cell surface markers can be used to isolate/enrich tumor-reactive TCRs by FACS. In other words, cells with high expression of one or a combination of these markers can be used as the input for the identifying cognate pairs of TCRs.

### Sequencing

Various sequencing methods can be used herein. Various sequencing methods include, but are not limited to, Sanger sequencing, high-throughput sequencing, sequencing-by-synthesis, single-molecule sequencing, sequencing-by-ligation, RNA-Seq, Next generation sequencing (NGS), Digital Gene Expression, Clonal Single MicroArray, shotgun sequencing, Maxim-Gilbert sequencing, or massively-parallel sequencing. The TCR-expressing cells can be used as input for single-cell RNA-Seq methods such as inDrop or DropSeq. For example, the sequencing may use single cell barcoding (e.g., partitioning the cells into individual compartment, barcoding nucleic acids released from a single cell, sequencing the nucleic acids, and pair the TCR chains from a single cell based on a same barcode). The sequencing may not comprise using a barcode if the sequence encoding the paired TCR chains within a cell has been fused or linked in a single continuous polynucleotide chain.

Sequencing described herein can be single cell sequencing. Single cell sequencing refers to obtaining sequence information from individual cells. It can be used to detect the genome, transcriptome and other multi-omics of single cells. In single cell sequencing, a population of cells can be made into single cell suspension and compartmentalized into individual partitions. Within each partition, the sequences released from a single cell can be barcoded and later sequenced. Various single cell sequencing methods can be used for TCR reconstruction (see De Simone M, Rossetti G and Pagani M (2018) Single Cell T Cell Receptor Sequencing: Techniques and Future Challenges. Front. Immunol. 9:1638).

In some cases, the source T cells isolated from a sample from a subject can be sequenced to obtain information regarding native pairs of TCR alpha chains and TCR beta chains, or TCR gamma chains and TCR delta chains. In some cases, the source T cells can be sequenced to obtain gene expression levels of certain gene markers. The gene markers can include, but are not limited to, CXCL13, CTLA4, DUSP4, ENTPD1, RBPJ, TNFSF4, GK, PDCD1, LAG3, FKBPIA, MYO7A, FABP5, GAPDH, TIGIT, CDCA7, ZBED2, RGS1, HAVCR2, MKI67, GZMB, SPRY1, PHLDA1, ASPM, MYO1E, TOX, LSP1, MCM10, MAD2L1, OGN, PCLAF, UBE2T, GNG4, LY6E, TPI1, TNFRSF18, RGS2, CLEC2D, SAMSN1, TSHZ2 and HMGN2.

### Oligonucleotide Synthesis

The polynucleotides encoding various cognate pairs of TCRs can be synthesized chemically de novo. In some cases, the polynucleotides can be synthesized by phosphoramidite chemistry. For example, based on the sequencing information, the sequences encoding cognate pairs of TCRs can be obtained, and each sequence can be synthesized chemically, e.g., by phosphoramidite chemistry. In some cases, fragments of each sequence encoding the cognate pair of TCR can be synthesized chemically, and the fragments for each cognate pair can be assembled individually such as in a separate compartment as other sequences encoding different TCRs.

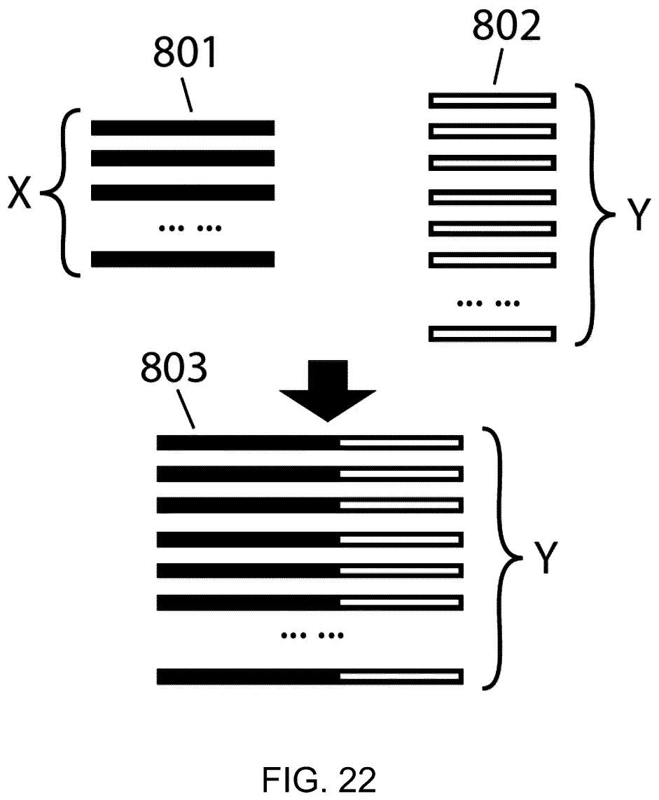

The polynucleotides encoding different cognate pairs of TCRs can also be synthesized in bulk or in a mixture (e.g., a same mixture) using the methods disclosed in International Application No. PCT/US2020/026558, the entire content of which is incorporated herein by reference. In this method, polynucleotides, each comprising a sequence derived from a TCR V gene or a portion thereof can be pre-synthesized and mixed together as a pool. The sequence derived from a TCR V gene can be a portion of the TCR V gene. The sequence derived from a TCR V gene can be a codon-optimized sequence or comprise one or more modified nucleotides. For example, the sequence derived from the TCR V gene comprising coding sequences for L-PART1 (first part of the leader peptide), L-PART2 (second part of the leader peptide), FR1, CDR1, FR2, CDR2 and FR3, referred to as L-V-REGION, can be pre-synthesized. For another example, the sequence derived from the TCR V gene comprising coding sequences for FR1, CDR1, FR2, CDR2 and FR3, referred to as V-REGION, can be pre-synthesized. The nucleic acid sequence segment of L-PART1, L-PART2, FR1, CDR1, FR2, CDR2, or FR3, can be defined according to the IMGT unique numbering system (http://www.imgt.org). In some cases, the sequence derived from the TCR V gene can comprise a sequence starting from the sequence encoding L-PART1 and ending at the codon encoding the second conserved cysteine (e.g., 2nd-CYS, as defined by IMGT, corresponds to codon for the conserved cysteine at position 104 of the V-DOMAIN). Since there are about 80 or more TCR V genes (e.g., TRAV and TRBV genes) in human genome, synthesis of such “V gene germline polynucleotide library” (as shown in FIG. 22, 801 and bracket X) can be feasible. In some cases, a subset of TCR V genes of a species (e.g., a human or a mouse) are synthesized to generate the “V gene germline polynucleotide library.” All identified or a subset of TCR V genes may be synthesized. For example, at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 61, 62, 63, 64, 65, 66, 67, 68, 69, 70, 71, 72, 73, 74, 75, 76, 77, 78, 79, 80 or more TCR V genes of the species can be synthesized to generate the library. In some cases, all identified TCR V genes of a species are synthesized to generate the library. The TCR V gene can be TRAV, TRBV, TRGV, or TRDV. As described herein, in some cases, a “V gene germline polynucleotide” refers to a portion of the genomic or codon-optimized polynucleotide of a TCR V gene. The sequence derived from the TCR V gene can be the V gene germline polynucleotide. The sequence between FR3 and constant region (e.g., CDR3 plus the remaining of the J region, referred to as “CDR3-J” herein) can be at least about 10, 20, 30, 40, 50, 60, 70, 80, 90 or more nucleotides long, or in some cases, can be up to about 90 nucleotides long. The CDR3-J sequence of the alpha chain and beta chain of a TCR can be at least about 50, 60, 70, 80, 90, 100, 120, 150, 180 or more nucleotides long. The CDR3-J sequence of the alpha chain and beta chain of a TCR (in some cases, in total up to about 180 nucleotides long) can be included into an oligonucleotide (referred to as a “paired CDR3-J oligo”, a “paired CDR3-J oligonucleotide” or a “paired CDR3-J polynucleotide”, which can be used interchangeably) that can be amenable to chip-based synthesis (as shown in FIG. 22, 802 and bracket Y encompassing 802). In some cases, the paired CDR3-J polynucleotide can comprise a CDR3-J sequence of a TCR gamma chain and a CDR3-J polynucleotide of a TCR delta chain. As used herein, the terms “CDR3-J polynucleotide,” “CDR3-J oligonucleotide,” and “CDR3-J oligo” (which can be used interchangeably) refer to a polynucleotide sequence comprising one or more CDR3-J sequences. A CDR3-J polynucleotide may be a paired CDR3-J polynucleotide (e.g., comprising CDR3-J sequences from a paired TCR chains). The CDR3-J polynucleotide (e.g., non-paired) may contain only the CDR3-J sequence from one of the paired TCR chains. For example, the CDR3-J polynucleotide may contain only the CDR3-J sequence from a TCR alpha chain, a TCR beta chain, a TCR gamma chain, or a TCR delta chain. The remaining challenge can be to convert such paired CDR3-J oligonucleotide into expressible TCR construct in high throughput (e.g., constructing >1,000 TCRs in one batch). Using the methods described herein, the paired CDR3-J oligonucleotide can be linked to their corresponding V gene germline polynucleotides in a bulk reaction (e.g., FIG. 22, 803). In some cases, the CDR3-J polynucleotide pool (e.g., paired or non-paired) can comprise at least about 2, 5, 10, 20, 50, 100, 200, 300, 400, 500, 1,000, 1,500, 2,000, 2,500, 3,000, 3,500, 4,000, 5,000, 6,000, 7,000, 8,000, 9,000, 10,000, 12,000, 15,000, 20,000, 100,000, 1,000,000, 10,000,000, or more different sequences. The V gene germline polynucleotide library can comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 61, 62, 63, 64, 65, 66, 67, 68, 69, 70, 71, 72, 73, 74, 75, 76, 77, 78, 79, 80 or more TCR V genes. When using the methods described herein, a plurality of at least 2, 5, 10, 20, 50, 100, 200, 300, 400, 500, 1,000, 1,500, 2,000, 2,500, 3,000, 3,500, 4,000, 5,000, 6,000, 7,000, 8,000, 9,000, 10,000, 12,000, 15,000, 20,000, 100,000, 1,000,000, 10,000,000, or more different sequences encoding different natively paired TCRs can be generated. The natively paired TCRs can be generated in bulk in a single compartment.

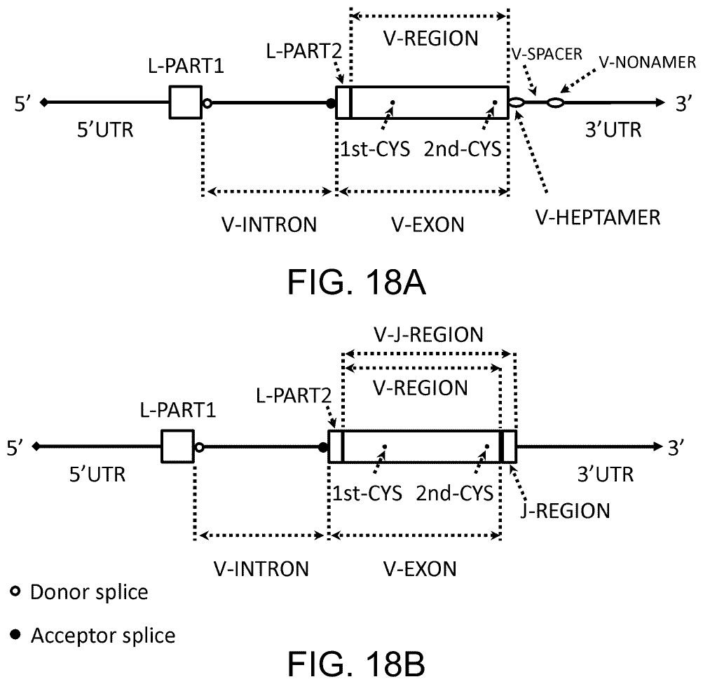

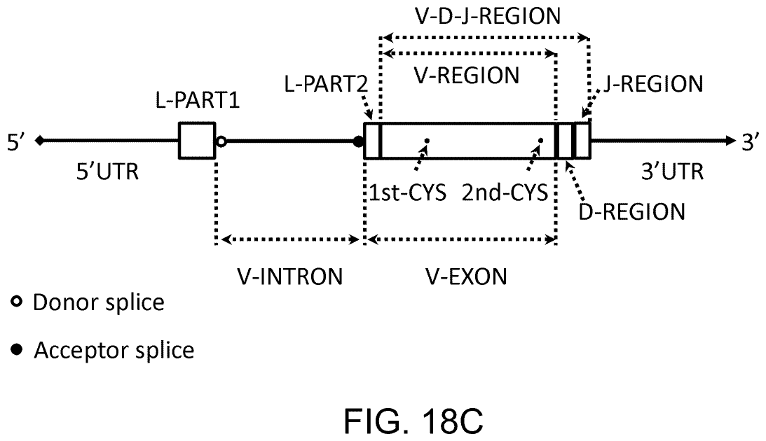

Examples of germline or rearranged gene construct of a nucleic acid molecule comprising a TCR V gene sequence are shown in FIGS. 18A-18C. For example, FIG. 18A shows germline genomic DNA of a TCR V gene, comprising L-PART1, V-INTRON, V-EXON, and recombination signal sequences (V-HEPTAMER, V-SPACER, and V-NONAMER). The two conserved cysteines are also shown in FIG. 18A. After V-(D)-J recombination, an example construct of the rearranged genomic DNA is shown in FIG. 18B or FIG. 18C. The CDR3 can be encoded by (i) the junction (or junctional region) between V-REGION and J-REGION or (ii) the junction between V-REGION and D-REGION and the junction between D-REGION and J-REGION.

The TCR V genes can be very diverse. In human, more than 40 functional V genes for TRA have been identified, including, for example, TRAV1-1, TRAV1-2, TRAV2, TRAV3, TRAV4, TRAV5, TRAV6, TRAV7, TRAV8-1, TRAV8-2, TRAV8-3, TRAV8-4, TRAV8-6, TRAV9-1, TRAV9-2, TRAV10, TRAV12-1, TRAV12-2, TRAV12-3, TRAV13-1, TRAV13-2, TRAV14, TRAV16, TRAV17, TRAV18, TRAV19, TRAV20, TRAV21, TRAV22, TRAV23, TRAV24, TRAV25, TRAV26-1, TRAV26-2, TRAV27, TRAV29, TRAV30, TRAV34, TRAV35, TRAV36, TRAV38-1, TRAV38-2, TRAV39, TRAV40, and TRAV41. Among these V genes, some of them can be classified into a same subgroup and they are indicated by a same subgroup number immediately following “TRAV” but a different number following “-” sign. For example, TRAV1-1 and TRAV1-2 are from a same subgroup. As used herein, a “group” is a set of genes that share the same “gene type” (e.g., V, D, J or C type) and participate potentially in the synthesis of a polypeptide of the same “chain type”. By extension, a group includes the related pseudogenes and orphans. A “subgroup” means a set of genes that belong to the same group, in a given species, and that share at least 75% identity at the nucleotide level (in the germline configuration for V, D, and J).

In human, more than 40 functional V genes for TRB have been identified, including, for example, TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1, and TRBV30. V genes for other species, e.g., mouse, can be found in IMGT database.

### Diversify Connector Sequences

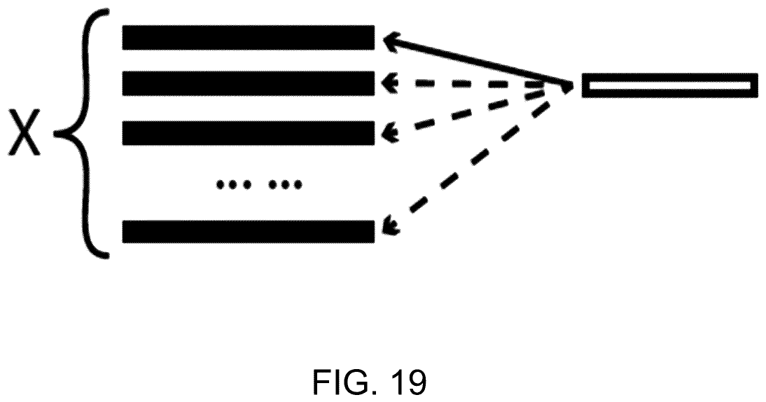

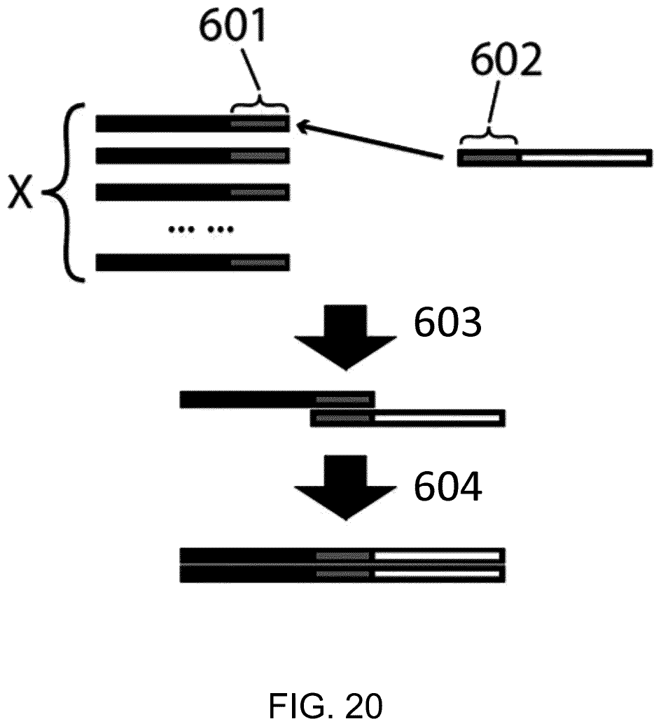

Connecting a V gene germline polynucleotide and a CDR3-J polynucleotide can be achieved by molecular biology techniques such as ligation and overlapping primer extension (FIG. 20). However, to fully utilize the power of chip-based oligonucleotide synthesis, one may connect thousands of or more CDR3-J oligonucleotides with their corresponding V gene germline polynucleotides in a bulk reaction (as shown by the arrow of FIG. 22, 803). The major challenge in doing this can be that the connector region between the V gene germline polynucleotide (FIG. 20, 601) and the CDR3-J (FIG. 20, 602) may be the conserved FR3 region. Therefore, in a bulk reaction, it can be difficult to control which V gene germline polynucleotide is connected to which CDR3-J (as depicted in FIG. 19 where the solid arrow depicts linking to the correct V gene germline polynucleotide and the dashed arrows depict linking to the incorrect V gene germline polynucleotide). For example, a TCR sequence may be formed by TRBV4-1 connected to a particular CDR3-J beta sequence. In the bulk reaction, the V gene germline polynucleotides for both TRBV4-1 and TRBV4-2 can be present, and the FR3 regions for these TRBV genes can be highly similar. Therefore, the CDR3-J oligonucleotide for this TCR may be incorrectly connected to the TRBV4-2 germline polynucleotide. To alleviate this problem, codon diversification can be used to create dissimilarities among different FR3 sequences. For example, the connector sequences (also called “Zip” sequence in the present disclosure) can be codon-diversified such that they can have different nucleic acid sequences, even though they may encode an identical amino acid sequence. Codon diversification can be achieved by computational methods. A plurality of nucleic acid sequences can be generated by assigning a codon to an amino acid randomly or according to an arbitrary rule, where each nucleic acid sequence can encode the same amino acid sequence. Next, the plurality of nucleic acid sequences can be evaluated computationally to assign a score according to an arbitrary rule. The arbitrary rule may consider factors such as restriction site, propensity to hybridize with an unwanted sequence, propensity to hybridize with a given sequence, or unwanted secondary structure in the sequence. Next, based on the score, a nucleic acid sequence can be selected from the plurality of nucleic acid sequences as a codon-diversified connector sequence. The codon-diversified connector sequence can be used to achieve correct linking of a CDR3-J polynucleotide and the designated pre-synthesized portion of a TCR V gene. In a “V gene germline polynucleotide library” comprising some or all the known TCR V genes, for example, each different TCR V gene can have a different connector sequence, which can be used to correctly connect to the corresponding CDR3-J oligonucleotide to form a TCR chain according to a reference sequence. The reference sequence can be generated by sequencing cognate pairs of TCR chains. However, in some cases, it may be unclear to what extent the connector sequences can be diversified and to what extent the connection between the V gene germline polynucleotides and CDR3-J oligonucleotides can be correct in a bulk reaction. It may be possible to diversify the FR3 regions of human TCR V genes so that the ‘mis-connection probability’ for any given CDR3-J sequence is practically undetectable.

Different connector sequences or Zips used in one homogenous assembly reaction may have different length or GC content, but may have similar melting temperature. In some cases, hundreds to thousands of Zip sequences are used in a homogenous assembly reaction. Designing the Zip sequences may follow similar rules as designing primers for PCR reaction, such as: all Zips used in one assembly reaction can have similar melting temperature, a Zip may not form strong hairpin at 5° C. below melting temperature, one Zip may not hybridize strongly to another Zip at 5° C. below melting temperature, one Zip may not hybridize strongly to the complement of another Zip at 5° C. below melting temperature.

For example, to generate a set of 1,000 Zips that can be used in the same assembly reaction, one million random 50-mer sequences can be generated first. Next, a desired melting temperature (e.g., 60° C.) can be chosen. Then, the shortest sub-sequence of each of the 30-mer sequence (starting from the 5′ end) whose melting temperature is above the desired melting temperature can be kept, while the rest of the bases can be removed. The resultant one million sequences (with various length) can be called “trimmed random sequences.” Next, the secondary structure of each trimmed random sequence can be evaluated and ranked based on the Gibbs free energy of the minimum free-energy (MFE) structure at 5° C. below the desired melting temperature. The top 10,000 trimmed random sequences, with the highest (e.g., least negative) Gibbs free energy, can be kept. Each of these kept sequences can be called a Zip candidate. If restriction enzymes are used in the assembly reactions, Zip candidate sequences containing such restriction sites may be removed. Next, each of the Zip candidate can be evaluated based on how strongly it forms primer dimer with all other Zip candidates and their complements. A penalty score can be assigned if a strong primer dimer is formed. The penalty score can be positively correlated with the strength of the primer dimer. The sum of all penalty scores can be the final penalty score for each Zip. The top 3,000 Zip candidates with the lowest final penalty score can be kept which can be called Zip finalists. Then this primer dimer evaluation process can be repeated for the 3,000 finalists to choose the top 1,000 sequences with the lowest final penalty score, which can be used as Zips. A number of web-based and stand-alone software packages such as Primer3, UNAfold, NUPACK, PrimerROC, Pythia, Multiple Primer Analyzer (Thermo Fisher), and OligoEvaluator (Sigma-Aldrich) can be used to implement this process.

Once a diverse set of connector sequences are found, many methods using molecular biology techniques (e.g., ligation, restriction digestion, circularization) can be used to convert a CDR3-J oligonucleotide pool to a full-length, expressible TCR pool. Example 1 provides an example workflow. The methods provided herein can also be used to generate a pool of individual TCR chains (e.g., not paired chains) in a bulk reaction. For example, to generate a pool of TCR alpha chains, each individual CDR3-J oligonucleotide may comprise CDR3 and J region from TCR alpha chain but may not comprise another CDR3 and J region from a TCR beta chain, and then the CDR3-J oligonucleotide can be used to link with corresponding TRAV gene to form the TCR alpha chain.

### Methods for Constructing Nucleic Acid Molecules Encoding TCRs

The nucleic acid molecules encoding TCRs described herein can be constructed from two or more nucleic acid fragments. In some embodiments, the two or more nucleic acid fragments can be referred to as a first nucleic acid molecule, a second nucleic acid molecule, a third nucleic acid molecule, a fourth nucleic acid molecule, etc. When constructing the nucleic acid molecules, standard molecular biology techniques, including but not limited to hybridization, extension, ligation, and enzymatic digestion/cleavage, may be used.

The nucleic acid fragment described herein can encode a TCR chain or portion thereof. For example, the portion of the TCR chain encoded by the nucleic acid fragment can comprise greater than or equal to about 10, 15, 20, 25, 30, 35, 40, 45, 50, 60, 70, 80, 90, 100, 110, 120, 130, 140, 150, 200, 250, or more amino acids. The nucleic acid fragment can comprise a sequence encoding a functional TCR chain. The functional TCR chain may or may not be a full length TCR chain. The functional TCR chain may comprise one or more mutations or modifications. In some cases, a functional TCR chain, when expressed in a host cell, can incorporate into a TCR complex (e.g., a complex having TCRα, TCRβ, CD3γ, CD3δ, CD3ε, and (chains). In some cases, a functional TCR can bind to its target ligand. In some cases, a functional TCR, when expressed in a host cell, can incorporate into the cell membrane. In some cases, a functional TCR can be expressed in a host cell.

The nucleic acid fragment used to construct nucleic acid molecule encoding a TCR or portion thereof can comprise a sequence encoding a CDR3.

The nucleic acid fragment used to construct nucleic acid molecule encoding a TCR or portion thereof can comprise a sequence encoding a first CDR3 of a first TCR chain and a second CDR3 of a second TCR chain, wherein the first CDR3 and the second CDR3 are derived from a cognate pair of TCR chains. In some embodiments, the sequence encoding the first CDR3 and the sequence encoding the second CDR3 are separated by at most about 100, 90, 80, 70, 60, 50, 40, 30, 20, 10, or 5 nucleotides.

The nucleic acid fragment used to construct nucleic acid molecule encoding a TCR or portion thereof can comprise a TCR V gene sequence or portion thereof. The nucleic acid fragment used to construct nucleic acid molecule encoding a TCR or portion thereof can comprise a sequence derived from a TCR V gene sequence. The sequence derived from a TCR V gene can comprise a V-REGION nucleic acid sequence. The sequence derived from a TCR V gene can comprise a sequence encoding FR1, CDR1, FR2, CDR2 and/or FR3 nucleic acid sequence. The sequence derived from a TCR V gene can comprise a sequence encoding a leader peptide. The sequence derived from a TCR V gene can comprise a sequence encoding L-PART1, L-PART2, FR1, CDR1, FR2, CDR2 and/or FR3 nucleic acid sequence. The sequence derived from a TCR V gene can comprise or can be a portion of the TCR V gene. The portion of the TCR V gene can be at least 10 nucleotides in length. For example, the portion of the TCR V gene may be greater than or equal to about 10, 20, 30, 50, 60, 70, 80, 90, 100, 200, 300, 400, 500, 600, 700, 800, 900, 1000, or more nucleotides in length. The sequence derived from a TCR V gene may comprise one or more modified nucleotides. The sequence derived from a TCR V gene may be codon-optimized (or codon-diversified) such that it has a different sequence than the TCR V gene or portion thereof but it can encode a same amino acid sequence. The sequence derived from a TCR V gene may not comprise a sequence encoding a portion of a CDR3. The sequence derived from a TCR V gene may not comprise a sequence of a junctional region of a rearranged gene.

The nucleic acid fragment used to construct nucleic acid molecule encoding a TCR or portion thereof can comprise a sequence encoding a constant domain or portion thereof. The nucleic acid fragment used to construct nucleic acid molecule encoding a TCR or portion thereof can comprise a sequence encoding a constant region or portion thereof. In some cases, the constant domain or constant region is a TCR alpha constant domain or constant region, a TCR beta constant domain or constant region, a TCR gamma constant domain or constant region, or a TCR delta constant domain or constant region. In some cases, the constant region comprises a constant domain. In some cases, the constant region further comprises a transmembrane region, a connecting region, a cytoplasmic region, or a combination thereof.

The nucleic acid fragment used to construct nucleic acid molecule encoding a TCR or a portion thereof can comprise a connector sequence. The connector sequence can be used to link one nucleic acid molecule to another nucleic acid molecule. The connector sequence of one nucleic acid molecule can hybridize (e.g., form base pair or base pairs) with an anti-connector sequence of another nucleic acid molecule. The anti-connector sequence can be complementary (e.g., fully or substantially complementary) with the connector sequence. The anti-connector sequence can be hybridizable with the connector sequence under certain conditions (e.g., temperature, buffer condition, pH, etc.). The anti-connector sequence can be a reverse complement sequence (or complementary sequence) of the connector sequence. When the connector sequence hybridizes with the anti-connector sequence, the base pair(s) formed can be at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 100, or more base pairs. The base pairs formed between the connector sequence and the anti-connector sequence can be contiguous or non-contiguous. For example, in the cases where non-contiguous base pairs are formed, there may be unpaired region or regions separating paired regions. If a first nucleic acid molecule comprises a connector sequence, then a complementary sequence of the connector sequence on a second nucleic acid molecule can be referred to as an anti-connector sequence. The connector sequence (or anti-connector sequence) can be of various lengths. For example, the connector sequence (or anti-connector sequence) can be greater than or equal to about 2, 3, 4, 5, 6, 7, 8, 9, 10, 11, 12, 13, 14, 15, 20, 25, 30, 35, 40, 45, 50, 55, 60, 65, 70, 75, 80, 85, 90, 100, or more nucleotides in length. The connector sequence (or anti-connector sequence) can be less than or equal to about 300, 250, 200, 150, 100, 90, 85, 80, 75, 70, 65, 60, 55, 50, 45, 40, 35, 30, 25, 20, 15, 14, 13, 12, 11, 10, 9, 8, 7, 6, 5, 4, 3 or 2 nucleotides in length. The connector sequence (or anti-connector sequence) can be at 5′ end or 3′ end of a nucleic acid molecule. The connector sequence (or anti-connector sequence) can also be an internal sequence of a nucleic acid molecule. For example, the connector sequence can be an internal connector sequence and can be exposed at 5′ end or 3′ end by cutting an internal sequence (e.g., a sequence adjacent to the internal connector sequence) of the nucleic acid molecule. An example of the internal connector sequence is provided in Example 1, the inter-chain connector (ICC). In some cases, a connector sequence and an anti-connector sequence are used to link a nucleic acid molecule encoding a CDR3 or a portion thereof of a TCR chain with another nucleic acid molecule comprising a TCR V gene or a portion thereof. In some cases, a connector sequence and an anti-connector sequence are used to link a nucleic acid molecule comprising a J region of a TCR with another nucleic acid molecule comprising a TCR V gene or a portion thereof. In some cases, a connector sequence and an anti-connector sequence are used to link a nucleic acid molecule comprising a sequence encoding a CDR3 or a portion thereof and a J region of a TCR with another nucleic acid molecule comprising a TCR V gene or a portion. In some cases, a connector sequence and an anti-connector sequence are used to link a nucleic acid molecule comprising a sequence encoding a CDR3 or a portion thereof, a J region, and a TCR V gene or a portion thereof with another nucleic acid molecule encoding a constant domain or a portion thereof of a TCR.

The connector sequence (or the anti-connector sequence) can be a sequence encoding a portion of a TCR V gene (e.g., the portion of the TCR V gene adjacent to the sequence encoding a CDR3 in the rearranged gene). And in such cases, the connector sequence and one or more other connector sequences in a pool of connector sequences may encode a same amino acid sequence (e.g., the conserved portion of the TCR V gene adjacent to the CDR3). When the connector sequence encodes a conserved portion of a TCR V gene, the connector sequence can be codon-diversified such that the connector sequence can be used to link a nucleic acid molecule to another nucleic acid molecule specifically, resulting in a constructed nucleic acid molecule encoding a cognate pair of a TCR. In some embodiments, the connector sequence (or anti-connector sequence) comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 150, 200, or more nucleotides of the TCR V gene adjacent to a sequence encoding a CDR3 in a rearranged gene. Because of the specificity of the connector sequence and the anti-connector sequence, a pool of nucleic acid molecules having different sequences which encode different TCRs can be constructed in a bulk reaction (e.g., in a same compartment). The connector sequence can prescribe which TCR V gene the sequence encoding a CDR3 should be linked to according to a reference sequence (e.g., a native sequence of a TCR chain determined by sequencing). The connector sequence (or the anti-connector sequence) can be an arbitrary (e.g., pre-determined) sequence which may not encode a portion of a TCR V gene. And in such cases, the arbitrary sequence can be removed after linking two nucleic acid fragments together.

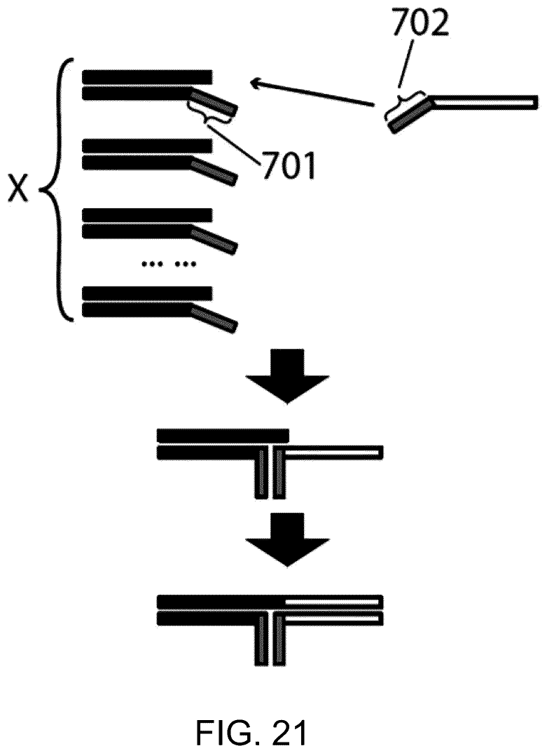

FIG. 21 depicts an example to use arbitrary connector (701) and anti-connector (702) sequence to link a CDR3-J polynucleotide to a designated V gene germline polynucleotide (thin arrow). Here each V gene germline polynucleotide has as partially double-stranded structure. The top strand, with its 3′ end to its right in this figure, has a single-stranded region at its 3′ end. The connector and the anti-connector sequences may be single stranded and may hybridize to each other. The connector and anti-connector sequence only serves the purpose of specific hybridization and may not be related to TCR whatsoever, hence arbitrary. After the hybridization between the connector and the anti-connector, the 3′ end of the top strand of the V gene germline polynucleotide may hybridize to the CDR3-J polynucleotide and may be extended by a DNA polymerase. The number of nucleotides on the 3′ end of the top strand of the V gene germline polynucleotide that are hybridized to the CDR3-J polynucleotide may be 1, 2, 3, 4, 5, 6, 7, 8, 9, 10, 11, 12, 13, 14, or 15, or up to 20.

The nucleic acid fragment used to construct nucleic acid molecule encoding a TCR or a portion thereof can comprise a self-cleaving peptide. The self-cleaving peptide can be a 2A peptide, an intein peptide, or a hedgehog peptide. Examples of 2A peptide include, but are not limited to, P2A (e.g., sequence: ATNFSLLKQAGDVEENPGP), E2A (e.g., sequence QCTNYALLKLAGDVESNPGP), F2A (e.g., sequence VKQTLNFDLLKLAGDVESNPGP), and T2A (e.g., sequence EGRGSLLTCGDVEENPGP) peptide.

The nucleic acid fragment used to construct nucleic acid molecule encoding a TCR or a portion thereof can comprise a restriction enzyme recognition site. For example, the restriction enzyme recognition site can be a recognition site for Type IIS restriction enzyme. Examples of Type-IIS restriction enzymes which can be useful in the present disclosure include, but are not limited to, EarI, MnlI, PleI, AlwI, BbsI, BbvI, BcoDI, BsaI, BseRI, BsmAI, BsmBI, BspMI, Esp3I, HgaI, SapI, SfaNI, BbvI, BsmFI, BsrDI, BtsI, FokI, BseRI, HphI, MlyI and MboII. In some cases, two or more different restriction enzymes can be used during nucleic acid construction process. In some cases, a restriction enzyme that create a 4-bp 5′ overhang (for example, BbsI, BbvI, BcoDI, BsaI, BsmBI, FokI, etc.) can be used. In some cases, a restriction enzyme that creates a blunt end or 3′ overhang (for example, BseRI, BsrDI, BtsI, MlyI, etc.) can be used.

A nucleic acid fragment used to construct nucleic acid molecule encoding a TCR or a portion thereof can be circularized. For example, the nucleic acid fragment can be circularized by joining two ends of the nucleic acid fragment by ligation. The ligation can be blunt end ligation. The ligation can be performed after creating sticky ends using 5′-to-3′ exonuclease (e.g., Gibson Assembly), 3′-to-5′ exonuclease (e.g., sequence and ligase independent cloning or SLIC), or USER enzyme mix (e.g., USER friendly DNA recombination or USERec). Additional examples of circularization methods include, but are not limited to, circular polymerase extension cloning (CPEC) and seamless ligation cloning extract (SLiCE) assembly. Alternatively, these two ends can be joined by overlapping PCR. A variety of ligases can be used for ligation, for example, including but not limited to, T4 DNA ligase, T4 RNA ligase, E. coli DNA ligase.

The nucleic acid fragment used to construct the nucleic acid molecule encoding a TCR chain or portion thereof can be synthesized chemically. For example, the nucleic acid fragment can be pre-synthesized by chip-based synthesis. In some cases, the nucleic acid fragment synthesized can be equal to or greater than about 10, 20, 30, 40, 50, 60, 70, 80, 90, 100, 150, 200, 250, 300, 350, 400, or more nucleotides in length. In some cases, the nucleic acid fragment synthesized by can be equal to or less than about 500, 450, 400, 350, 300, 250, 200, 150, 100, 90, 80, 70, 60, 50, 40, 30, 20, or 10 nucleotides in length.

The two nucleic acid sequences encoding two peptide chains of a TCR can be constructed in several orientations, for example, head-to-head, head-to-tail, and tail-to-tail. As described herein, “head” refers to “5′ end” of a sense nucleic acid strand and “tail” refers to “3′ end” of a sense nucleic acid strand. In some cases, the orientation is head-to-tail, the order of the paired nucleic acid sequences encoding a TCR (e.g., TRA followed by TRB, or TRB followed by TRA) can be controlled.

Any nucleic acid molecule described herein can be a double-stranded nucleic acid molecule or single-stranded nucleic acid molecule. In some cases, a nucleic acid molecule may comprise a double-stranded region and a single-stranded region. For example, the nucleic acid molecule having a connector sequence or anti-connector sequence may be a double-stranded nucleic acid molecule having the connector sequence or anti-connector sequence region as a single-stranded region (e.g., an overhang or sticky end). The overhang can be at least 1, 2, 3, 4, 5, 6, 7, 8, 9, 10, or more nucleotides long. The overhang can be at 5′ end or 3′ end of a nucleic acid molecule.

Any nucleic acid molecule describe herein can comprise one or more modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs can be compatible with natural and mutant polymerases for de novo and/or amplification synthesis.

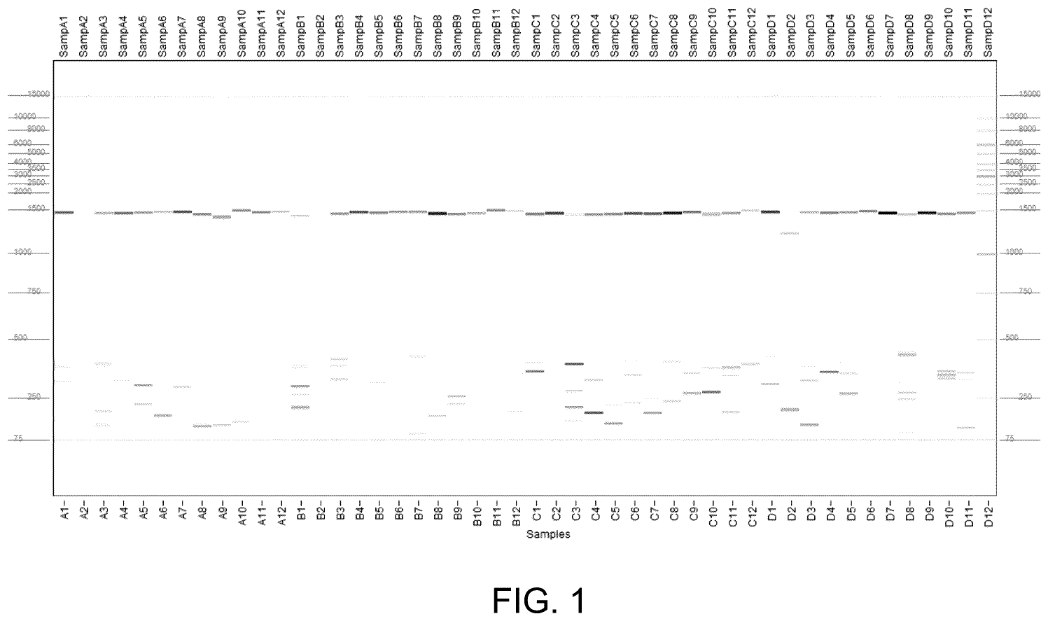

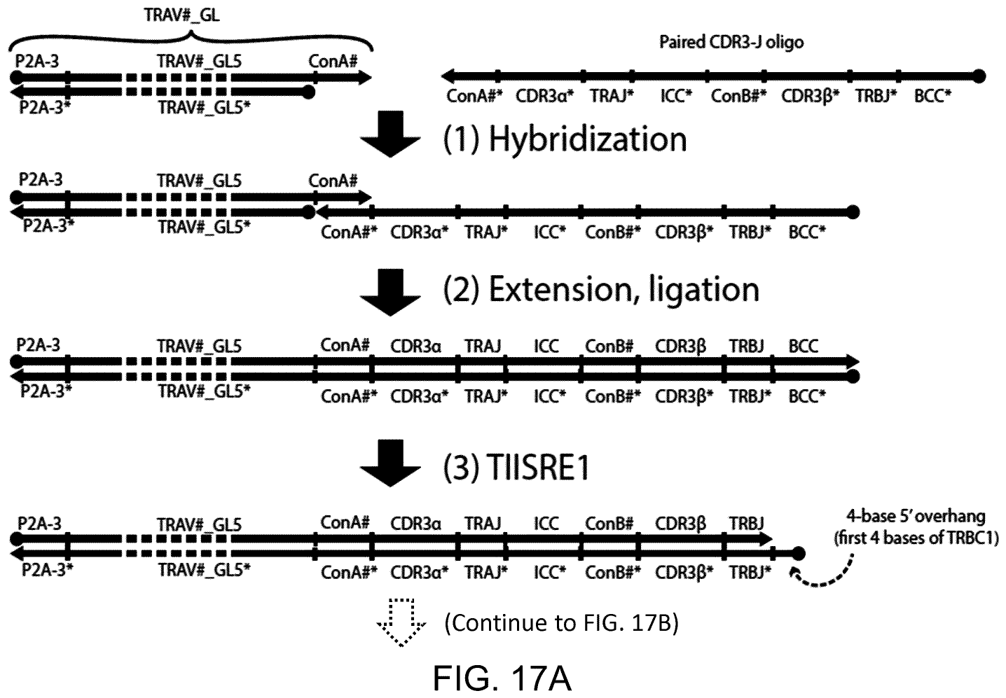

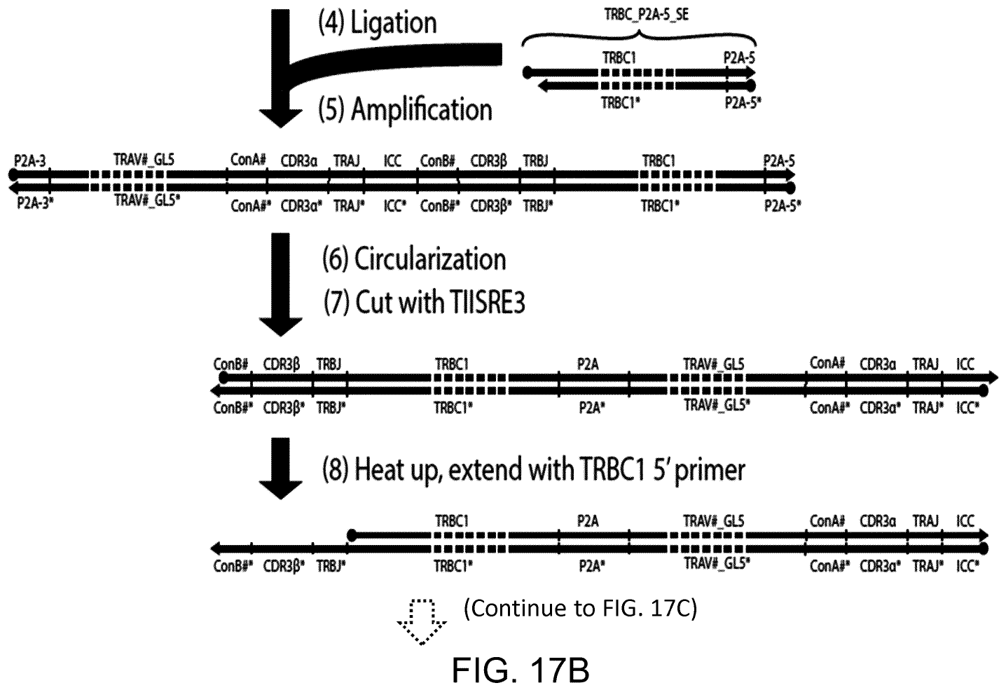

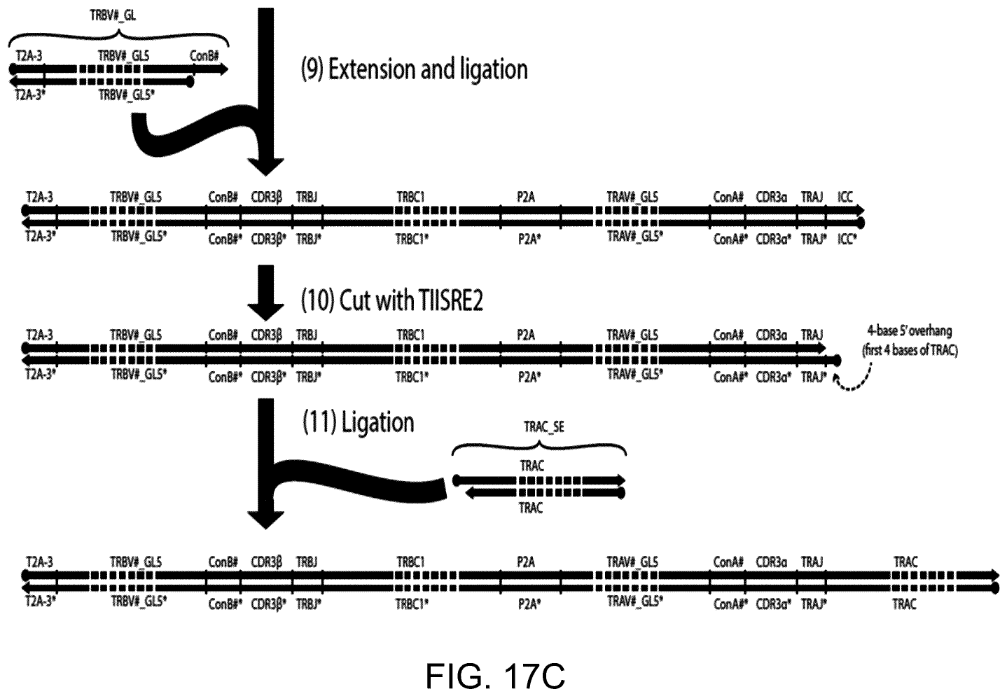

An example workflow of constructing nucleic acid molecules encoding TCRs is shown in FIGS. 17A-17C. A plurality of cognate pairs of TCRs can be pre-determined using various existing methods (e.g., single cell barcoding and sequencing) prior to using the methods described herein to construct the nucleic acid molecules encoding TCRs. Various sequencing methods can be used to determine sequences of paired TCR chains, for example, Sanger sequencing, high-throughput sequencing, sequencing-by-synthesis, single-molecule sequencing, sequencing-by-ligation, RNA-Seq (Illumina), Next generation sequencing, Digital Gene Expression (Helicos), Clonal Single MicroArray (Solexa), shotgun sequencing, Maxim-Gilbert sequencing, or massively-parallel sequencing. The paired sequences from the sequencing library can serve as reference sequences for the cognate pairs of TCR chains such that one can know which CDR3 is paired with which V gene through specific interactions between a connector sequence and an anti-connector sequence. A plurality of nucleic acid molecules encoding different TCRs can be constructed in a bulk using the methods described herein, but the construction of one molecule is shown in FIGS. 1A-1C as an example. A first nucleic acid molecule comprising a sequence encoding a first CDR3 (e.g., CDR3a) and a second CDR3 (e.g., CDR3β) can be contacted with a second nucleic acid molecule comprising a sequence derived from a first TCR V gene (e.g., TRAV). The connector sequence (e.g., ConA #*) of the first nucleic acid molecule can hybridize with the anti-connector sequence (ConA #) of the second nucleic acid molecule to link the two nucleic acid molecules. Extension and ligation can be performed to generate a third nucleic acid molecule comprising the sequence derived from the first TCR V gene and the sequence encoding the first CDR3 and the second CDR3. Next, a restriction enzyme (e.g., TIISRE1 of FIG. 17A) can be used to generate an overhang (or sticky end) of the third nucleic acid molecule. Next, the third nucleic acid molecule can be contacted with a fourth nucleic acid molecule comprising a sequence encoding a first constant region or constant domain (e.g., TRBC). The third nucleic acid molecule can then be ligated to the fourth nucleic acid molecule through the overhang to generate a fifth nucleic acid molecule comprising the sequence derived from the first TCR V gene, the sequence encoding the first CDR3 and the second CDR3, and the sequence encoding the first constant region. The fifth nucleic acid molecule can be circularized and cut with a restriction enzyme (e.g., TIISRE3) to expose an internal connector sequence (e.g., ICC). Next, the fifth nucleic acid molecule can be contacted with a sixth nucleic acid molecule comprising a sequence derived from a second TCR V gene (e.g., TRBV). The fifth nucleic acid molecule can be ligated to the sixth nucleic acid molecule through the interaction between a connector sequence and an anti-connector sequence. Next, the sixth nucleic acid molecule can be cut by a restriction enzyme (e.g., TIISRE2) to generate an overhang. Next, the sixth nucleic acid molecule can be contacted with a seventh nucleic acid molecule comprising a sequence encoding a second constant region or constant domain (e.g., TRAC). The sixth nucleic acid molecule and the seventh nucleic acid molecule can be ligated to form an eighth nucleic acid molecule comprising all regions encoding paired TCR chains. The eighth nucleic acid molecule can be further constructed into an expression vector for TCR chain expression in a host cell. It should be understood that the nucleic acid fragment comprising the sequence derived from a TCR V gene may be single-stranded and in such case, the 3′ end of the connector sequence of the nucleic acid fragment encoding the CDR3 can be extended upon hybridizing with the anti-connector sequence.

The methods described herein can be used to generate a pool of individual TCR chains, for example, a pool of TCR alpha chains or TCR beta chains.

The methods for generating a plurality of nucleic acid molecules described herein can comprise providing a first plurality of nucleic acid molecules (or nucleic acid fragments). A nucleic acid molecule of the first plurality of nucleic acid molecules can comprise a sequence encoding a first CDR3 of a first T-cell receptor (TCR) chain and a second CDR3 of a second TCR chain. The first CDR3 and the second CDR3 can be from a cognate pair of TCR chains. Next, a second plurality of nucleic acid molecules can be provided. A nucleic acid molecule of the second plurality of nucleic acid molecules can comprise a sequence derived from a TCR V gene. The nucleic acid molecule may not comprise a sequence encoding a constant domain. Next, the first plurality of nucleic acid molecules and the second plurality of nucleic acid molecules can be contacted. The nucleic acid molecule of the first plurality of nucleic acid molecules can link with the nucleic acid molecule of the second plurality of nucleic acid molecules to form a nucleic acid molecule comprising the sequence encoding the first CDR3 and the second CDR3 and the sequence derived from the TCR V gene. The sequence encoding the first CDR3 and the second CDR3 and the TCR V gene can be derived from the cognate pair of TCR chains.

The method for generating a plurality of nucleic acid molecules, each nucleic acid molecule of the plurality encoding a T-cell receptor (TCR) chain or region thereof, can comprise contacting a first plurality of nucleic acid molecules and a second plurality of nucleic acid molecules to generate a third plurality of nucleic acid molecules comprising at least two different nucleic acid molecules. Each of the at least two different nucleic acid molecules can have a different sequence encoding a different TCR chain or region thereof. The at least two different nucleic acid molecules can be generated in a same compartment. In some cases, at least about 5, 10, 20, 50, 100, 200, 300, 400, 500, 1,000, 1,500, 2,000, 2,500, 3,000, 3,500, 4,000, 5,000, 6,000, 7,000, 8,000, 9,000, 10,000, 12,000, 15,000, 20,000, 100,000, 1,000,000, 10,000,000, or more different sequences encoding different TCRs can be generated in the same compartment.

The method for generating a plurality of nucleic acid molecules described herein can comprise providing a first plurality of nucleic acid molecules. A nucleic acid molecule of the first plurality of nucleic acid molecules can comprise a sequence encoding a first CDR3 of a first T-cell receptor (TCR) chain and a second CDR3 of a second TCR chain. The first CDR3 and the second CDR3 can be from a cognate pair of TCR chains. Next, a second plurality of nucleic acid molecules can be provided. A nucleic acid molecule of the second plurality of nucleic acid molecules can comprise a sequence derived from a TCR V gene. Next, the first plurality of nucleic acid molecules and the second plurality of nucleic acid molecules can be contacted. The nucleic acid molecule of the first plurality of nucleic acid molecules can link with the nucleic acid molecule of the second plurality of nucleic acid molecules to form a linear nucleic acid molecule comprising the sequence encoding the first CDR3 and the second CDR3 and the sequence derived from the TCR V gene. The sequence encoding the first CDR3 and the second CDR3 and the TCR V gene can be derived from the cognate pair of TCR chains.

The method for generating a plurality of nucleic acid molecules can comprise providing a first plurality of nucleic acid molecules. A nucleic acid molecule of the first plurality of nucleic acid molecules can comprise (i) a synthetic sequence encoding a first CDR3 of a first T-cell receptor (TCR) chain and a second CDR3 of a second TCR chain and (ii) a synthetic sequence encoding a third CDR3 of a third T-cell receptor (TCR) chain and a fourth CDR3 of a fourth TCR chain. The first CDR3 and the second CDR3 can be from a first cognate pair of TCR chains and the third CDR3 and the fourth CDR3 can be from a second cognate pair of TCR chains. Next, a second plurality of nucleic acid molecules can be provided. A nucleic acid molecule of the second plurality of nucleic acid molecules can comprise a sequence derived from a TCR V gene. Next, the first plurality of nucleic acid molecules and the second plurality of nucleic acid molecules can be contacted. The nucleic acid molecule of the first plurality of nucleic acid molecules can link with the nucleic acid molecule of the second plurality of nucleic acid molecules to form a nucleic acid molecule comprising the sequence encoding the first CDR3 and the second CDR3 and the sequence derived from the TCR V gene. The sequence encoding the first CDR3 and the second CDR3 and the TCR V gene can be derived from the cognate pair of TCR chains.

The method for generating a nucleic acid molecule encoding a T-cell receptor (TCR) chain or portion thereof can comprise providing at least one nucleic acid molecule comprising a sequence encoding a CDR3 of a TCR chain. Next, a plurality of nucleic acid molecules can be provided. Each nucleic acid molecule of the plurality can comprise a sequence derived from a TCR V gene. The plurality of nucleic acid molecules can comprise at least two different sequences derived from at least two different TCR V genes. In some cases, the plurality of nucleic acid molecules can comprise at least 2, 5, 10, 15, 20, 25, 30, 35, 40 or more different sequences derived from at least 2, 5, 10, 15, 20, 25, 30, 35, 40 or more different TCR V genes. Next, the at least one nucleic acid molecule comprising a sequence encoding a CDR3 of a TCR chain can be contacted to the plurality of nucleic acid molecules, each comprising a sequence derived from a TCR V gene, in a same compartment. The at least one nucleic acid molecule comprising a sequence encoding a CDR3 of a TCR chain can be capable of linking to a nucleic acid molecule of the plurality of nucleic acid molecules to generate a third nucleic acid molecule comprising the sequence encoding the CDR3 and a sequence derived from one of the at least two different TCR V genes, thereby generating the nucleic acid molecule encoding the TCR chain or portion thereof.

The composition described herein that can be used for the methods described herein can comprise a first plurality of nucleic acid molecules. Each nucleic acid molecule of the first plurality of nucleic acid molecules can comprise a sequence encoding a first CDR3 of a first T-cell receptor (TCR) chain and a second CDR3 of a second TCR chain. The first CDR3 and the second CDR3 can be from a cognate pair of TCR chains. The composition can further comprise a second plurality of nucleic acid molecules. Each nucleic acid molecule of the second plurality of nucleic acid molecules can comprise a sequence derived from a TCR V gene. Each nucleic acid molecule of the second plurality of nucleic acid molecules may not comprise a sequence encoding the first CDR3 and the second CDR3. In this composition, (i) each nucleic acid molecule of the first plurality of nucleic acid molecules can comprise a sequence encoding a different first CDR3 and/or second CDR3, and/or (ii) each nucleic acid molecule of the second plurality of nucleic acid molecules comprises a sequence derived from a different TCR V gene.

The composition described herein that can be used for the methods described herein can comprise a plurality of nucleic acid molecules. Each nucleic acid molecule of the plurality of nucleic acid molecules can comprise a sequence derived from a T-cell receptor (TCR) V gene. The plurality of nucleic acid molecules can comprise a first nucleic acid molecule having a first connector sequence and a second nucleic acid molecule having a second connector sequence. The first connector sequence can be different from the second connector sequence.

The composition described herein that can be used for the methods described herein can comprise a plurality of nucleic acid molecules. Each nucleic acid molecule of the plurality of nucleic acid molecules can encode a CDR3 of a T-cell receptor (TCR) chain. A first nucleic acid molecule of the plurality can comprise a first connector sequence and a second nucleic acid molecule of the plurality can comprise a second connector sequence. The first connector sequence can be different from the second connector sequence.

The composition described herein that can be used for the methods described herein can comprise a plurality of nucleic acid molecules. Each nucleic acid molecule of the plurality can comprise a sequence encoding at least ten amino acids of a T-cell receptor (TCR) chain. A first nucleic acid molecule of the plurality can comprise a first connector sequence and a second nucleic acid molecule of the plurality can comprise a second connector sequence. The first connector sequence can be different from the second connector sequence. The first connector sequence or the second connector sequence can encode a portion of a TCR chain. The first connector sequence or the second connector sequence can be in frame with the sequence encoding at least ten amino acids of a TCR chain.

The composition described herein that can be used for the methods described herein can comprise a plurality of nucleic acid molecules. Each nucleic acid molecule of the plurality of nucleic acid molecules can comprise a sequence derived from a T-cell receptor (TCR) V gene and may not comprise a CDR3 sequence. A first nucleic acid molecule of the plurality can comprise a first anti-connector sequence and a second nucleic acid molecule of the plurality can comprise a second anti-connector sequence. The first anti-connector sequence can be different from the second anti-connector sequence. The sequence derived from a TCR V gene of the first nucleic acid molecule and the second nucleic acid molecule can be derived from a different TCR V gene. The composition can further comprise at least one nucleic acid molecule comprising a sequence encoding a CDR3 of a TCR chain. The at least one nucleic acid molecule can further comprise a first connector sequence complementary to the first anti-connector sequence.

The present disclosure provides compositions and methods for the assembly or synthesis of a TCR library comprising a plurality of TCRs. In some cases, it may be useful to isolate or purify a particular TCR sequence (e.g., a TCR-of-interest) from the TCR library for further characterization or manipulation. To do this, a barcode can be included in the nucleic acid molecules or fragments used to construct the sequence encoding a TCR or portion thereof. In some cases, a nucleic acid fragment comprising a sequence encoding a CDR3 comprises a barcode. In some cases, a nucleic acid fragment comprising a sequence encoding a first CDR3 of a first TCR chain and a second CDR3 of a second TCR chain comprises a barcode. For example, a CDR3-J oligo or paired CDR3-J oligo can comprise a barcode. The connector sequence (or in some cases, the anti-connector sequence) can comprise a barcode. The inter-chain connector (or ICC) of the CDR3-J oligo can comprise a barcode. The barcode can be a primer binding site, e.g., a TCR-specific primer-binding site or DOPBS.

For example, each sequence encoding a unique paired CDR3-J in the paired CDR3-J oligo pool (e.g., FIG. 17A) can comprise a unique barcode (or a unique DOPBS). The sequences of the DOPBSes can be arbitrarily designed. The sequences of the DOPBSes can be designed to avoid common pitfalls such as unwanted secondary structures, restriction sites, similarity with other sequences in the TCR genes, or similarities between primer-binding sites. The barcode (or DOPBS) can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more nucleotides long. The DOPBS can be an additional sequence included in each sequence of the paired CDR3-J pool. The DOPBS can be a sequence already included in each sequence of the paired CDR3-J pool. For example, a connector sequence or portion thereof can be used as a DOPBS. The sequences listed in Table 3 can be used as DOPBSes. The product of Step (9) of FIG. 17C can be used as the template in a dial-out PCR using a forward primer corresponding to T2A-3, and a reverse primer corresponding to the DOPBS associated with the TCR-of-interest. The PCR product can be subject to Steps (10) and (11) of FIG. 17C. The final product can contain primarily the TCR-of-interest.

### Expression of TCRs

The present disclosure provides a sequence encoding an exogenous TCR. For example, the methods provided herein, in various embodiments, comprise delivering a plurality of polynucleotide sequences or derivative thereof encoding natively paired TCRs into a plurality of recipient cells to produce the plurality of synthetic TILs. The plurality of polynucleotide sequences or derivative thereof can be synthesized using the methods described herein. The derivative of a polynucleotide sequence can be a copy (e.g., amplified product or a chemically synthesized copy) of the polynucleotide sequence. Each polynucleotide sequence of the plurality of polynucleotide sequence can encode a natively paired TCR. The plurality of polynucleotide sequences or derivative thereof can be delivered in a pool or a mixture by contacting the pool or mixture containing the plurality of polynucleotide sequences or derivative thereof with the plurality of recipient cells.

The exogenous TCR of the present disclosure can be obtained from a subject (e.g., isolated from a sample from a subject) and can be referred to as subject-derived or subject-specific TCR. The cell can comprise a TCR alpha chain and a TCR beta chain or a TCR gamma chain and a TCR delta chain. Accordingly, the present disclosure provides a nucleic acid encoding a TCR alpha chain (or a TCR gamma chain), and a nucleic acid encoding a TCR beta chain (or a TCR delta chain). In some embodiments, the nucleic acid encoding a TCR alpha chain (or a TCR gamma chain) is separate from the nucleic acid encoding a TCR beta chain (or a TCR delta chain). In some embodiments, the nucleic acid encoding a TCR alpha chain (or a TCR gamma chain), and the nucleic acid encoding a TCR beta chain (or a TCR delta chain), resides within the same nucleic acid.

A polynucleotide sequence encoding a subject-derived or subject-specific TCR may be delivered into a cell for expression. A polynucleotide sequence encoding a subject-derived or subject-specific TCR may be delivered into a cell as a linear or circular nucleic acid molecule to generate the modified cell. In some cases, the polynucleotide can be delivered (e.g., electroporated, transfected, transduced, or transformed) into a cell by electroporation. In some cases, the polynucleotide can be delivered into a cell by a carrier such as a cationic polymer. In some cases, a vector comprising a sequence encoding a subject-derived or subject-specific TCR can be delivered into a cell. In some cases, the subject-derived or subject-specific TCR can be expressed in the cell. The TCR can be expressed from a vector (or an expression vector) such as plasmid, transposon (e.g., Sleeping Beauty, Piggy Bac), and a viral vector (e.g., adenoviral vector, adeno-associated virus (AAV) vector, retroviral vector and lentiviral vector). Additional examples of a vector include a shuttle vector, a phagemide, a cosmid and an expression vector. Non-limiting examples of plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some cases, a vector is a nucleic acid molecule as introduced into a cell, thereby producing a transformed cell (e.g., an engineered cell). A vector may include nucleic acid sequences that permit it to replicate in a cell, such as an origin of replication. A vector may also include one or more selectable marker genes and other genetic elements. A vector can be an expression vector that includes a paired TCR-encoding polynucleotide according to the present disclosure operably linked to sequences allowing for the expression of the TCR. A vector can be a viral or a non-viral vector, such a retroviral vector (including lentiviral vectors), adenoviral vectors including replication competent, replication deficient and gutless forms thereof, AAV vectors, simian virus 40 (SV-40) vectors, bovine papilloma vectors, Epstein-Barr vectors, herpes vectors, vaccinia vectors, Moloney murine leukemia vectors, Harvey murine sarcoma virus vectors, murine mammary tumor virus vectors, Rous sarcoma virus vectors and non-viral plasmids.

In some cases, the vector is a AAV vector (e.g., AAV6 vector). Besides the sequence encoding the exogenous TCR, the AAV vector can comprise a pair of homology arms (HAs) flanking the sequence encoding the exogenous TCR. The HAs of the AAV vector can further be flanked by upstream and downstream Inverted terminal repeats (ITRs).

In some cases, the vector is a lentiviral vector. The recipient cells (e.g., the patient's peripheral T cells) can be delivered with a mixture of lentiviral particles at low multiplicity of infection (MOI). The dilution factor of the lentiviral particles can be determined first so that the functional MOI (e.g., the fraction of T cells that expresses one or more exogenous TCRs) is from about 5% to 15%. In such cases, the recipient cells can express only a single copy of the sequence encoding a TCR.

In some cases, the vector is a self-amplifying RNA replicon, also referred to as self-replicating (m) RNA, self-replication (m) RNA, self-amplifying (m) RNA, or RNA replicon. The self-amplifying RNA replicon is an RNA that can replicate itself. In some embodiments, the self-amplifying RNA replicon can replicate itself inside of a cell. In some embodiments, the self-amplifying RNA replicon encodes an RNA polymerase and a molecule of interest. The RNA polymerase may be a RNA-dependent RNA polymerase (RDRP or RdRp). The self-amplifying RNA replicon may also encode a protease or an RNA capping enzyme. In some embodiments, the self-amplifying RNA replicon vector is of or derived from the Togaviridae family of viruses known as alphaviruses which can include Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, South African Arbovirus No. 86, Semliki Forest virus, Middelburg virus, Chikungunya virus, Onyong-nyong virus, Ross River virus, Barmah Forest Virus, Getah Virus, Sagiyama virus, Bebaru virus, Mayaro virus, Una virus, Aura virus, Whataroa virus, Babanki virus, Kyzylagach virus, Highlands J Virus, Fort Morgan virus, Ndumu virus, Buggy Creek virus, and any other virus classified by the International Committee on Taxonomy of Viruses (ICTV) as an alphavirus. In some embodiments, the self-amplifying RNA replicon is or contains parts from an attenuated form of the alphavirus, such as the VEE TC-83 vaccine strain. In some embodiments, the self-amplifying RNA replicon vector is an attenuated form of the virus that allows for expression of the molecules of interests without cytopathic or apoptotic effects to the cell. In some embodiments, the self-amplifying RNA replicon vector has been engineered or selected in vitro, in vivo, ex vivo, or in silica for a specific function (e.g., prolonged or increased TCR expression) in the host cell, target cell, or organism. For example, a population of host cells harboring different variants of the self-amplifying RNA replicon can be selected based on the expression level of one or more molecules of interested (encoded in the self-amplifying RNA replicon or in the host genome) at different time point. In some embodiments, the selected or engineered self-amplifying RNA replicon has been modified to reduce the type I interferon response, the innate antiviral response, or the adaptive immune response from the host cell or organism which results in the RNA replicon's protein expression persisting longer or expressing at higher levels in the host cell, target cell, or organism. In some embodiments, this optimized self-amplifying RNA replicon sequence is obtained from an individual cell or population of cells with the desired phenotypic trait (e.g., higher or more sustained expression of the molecules of interest, or reduced innate antiviral immune response against the vector compared to the wildtype strains or the vaccine strains). In some embodiments, the cells harboring the desired or selected self-amplifying RNA replicon sequence are obtained from a subject (e.g., a human or an animal) with beneficial response characteristics (e.g., an elite responder or subject in complete remission) after being treated with a therapeutic agent comprising a self-amplifying RNA replicon. In some embodiments, the self-amplifying RNA replicon vector can express additional agents. In some embodiments, the additional agents include cytokines such as IL-2, IL-12, IL-15, IL-10, GM-CSF, TNF alpha, granzyme B, or a combination thereof. In some embodiments, the additional agent is capable of modulating the expression of the TCR, either by directly affecting the expression of the TCR or by modulating the host cell phenotype (e.g., inducing apoptosis or expansion). In some embodiments, the self-amplifying RNA replicon can contain one or more sub-genomic sequence(s) to produce one or more sub-genomic polynucleotide(s). In some embodiments, the sub-genomic polynucleotides act as functional mRNA molecules for translation by the cellular translation machinery. A sub-genomic polynucleotide can be produced via the function of a defined sequence element (e.g., a sub-genomic promoter or SGP) on the self-amplifying RNA replicon that directs a polymerase to produce the sub-genomic polynucleotide from a sub-genomic sequence. In some embodiments, the SGP is recognized by an RNA-dependent RNA polymerase (RDRP or RdRp). In some embodiments, multiple SGP sequences are present on a single self-amplifying RNA replicon and can be located upstream of sub-genomic sequence encoding for a TCR, a constituent of the TCR, or an additional agent. In some embodiments, the nucleotide length or composition of the SGP sequence can be modified to alter the expression characteristics of the sub-genomic polynucleotide. In some embodiments, non-identical SGP sequences are located on the self-amplifying RNA replicon such that the ratios of the corresponding sub-genomic polynucleotides are different from instances where the SGP sequences are identical. In some embodiments, non-identical SGP sequences direct the production of a TCR and an additional agent (e.g., a cytokine) such that they are produced at a ratio relative to one another that leads to increased expression of the TCR, increased or faster expansion of the target cell without cytotoxic effects to the target cell or host, or dampens the innate or adaptive immune response against the RNA replicon. In some embodiments, the location of the sub-genomic sequences and SGP sequences relative to one another and the genomic sequence itself can be used to alter the ratio of sub-genomic polynucleotides relative to one another. In some embodiments, the SGP and sub-genomic sequence encoding the TCR can be located downstream of an SGP and sub-genomic region encoding the additional agent such that the expression of the TCR is substantially increased relative to the additional agent. In some embodiments, the RNA replicon or SGP has been selected or engineered to express an optimal amount of the cytokine such that the cytokine promotes the expansion of the T cell or augments the therapeutic effect of the TCR but does not cause severe side effects such as cytokine release syndrome, cytokine storm, or neurological toxicity.

In some embodiments, provided herein is a vector comprising a paired TCR-encoding polynucleotide encoding a TCRα chain and a TCRβ chain. In some embodiments, provided herein is a vector comprising a paired TCR-encoding polynucleotide encoding a TCRγ chain and a TCR8 chain. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccina virus, hepatitis B virus, human papillomavirus or a pseudotype thereof. In some embodiments, the vector is a non-viral vector. In some embodiments, the non-viral vector can be formulated into a nanoparticle, a cationic lipid, a cationic polymer, a metallic nanopolymer, a nanorod, a liposome, a micelle, a microbubble, a cell-penetrating peptide, or a liposphere.

The expression of the two TCR chains can be driven by two promoters or by one promoter. In some cases, two promoters are used. In some cases, the two promoters, along with their respective protein-coding sequences for the two chains, can be arranged in a head-to-head, a head-to-tail, or a tail-to-tail orientation. In some cases, one promoter is used. The two protein-coding sequences can be linked, optionally in frame, such that one promoter can be used to express both chains. And in such cases, the two protein-coding sequences can be arranged in a head-to-tail orientation and can be connected with ribosome binding site (e.g., internal ribosomal binding site or IRES), protease cleavage site, or self-processing cleavage site (such as a sequence encoding a 2A peptide) to facilitate bicistronic expression. In some cases, the two chains can be linked with peptide linkers so that the two chains can be expressed as a single-chain polypeptide. Each expressed chain may contain the full variable domain sequence including the rearranged V(D)J gene. Each expressed chain may contain the full variable domain sequence including CDR1, CDR2, and CDR3. Each expressed chain may contain the full variable domain sequence including FR1, CDR1, FR2, CDR2, FR3, and CDR3. In some cases, each expressed chain may further contain a constant domain sequence.

To create expression vectors, additional sequences may be added to the sequence encoding the gene of interest such as the TCR. These additional sequences can include vector backbone (e.g., elements for the vector's replication in target cell or in temporary host such as E. coli), promoters, IRES, sequence encoding the self-cleaving peptide, terminators, accessory genes (such as payloads), as well as partial sequences of the paired TCR-encoding polynucleotides (such as part of the sequences encoding the constant domains).

Protease cleavage sites include, but are not limited to, an enterokinase cleavage site: (Asp) 4Lys; a factor Xa cleavage site: Ile-Glu-Gly-Arg; a thrombin cleavage site, e.g., Leu-Val-Pro-Arg-Gly-Ser; a renin cleavage site, e.g., His-Pro-Phe-His-Leu-Val-Ile-His; a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picornavirus, a Hepatitis A virus 3C cleavage site, human rhinovirus 2A protease cleavage site, a picornavirus 3 protease cleavage site; and a caspase protease cleavage site, e.g., DEVD recognized and cleaved by activated caspase-3, where cleavage occurs after the second aspartic acid residue. In some embodiments, the present disclosure provides an expression vector comprising a protease cleavage site, wherein the protease cleavage site comprises a cellular protease cleavage site or a viral protease cleavage site. In some embodiments, the first protein cleavage site comprises a site recognized by furin; VP4 of IPNV; tobacco etch virus (TEV) protease; 3C protease of rhinovirus; PC5/6 protease; PACE protease, LPC/PC7 protease; enterokinase; Factor Xa protease; thrombin; genenase I; MMP protease; Nuclear inclusion protein a (N1a) of turnip mosaic potyvirus; NS2B/NS3 of Dengue type 4 flaviviruses, NS3 protease of yellow fever virus; ORF V of cauliflower mosaic virus; KEX2 protease; CB2; or 2A. In some embodiments, the protein cleavage site is a viral internally cleavable signal peptide cleavage site. In some embodiments, the viral internally cleavable signal peptide cleavage site comprises a site from influenza C virus, hepatitis C virus, hantavirus, flavivirus, or rubella virus.

A suitable IRES element to include in the vector of the present disclosure can comprise an RNA sequence capable of engaging a eukaryotic ribosome. In some embodiments, an IRES element of the present disclosure is at least about 250 base pairs, at least about 350 base pairs, or at least about 500 base pairs. An IRES element of the present disclosure can be derived from the DNA of an organism including, but not limited to, a virus, a mammal, and a Drosophila. In some cases, a viral DNA from which an IRES element is derived includes, but is not limited to, picornavirus complementary DNA (cDNA), encephalomyocarditis virus (EMCV) cDNA and poliovirus cDNA. Examples of mammalian DNA from which an IRES element is derived includes, but is not limited to, DNA encoding immunoglobulin heavy chain binding protein (BiP) and DNA encoding basic fibroblast growth factor (bFGF). An example of Drosophila DNA from which an IRES element is derived includes, but is not limited to, an Antennapedia gene from Drosophila melanogaster. Addition examples of poliovirus IRES elements include, for instance, poliovirus IRES, encephalomyocarditis virus IRES, or hepatitis A virus IRES. Examples of flaviviral IRES elements include hepatitis C virus IRES, GB virus B IRES, or a pestivirus IRES, including but not limited to bovine viral diarrhea virus IRES or classical swine fever virus IRES.

Examples of self-processing cleavage sites include, but are not limited to, an intein sequence; modified intein; hedgehog sequence; other hog-family sequence; a 2A sequence, e.g., a 2A sequence derived from Foot and Mouth Disease Virus (FMDV); and variations thereof for each.

A vector for recombinant gene expression (e.g., TCR expression) may include any number of promoters, wherein the promoter is constitutive, regulatable or inducible, cell type specific, tissue-specific, or species specific. Further examples include tetracycline-responsive promoters. The vector can be a replicon adapted to the host cell in which the TCR is to be expressed, and it can comprise a replicon functional in a bacterial cell as well, for example, Escherichia coli. The promoter can be constitutive or inducible, where induction is associated with the specific cell type or a specific level of maturation, for example. Alternatively, a number of viral promoters can be suitable. Examples of promoters include the β-actin promoter, SV40 early and late promoters, immunoglobulin promoter, human cytomegalovirus promoter, retrovirus promoter, elongation factor 1A (EF-1A) promoter, phosphoglycerate kinase (PGK) promoter, and the Friend spleen focus-forming virus promoter. The promoters may or may not be associated with enhancers, wherein the enhancers may be naturally associated with the particular promoter or associated with a different promoter.

Promoters used in mammalian cells can be constitutive (Herpes virus TK promoter; SV40 early promoter; Rous sarcoma virus promoter; cytomegalovirus promoter; mouse mammary tumor virus promoter) or regulated (metallothionein promoter, for example). Vectors can be based on viruses that infect particular mammalian cells, e.g., retroviruses, vaccinia and adenoviruses and their derivatives. Promoters can include, without limitation, cytomegalovirus, adenovirus late, and the vaccinia 7.5K promoters. Enolase is an example of a constitutive yeast promoter, and alcohol dehydrogenase is an example of regulated promoter. The selection of the specific promoters, transcription termination sequences and other optional sequences, such as sequences encoding tissue specific sequences, can be determined by the type of cell in which expression is carried out.

In some cases, the vector for recombinant gene expression (e.g., TCR expression) does not comprise a promoter. In such cases, the gene of interest can be expressed under the control of endogenous promoter of the genomic locus. For example, the vector may comprise homology arms (HAs) flanking the gene of interest to facilitate inserting the gene of interest into a genomic locus through homologous recombination after cutting the genomic locus using a selected gene editing method. The gene of interest can be inserted in-frame into the genomic locus and controlled by the endogenous promoter for expression.

### Gene Delivery

Various methods of delivering (or introducing) and expressing genes or genetic materials (e.g., nucleic acid molecules encoding proteins of interest) into a cell can be used. The proteins of interest described herein can be exogenous TCR chains. In the context of an expression vector, the vector can be readily introduced into a host cell, e.g., T cell. For example, the expression vector can be transferred into a host cell by physical, chemical, or biological methods. Physical methods for introducing a nucleic acid molecule into a host cell include, but are not limited to, calcium phosphate precipitation, lipofection, particle bombardment, microinjection, electroporation, and the like. Biological methods for introducing a nucleic acid molecule of interest into a host cell include, but are not limited to, the use of DNA and RNA vectors. Viral vectors such as retroviral vectors, lentiviral vectors, adenoviral vectors and adeno-associated viral vectors, can be used for delivering genes into mammalian cells, e.g., human cells. Chemical methods for introducing a nucleic acid molecule into a host cell can include colloidal dispersion systems, such as macromolecule complexes, nanocapsules, microspheres, beads, and lipid-based systems including oil-in-water emulsions, micelles, mixed micelles, and liposomes. An example colloidal system for use as a delivery vehicle in vitro and in vivo is a liposome (e.g., an artificial membrane vesicle). Other methods can include, but are not limited to, delivery of nucleic acids with targeted nanoparticles or other suitable sub-micron sized delivery system.

In the case where a non-viral delivery system is utilized, an example delivery vehicle is a liposome. The use of lipid formulations can be contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They can form spontaneously when phospholipids are suspended in an excess of aqueous solution. The lipid components can undergo self-rearrangement before the formation of closed structures and entrap water and dissolved solutes between the lipid bilayers. However, compositions that have different structures in solution than the normal vesicular structure are also encompassed. For example, the lipids may assume a micellar structure or merely exist as nonuniform aggregates of lipid molecules. Also contemplated herein can include lipofectamine-nucleic acid complexes.

### Modified Cells

Provided herein, in various embodiments, are modified cells expressing one or more exogenous cognate pairs of TCRs generated using the methods described herein. The modified cells can be engineered cells or synthetic cells. The modified cells can be genetically engineered cells. The modified cells can be synthetic tumor-infiltrating lymphocytes (TILs). In the present disclosure including figures, the synthetic TILs may be referred to as InfiniTIL or DisTIL. The synthetic TILs can be prepared by delivering and/or expressing the polynucleotides encoding cognate pairs of TCRs from TILs obtained from a subject into recipient cells (or host cells). The recipient cells can be T cells. In some cases, the T cells can be CD4+ T cells, CD8+ T cells, or CD4+/CD8+ T cells. In some cases, the T cells can be helper T cells, cytotoxic T cells, memory T cells, regulatory T cells, natural killer T cells, alpha beta T cells, or gamma delta T cells. The modified cell can be a modified T cell.

The recipient cells can be isolated from a subject (e.g., a subject having a tumor) to whom the modified cells will be administered. The recipient cells may be, in some cases, obtained from a healthy donor.

In some cases, the present disclosure provides a mixture of at least 2, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 10,000, 100,000 or more modified cells, each modified cell of the mixture exogenously expressing a polynucleotide sequence encoding a distinct cognate pair of a TCR alpha chain and a TCR beta chain. The polynucleotide sequence can be knocked into a genomic locus for monoallelic expression of the polynucleotide sequence. In some cases, an endogenous TCR of the modified cell can be knocked out. Each distinct cognate pair of a TCR alpha chain and a TCR beta chain can be subject-specific TCR or can be from a subject having a solid tumor lesion.

The recipient cells described herein can be of various cell types. The recipient cells can be T cells, B cells, NK cells, macrophages, neutrophils, granulocytes, eosinophils, red blood cells, platelets, stem cells, iPSCs, or mesenchymal stem cells. In addition, the recipient cell can be a cell line cell. The cell line can be tumorigenic or artificially immortalized cell line. Examples of cell lines include, but are not limited to, CHO-K1 cells, HEK293 cells, Caco2 cells, U2-OS cells, NIH 3T3 cells, NSO cells, SP2 cells, CHO-S cells, DG44 cells, K-562 cells, U-937 cells, MRC5 cells, IMR90 cells, Jurkat cells, HepG2 cells, HeLa cells, HT-1080 cells, HCT-116 cells, Hu-h7 cells, Huvec cells, and Molt 4 cells. The recipient cell can be an autologous T cell (e.g., patient-derived T cell) or an allogeneic T cell (e.g., donor T cell from a healthy subject). The recipient cell can be a genetically modified or engineered cell. In some cases, the recipient cell is autologous to the subject in need of treatment, e.g., cancer treatment. In other cases, the recipient cell is allogenic to the subject in need of treatment.

The recipient cells can be primary T cells isolated from a subject. The subject can be the same subject from whom the source T cells are obtained. The recipient cells can be primary T cells isolated from a blood sample. The recipient cells can be obtained from a peripheral blood mononuclear cell (PBMC) sample. The recipient cells can be peripheral T cells. The recipient cells can be obtained from a unit of blood collected from a subject using a variety of techniques, such as Ficoll™ separation. Cells from the circulating blood of an individual can be obtained by apheresis. The apheresis product may contain lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. In some cases, the cells can be washed with phosphate buffered saline (PBS). The wash solution may lack calcium or magnesium or other divalent cations. Initial activation steps in the absence of calcium can lead to magnified activation. A washing step may be accomplished by methods such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In an aspect, T cells are isolated from peripheral blood lymphocytes or tissues by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least or equal to about 1, 2, 3, 4, 5, or 6 hours. In yet another aspect, the time period is 10 to 24 hours. In an aspect, the incubation time period is about 24 hours. Longer incubation times may be used to isolate T cells in any situation where there are few T cells as compared to other cell types, such as in isolating tumor infiltrating lymphocytes (TILs) from tumor tissue or from immunocompromised individuals. Further, use of longer incubation times can increase the efficiency of capture of CD8+ T cells. Thus, by simply shortening or lengthening the time T cells are allowed to bind to the anti-CD3/anti-CD28 beads and/or by increasing or decreasing the ratio of beads to T cells, subpopulations of T cells can be selected for or against at culture initiation or at other time points during the process. Additionally, by increasing or decreasing the ratio of anti-CD3 and/or anti-CD28 antibodies on the beads or other surface, subpopulations of T cells can be selected for or against at culture initiation or at other desired time points. In some cases, multiple rounds of selection can be used. In certain aspects, the selection procedure can be performed and the “unselected” cells (cells that may not bind to the anti-CD3/anti-CD28 beads) can be used in the activation and expansion process. “Unselected” cells can also be subjected to further rounds of selection.

Enrichment of a T cell population by negative selection can be accomplished with a combination of antibodies directed to surface markers unique to the negatively selected cells. One example method is cell sorting and/or selection via negative magnetic immune adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be useful to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

The recipient cells can be obtained in a similar way as the source T cells. For example, the recipient cells can be TILs, e.g., tumor-infiltrating T cells (TITs). A TIL can be isolated from an organ afflicted with a cancer. One or more cells can be isolated from an organ with a cancer that can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. One or more TILs can be from a brain, heart, liver, skin, intestine, lung, kidney, eye, small bowel, or pancreas. TILs can be from a pancreas, kidney, eye, liver, small bowel, lung, or heart. The one or more cells can be pancreatic islet cells, for example, pancreatic β cells. In some cases, a TIL can be from a gastrointestinal cancer. A TIL culture can be prepared a number of ways. For example, a tumor can be trimmed from non-cancerous tissue or necrotic areas. A tumor can then be fragmented to about 2-3 mm in length. In some cases, a tumor can be fragmented from about 0.5 mm to about 5 mm in size, from about 1 mm to about 2 mm, from about 2 mm to about 3 mm, from about 3 mm to about 4 mm, or from about 4 mm to about 5 mm. Tumor fragments can then be cultured in vitro utilizing media and a cellular stimulating agent such as a cytokine. In some cases, IL-2 can be utilized to expand TILs from a tumor fragment. A concentration of IL-2 can be about 6000 IU/mL. A concentration of IL-2 can also be about 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL. Once TILs are expanded, they can be subject to in vitro assays to determine tumor reactivity. For example, TILs can be evaluated by FACs for CD3, CD4, CD8, and CD58 expression. TILs can also be subjected to cocultured, cytotoxicity, ELISA, or ELISPOT assays. In some cases, TIL cultures can be cryopreserved or undergo a rapid expansion. A cell, such as a TIL, can be isolated from a donor of a stage of development including, but not limited to, fetal, neonatal, young and adult.

The recipient cells can be TITs isolated from surgically removed tumor. For example, the tumor tissue can be cut up into 3-5 mm² fragments after trimming away fat and connective tissue and disaggregated in cold RPMI 1640 using gentle mechanical pulverization using a Seward Stomacher device (Fisher, Pittsburgh, PA). This process can rapidly produce a single cell suspension without enzymatic digestion. The cell suspension can be filtered through a 75 μm pore-size screen (BD Biosciences, San Jose CA) and washed in culture medium. A portion of the cells used for immediate staining and analysis by flow cytometry can be washed in culture medium and the cell suspension can be layered over a discontinuous 70% followed by a 100% Ficoll Isopaque gradient, and centrifuged to separate the tumor cells (70% interface) from the enriched TITs (100% interface). The enriched TITs can be then washed in D-PBS, 1% BSA and then processed. In some cases, the TITs from tumor samples can be expanded in TIT culture medium (TIL-CM) containing RPMI 1640 with Glutamax (Invitrogen), 10% human AB serum (Sigma, St. Louis, MI), 50 mM 2-mercaptoethanol (Invitrogen), 1 mM pyruvate, 1× Penicillin/Streptomycin (Invitrogen) using 3,000 IU/ml recombinant IL-2.

### Activation and Expansion

Whether prior to or after delivering the sequence encoding the exogenous TCRs to the recipient T cells, the cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

The T cells can be expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell. As non-limiting examples, T cell populations may be stimulated in vitro such as by contact with an anti-CD3 antibody or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. For example, the agents providing each signal may be in solution or coupled to a surface. The ratio of particles to cells may depend on particle size relative to the target cell. In further embodiments, the cells, such as T cells, are combined with agent-coated beads, the beads and the cells are subsequently separated, and then the cells are cultured. In an alternative embodiment, prior to culture, the agent-coated beads and cells are not separated but are cultured together. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFβ, and TNF-α or any other additives for the growth of cells. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. The target cells can be maintained under conditions necessary to support growth, for example, an appropriate temperature (e.g., 37° C.) and atmosphere (e.g., air plus 5% CO₂). T cells that have been exposed to varied stimulation times may exhibit different characteristics.

In another embodiment, the cells can be activated or expanded by co-culturing with tissue or cells. The cells used to activate T cells can be APC or artificial APC (aAPC). The APC can be professional APC such as dendritic cell, macrophage, or B cell. The APC can be a monocyte or monocyte-derived dendritic cell. An aAPC can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof.

In some cases, an aAPC may be used to activate and/or expand a cell population. In some cases, an artificial may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

In some cases, restimulation of cells can be performed with antigen and irradiated, histocompatible APCs, such as feeder PBMCs. In some cases, cells can be grown using non-specific mitogens such as PHA and allogenic feeder cells. Feeder PBMCs can be irradiated at 40 Gy. Feeder PBMCs can be irradiated from about 10 Gy to about 15 Gy, from about 15 Gy to about 20 Gy, from about 20 Gy to about 25 Gy, from about 25 Gy to about 30 Gy, from about 30 Gy to about 35 Gy, from about 35 Gy to about 40 Gy, from about 40 Gy to about 45 Gy, from about 45 Gy to about 50 Gy. In some cases, a control flask of irradiated feeder cells only can be stimulated with anti-CD3 and IL-2.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a poly(lactic-co-glycolic acid) (PLGA) microsphere, a nonspherical particle, a carbon nanotube bundle, a ellipsoid PLGA microparticle, a nanoworms, a fluidic lipid bilayer-containing system, a 2D-supported lipid bilayer (2D-SLBs), a liposome, a RAFTsomes/microdomain liposome, an SLB particle, or any combination thereof.

In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells.

In some cases, the use of aAPCs can be combined with exogenously introduced cytokines for T cell activation, expansion, or any combination. Cells can also be expanded in vivo, for example in the subject's blood after administration of genomically transplanted cells into a subject.

The cells (e.g., recipient cells) can also be expanded in vivo, for example in the subject's blood after administrating the cells into the subject.

### Gene Editing

The recipient cells disclosed herein can be delivered with sequences encoding exogenous cognate pairs of TCRs for expression. The sequences can be knocked into a genomic locus for expression. The knock-in can be site-specific knock-in. For example, a sequence encoding an exogenous cognate pair of TCR can be site-specifically knocked into a genomic locus for monoallelic expression. The genomic locus can be located on a sex chromosome. The genomic locus can be a TRAC locus. The knock-in can be performed by various gene editing methods described herein. For example, the knock-in can be performed by clustered regularly interspaced short palindromic repeats (CRISPR), transcription activator-like effector (TALE) nucleases (TALENs), meganucleases, zinc finger nuclease (ZFN), megaTAL nucleases method, or any combination thereof. A vector containing the sequence encoding the exogenous cognate pair of TCR can be delivered into the recipient cell together with the agents (e.g., nuclease such as Cas9, and/or sgRNA) for gene editing. The gene editing agents can introduce a site-specific cut on the genomic locus, and the sequence encoding the exogenous cognate pair of TCR can be inserted into the specific genomic locus via homologous recombination.

The recipient cell disclosed herein can be modified to inactivate one or more endogenous TCR chains. The endogenous TCR gene encoding a TCR alpha chain, a TCR beta chain, or a TCR alpha chain and a TCR beta chain can be inactivated in the modified cell (e.g., modified T cell) described herein. In some cases, the endogenous TCR gene encoding a TCR gamma chain, a TCR delta chain, or a TCR gamma chain and a TCR delta chain can be inactivated in the modified cell (e.g., modified T cell) described herein. The inactivation of the endogenous TCR gene within the recipient cell can be done prior to, concurrently, or after delivering the polynucleotide or sequence encoding the exogenous TCR. In some cases, the endogenous TCR gene within the recipient cell is inactivated concurrently with delivering the polynucleotide or sequencing encoding the exogenous TCR. In some cases, the endogenous TCR gene within the recipient cell is inactivated concurrently with site-specific knock-in of the sequence encoding the exogenous TCR into a genomic locus. For example, the recipient cell can be delivered with a vector containing the sequence encoding the exogenous TCR, and a nuclease protein or a nucleic acid sequence encoding a nuclease protein that targets the endogenous gene encoding a TCR alpha chain, a TCR beta chain, or a TCR alpha chain and a TCR beta chain. The nuclease protein can introduce a cut of the genomic locus. The vector can further comprise a pair of HAs flanking the sequence encoding the exogenous TCR, which facilitate the site-specific knock in of the sequence via homologous recombination. The nuclease can be a meganuclease, a zinc-finger nuclease (ZFN), a transcription activator-like effector nuclease (TALEN), a CRISPR/Cas nuclease, or a megaTAL nuclease.

The inactivation can include disruption of genomic gene locus, gene silencing, inhibition or reduction of transcription, or inhibition or reduction of translation. In some cases, the endogenous TCR gene can be knocked down. The endogenous TCR gene can be knocked down (or silenced), for example, by inhibitory nucleic acids such as siRNA and shRNA. The endogenous TCR gene can be knocked down by RNase-H dependent antisense. The translation of the endogenous TCR gene can be inhibited by inhibitory nucleic acids such as microRNA. In some embodiments, gene editing techniques are employed to disrupt an endogenous TCR gene or knocked out the endogenous gene. For example, an endogenous TCR gene can be knocked out using CRISPR/Cas9 editing of the TRAC or TRBC loci. In some embodiments, gene editing techniques pave the way for multiplex genomic editing, which allows simultaneous disruption of multiple genomic loci in endogenous TCR gene. In some embodiments, multiplex genomic editing techniques are applied to generate gene-disrupted T cells that are deficient in the expression of endogenous TCR, and/or human leukocyte antigens (HLAs), and/or programmed cell death protein 1 (PD1), and/or other genes.

A gene encoding the endogenous TCR chain can be inactivated using a gene editing technique such as clustered regularly interspaced short palindromic repeats (CRISPR®, see, e.g., U.S. Pat. No. 8,697,359), transcription activator-like effector (TALE) nucleases (TALENS, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. Alternatively, a gene of interest described herein can be knocked down using techniques such as RNA interference (RNAi).

These gene-editing techniques may share a common mode of action in binding a user-defined sequence of DNA and mediating a double-stranded DNA break (DSB). DSB may then be repaired by either non-homologous end joining (NHEJ) or—when donor DNA is present—homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental methods to produce a protein that binds to a pre-determined DNA sequence (e.g., sequence with 18 base pairs in length). By fusing this engineered protein domain to the Fokl nuclease, it is possible to target DNA breaks with genome-level specificity. ZFNs can be used to target gene addition, removal, and substitution in a wide range of eukaryotic organisms. Likewise, TAL-effector nucleases (TALENs) can be generated to cleave specific sites in genomic DNA. Like a ZFN, a TALEN comprises an engineered, site-specific DNA-binding domain fused to the Fokl nuclease domain. In this case, however, the DNA binding domain comprises a tandem array of TAL-effector domains, each of which specifically recognizes a single DNA base pair. Compact TALENs have an alternative endonuclease architecture that avoids the need for dimerization. A Compact TALEN can comprise an engineered, site-specific TAL-effector DNA-binding domain fused to the nuclease domain from the I-TevI homing endonuclease. Unlike Fokl, I-TevI may not dimerize to produce a double-strand DNA break so a Compact TALEN can function as a monomer.

Engineered endonucleases based on the CRISPR/Cas9 system can also be used. The CRISPR gene-editing technology can comprise an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or a RNA duplex comprising a 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems, each containing multiple CRISPR types. Class I contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed for genome editing. More than 10 different CRISPR/Cas proteins have been remodeled within last few years. For example, Cas12a (Cpf1) proteins from Acid-aminococcus sp (AsCpf1) or Lachnospiraceae bacterium (LbCpf1) can be used.

Homing endonucleases are a group of naturally occurring nucleases that recognize 15-40 base-pair cleavage sites commonly found in the genomes of plants and fungi. They can be associated with parasitic DNA elements, such as group 1 self-splicing introns and inteins. They can naturally promote homologous recombination or gene insertion at specific locations in the host genome by producing a double-stranded break in the chromosome, which recruits the cellular DNA-repair machinery. Specific amino acid substations can reprogram DNA cleavage specificity of homing nucleases. Meganucleases (MN) are monomeric proteins with innate nuclease activity that are derived from bacterial homing endonucleases and engineered for a unique target site. In some cases, meganuclease is engineered I-CreI homing endonuclease. In other cases, meganuclease is engineered I-SceI homing endonuclease.

In addition to above mentioned gene editing technologies, chimeric proteins comprising fusions of meganucleases, ZFNs, and TALENs can be engineered to generate novel monomeric enzymes that take advantage of the binding affinity of ZFNs and TALENs and the cleavage specificity of meganucleases. For example, megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, can be delivered to the cells of interest. Delivery methods include but are not limited to physical, chemical, and viral methods. In some instances, physical delivery methods can be selected from the methods including but not limited to electroporation, microinjection, or use of ballistic particles. On the other hand, chemical delivery methods may use molecules such calcium phosphate, lipid, or protein. In some embodiments, viral delivery methods can use viruses such as adenovirus, lentivirus, or retrovirus.

The modified cells (e.g., synthetic TILs) described herein can be administered into a subject (e.g., a human) having a disease such as cancer. The modified cells can proliferate or expand in the subject. The modified cells (or progeny thereof) can persist in the body of the subject. For example, after infusion, the percentage of the modified cells in all peripheral T cells from the subject can be at least about 5%, 10%, 15%, 20%, 25%, 30%, 35%, 40%, 45%, 50%, 55%, 60%, 65%, 70%, 75%, 80%, 85%, 90% or more. In some cases, after infusion, the percentage of the modified cells in all peripheral T cells from the subject can be at least about 30%, 40%, 50%, 60% or more. The percentage of CD62L⁺ cells of the modified cells among the subject's peripheral T cells can be at least about 50%, 60%, 70%, 80%, 90% or more. The percentage of the modified cells described herein can be persistent in the body of the subject. For example, the percentage of the modified cells can persist for at least 10 days, 15 days, 20 days, 25 days, 30 days, 35 days, 40 days, 50 days, 60 days, 70 days, 80 days or more; or in some cases, persist for at least 1 month, 2 months, 3 months, 4 months, 5 months, 6 months, 7 months, 8 months, 9 months, 10 months, 11 months, 12 months, 13 months, 14 months, 15 months, 16 months, 17 months, or more. In some other cases, the percentage of the modified cells can persist for at least 12 months, 13 months, 14 months, 15 months, 16 months, 17 months, 18 months, 19 months, 20 months, 21 months, 22 months, 23 months, 24 months, or more. In some other cases, the percentage of the modified cells can persist for at least 20 months, 21 months, 22 months, 23 months, 24 months, 25 months, 26 months, 27 months, 28 months, 29 months, 30 months, 31 months, 32 months, or more. In some cases, the percentage of the modified cells in all peripheral T cells in the peripheral blood mononuclear cell (PBMC) sample from the subject is at least about 30%, 40%, 50%, 60%, 70%, 80%, 90% or more. In some cases, after infusion, the modified cells can maintain the concentration of at least about 0.2×10⁶, 0.3×10⁶, 0.4×10⁶, 0.5×10⁶, 0.6×10⁶, 0.7×10⁶, 0.8×10⁶, 0.9×10⁶, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 9×10⁶, 1×10⁷ or more cells per mL of peripheral blood in the subject (or the subject's PBMC sample). In some cases, after infusion, the modified cells can maintain the concentration of at least about 0.2×10⁶, 0.3×10⁶, 0.4×10⁶, 0.5×10⁶, 0.6×10⁶, 0.7×10⁶, 0.8×10⁶, 0.9×10⁶, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 9×10⁶, 1×10⁷ or more cells per mL of peripheral blood in the subject (or the subject's PBMC sample) for at least 10 days, 15 days, 20 days, 25 days, 30 days, 35 days, 40 days, 50 days, 60 days, 70 days, 80 days or more; or in some cases, maintain for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or more. The percentage or concentration for the modified cells can persist as described herein with or without IL-2 infusion. The detection of the modified cells can be based on a marker unique to the modified cells but not other endogenous T cells from a subject. For example, a recombinant marker protein can be exogenously expressed by the modified cells. For another example, an RNA-FISH method can be used to detect the exogenous TCR transcript within the modified cells.

The modified cell can proliferate at a substantially equal rate compared to a comparable cell (e.g., an immune cell, a T cell, or a TIL).

The present disclosure also provides a mixture of cells comprising a plurality of modified cells. The mixture can comprise at least 2, 5, 10, 15, 20, 25, 30, 50, 100, 200, 500, or 1000 modified cells, each modified cell of the mixture exogenously expressing a polynucleotide sequence encoding a distinct cognate pair of a TCR alpha chain and a TCR beta chain. The polynucleotide sequence can be knocked into a genomic locus for monoallelic expression of the polynucleotide sequence. An endogenous TCR gene of the modified cell can be inactivated.

In some cases, a mixture of cells can comprise a plurality of modified cells, each modified cell of the plurality exogenously expressing a polynucleotide sequence encoding a distinct cognate pair of a TCR alpha chain and a TCR beta chain. The polynucleotide sequence can be knocked into a genomic locus for monoallelic expression of the polynucleotide sequence. An endogenous TCR gene of the modified cell can be inactivated.

Each distinct cognate pair of a TCR alpha chain and a TCR beta chain can be from a subject having a solid tumor lesion or can be expressed endogenously in a T cell from a subject having a solid tumor lesion. The subject (e.g., a cancer patient) can be intended to be administered with the mixture of modified cells or a pharmaceutical composition comprising the mixture of modified cells. The mixture can comprise at least 10, 20, 30, 50 or more modified cells, each modified cell of the mixture exogenously expressing a polynucleotide sequence encoding a distinct cognate pair of a TCR alpha chain and a TCR beta chain.

The genomic locus can be TRAC locus. The endogenous TCR gene of the modified cell can be knocked out or knocked down. The endogenous TCR gene can comprise a TRBC locus.

The modified cell can be a T cell. The T cell can be an allogeneic T cell or an autologous T cell. The distinct cognate pair of a TCR alpha chain and a TCR beta chain of each modified cell can be selected by sequencing TCRs from a plurality of T cells from a subject. The distinct cognate pair of a TCR alpha chain and a TCR beta chain of each modified cell can be selected by a pre-designed scoring method based on sequencing data of TCRs from a plurality of T cells from a subject. The distinct cognate pair of a TCR alpha chain and a TCR beta chain of each modified cell may have been validated to be functional. The distinct cognate pair of a TCR alpha chain and a TCR beta chain of each modified cell can be tumor-reactive. The tumor-reactivity of the mixture may or may not be tested. The mixture can comprise both functional and non-functional TCRs. The mixture can comprise both tumor-reactive and tumor-nonreactive TCRs. The mixture of modified cells may have been expanded in vitro.

A mixture of cells provided herein can comprise a plurality of modified cells, each modified cell of the plurality exogenously expressing a cognate pair of a TCR from a subject. The subject can be intended to be administered with the mixture or a pharmaceutical composition comprising the mixture. The mixture of cells can express at least 10 different cognate pairs of TCRs. Each TCR of the at least 10 different cognate pairs of TCRs can be expressed in at least 2% of the mixture of cells. The relative abundance of each unique TCR in the mixture may be similar to any other TCRs. Cells expressing a given TCR may not outgrow cells expressing any other TCRs. In some cases, at least 40%, 50%, 60%, 70%, 80%, 90%, 99% or 100% of the mixture of cells each exogenously expresses only one cognate pair of TCR.

A polynucleotide sequence encoding a cognate pair of TCR can be knocked into a genomic locus for monoallelic expression of the poly-nucleotide sequence in each modified cell of the at least 40%, 50%, 60%, 70%, 80%, 90%, 99% or 100% of the mixture exogenously express only one cognate pair of TCR. The modified cells can persist or maintain at a concentration of at least about 0.2×10⁶ (in some cases, 0.4×10⁶ to 1×10⁶ per mL of peripheral blood for at least about 30 days (e.g., 40 days, 45 days, 50 days, 55 days, 60 days or more) in the subject when administered into the subject.

### Treatment Regimes

Disclosed herein can be cells (e.g., synthetic TILs) used in a treatment regime. For example, a subject can receive the cells as part of a treatment regime for treatment of a cancer or disease. Treatment regimes can include: surgery, chemotherapy, radiation, immunosuppressive agents, immunostimulatory agents, antifungals, antivirals, antibiotics, or antiemetics, to name a few. In some cases, cellular compositions can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. A surgery can be a tumor resection in some cases. A surgery can be performed to isolate a TIL or TIT.

In some cases, the present disclosure provides a method of treating cancer in a subject in need thereof. The method can comprise administering to the subject a therapeutically effective amount of a pharmaceutical composition described herein or a pharmaceutical composition comprising a mixture of modified cells described herein.

In some cases, the present disclosure provides a method of treating cancer in a subject in need thereof. The method can comprise administering to the subject a pharmaceutical composition comprising (a) a synthetic TIL produced according to the method described herein; and (b) a pharmaceutically acceptable carrier.

In some cases, the present disclosure provides a method of treating a cancer in a subject in need thereof. The method can comprise administering a pharmaceutical composition comprising a mixture of cells into the subject in need thereof. The mixture of cells can exogenously express at least 10 (e.g., 20, 30, 40, 50, 60, 70, 80, 90, 100, 200, 300, 400, 500, 600, 700, 800, 900, 1,000, 1200, 1,500, 1,800, 2,000, 5,000, 10,000, 20,000, 50,000, 100,000 or more) different cognate pairs of TCRs from a subject. In some cases, at least 40%, 50%, 60%, 70%, 80%, 90%, 99% or 100% of the mixture of cells each can exogenously express only one cognate pair of TCR. The mixture of cells can be a mixture of modified cells. The mixture of cells can comprise a plurality of modified cells and a plurality of unmodified cells. Each TCR of the at least 10 different cognate pairs of TCRs can be expressed in at least 2% of the mixture of modified cells. The polynucleotide sequence can be knocked into a genomic locus for monoallelic expression of the polynucleotide sequence. The subject from whom the TCRs are obtained can be the same as the subject to be treated.

In some cases, an endogenous TCR gene of the modified cell is inactivated. The modified cell can be a T cell. The T cell can be a CD4+ T cell, a CD8+ T cell, a CD4+/CD8+ T cell, a helper T cell, a cytotoxic T cell, a memory T cell, a regulatory T cell, a natural killer T cell, an alpha beta T cell, or a gamma delta T cell. The T cell can be an allogenic T cell or an autologous T cell.

The modified cell can be isolated from the subject being administered with the composition. The distinct cognate pair of each modified cell can be from the subject in need thereof or can be expressed endogenously by a T cell from the subject in need thereof. The mixture can comprise at least 10 modified cells. The genomic locus can be a TRAC locus. The endogenous TCR gene of the modified cell can be knocked out or knocked down. The endogenous TCR gene can comprise a TRBC locus.

The subject in need thereof may have been subjected to one or more lines (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more lines) of prior therapy or may have been failed one or more lines of prior therapy. The cancer can be metastatic or refractory. The cancer can be a solid tumor.

The method can further comprise, optionally, administering an IL-2 into the subject after administering the pharmaceutical composition. Prior to administering, tumor reactivity of the mixture of cells can be detected. The mixture of modified cells can persist in the subject for at least about 30 days. After administering, the percentage of the modified cells or progeny thereof among the subject's peripheral T cells can be at least about 20% (e.g., 30%, 40%, 50%, 60%, 70%, 80%, 90% or more). The percentage of CD62L* cells of the synthetic TILs or progeny thereof among the subject's peripheral T cells can be at least about 50% (60%, 70%, 80%, 90% or more). The percentage can persist for at least about 30 days. In some cases, a concentration of the synthetic TILs in a peripheral blood sample of the subject can be at least about 0.2×10⁶ cells per mL of peripheral blood. The concentration can persist for at least about 30 days with or without IL-2 infusion.

In some cases, lymphodepletion may be performed. Pretreatment with lymphodepleting chemotherapy can enhance the efficacy of adoptively transferred T-cell therapy. Chemotherapy pretreatment prior to infusion of T cell products can significantly enhance the duration and therapeutic effect of T cells in vivo, and can be used in clinical studies of genetically modified T cells. For example, fludarabine and cyclophosphamide can be used for chemotherapy pretreatment of T cells (including TCR-T cells, CAR-T cells and TIL cells) in the treatment of malignant tumors. For example, subjects can be given cyclophosphamide for 2 consecutive days on the −6th day before TCR-T cell infusion, followed by 3 days of fludarabine for lymphodepleting chemotherapy drug pretreatment (see Table 3).

A therapeutically effective amount of cells can be used for administration. In some cases, about 5×10¹⁰ cells are administered to a subject. In some cases, about 5×10¹⁰ cells represent the median amount of cells administered to a subject. In some embodiments, about 5×10¹⁰ cells are necessary to effect a therapeutic response in a subject. In some embodiments, at least about 1×10⁶ cells, at least about 2×10⁶ cells, at least about 3×10⁶ cells, at least about 4×10⁶ cells, at least about 5×10⁶ cells, at least about 6×10⁶ cells, at least about 6×10⁶ cells, at least about 8×10⁶ cells, at least about 9×10⁶ cells, 1×10⁷ cells, at least about 2×10⁷ cells, at least about 3×10⁷ cells, at least about 4×10⁷ cells, at least about 5×10⁷ cells, at least about 6×10⁷ cells, at least about 6×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 6×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 6×10¹¹ cells, at least about 8×10¹¹ cells, at least about 9×10¹¹ cells, or at least about 1×10¹² cells are administered to a subject. For example, about 5×10¹⁰ cells may be administered to a subject. In another example, starting with 3×10⁶ cells, the cells may be expanded to about 5×10¹⁰ cells and administered to a subject. In some cases, cells are expanded to sufficient numbers for therapy. For example, 5×10⁷ cells can undergo rapid expansion to generate sufficient numbers for therapeutic use. In some cases, sufficient numbers for therapeutic use can be 5×10¹⁰. Any number of cells can be infused for therapeutic use. For example, a patient may be infused with a number of cells between 1×10⁶ to 5×10¹², inclusive. A patient may be infused with as many cells that can be generated for them. In some cases, cells that are infused into a patient are not all engineered. For example, at least 90% of cells that are infused into a patient can be engineered. In other instances, at least 40% of cells that are infused into a patient can be engineered. The amount of cells that are necessary to be therapeutically effective in a patient may vary depending on the viability of the cells, and the efficiency with which the cells have been genetically modified. In some cases, the product (e.g., multiplication) of the viability of cells post genetic modification may correspond to the therapeutic aliquot of cells available for administration to a subject. In some cases, an increase in the viability of cells post genetic modification may correspond to a decrease in the amount of cells that are necessary for administration to be therapeutically effective in a patient.

In some cases, a method can comprise calculating and/or administering to a subject an amount of engineered cells necessary to effect a therapeutic response in the subject. In some embodiments, calculating the amount of engineered cells necessary to effect a therapeutic response comprises determining the viability of the engineered cells. In some embodiments, in order to effect a therapeutic response in a subject, the cells administered to the subject are viable cells. In some embodiments, in order to effect a therapeutic response in a subject, at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10% of the cells are viable cells. In some embodiments, in order to effect a therapeutic response in a subject, at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10% of the cells have introduced with the polynucleotides encoding cognate pairs of TCRs.

In some cases, adoptively transplanted cells can be monitored by quantitative PCR (qPCR). A qPCR assay of adoptively transplanted cells can indicate a level of modified cells that exist in a subject after an introduction. In some cases, adoptively transferred cells can be monitored using flow cytometry. For example, a flow cytometry assay may determine a level of 4-1BB vs TCR. In some cases, a single-cell TCR PCR can be performed. Levels of adoptively transferred cells can be identified on day 40 post infusion. Levels of adoptively transferred cells, such as modified cells, can be identified of day 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175,180, 185, 190, 195, or up to day 200 post infusion.

### Immunostimulants

In some cases, an immunostimulant can be introduced to cells or a subject. An immunostimulant can be specific or non-specific. A specific immunostimulant can provide antigenic specificity such as a vaccine or an antigen. A non-specific immunostimulant can augment an immune response or stimulate an immune response. A non-specific immunostimulant can be an adjuvant. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with cells of the present disclosure. Cytokines can be utilized to boost cytotoxic T lymphocytes (including adoptively transferred tumor-specific cytotoxic T lymphocytes) to expand within a tumor microenvironment. In some cases, IL-2 can be used to facilitate expansion of the cells described herein. Cytokines such as IL-15 can also be employed. Other relevant cytokines in the field of immunotherapy can also be utilized, such as IL-2, IL-7, IL-12, IL-15, IL-21, or any combination thereof. In some cases, IL-2, IL-7, and IL-15 are used to culture cells of the present disclosure. An interleukin can be IL-2, or aldesleukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg. An immunostimulant (e.g., aldesleukin) can be administered from 1 dose to about 14 doses. An immunostimulant (e.g., aldesleukin) can be administered from at least about 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, or up to about 20 doses. In some cases, an immunostimulant such as aldesleukin can be administered from about 1 dose to 3 doses, from 3 doses to 5 doses, from 5 doses, to 8 doses, from 8 doses to 10 doses, from 10 doses to 14 doses, from 14 doses to 20 doses. In some cases, aldeskeukin is administered over 20 doses. In some cases, an immunostimulant, such as aldesleukin, can be administered in sequence or concurrent with a cellular administration. For example, an immunostimulant can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14. In some cases, an immunostimulant, such as aldesleukin, is administered from day 0 to day 4 after administration of a population of cells. In some cases, an immunostimulant (e.g., aldesleukin) is administered over a period of about 10 min, 15 min, 20 min, 30 min, 40 min, 50 min, 1 hour, 2 hours or up to about 3 hours. In some cases, an immunostimulant (e.g., aldesleukin) can be administered from about 24 hours prior to an administration of engineered cell to about 4 days after an administration of engineered cells. An immunostimulant (e.g., aldesleukin) can be administered from day −7, −6, −5, −4, −3, −2, −1, 0, 1, 2, 3, 4, 5, 6, 7, 8, 9, 10, 11, 12, 13, 14, 15, 16, 17, 18, 19, or up to about 20 days after an administration of engineered cells.

Immunostimulants such as aldesleukin can be provided as single-use vials containing 22 million IU (−1.3 mg) IL-2 as a sterile, white to off-white lyophilized cake plus 50 mg mannitol and 0.18 mg sodium dodecyl sulfate, buffered with approximately 0.17 mg monobasic and 0.89 mg dibasic sodium phosphate to a pH of 7.5 (range 7.2 to 7.8). The vial can be reconstituted with 1.2 mL of Sterile Water for Injection, USP, and the resultant concentration is 18 million IU/ml or 1.1 mg/mL. Diluent should be directed against the side of the vial to avoid excess foaming. Since vials contain no preservative, reconstituted solution should be used with 24 hours. Reconstituted aldesleukin can be further diluted with 50 mL of 5% Human Serum Albumin (HSA). The HSA can be added to the diluent prior to the addition of RIL-2. Dilutions of the reconstituted solution over a 1000-fold range (i.e., 1 mg/mL to 1 mcg/mL) can be acceptable in either glass bottles or polyvinyl chloride bags. Aldesleukin may be chemically stable for 48 hours at refrigerated and room temperatures, 2°-30° C. Administration of aldesleukin can be calculated based on total body weight. The final dilution of aldesleukin can be infused over 15 minutes.

In some cases, an immunostimulant is a colony stimulating factor. A colony stimulating factor can be G-CSF (filgrastim). Filgrastim can be stored in 300 mcg/ml and 480 ug/1.6 ml vials. Filgrastim can be administered daily as a subcutaneous injection. A filgrastim administration can be from about 5 mcg/kg/day. A filgrastim administration can be from about 1 mcg/kg/day, a filgrastim administration can be from about 2 mcg/kg/day, a filgrastim administration can be from about 3 mcg/kg/day, a filgrastim administration can be from about 4 mcg/kg/day, a filgrastim administration can be from about 5 mcg/kg/day, a filgrastim administration can be from about 6 mcg/kg/day, a filgrastim administration can be from about 7 mcg/kg/day, a filgrastim administration can be from about 8 mcg/kg/day, a filgrastim administration can be from about 9 mcg/kg/day, a filgrastim administration can be from about 10 mcg/kg/day. In some cases, Filgrastim can be administered at a dose ranging from about 0.5 mcg/kg/day to about 1.0 mcg/kg/day, from about 1.0 mcg/kg/day to 1.5 mcg/kg/day, from about 1.5 mcg/kg/day to about 2.0 mcg/kg/day, from about 2.0 mcg/kg/day to about 3.0 mcg/kg/day, from about 2.5 mcg/kg/day to about 3.5 mcg/kg/day, from about 3.5 mcg/kg/day to about 4.0 mcg/kg/day, from about 4.0 mcg/kg/day to about 4.5 mcg/kg/day. Filgrastim administration can continue daily until neutrophil count is at least about 1.0×10⁹/L×3 days or at least about 5.0×10⁹/L. An immunostimulant such as Filgrastim can be administered from day −7, −6, −5, −4, −3, −2, −1, 0, 1, 2, 3, 4, 5, 6, 7, 8, 9, 10, 11, 12, 13, 14, 15, 16, 17, 18, 19, or up to about 20 days after an administration of engineered cells.

### Chemotherapeutic Agents

A chemotherapeutic agent or compound can be a chemical compound useful in the treatment of cancer. The chemotherapeutic cancer agents that can be used in combination with the modified cells include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein includes antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The modified cells described herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed engineered cells can include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer. Any of the aforementioned chemotherapeutics can be administered at a clinically effective dose. A chemotherapeutic can also be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of a population of cells. In some cases, a subject can have a refractory cancer that is unresponsive to a chemotherapeutic.

### Antifungal Agents

In some cases, an antifungal therapy is administered to a subject receiving immunoreceptor-programmed recipient cells. Antifungals can be drugs that can kill or prevent the growth of fungi. Targets of antifungal agents can include sterol biosynthesis, DNA biosynthesis, and β-glucan biosynthesis. Antifungals can also be folate synthesis inhibitors or nucleic acid cross-linking agents. A folate synthesis inhibitor can be a sulpha based drug. For example, a folate synthesis inhibitor can be an agent that inhibits a fungal synthesis of folate or a competitive inhibitor. A sulpha based drug, or folate synthesis inhibitor, can be methotrexate or sulfamethaxazole. In some cases, an antifungal can be a nucleic acid cross-linking agent. A cross-linking agent may inhibit a DNA or RNA process in fungi. For example, a cross-linking agent can be 5-fluorocytosine, which can be a fluorinated analog of cytosine. 5-fluorocytosine can inhibit both DNA and RNA synthesis via intracytoplasmic conversion to 5-fluorouracil. Other anti-fungal agents can be griseofulvin. Griseofulvin is an antifungal antibiotic produced by Penicillium griseofulvum. Griseofulvin inhibits mitosis in fungi and can be considered a cross linking agent. Additional cross linking agent can be allylamines (naftifine and terbinafine) inhibit ergosterol synthesis at the level of squalene epoxidase; one morpholene derivative (amorolfine) inhibits at a subsequent step in the ergosterol pathway.

In some cases, an antifungal agent can be from a class of polyene, azole, allylamine, or echinocandin. In some embodiments, a polyene antifungal is amphotericin B, candicidin, filipin, hamycin, natamycin, nystatin, or rimocidin. In some cases, an antifungal can be from an azole family. Azole antifungals can inhibit lanosterol 14 α-demethylase. An azole antifungal can be an imidazole such as bifonazole, butoconazole, clotrimazole, econazole, fenticonazole, isoconazole, ketoconazole, luliconazole, miconazole, omoconazole, oxiconazole, sertaconazole, sulcoazole, or tioconazole. An azole antifungal can be a triazole such as albaconazole, efinaconazole, epoxiconazole, fluconazole, isavuvonazole, itraconazole, posaconazole, propiconazole, ravuconazole, terconazole, or voriconazole. In some cases an azole can be a thiazole such as abafungin. An antifungal can be an allylamine such as amorolfin, butenafine, naftifine, or terbinafine. An antifungal can also be an echinocandin such as anidulafungin, caspofungin, or micafungin. Additional agents that can be antifungals can be aurones, benzoic acid, ciclopirox, flucytosine, griseofulvin, haloprogin, tolnaftate, undecylenic acid, cystal violet or balsam of Peru.

A person of skill in the art can appropriately determine which known antifungal medication to apply based on the fungus infecting the individual. In some cases, a subject can receive fluconazole in combination with engineered cells. An anti-fungal therapy can be administered prophalaytically.

Fluconazole can be available in 200 mg tablets. In some cases, fluconazole can be administered as a 50 mg, 100 mg, 150 mg, 200 mg, 250 mg, 300 mg, 350 mg, or up to about 400 mg tablet. For IV administration in subjects who cannot tolerate an oral preparation, fluconazole comes in 2 MG/ML solution for injection. It should be administered at a maximum IV rate of 200 mg/hr. In some cases, an infusion rate can be from about 50 mg/hr to about 500 mg/hr. An infusion rate can also be from about 20 mg/hr to about 30 mg/hr, from about 30 mg/hr to about 40 mg/hr, from about 40 mg/hr to about 50 mg/hr, from about 50 mg/hr to about 60 mg/hr, from about 60 mg/hr to about 70 mg/hr, from about 70 mg/hr to about 80 mg/hr, from about 80 mg/hr to about 90 mg/hr, from about 90 mg/hr to about 100 mg/hr, from about 100 mg/hr to about 120 mg/hr, from about 120 mg/hr to about 140 mg/hr, from about 140 mg/hr to about 160 mg/hr, from about 160 mg/hr to about 180 mg/hr, from about 180 mg/hr to about 200 mg/hr, from about 180 mg/hr to about 220 mg/hr, from about 220 mg/hr to about 240 mg/hr, or from about 240 mg/hr to about 275 mg/hr.

Antifungals can be administered at therapeutically effective doses. A therapeutically effective dose is a dose that treats or prevents a fungal infection but that is not effective for treating a cancer. For example an antifungal such as fluconazole can be administered from about 10 mg to about 1000 mg. Fluconazole can be administered from about 10 mg, 20 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or up to about 1000 mg. Fluconazole can be administered at 400 mg. In some cases, an antifungal administration can be before a cellular therapy, during a cellular therapy or after a cellular therapy has been administered. For example a fluconazole administration can be from about day 0 (day a cellular therapy is introduced into a subject) to about day 4 after administration of a cellular therapy. An antifungal can be administered from about 14 days leading up to a cellular therapy administration to about 14 days after a cellular therapy is completed. An antifungal can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14.

### Immunosuppressive Agents

In some cases, a subject may receive an immunosuppressive agent as part of a therapy regime. An immunosuppressive agent can refer to a radiotherapeutic, a biologic, or a chemical agent. In some cases, an immunosuppressive agent can include a chemical agent. For example, a chemical agent can comprise at least one member from the group consisting of: cyclophosphamide, mechlorethamine, chlorambucil, melphalan, ifosfamide, thiotepa, hexamethylmelamine, busulfan, fludarabine, nitrosoureas, platinum, methotrexate, azathioprine, mercaptopurine, procarbazine, dacarbazine, temozolomide, carmustine, lomustine, streptozocin, fluorouracil, dactinomycin, anthracycline, mitomycin C, bleomycin, and mithramycin. A chemical agent can be cyclophosphamide or fludarabine.

Additionally, immunosuppressive agents can include glucocorticoids, cytostatic, antibodies, anti-immunophilins, or any derivatives thereof. A glucocorticoid can suppress an allergic response, inflammation, and autoimmune conditions. Glucocorticoids can be prednisone, dexamethasone, and hydrocortisone. Immunosuppressive therapy can comprise any treatment that suppresses the immune system. Immunosuppressive therapy can help to alleviate, minimize, or eliminate transplant rejection in a recipient. For example, immunosuppressive therapy can comprise immuno-suppressive drugs. Immunosuppressive drugs that can be used before, during and/or after transplant, but are not limited to, MMF (mycophenolate mofetil (Cellcept)), ATG (anti-thymocyte globulin), anti-CD154 (CD40L), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-OX40 antibody, and anti-CD122 antibody. Furthermore, one or more than one immunosuppressive agents/drugs can be used together or sequentially. One or more than one immunosuppressive agents/drugs can be used for induction therapy or for maintenance therapy. The same or different drugs can be used during induction and maintenance stages. In some cases, daclizumab (Zenapax) can be used for induction therapy and tacrolimus (Prograf) and sirolimus (Rapimune) can be used for maintenance therapy. Daclizumab (Zenapax) can also be used for induction therapy and low dose tacrolimus (Prograf) and low dose sirolimus (Rapimune) can be used for maintenance therapy. Immunosuppression can also be achieved using non-drug regimens including, but not limited to, whole body irradiation, thymic irradiation, and full and/or partial splenectomy. In some cases, a cytostatic agent can be administered for immunosuppression.

Cytostatic agents can inhibit cell division. A cytostatic agent can be a purine analog. A cytostatic agent can be an alkylating agent, an antimetabolite such as methotrexate, azathioprine, or mercaptopurine. A cytostatic agent can be at least one of cyclophosphamide, mechlorethamine, chlorambucil, melphalan, ifosfamide, thiotepa, hexamethylmelamine, busulfan, fludarabine, nitrosoureas, platinum, methotrexate, azathioprine, mercaptopurine, procarbazine, dacarbazine, temozolomide, carmustine, lomustine, streptozocin, fluorouracil, dactinomycin, anthracycline, mitomycin C, bleomycin, and mithramycin.

In some cases, an immunosuppressive agent such as fludarabine can be administered as part of a treatment regime. Fludarabine phosphate can be a synthetic purine nucleoside that differs from physiologic nucleosides in that the sugar moiety can be arabinose instead of ribose or deoxyribose. Fludarabine can be a purine antagonist antimetabolite. Fludarabine can be supplied in a 50 mg vial as a fludarabine phosphate powder in the form of a white, lyophilized solid cake. Following reconstitution with 2 mL of sterile water for injection to a concentration of 25 mg/ml, the solution can have a pH of 7.7. The fludarabine powder can be stable for at least 18 months at 2-8° C.; when reconstituted, fludarabine is stable for at least 16 days at room temperature. Because no preservative is present, reconstituted fludarabine can be administered within 8 hours. Specialized references should be consulted for specific compatibility information. Fludarabine can be dephosphorylated in serum, transported intracellularly and converted to the nucleotide fludarabine triphosphate; this 2-fluoro-ara-ATP molecule is thought to be required for the drug's cytotoxic effects. Fludarabine inhibits DNA polymerase, ribonucleotide reductase, DNA primase, and may interfere with chain elongation, and RNA and protein synthesis. Fludarabine can be administered as an IV infusion in 100 ml 0.9% sodium chloride, USP over 15 to 30 minutes. The doses can be based on body surface area (BSA). If patient is obese (BMI>35) drug dosage can be calculated using practical weight. In some cases, an immunosuppressive agent such as fludarabine can be administered from about 20 mg/m² to about 30 mg/m² of body surface area of a subject. In some cases, an immunosuppressive agent such as fludarabine can be administered from about 5 mg/m² to about 10 mg/m² of body surface area of a subject, from about 10 mg/m² to about 15 mg/m² of body surface area of a subject, from about 15 mg/m² to about 20 mg/m² of body surface area of a subject, from about 20 mg/m² to about 25 mg/m² of body surface area of a subject, from about 25 mg/m² to about 30 mg/m² of body surface area of a subject, from about 30 mg/m² to about 40 mg/m² of body surface area of a subject. In some cases, an immunosuppressive agent such as fludarabine can be administered from about 1 mg/m², 2 mg/m², 3 mg/m², 4 mg/m², 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m², 30 mg/m², 31 mg/m², 32 mg/m², 33 mg/m², 34 mg/m², 35 mg/m², 36 mg/m², 37 mg/m², 38 mg/m², 39 mg/m², 40 mg/m², 41 mg/m², 42 mg/m², 43 mg/m², 44 mg/m², 45 mg/m², 46 mg/m², 47 mg/m², 48 mg/m², 49 mg/m², 50 mg/m², 51 mg/m², 52 mg/m², 53 mg/m², 54 mg/m², 55 mg/m², 56 mg/m², 57 mg/m², 58 mg/m², 59 mg/m², 60 mg/m², 61 mg/m², 62 mg/m², 63 mg/m², 64 mg/m², 65 mg/m², 66 mg/m², 67 mg/m², 68 mg/m², 69 mg/m², 70 mg/m², 71 mg/m², 72 mg/m², 73 mg/m², 74 mg/m², 75 mg/m², 76 mg/m², 77 mg/m², 78 mg/m², 79 mg/m², 80 mg/m², 81 mg/m², 82 mg/m², 83 mg/m², 84 mg/m², 85 mg/m², 86 mg/m², 87 mg/m², 88 mg/m², 89 mg/m², 90 mg/m², 91 mg/m², 92 mg/m², 93 mg/m², 94 mg/m², 95 mg/m², 96 mg/m², 97 mg/m², 98 mg/m², 99 mg/m², up to about 100 mg/m² of body surface area of a subject. In some cases, an immunosuppressive agent such as fludarabine is at a dose of 25 mg/m² in 100 ml 0.9% sodium chloride, USP and infused over about 15 to about 30 minutes.

In some cases, an immunosuppressive agent such as cyclophosphamide can be administered as part of a treatment regime. Cyclophosphamide can be a nitrogen mustard-derivative alkylating agent. Following conversion to active metabolites in the liver, cyclophosphamide functions as an alkylating agent; the drug also possesses potent immunosuppressive activity. The serum half-life after IV administration ranges from 3-12 hours; the drug and/or its metabolites can be detected in the serum for up to 72 hours after administration. Following reconstitution as directed with sterile water for injection, cyclophosphamide can be stable for 24 hours at room temperature or 6 days when kept at 2-8° C. Cyclophosphamide can be diluted in 250 ml D5W and infused over one hour. The dose can be based on a subject's body weight. If a subject is obese (BMI>35) drug dosage can be calculated using practical weight as described in. In some cases, an immunosuppressive agent such as cyclophosphamide can be administered from about 1 mg/kg to about 3 mg/kg, from about 3 mg/kg to about 5 mg/kg, from about 5 mg/kg to about 10 mg/kg, from about 10 mg/kg to about 20 mg/kg, 20 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, from about 90 mg/kg to about 100 mg/kg. In some cases, an immunosuppressive agent such as cyclophosphamide is administered in excess of 50 mg/kg of a subject. In some cases, an immunosuppressive agent such as cyclophosphamide can be administered from about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, up to about 100 mg/kg of a subject. In some cases, an immunosuppressive agent such as cyclophosphamide can be administered over at least about 1 day to about 3 days, from 3 days to 5 days, from 5 days to 7 days, from 7 days to about 10 days, from 10 days to 14 days, from 14 days to about 20 days. In some cases, cyclophosphamide can be at a dose of about 60 mg/kg and is diluted in 250 ml 5% dextrose in water and infused over one hour.

An immunosuppressive agent can be, for example, a regime of cyclophosphamide and fludarabine. For example, a cyclophosphamide fludarabine regimen can be administered to a subject receiving an engineered cellular therapy. A cyclophosphamide fludarabine regimen can be administered at a regime of 60 mg/kg qd for 2 days and 25 mg/m² qd for 5 days. A chemotherapeutic regime, for example, cyclophosphamide fludarabine, can be administered from 1 hour to 14 days preceding administration of engineered cells of the present disclosure. A chemotherapy regime can be administered at different doses. For example, a subject may receive a higher initial dose followed by a lower dose. A subject may receive a lower initial dose followed by a higher dose.

In some cases, an immunosuppressive agent can be an antibody. An antibody can be administered at a therapeutically effective dose. An antibody can be a polyclonal antibody or a monoclonal antibody. A polyclonal antibody that can be administered can be an antilymphocyte or antithymocyte antigen. A monoclonal antibody can be an anti-IL-2 receptor antibody, an anti-CD25 antibody, or an anti-CD3 antibody. An anti-CD20 antibody can also be used. B-cell ablative therapy such as agents that react with CD20, e.g., Rituxan can also be used as immunosuppressive agents.

An immunosuppressive can also be an anti-immunophilin. Anti-immunophilins can be ciclosporin, tacrolimus, everolimus, or sirolimus. Additional immunosuppressive agents can be interferons such as IFN-beta, opiods, anti-TNF binding agents, mycophenolate, or fingolimod.

Immunosuppressive agents can also refer to radiotherapeutics. Radiotherapy can include radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips. For example, in one embodiment, subjects may undergo standard treatment with high dose chemotherapy followed by peripheral blood stem cell transplantation. In certain embodiments, following the transplant, subjects receive an infusion of the expanded cells (e.g., immunoreceptor-programmed recipient cells) of the present disclosure. The dosage of the above treatments to be administered to a patient can vary with the precise nature of the condition being treated and the recipient of the treatment. The scaling of dosages for human administration can be performed according to art-accepted practices. The dose for CAMPATH, for example, can be in the range 1 to about 100 mg for an adult patient, usually administered daily for a period between 1 and 30 days. The daily dose can be 1 to 10 mg per day although in some instances larger doses of up to 40 mg per day may be used (described in U.S. Pat. No. 6,120,766).

### Antibiotic Agents

An antibiotic can be administered to a subject as part of a therapeutic regime. An antibiotic can be administered at a therapeutically effective dose. An antibiotic can kill or inhibit growth of bacteria. An antibiotic can be a broad spectrum antibiotic that can target a wide range of bacteria. Broad spectrum antibiotics, either a 3^{rd} or 4^{th} generation, can be cephalosporin or a quinolone.

An antibiotic can also be a narrow spectrum antibiotic that can target specific types of bacteria. An antibiotic can target a bacterial cell wall such as penicillins and cephalosporins. An antibiotic can target a cellular membrane such as polymyxins. An antibiotic can interfere with essential bacterial enzymes such as antibiotics: rifamycins, lipiarmycins, quinolones, and sulfonamides. An antibiotic can also be a protein synthesis inhibitor such as macrolides, lincosamides, and tetracyclines. An antibiotic can also be a cyclic lipopeptide such as daptomycin, glycylcyclines such as tigecycline, oxazolidiones such as linezolid, and lipiarmycins such as fidaxomicin.

In some cases, an antibiotic can be 1^{st} generation, 2^{nd} generation, 3^{rd} generation, 4^{th} generation, or 5^{th} generation. A first generation antibiotic can have a narrow spectrum. Examples of 1^{st} generation antibiotics can be penicillins (Penicillin G or Penicillin V), Cephalosporins (Cephazolin, Cephalothin, Cephapirin, Cephalethin, Cephradin, or Cephadroxin). In some cases, an antibiotic can be 2^{nd} generation. 2^{nd} generation antibiotics can be a penicillin (Amoxicillin or Ampicillin), Cephalosporin (Cefuroxime, Cephamandole, Cephoxitin, Cephaclor, Cephrozil, Loracarbef). In some cases, an antibiotic can be 3^{rd} generation. A 3^{rd} generation antibiotic can be penicillin (carbenicillin and ticarcillin) or cephalosporin (Cephixime, Cephtriaxone, Cephotaxime, Cephtizoxime, and Cephtazidime). An antibiotic can also be a 4^{th} generation antibiotic. A 4^{th} generation antibiotic can be Cephipime. An antibiotic can also be 5^{th} generation. 5^{th} generation antibiotics can be Cephtaroline or Cephtobiprole.

In some cases, an antibiotic can be a bacterial wall targeting agent, a cell membrane targeting agent, a bacterial enzyme interfering agent, a bactericidal agent, a protein synthesis inhibitor, or a bacteriostatic agent. A bacterial wall targeting agent can be a penicillin derivatives (penams), cephalosporins (cephems), monobactams, and carbapenems. β-Lactam antibiotics are bactericidal or bacteriostatic and act by inhibiting the synthesis of the peptidoglycan layer of bacterial cell walls. In some cases, an antibiotic may be a protein synthesis inhibitor. A protein synthesis inhibitor can be ampicillin which acts as an irreversible inhibitor of the enzyme transpeptidase, which is needed by bacteria to make the cell wall. It inhibits the third and final stage of bacterial cell wall synthesis in binary fission, which ultimately leads to cell lysis; therefore, ampicillin is usually bacteriolytic. In some cases, a bactericidal agent can be cephalosporin or quinolone. In other cases, a bacteriostatic agent is trimethoprim, sulfamethoxazole, or pentamidine.

In some cases, an agent for the prevention of PCP pneumonia may be administered. For example, Trimethoprim and Sulfamethoxazole can be administered to prevent pneumonia. A dose of trimethoprim and sulfamethoxazole (TMP/SMX; an example sulfa drug) can be 1 tablet PO daily three times a week, on non-consecutive days, on or after the first dose of chemotherapy and continuing for at least about 6 months and until a CD4 count is greater than 200 on at least 2 consecutive lab studies. In some cases, trimethoprim can be administered at 160 mg. Trimethoprim can be administered from about 100 to about 300 mgs. Trimethoprim can be administered from about 100 mg, 125 mg, 150 mg, 175 mg, 200 mg, 225 mg, 250 mg, 275 mg, or up to about 300 mg. In some cases, sulfamethoxazole is administered at 800 mg. Sulfamethoxazole can be administered from about 500 mg to about 1000 mg. Sulfamethoxazole can be administered from about 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or up to about 1000 mgs. In some cases, a TMP/SMX regime can be administered at a therapeutically effective amount. TMP/SMX can be administered from about 1× to about 10× daily. TMP/SMX can be administered 1×, 2×, 3×, 4×, 5×, 6×, 7×, 8×, 9×, 10×, 11×, 12×, 13×, 14×, 15×, 16×, 17×, 18×, 19×, or up to about 20× daily. In some cases, TMP/SMX can be administered on a weekly basis. For example, TMP/SMX can be administered from 1×, 2×, 3×, 4×, 5×, 6×, or up to about 7× a week. A TMP/SMX regime can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of a cellular therapy, such as the recipient cells described herein.

In some cases, a subject that has a sulfa allergy may receive pentamidine. Pentamidine can be administered by aerosol. Pentamidine 300 mg per nebulizer within one week prior to admission and continued monthly until the CD4 count is above 200 on two consecutive follow up lab studies and for at least 6 months post chemotherapy. Pentamidine can be used to prevent the occurrence of PCP infections. It can be supplied in 300 mg vials of lyophilized powder and can be administered via nebulizer. Pentamidine can be administered from about 300 mg to about 500 mgs. In some cases, pentamidine can be administered from about 100 mg, 200 mg, 300 mg, 400 mg, 500 mg, 600 mg, 700 mg, or up to about 800 mgs.

In some cases, a bacteriostatic agent, such as an antibiotic can be administered prior to the administration of immunoreceptor-programmed recipient cells, concurrent with these cells, or after these cells. In some cases, a bacteriostatic agent can be administered from about 14 days prior to an administration of the immunoreceptor-programmed recipient cells to about 6 months after the administration of these cells.

### Anti-Viral Agents

In some cases, an anti-viral agent may be administered as part of a treatment regime. In some cases, a herpes virus prophylaxis can be administered to a subject as part of a treatment regime. A herpes virus prophylaxis can be valacyclovir (Valtrex). Valtrex can be used orally to prevent the occurrence of herpes virus infections in subjects with positive HSV serology. It can be supplied in 500 mg tablets. Valacyclovir can be administered at a therapeutically effective amount. For example, valacyclovir can be administered from about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, or up to about 700 mg tablets. Valacyclovir can be started the day after the last dose of fludarabine at a dose of 500 mg orally daily if a subject is able to tolerate oral intake. An antiviral therapy can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of a cellular therapy.

In some cases, a subject may not be able to take oral medication for the prophylaxis of herpes. In those cases, acyclovir can be administered. Acyclovir can be supplied as a powder for injection in 500 mg/vials. In some cases, acyclovir can be administered at a therapeutically effective amount. Acyclovir can be administered orally from about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, or up to about 700 mgs. Acyclovir can be administered 1×, 2×, 3×, 4×, 5×, 6×, or up to about 7× per day. Acyclovir can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of a cellular therapy. In some cases, acyclovir can be administered intravenously. For example, acyclovir can be administered at 1 mg/kg to about 3 mg/kg, from about 3 mg/kg to about 5 mg/kg, from about 5 mg/kg to about 10 mg/kg, from about 10 mg/kg to about 20 mg/kg, 20 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, from about 90 mg/kg to about 100 mg/kg. In some cases, acyclovir is administered in excess of 50 mg/kg. Acyclovir can be reconstituted in 10 mL of sterile water for injection to a concentration of 50 mg/mL. Reconstituted solutions should be used within 12 hours. IV solutions can be diluted to a concentration of 7 mg/mL or less and infused over 1 hour to avoid renal damage.

### Patient Population

The modified cells, synthetic TILs, or pharmaceutical compositions comprising such cells described herein can be administered into a subject in need thereof. The subject can be a patient having a disease or a condition. The subject can be a human subject. In some embodiments, a patient or population of patients to be treated with modified cells, synthetic TILs, or a pharmaceutical composition of the present disclosure have a solid tumor. In some embodiments, a solid tumor is a melanoma, renal cell carcinoma, lung cancer, bladder cancer, breast cancer, cervical cancer, colorectal cancer, head and neck cancer, colon cancer, gall bladder cancer, laryngeal cancer, liver cancer (e.g., hepatocellular carcinoma), thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer.

In various cases, the subject described herein may have received a first line of treatment for cancer prior to being administered with a composition described herein. In some cases, the subject may have received two or more lines of treatment for cancer prior to being administered with a composition described herein. The cancer may be a relapsed cancer after the one or more lines of prior therapy. The cancer may be highly refractory or highly resistant to the one or more lines of prior therapy.

Provided herein are methods of treating a subject in need thereof after one or more lines of prior therapy for treating a cancer. The method can comprise administering to the subject one or more doses of a population of modified cells or synthetic TILs described herein, or pharmaceutical compositions comprising such cells described herein. In various embodiments, the methods provided herein can further comprise selecting the subject for the treatment provided herein. The selection of the subject can be determined by the stage of the cancer, whether the cancer is a metastatic cancer, and/or if the subject has received any prior therapy.

In some aspects, at least one of the one or more lines of prior therapy can comprise surgery, chemotherapy, hormonal therapy, biological therapy, antibody therapy, radiation therapy, or any combinations thereof. Any combinations described herein can be referred to as a combination therapy. At least one of the one or more lines of prior therapy can comprise a systemic therapy. The systemic therapy can comprise a biologic or chemotherapy. The biologic can comprise an antibody, antibody drug conjugate (ADC), cellular therapy, peptide, polypeptide, enzyme, vaccine, oligonucleotide, oncolytic virus, polysaccharide, or gene therapy.

The biologic can comprise an antibody or antibody drug conjugate (ADC). The antibody or ADC can comprise a checkpoint inhibitor antibody or ADC. The checkpoint inhibitor antibody can comprise nivolumab, pembrolizumab, cemiplimab, atezolizumab, avelumab, durvalumab, ipilimumab, or a biosimilar thereof. The biologic can comprise a cellular therapy. The systemic therapy can comprise a chemotherapy agent. The chemotherapy agent can comprise an alkylating agent (e.g., a nitrogen mustard analog or a platinum based compound), a targeted therapy, a protein kinase inhibitor, an antimetabolite (e.g., a folic acid analog or a pyrimidine analog), an anti-microtubule agent, an anthracycline, a topoisomerase inhibitor, a PARP inhibitor, a hormone modifying treatment, a taxane, a rapalog, or an epigenetic therapy (e.g., a DNMT inhibitor or an HDAC inhibitor). In some aspects, at least one of the one or more lines of prior therapy can comprise a targeted therapy. In some aspects, at least one of the one or more lines of prior therapy can comprise an antiangiogenic. The anti-angiogenic can comprise axitinib, bevacizumab, cabozantinib, cediranib, everolimus, lenalidomide, lenvatinib mesylate, nintedanib, pazopanib, ramucirumab, regorafenib, sorafenib, sunitinib, thalidomide, vandetanib, or ziv-aflibercept.

The combination therapy can comprise one or more systemic therapies or a systemic therapy in combination with chemotherapy or radiation therapy. The combination therapy can comprise a chemotherapy and a biologic.

### Administration

Provided herein can be methods for administering a therapeutic regime to a subject having a condition such as cancer. In some instances, a cellular composition (for example, comprising modified cells or synthetic TILs described herein) can be provided in a unit dosage form. A cellular composition can be resuspended in solution and administered as an infusion. Provided herein can also be a treatment regime that includes immunostimulants, immunosuppressants, antibiotics, antifungals, antiemetics, chemotherapeutics, radiotherapy, and any combination thereof. A treatment regime that includes any of the above can be lyophilized and reconstituted in an aqueous solution (e.g., saline solution). In some instances, a treatment (for example, a cellular treatment) is administered by a route selected from subcutaneous injection, intramuscular injection, intradermal injection, percutaneous administration, intravenous (“i.v.”) administration, intranasal administration, intralymphatic injection, and oral administration. In some instances, a subject is infused with a cellular composition comprising immunoreceptor-programmed recipient cells by an intralymphatic microcatheter.

Many drugs can be administered orally as liquids, capsules, tablets, or chewable tablets. Because the oral route may be the most convenient and usually the safest and least expensive, it can be the one most often used. However, it may have limitations because of the way a drug typically moves through the digestive tract. For drugs administered orally, absorption may begin in the mouth and stomach. However, most drugs can be absorbed from the small intestine. The drug passes through the intestinal wall and travels to the liver before being transported via the bloodstream to its target site. The intestinal wall and liver can chemically alter (metabolize) many drugs, decreasing the amount of drug reaching the bloodstream. Consequently, these drugs can be given in smaller doses when injected intravenously to produce the same effect.

For a subcutaneous route, a needle may be inserted into fatty tissue just beneath the skin. After a drug is injected, it then moves into small blood vessels (capillaries) and is carried away by the bloodstream. Alternatively, a drug can reach the bloodstream through the lymphatic vessels. The intramuscular route may be used when larger volumes of a drug product are needed. Because the muscles lie below the skin and fatty tissues, a longer needle may be used. Drugs are usually injected into the muscle of the upper arm, thigh, or buttock. For the intravenous route, a needle can be inserted directly into a vein. A solution containing the drug may be given in a single dose or by continuous infusion. For infusion, the solution can be moved by gravity (from a collapsible plastic bag) or, more commonly, by an infusion pump through thin flexible tubing to a tube (catheter) inserted in a vein, usually in the forearm. In some cases, cells or therapeutic regimes are administered as infusions. An infusion can take place over a period of time. For example, an infusion can be an administration of a cell or therapeutic regime over a period of about 5 minutes to about 5 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, or up to about 5 hours.

In some embodiments, intravenous administration is used to deliver a precise dose quickly and in a well-controlled manner throughout the body. It can also be used for irritating solutions, which can cause pain and damage tissues if given by subcutaneous or intramuscular injection. An intravenous injection may be more difficult to administer than a subcutaneous or intramuscular injection because inserting a needle or catheter into a vein may be difficult, especially if the person is obese. When given intravenously, a drug can be delivered immediately to the bloodstream and tend to take effect more quickly than when given by any other route. Consequently, health care practitioners can closely monitor people who receive an intravenous injection for signs that the drug is working or is causing undesired side effects. Also, the effect of a drug given by this route may tend to last for a shorter time. Therefore, some drugs can be given by continuous infusion to keep their effect constant. For the intrathecal route, a needle can be inserted between two vertebrae in the lower spine and into the space around the spinal cord. The drug can then be injected into the spinal canal. A small amount of local anesthetic can be used to numb the injection site. This route can be used when a drug is needed to produce rapid or local effects on the brain, spinal cord, or the layers of tissue covering them (meninges) for example, to treat infections of these structures.

Drugs administered by inhalation through the mouth can be atomized into smaller droplets than those administered by the nasal route, so that the drugs can pass through the windpipe (trachea) and into the lungs. How deep into the lungs they go can depend on the size of the droplets. Smaller droplets can go deeper, which can increase the amount of drug absorbed. Inside the lungs, they can be absorbed into the bloodstream.

In some cases, a treatment regime may be dosed according to a body weight of a subject. In subjects who are determined obese (BMI>35) a practical weight may need to be utilized. BMI is calculated by: BMI=weight (kg)/[height (m)]².

An ideal body weight may be calculated for men as 50 kg+2.3×(number of inches over 60 inches) or for women 45.5 kg+2.3×(number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases a body surface area may be utilized to calculate a dosage. A body surface area (BSA) may be calculated by: BSA (m2)=√Height (cm)*Weight (kg)/3600.

In some cases, a pharmaceutical composition comprising a cellular therapy can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

In some cases, a therapeutic regime can be administered along with a carrier or excipient. Examples of carriers and excipients can include dextrose, sodium chloride, sucrose, lactose, cellulose, xylitol, sorbitol, malitol, gelatin, PEG, PVP, and any combination thereof. In some cases, an excipient such as dextrose or sodium chloride can be at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%. In some cases, a method of treating a disease in a subject may comprise transplanting to the subject one or more cells (including organs and/or tissues) comprising modified cells (e.g., synthetic TILs). Cells prepared by intracellular genomic transplant can be used to treat cancer.

### Pharmaceutical Compositions

The present disclosure also provides pharmaceutical compositions comprising a modified cell (e.g., a synthetic TIL) in combination with one or more pharmaceutically or physiologically acceptable carriers, diluents or excipients. In some cases, the pharmaceutical composition comprises a mixture comprising a plurality of modified cells. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for intravenous administration.

Also provided herein is a composition for use as a medicament, the composition comprising a modified cell (e.g., synthetic TILs, a mixture of modified cells, or a mixture of modified cells expressing polyclonal TCRs) produced by the methods described herein. The composition can be a pharmaceutical composition comprising one or more pharmaceutically or physiologically acceptable carriers, diluents or excipients. The composition can be used to treat a disease such as cancer.

Pharmaceutical compositions of the present disclosure may be administered in a manner appropriate to the disease to be treated (or prevented). The quantity and frequency of administration can be determined by such factors as the condition of the patient, and the type and severity of the patient's disease, although appropriate dosages may be determined by clinical trials.

The pharmaceutical composition can be substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some cases, the bacterium can be at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, Streptococcus pyogenes group A, and any combinations thereof.

### EXAMPLES

### Example 1: Therapeutic-Grade TCRs can be Made in High Throughput

In the International Application No. PCT/US2020/026558, Chen and Porter described a technology to synthesize TCR genes using oligonucleotide pools encoding the CDR3-J (e.g., CDR3 and the J gene-derived FR4 sequence, sometimes written as CDR3J) sequences and pre-synthesized pools of DNA fragments encoding the germline sequences of all common TCR V genes. While the TCR genes produced using this method has sufficiently high quality to enable downstream functional screening of the TCRs, in some cases, a fraction of the DNA molecules may contain errors due to the mutations introduced during the synthesis of the oligonucleotide pools. To improve the fidelity of TCR gene synthesis, an error correction step can be used. However, typical error correction methods (e.g., using CorrectASE, or MutS) are only applicable when one gene is made in one reaction, but is unsuitable when many (e.g., hundreds to thousands of or more) genes are made in one homogenous reaction. To reduce the errors, a barcode can be introduced for each TCR synthesized so that a pure TCR gene can be isolated from the TCR gene pool through a PCR reaction using a primer corresponding to the barcode. The barcode sequence can be the Zip sequence or contained within the Zip sequence. After isolation of single TCR genes, conventional error correction methods can be applied.

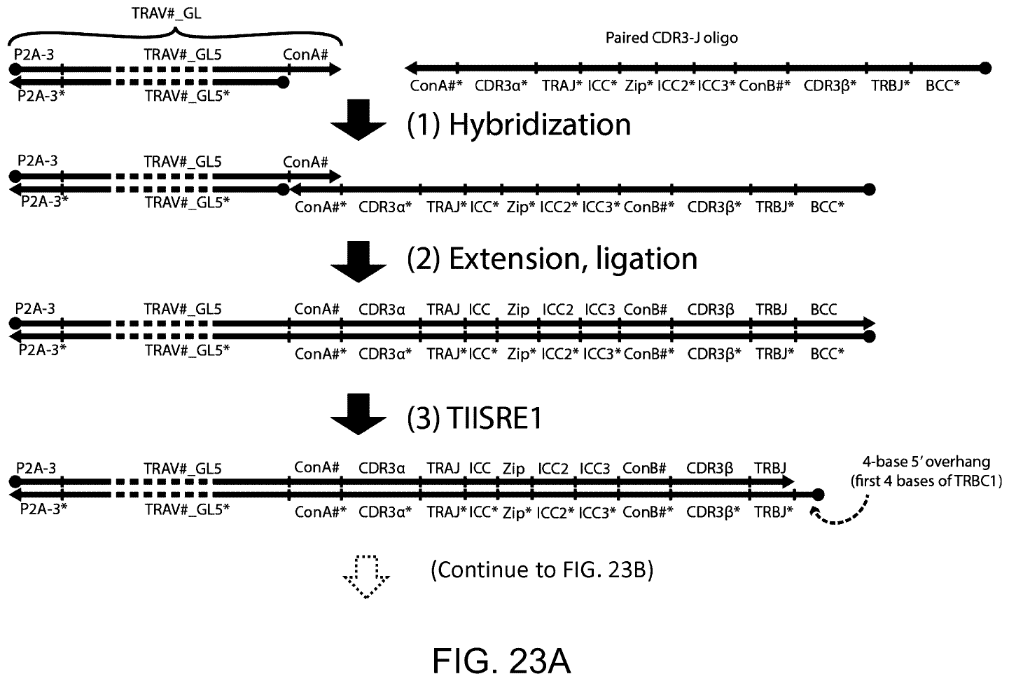

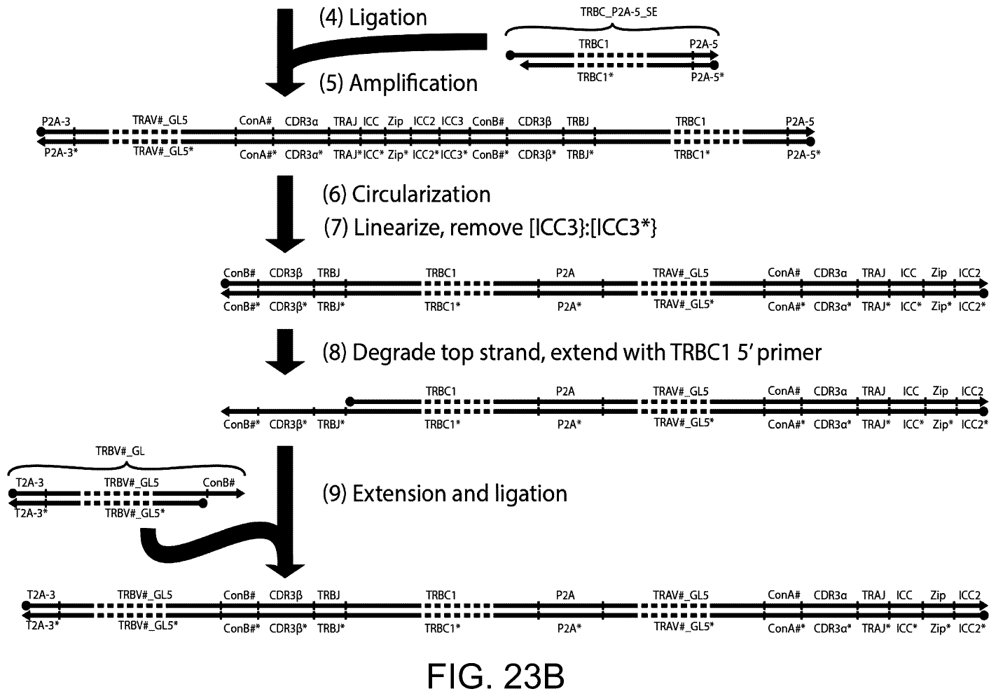

The design is shown in FIG. 23A and FIG. 23B. In the Paired CDR3-J oligo has domains [ICC3*|ICC2*|ICC*|Zip*} between ConB #* and TRAJ*. In the previous design, there is only [ICC*} in this region. Zip is a computationally designed ˜20-mer sequence whose sequence can be unrelated to TCR sequences. Different TCR genes have a different Zip sequence. Some of the Zip sequences used in this example are:

5′-CCGAGAGTTTGTTGTCCA-3′
5′-TGCAACAACAGGATCTCC-3′
5′-TCACTTGTTCACCATGGG-3′
5′-GCCTTTGAGCACAAGTGT-3′
5′-CGGTCTGAGACAATTGCA-3′
5′-CGGAGTCAATGTTGGTCA-3′
5′-TGTGTAGGATGTGTTGCC-3′
5′-GCGAGAATCAGTGCATTC-3′
5′-GGTTTTGCTCTGTGTTGC-3′
5′-CGCAGAGTCAATGTGTGT-3′

ICC2* is a common ˜20-nt domains whose sequences are identical for different TCRs. ICC3* is another common ˜20-nt domains whose sequences are identical for different TCRs. The sequences of ICC2 and ICC3 differ, but both are unrelated to TCR. We carried out this process to synthesize a TCR library. This process is similar to the methods (e.g., FIGS. 17A-17C) disclosed in International Application No. PCT/US2020/026558, but with certain distinctions. For example, after step (6) Circularization, the circular DNA molecules were linearized (Step 7) not by cutting with a Type IIS restriction enzyme (TIISRE), but by PCR amplification using primer pairs [ICC3} and [ICC2*}. In the primer [ICC3} all T nucleotides were replaced by deoxyuridine. In the primer [ICC2*}, the first five phosphodiester linkages at the 5′ end were replaced by phosphorothioate linkages. The PCR product was treated with USER enzyme mixture (New England Biolabs) and DNA end-repair kid to remove the [ICC3}:[ICC3*} domains. In Step 8, the product from the previous step was treated with lambda exonuclease to degrade the top strand and annealed to the TRBC1 5′ primer followed by primer extension by a DNA polymerase.

To demonstrate the feasibility to individually purify each TCR from the TCR pool, 96 PCRs were set up. Each PCR contains a common forward primer targeting the common sequence upstream of TRBV #_GL5 and a TCR-specific [Zip*} primer. As shown in FIG. 1, all PCRs produced clear ˜1.5 kb product. The shorter bands were impurities that can be easily removed using Solid Phase Reversible Immobilization (SPRI). Sanger sequencing confirmed that PCR products produced this way contain only 1 TCR sequence. Each of these TCR genes can be treated with CorrectASE in a separate reaction. These products can then be pooled again, amplified with the forward primer and ICC*. This produce can be ligated into a plasmid vector, e.g., one that already has AAV Inverted Terminal Repeat (ITR), homology arm (HA, for CRISPR/Cas9-guided homologous recombination), and TRAC, see Examples below for details. The ligation product can be transformed into E. coli and a large number of colonies can be picked. For example, if ˜100 TCRs are intended to be used to produce synthetic TILs for a patient, 200 to 300 colonies can be picked. Each colony can be (1) propagated in 1 mL LB media and (2) verified with Sanger sequencing, simultaneously. The LB media corresponding to an error-free plasmid (based on Sanger sequencing result) can be pooled and the plasmid can be extracted as a mixture. At least 70% can be recovered in an error-free LB culture. This mixture can be transfected into packing cells (e.g., a 293T-derived cell line) along with other packaging plasmids to produce AAV particle mixtures, which can be used in knock-in-based TCR delivery into primary T cells (e.g., peripheral T cells of the patient from whom the synthesized TCR genes are derived).

### Example 2: Multiclonal TCR-T Manufactured with Single Genome Insertion (Tetramer-Based Demonstration)

This example shows a workflow that can achieve site-specific knock-in of a single copy of TCR gene in human primary T cells by using a mixture of AAV6 particles encoding multiple TCRs. Two variants of AAV6 viral particles were prepared each carrying a model TCR: one carrying a TCR against an HLA-A*02:01-presented WT-1 epitope (peptide sequence: RMFPNAPYL), another carrying an HLA-A*02:01-presented NY-ESO-1 epitope (peptide sequence: SLLMWITQC). The full DNA and amino acid TCR sequences (Seq ID E5.1NT, E5.1AA) between the homology arms (HAs) for the NY-ESO-1 specific TCR are listed below. The full DNA and amino acid TCR sequences (Seq ID E5.2NT, E5.2AA) between the HAs for the WT-1 specific TCR are listed below. The TCR has the following format: T2A-TRBVDJ-TRBC-P2A-TRAVJ. The DNA sequences of upstream and downstream HAs are listed as Seq ID E5.3NT and Seq ID E5.4NT, respectively. The ˜1,000-bp upstream and the ˜700-bp downstream HA have the sequence upstream and downstream of the cutting site of the TRAC-targeting guide RNA, respectively. The inserted sequence is to be knocked-in in-frame with respect to the endogenous TRAC, and begins with a T2A peptide, which terminates the translation of endogenous TRA and initiates the translation of the inserted, exogenous TCR.

Next, AAV6 viral particles were made by co-transfection of helper plasmid, capsid plasmid, and a transfer plasmid in 293Ta package cell lines. The transfer plasmid encodes a model TCR flanked by a pair of HAs, and further flanked by upstream and downstream Inverted terminal repeats (ITRs). For example, in one packaging reaction, 15 micrograms of helper plasmid, 15 micrograms of capsid plasmid, and 15 micrograms of transfer plasmid were co-transfected in 15e6 293Ta packaging cells (AAV-6 Helper Free Packaging System from CELL BIOLABS, INC, Cat #VPK-406; 293 lentipack cells from Genecopoeia, Cat #LT-008). Three days after transfection AAV6 particles were harvest by lysis the cells with pure chloroform (1:10 volume) and raw virus were enriched by PEI/NaCl precipitation. Briefly, NaCl was added to cell lysate to a final concentration of 1 M. Cell debris were removed by centrifuging at 20,000 g at 4° C. for 15 min. The aqueous layer was mixed with 10% (w/v) PEG8000 and shaken until dissolved. The mixture was incubated at 4° C. for 1 h or longer for virus particles to precipitate and then centrifuged at 20,000 g at 4° C. for 15 min. Virus particles were collected as the pellet. The concentrations of the AAV6 particles (i.e., genome copy per microliter) were measured by a qPCR assay (QuickTiter™ AAV Quantitation Kit from CELL BIOLABS, INC, Cat #VPK-145). The viral particles were resuspended in 0.001% Pluronic F68/200 mM NaCl PBS and stored at −80° C. before use.

To demonstrate single-copy site-directed knock-in of polyclonal TCRs, the two variants of AAV6 particles were mixed at different ratios and then used as donor DNA to carry out knock-in (KI) of primary peripheral T cells. The rationale is that if the two exogenous TCRs are inserted independently, then a substantial fraction of the T cells can receive two exogenous TCRs, resulting in the T cell staining positive for both pMHC multimers (each corresponding to a TCR). Quantitatively, in the scenario of independent insertion, if x % of T cells stain positive for the first pMHC tetramer and y % of T cells stain positive for the second pMHC tetramer, (x %)*(y %) of T cells are expected to stain positive for both pMHC tetramers. However, if (a) the two exogenous TCRs compete for one genome location during KI, and (b) the TCR is only expressed after they are inserted into the genome, then each TCR can primarily only stain positive for one pMHC tetramer.

To test this rationale, a healthy donor's T cells were isolated by using EasySep™ Release Human CD3 Positive Selection Kit (StemCell, Cat #17751). Then, the T cells were activated by Dynabeads™ Human T-Expander CD3/CD28 with 0.5e6 T cells per 1 mL media in 1 well of a 24-well plate. The amount of beads used was 3 beads per cell (e.g., 3e6 beads per 1e6 T cells) (ThermoFisher Cat #11141D). After cells were fully activated, Cas9/TRAC+TRBC sgRNAs formulated RNP were electroporated together to the activated T cells. RNP was formulated freshly by adding water, Cas9 buffer, sgRNAs (one targeting TRAC and the other targeting TRBC) and Cas9 sequentially to form 20 μM working stock at room temperature and allow to stand for 30 min. AAV6 viral particle mixture were added to T cells at 5e4 genome copies per T cells 1-3 hours after electroporation. The following steps was then performed: Seed T cells/AAV6 back to 24-well plate at 2.5e5/mL/well; Collect T cells from each well, pool together in tube and debead on magnet; Count cells and adjust cell concentration to 2.5e5 to 5e5 per mL by adding fresh media with 200 U/mL IL2. At day 5 or 6, take a 200 μL cell suspension in 96-well U bottom plate for Live/Dead staining at room temperature for 30 min (LIVE/DEAD Fixable Near-IR stain kit (Thermal Cat #L34955), followed by tetramer staining (NY-ESO tetramer-APC: MBL Cat #TB-M011-2 and WT1 tetramer-PE: MBL Cat #TB-M016-1). After incubation at 4° C. for 30 min, cells were washed twice in the plate with FACS buffer (1×PBS, 2% FBS, 2 mM EDTA) and load on Cytoflex to allow collecting 30,000 live T cells.

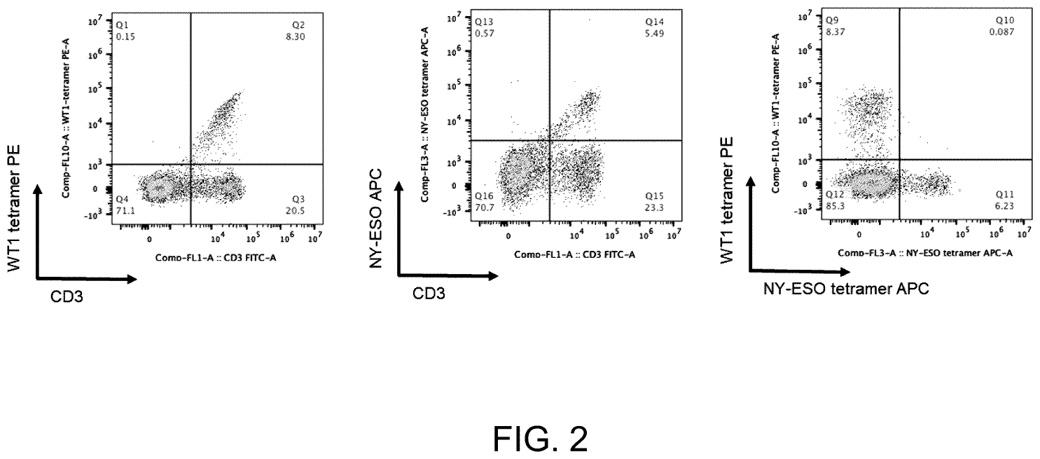

The following steps were performed: Gate T cells on FSC-SSC plot and further set the gate by staining controls (e.g., FMO controls, essentially all fluorescence stained minus one color); Choose the live cell population on full stained samples by gating LIVE/DEAD Near-IR negative population; Check APC-PE plot. FIG. 2 shows tetramer staining using antibodies against CD3, WT1, and NY-ESO. As shown in FIG. 2 (right panel), around 8.37% live T cells showed WT1 tetramer-PE positive but NY-ESO tetramer-APC negative staining while 6.23% live T cells showed NY-ESO tetramer-APC positive but WT1 tetramer-PE negative staining. Only 0.087% of the T cells showed APC and PE double positive staining, which is less than the expected double-positive rate in the independent-insertion scenario (8.37%*6.23%=0.52%), suggesting that for the majority of T cells, only one copy of TCR was productively inserted.

### Example 3. Synthetic TIL Cells (Multiclonal TCR-T with Autologous TCR Sequences) Manufactured with Single Genome Insertion (Single-Cell RNA-Seq (scRNA-Seg)-Based Demonstration)

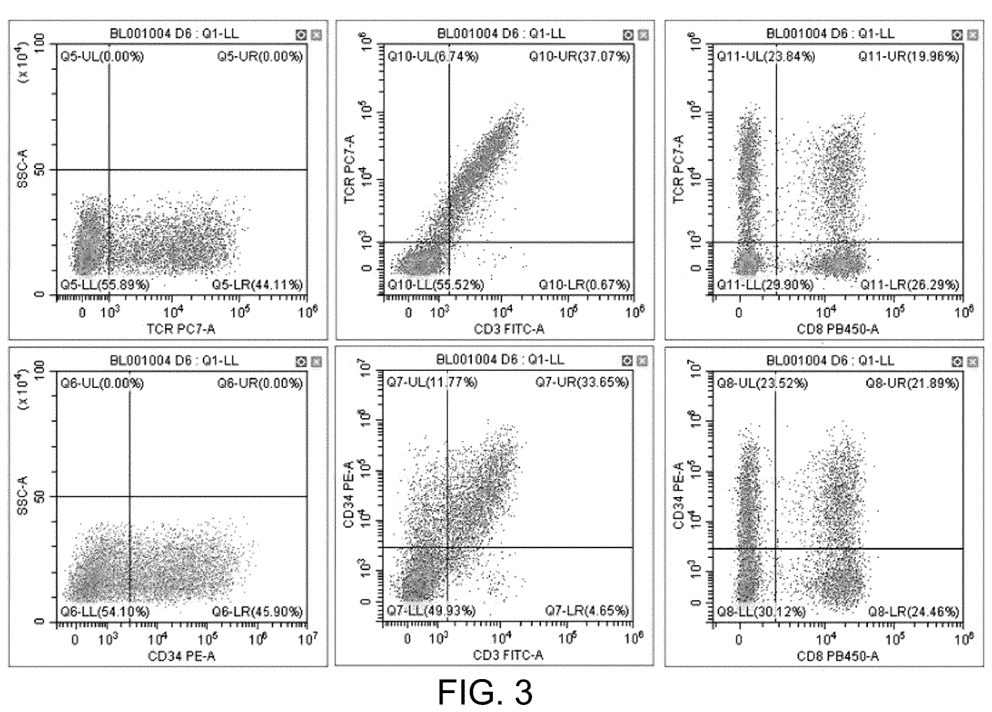

To test whether hundreds to thousands of TCRs can be delivered to a population of T cells while maintaining that the vast majority of T cells only express one exogenous TCR, we prepared a mixture of ˜1,000 TIL-derived TCR genes each in the format of the model TCR described above, except that a T2A-RQR8 sequence was appended in-frame upstream of the TCR alpha chain. The RQR8 is a designed short transmembrane protein that contains a minimal CD20 epitope and a minimal CD34 epitope. This mixture was ligated into the AAV6 transfer plasmid backbone that also contains the upstream and downstream HAs (sequence shown in Example 2). The ligation product was transformed into E. coli and the transformation product was grown in liquid LB media without picking single colonies (the process with picking colonies can be implemented as described in Example 1). The E. coli grown in liquid LB media underwent plasmid preparation to yield a mixture of AAV6 transfer plasmids containing ˜1,000 TCR genes, where each plasmid molecule contains only one TCR gene. This transfer plasmid mixture was used to produce a mixture of AAV6 viral particles containing ˜1,000 TCR genes, where each AAV6 viral particle only contains one TCR gene, using a similar process as described in Example 2. The AAV6 viral particle mixture was used along with Cas9 and human TRBC- and TRAC-targeting guide RNA to achieve simultaneous inactivation of endogenous TRB and TRA, while inserting the exogenous TCR at the TRAC locus, using a similar method as described in Example 2. The cells were analyzed by flowcytometry using antibodies against CD34, CD3, CD8, and TCR complex. After this process, about 45% of the T cells expressed the RQR8 epitope based on staining with anti-CD34 antibody (FIG. 3). Since the resultant T cells express a polyclonal repertoire of TIL-derived TCRs, these T cells can be called synthetic TILs.

This T cell population was enriched with anti-CD34 antibody in conjunction with magnetic beads to enrich the RQR8-positive T cells. About 90% of the resultant T cells were RQR8-positive. Since RQR8 was inserted in-frame with the exogenous TCR (and separated by 2A peptides), the RQR8-positive T cells expressed exogenous TCR as well.

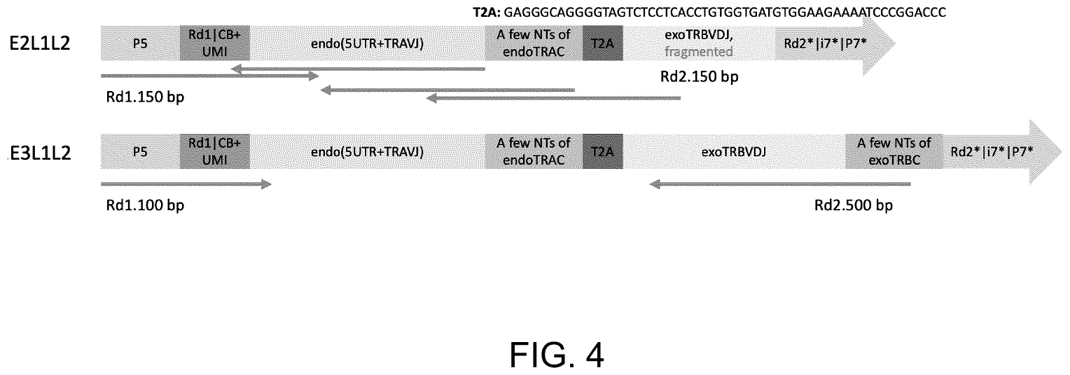

To study the percentage of RQR8-positive T cells that express more than one exogenous TCRs, the CD34-enriched T cells were analyzed with single-cell RNA-Seq using the 10× Genomics Chromium Next GEM single cell 5′ reagent kit. After the droplet formation, reverse transcription, template switching (which adds the cell barcode, abbreviated as CB, and unique molecule identifier, abbreviated as UMI, through the template-switching oligonucleotide, abbreviated as TSO), and demulsification, the full-length cDNA was amplified for a small number (typically 8) of cycles according to the user manual. Next, a forward primer targeting TSO and a reverse primer targeting the exogenous TRBC were used to amplify full-length cDNA molecules containing the exogenous TCR. Illumina sequencing adaptors were added in a nested PCR where the reverse primer targeted an upstream region of TRBC, resulting in an NGS library shown as E3L1L2 in FIG. 4. Standard Read 1 and Read 2 sequencing primers were used to read the barcodes (including CB and UMI) and the variable region (noted as TRBVDJ) of the exogenous TCR, respectively. Sequencing of this library established the linkage between the barcodes and the sequence (therefore clonotype) of the exogenous TCR. This data (see below) informed us how many exogenous TCR can be detected for each synthetic TIL cell. However, if two exogenous TCR clonotypes are detected in one cell, it does not necessarily mean that both TCRs can be translated into protein and produce mis-paired TRA and TRB chains (e.g., productively inserted, or productively expressed). This is because the TRAC locus into which one of the TCRs is inserted may be part of an unrearranged or unproductive endogenous TRA. For example, the endogenous TRAV and TRAC in this chromosome may be out of frame as a result of the V-J recombination. In this case, the inserted exogenous TCR, although detectable, cannot be expressed (since the exogenous TCR is used in-frame with the endogenous TRAC).

To understand which detected exogenous TCRs are productively expressed (e.g., can be translated into proteins), the DNA library E3L1L2 was fragmented to appropriate size distribution using the Nextera kit (or equivalent) to generate DNA library E2L1L2. In this process, multiple fragmented molecules were generated for each original cDNA molecule (identifiable by the CB+UMI barcode combination). During NGS, these molecules generated multiple reads, which were then digitally assembled into a contig. This contig shows the upstream sequence of the mRNA molecule that carries the exogenous TCR. An exogenous TCR was considered productive if the TRAV, TRAJ and TRAC sequences are connected in-frame without stop codon.

The sequencing of the E3L1L2 library detected 36,886 cDNA molecules (note: each cDNA molecule is represented by a consensus sequence built from reads sharing the same CB-UMI combination, or ‘CB-UMI-Consensus’ for brevity) containing an exogenous TCR.

Usually, each integration event (also called insertion event or KI event; identifiable by the CB-clonotype combination) may yield multiple detected cDNA molecules. Since the scRNA-Seq process may misassign barcodes to a read (e.g., due to template switching after demulsification or PCR chimera), CB-UMI-Consensuses were removed if only one CB-UMI-Consensus was detected. Moreover, CB-UMI-Consensuses were removed if more than 2 exogenous TCRs were identified for a single CB, which was likely caused by cell collision during droplet formation. After this process, 12,840 CB-UMI-Consensus sequences remained. By matching the CB-UMI between E2L1L2 and E3L1L2, we found 9,066 CB-UMI-Consensuses whose productivity status can be evaluated, the remaining ones failed due to reasons such as short/unmappable contig and off target KI (all information gleaned from the E2L1L2 run). Analysis of the CBs showed that these CB-UMI-Consensuses (or cDNA molecules) were derived from 1,846 cells. A totally of 2,022 KI events (into the TRAC loci) were detected, where each KI event was identified by a unique CB-clonotype combination. A KI event was deemed productive if the CB-UMI-Consensus (e.g., cDNA molecules) belonging to the KI events are predominately productive, defined here as [productive CB-UMI-Consensuses]/[unproductive CB-UMI-Consensuses]>5 or [unproductive CB-UMI-Consensuses]=0. A KI event was deemed unproductive if [productive CB-UMI-Consensuses]=0. Otherwise, the productivity status of the KI event was deemed undeterminable. According to these definitions, 1,542 KI events were productive, 303 were unproductive, and 177 were undeterminable.

When the KI events of each individual cell were analyzed, we found that 155 cells showed no KI event, 1537 cells showed one KI event, 154 cells showed two KI events. When the productivity status was taken into consideration, we saw that 370 cells showed no productive KI event, 1410 cells showed one KI event, and 66 cells showed two KI events.

Table 4 shows the CB-UMI-Consensus count (or UMI count for brevity) for each KI event in each cell. Cells (on the horizontal axis) are rank ordered based on total UMI count assigned to the cell. Based on the data above, it can be estimated that 1410/(1410+66)=95.5% of “exogenous TCR-positive” T cells (e.g., T cells that productively express an exogenous TCR) express only one exogenous TCR. However, this may be an overestimation because if only a small number of cDNAs are discovered from a cell, only cDNAs from one KI event may be seen in the NGS data and cDNAs from the other KI event may be missed. If we only examine cells that have 8 or more total UMIs (248 cells), 9 cells showed 0 productive KI events, 212 cells showed 1 productive KI events, and 27 cells showed 2 productive KI events. In these cells, 212/(212+27)=88.7% of exogenous TCR-positive T cells express only one exogenous TCR. It should be noted that, cell collision (e.g., droplets that contain more than one cells) may inflate the number of productive KI events.

### Example 4: Obtaining Potentially Tumor-Reactive TCR Sequences from Needle Biopsy Samples

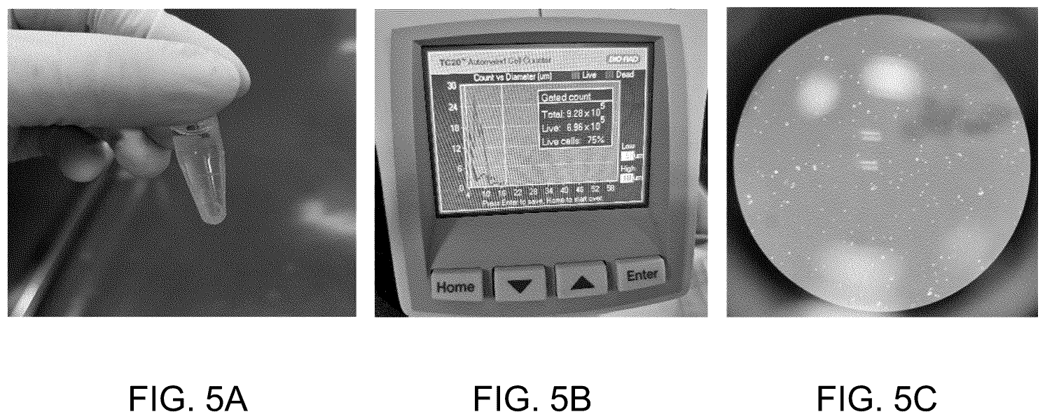

The following protocol was followed to perform single-cell RNA-Seq for fine needle aspiration samples obtained from Patient 1 (see Example 5). First, use 18G needles to puncture the tumor to obtained at least 2-3 pieces (about 1.5 cm in length). Avoid necrotic sites when retaining tissue samples. Immediately after taking out the tissue samples, transfer them into Miltenyi tissue preservation solution, and store and transport them at 2-8° C. (FIG. 5A). Tissue preservation solution was stored for no more than 20 h. Then the sample was put in 1.5 ml or 15 ml centrifuge tube, and then an appropriate volume of collagenase I (Collagenase I) solution was added to the sample, and then the sample was digested at 37° C. for 45 min-1 h on a shaker. Collagenase was dissolved in RPMI-1640 medium at a working concentration of 1 mg/ml.

After the digestion was completed, an equal volume of RPMI-1640 medium containing 2% FBS was added to each tube, which was inverted and mixed well to stop the collagenase reaction. Grind on a 70 μm nylon filter with the plastic end of a syringe (alternatively, a 300-400 mesh stainless steel filter can be used instead). After grinding and filtering, the single-cell suspension was examined using Bio-Rad TC20 Automated Cell Counter (FIG. 5B) and optical microscopy (FIG. 5C) then loaded on a 10× Chromium Controller.

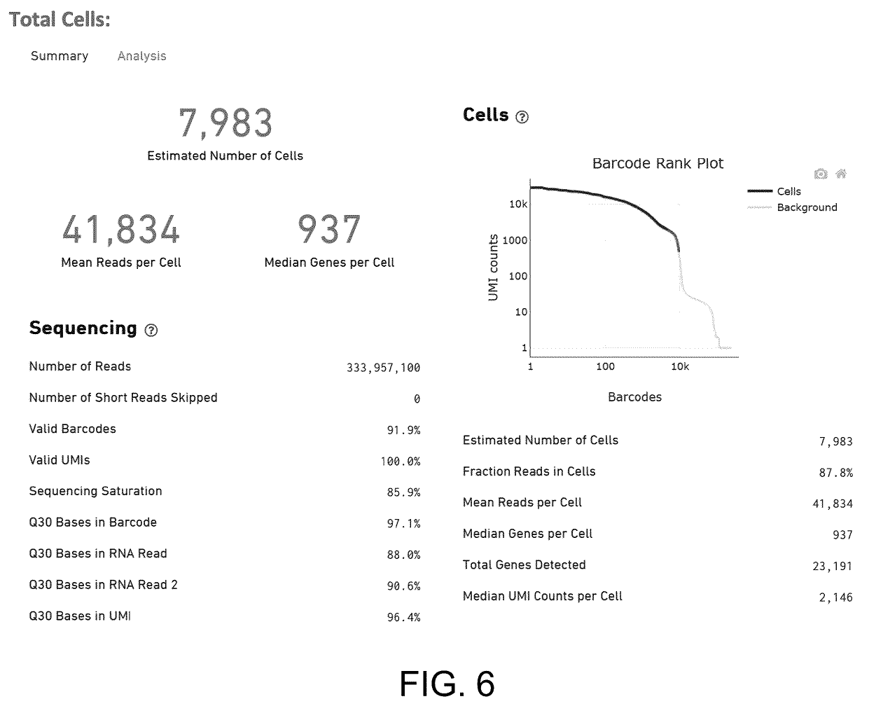

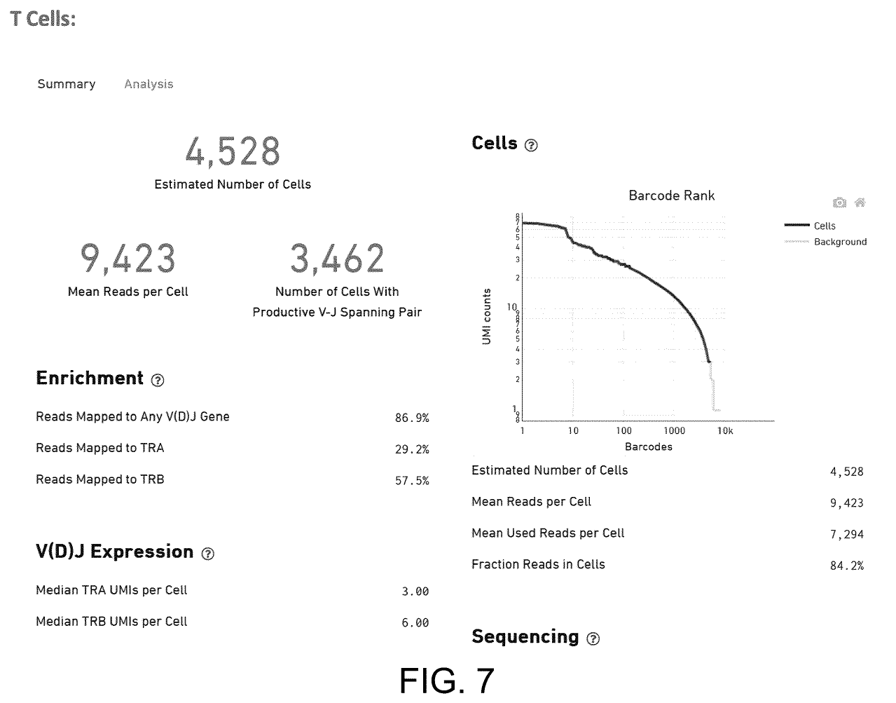

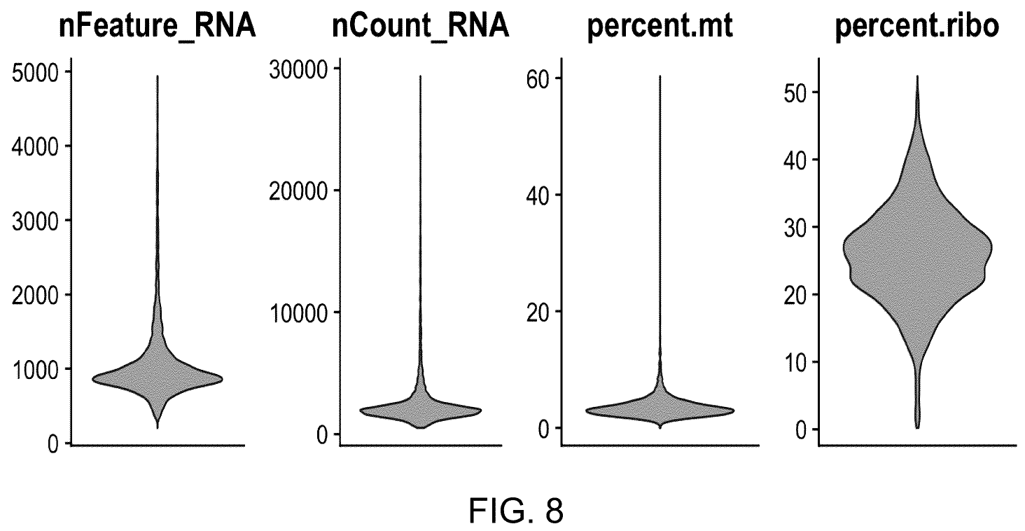

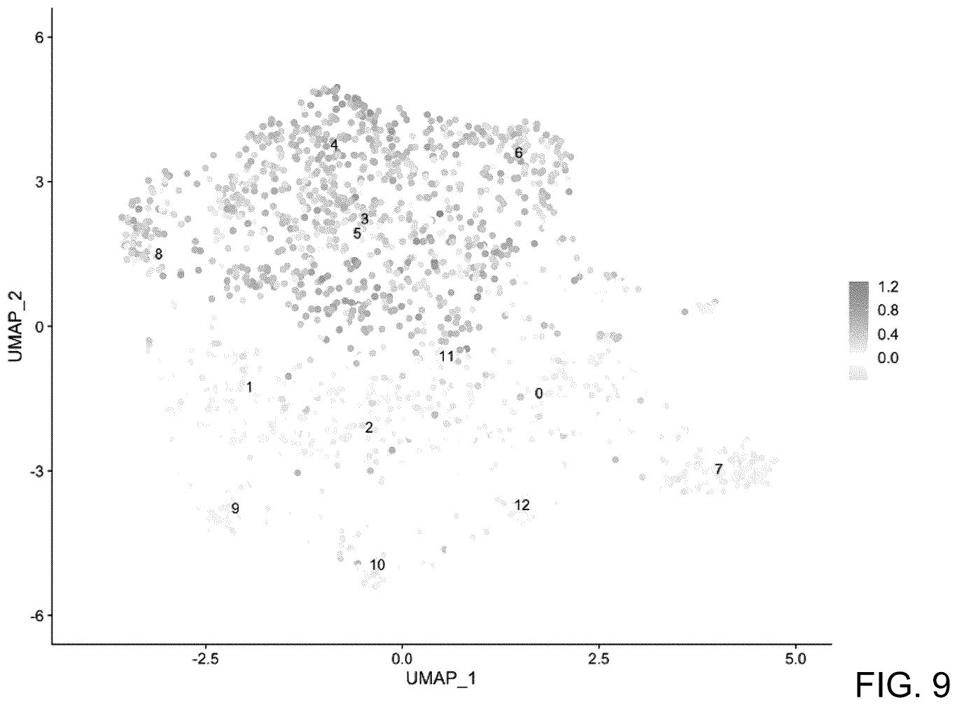

The single-cell sequencing run generated transcriptome information of 7,983 cells, including tumor cells, T cells and other cells in the tumor microenvironment (FIG. 6), as well as TCR sequences from 4,528 cells, out of which 3,462 cells contain productive V-J spanning pair (FIG. 7). The distribution of feature count (e.g., gene count), transcript count (e.g., UMI count), percentage of mitochondria-derived transcripts, and percentage of ribosome-derived transcripts are shown in FIG. 8. The UMAP position and ‘tumor-reactive signature score’ (calculated using the algorithm shown in Example 7) of each T cell is shown in FIG. 9.

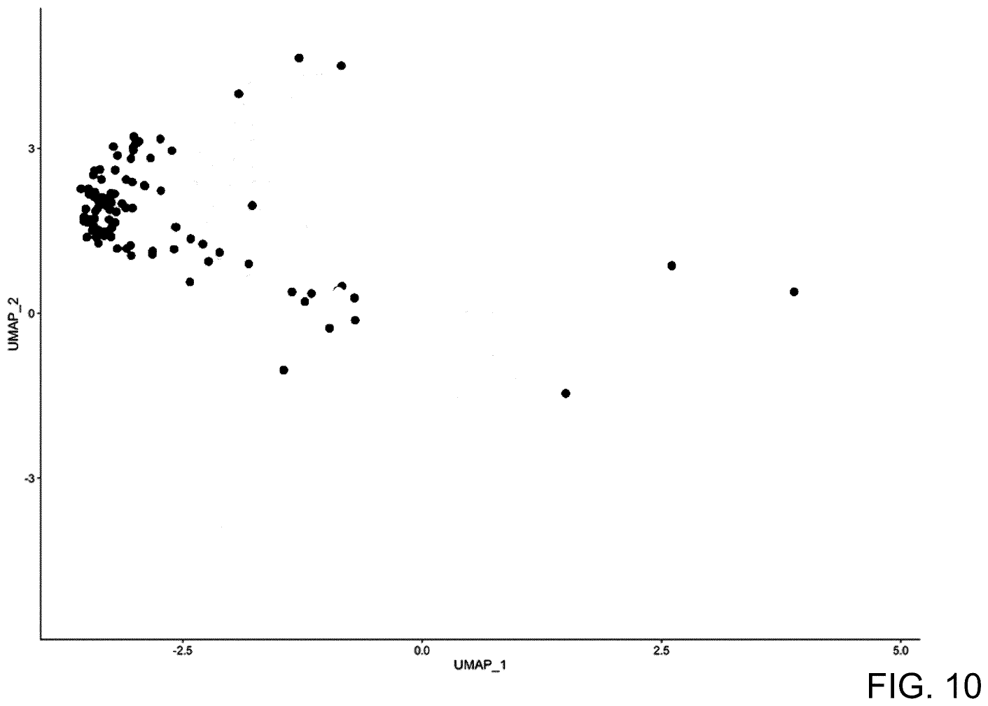

Three potentially tumor-reactive TCRs: TCR1 (FIG. 10), TCR2 (FIG. 11), and TCR3 (FIG. 12) were identified based on the fact that these TCRs are clonally expanded and the T cells carrying these TCRs have high tumor-reactive signature score. The sequences of these TCRs are shown in Table 2.

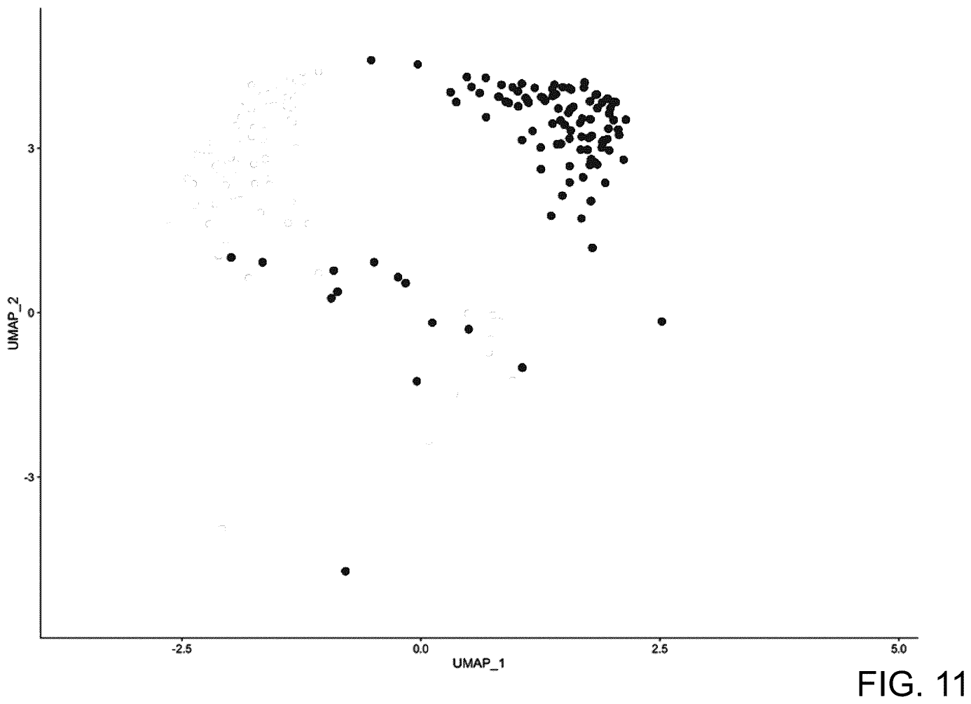

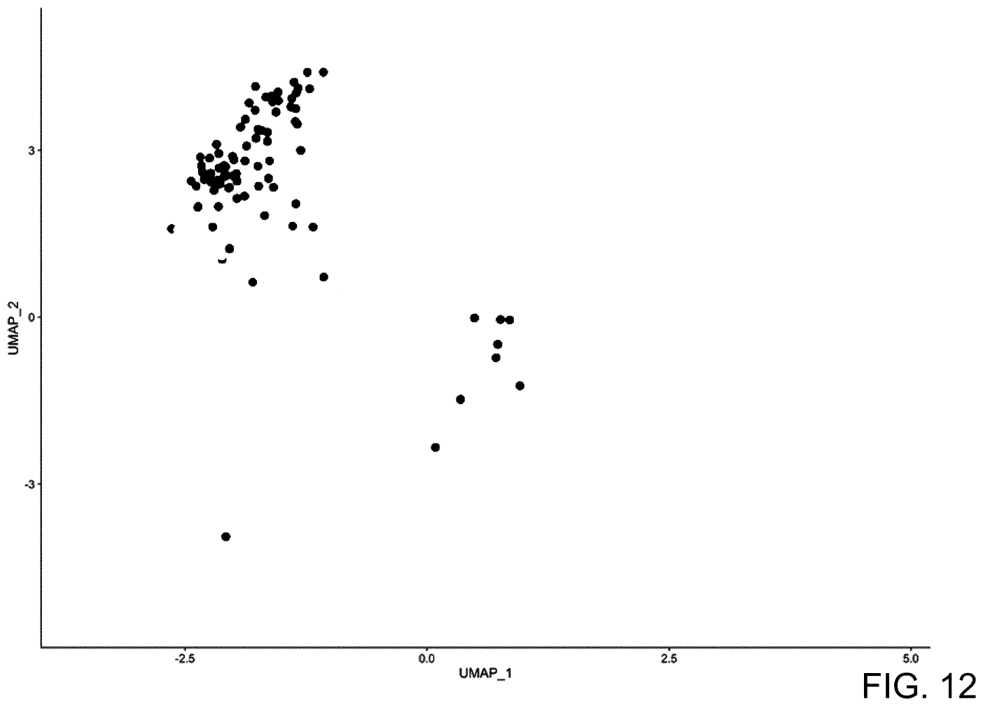

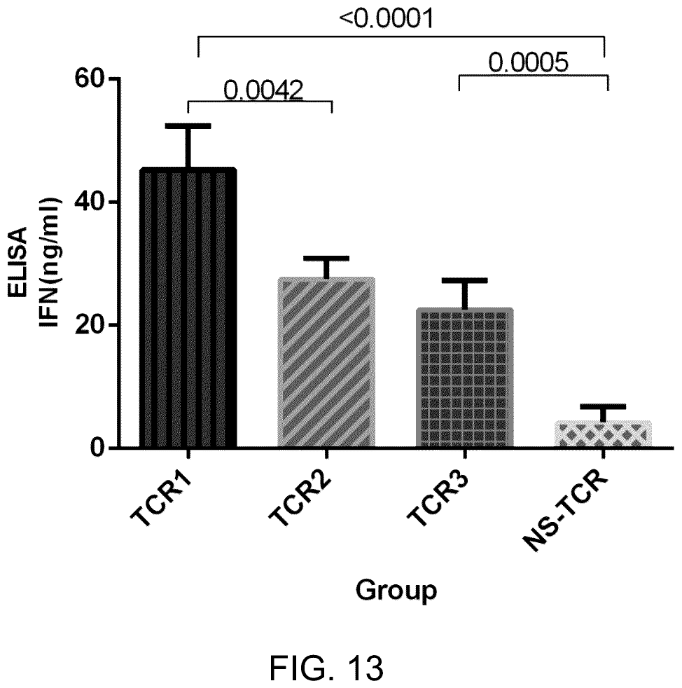

Another TCR, named NS-TCR, was also randomly selected as a control. The 4 TCRs were synthesized, cloned into lentivectors, made lentivirus and infected patient's peripheral T cells to make 4 clones of TCR-T cells. Each clone of TCR-T cells was incubated with primary tumor cells at the ratio of 1:1 in 96 well. IFNg ELISA show that TCR1, TCR2 and TCR3 recognized the tumor cells while the NS-TCR did not (FIG. 13).

The 18G needle has an inner diameter of 0.84 mm. If the biopsy sample has a length of 1.5 cm. The volume of each piece of biopsy sample is approximately 3.14*(0.84/2)\hat{ }2*15 mm³=8.3 mm³ or 8.3 μL. Three pieces of biopsy sample have a total volume of approximately 8.3*3=˜25 μL. If we assume the tumor sample has the density of 1 g/mL, i.e., 1 mg/μL, the total mass of 3 pieces of needle biopsy sample is approximately 25 mg. Thus, this example shows that paired TCR and transcriptome information of thousands of tumor-infiltrating T cells can be obtained and potentially tumor-reactive TCR sequences can be obtained from as little as 25 mg of tumor sample. Linear interpolation shows that as little as 5 mg or 10 mg of tumor material can generate sufficient T cell information to produce synthetic TILs using methods described herein.

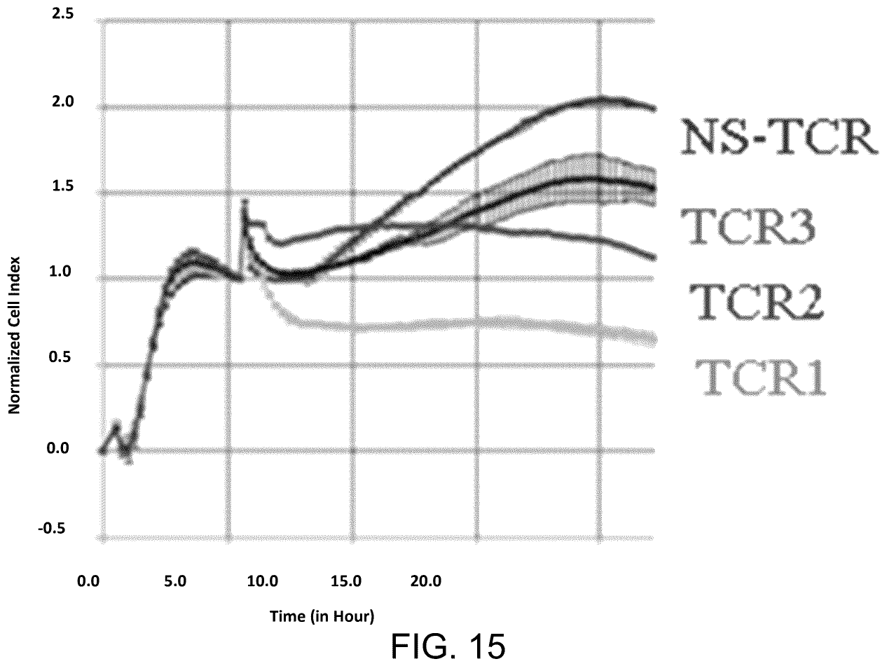

Three TCRs were identified for another patient (Patient 2) using a similar process. For this patient, patient-derived cancer cell line was obtained. And the TCR-T cells were used to conduct a RTCA-based T cell killing assays with the ratio of T cells to cancer cells being 10:1. It can be seen from FIG. 15 that while the three potentially tumor reactive TCRs (TCR1, TCR2, TCR3) showed tumor killing, the randomly chosen TCR (NS-TCR) did not.

### Example 5: Treatment of a Hepatocellular Carcinoma Patient (Patient 1) with Synthetic TIL

Patient 1 was diagnosed with hepatocellular carcinoma, and underwent liver cancer resection+cholecystectomy in November 2019. The postoperative pathological immunohistochemistry showed that the pathological stage was IIIA (T3, NO, MO), tumor cells GPC3 part+, CK19 part+, Ki-67 about 40%, AFP part+, VEGFR2−, PD-L1 (SP142) tumor 5% stroma 5%, PD1−, CD3 lymphocyte+, NY-ESO-1−, GLS1++; another block CK7+, Hep1−, SALL4−, Arg-1−, CD10+, CK19+, CD31 vessel+, CD34 vessel+, Des muscle wall+. Repeated CT scans after operation showed enlarged pulmonary nodules, and tumor metastasis in lung was considered. After taking Lenvatinib for more than three months, the drug was discontinued due to serious adverse reactions (diarrhea, loss of appetite, and peeling of the joints). Hepatic arterial infusion chemotherapy was performed in June 2020, July 2020 and September 2020 after surgery. The chemotherapy regimen was Raltitrexed plus Epirubicin hydrochloride.

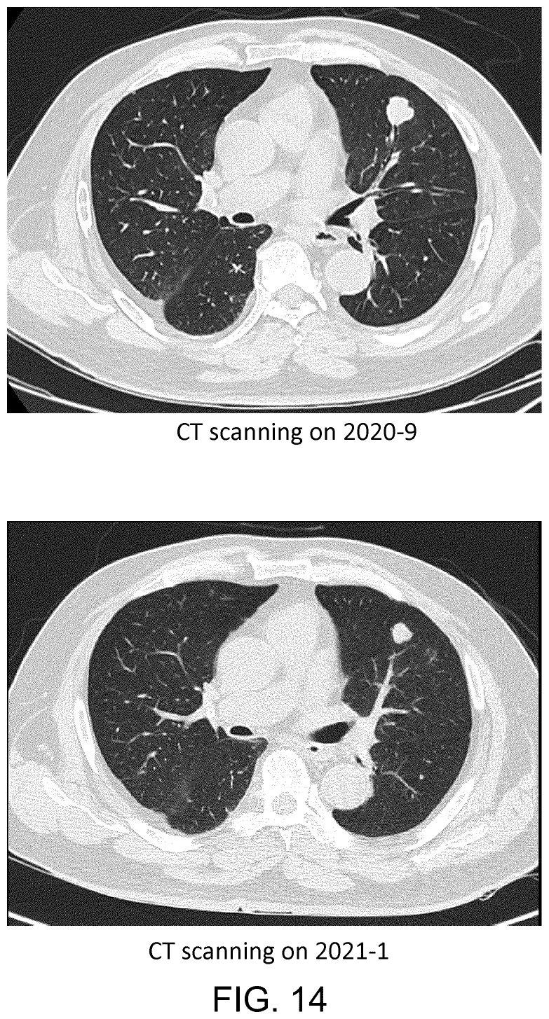

This patient was consented and enrolled in synthetic TIL clinical trial. There potentially tumor-reactive TCRs (TCR1, TCR2 and TCR3, sequence shown in Table 2) were identified as described in Example 4. Blood cell separation and apheresis was performed in June 2020. TCR-T cells was produced using each potentially tumor-reactive TCR using lentiviral vector. The synthetic TIL product was produced by mixing three clones of TCR-T cells (TCR1: 0.8e9 T cells, TCR2: 1e9 T cells, TCR3: 1.1e9 T cells). The patient was treated with the synthetic TIL product with i.v. infusion in September 2020 without lymphodepletion. There was no obvious adverse reaction after synthetic TIL treatment, and the patient reported feeling normal. CT scanning in January 2021 demonstrated a significant regression of lung metastasized lesion (FIG. 14).

### Example 6: Treatment of a Post-PD1 Urothelial Cancer Patient with Synthetic TIL

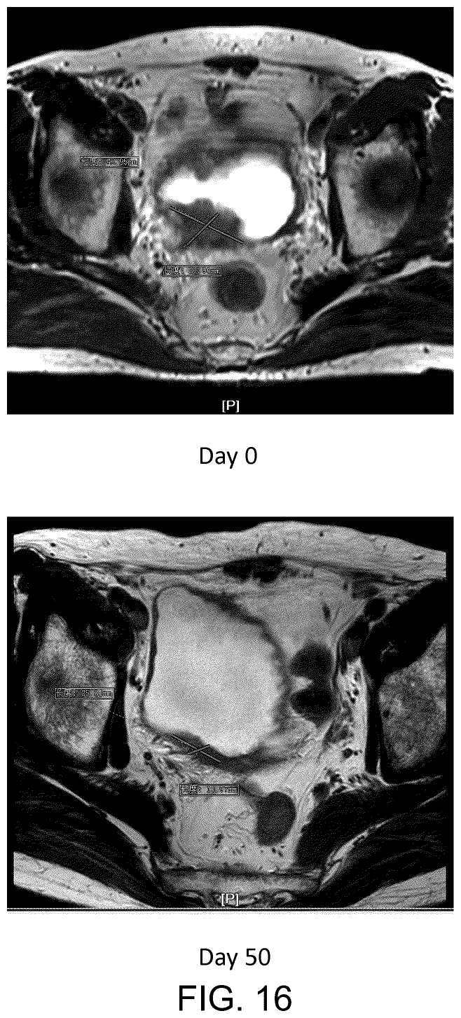

A patient with high grade upper tract urothelial cancer (Patient 3) presented multiple small legions in the lung, suggesting distal metastasis. The patient was treated with gemcitabine and cisplatin, which did not lead to tumor regression. The patient was then treated with checkpoint blockade Toripalimab. The tumor was still progressive after the treatment. The patient was consented and enrolled in an investigator-initiated interventional synthetic TIL clinical trial. A biopsy sample was obtained with cystourethroscopy. The biopsy sample was dissociated into single cell suspension. A portion of the suspension was cryopreserved for tumor-reactivity testing. Three potentially tumor-reactive TCRs were identified using a process similar to that shown in Example 4 and used to manufacture synthetic TIL as described in Example 5. The patient was then infused with 1e10 synthetic TIL cells intravenously without lymphodepletion. 50 days after the infusion the patient was evaluated with MRI. As shown in FIG. 16, the longest diameter of the target lesion was reduced from 48.95 mm to 25.31 mm.

### Example 7. Calculating Tumor-Reactive Signature Score Using Transcriptome Information

The following code was used to calculate tumor-reactive signature score using transcriptome information.

```
title: ″TCR-T analysis″
date: ″‘r format(Sys.time( ), ′%d %B, %Y′)‘″
output: html_document
---
‘‘‘{r setup, include=FALSE}
knitr::opts_knit$set(root.dir = $ROOT.DIR)
‘‘‘
‘‘‘{r, include=FALSE, message=FALSE, warning=FALSE}
library(plyr)
library(Seurat)
library(SeuratObject)
library(dplyr)
library(Matrix)
library(knitr)
library(ggplot2)
library(cowplot)
library(RColorBrewer)
library(ggrepel)
library(tidyr)
library(Biostrings)
library(ggpubr)
‘‘‘
‘‘‘{r, message=FALSE, echo=FALSE, warning=FALSE}
# Define paths and ids
rootpath <− $ROOT.DIR
rawdatapath <− file.path(rootpath, ″raw_data″)
figurepath <− file.path(rootpath, ″figures″)
datapath <− file.path(rootpath, ″data″)
gex_ids <− c(″WFL_NEG_T_GEX″)
tcr_ids <− c(″WFL_NEG_T_TCR″)
prefix <− gex_ids[1]
‘‘‘
TCR-T analysis of ″‘r gex_ids[1]‘″
‘‘‘{r, message=FALSE, echo=FALSE, warning=FALSE}
#Load data
name <− paste(prefix, ″_merged.obj.rds″, sep = ″″)
merged.obj <− readRDS(file = file.path(datapath, name))
‘‘‘
‘‘‘{r, message=FALSE, include=FALSE, warning=FALSE}
act.signature <− c(″CXCL13″, ″CTLA4″, ″DUSP4″, ″ENTPD1″, ″RBPJ″, ″TNFSF4″, ″GK″, ″PDCD1″,
″LAG3″, ″FKBP1A″, ″MYO7A″, ″FABP5″, ″GAPDH″, ″TIGIT″, ″CDCA7″, ″ZBED2″, ″RGS1″,
″HAVCR2″, ″MKI67″, ″GZMB″, ″SPRY1″, ″PHLDA1″, ″ASPM″, ″MYO1E″, ″TOX″, ″LSP1″,
″MCM10″, ″MAD2L1″, ″OGN″, ″PCLAF″, ″UBE2T″, ″GNG4″, ″LY6E″, ″TPI1″, ″TNFRSF18″,
″RGS2″, ″CLEC2D″, ″SAMSN1″, ″TSHZ2″, ″HMGN2″)
merged.obj <− AddModuleScore(object = merged.obj, features = list(act.signature), name =
″activation.score″)
act_scores_umap <− FeaturePlot(object = merged.obj, features = ′activation.score1′, label = T, pt.size = 2)
+ scale_colour_gradient2(low = ″#3399FF″, mid = ″white″, high = ″red″, midpoint = 0, na.value = ″red″)
name <− paste(prefix, ″_activation.score.UMAP.pdf″, sep = ″″)
ggsave(act_scores_umap, file=file.path(figurepath, name), width = 10, height = 8)
act_scores <− merged.obj@meta.data$activation.score1
act_p <− rank(−act_scores)/length(act_scores)
merged.obj@meta.data$act_p <− act_p
act_cut <− subset(merged.obj, act_p>=0.05)
cut_value_act <− max(act_cut@meta.data$activation.score1)
merged.obj.tcrt <− subset(merged.obj, activation.score1>cut_value_act)
tcrt_meta <− merged.obj.tcrt@meta.data
tcrt meta <− tcrt_meta %>% arrange(desc(tcr_proportion))
top3.clones <− unique(tcrt_meta$tcr_cdr3s_aa)[1:3]
merged.obj@meta.data[!merged.obj@meta.data$tcr_cdr3s_aa %in% top3.clones, ″tcr_cdr3s_aa″] <−
″non-TCR-T-cells″
orderby <− c(top3.clones, ″non-TCR-T-cells″)
merged.obj@meta.data <− merged.obj@meta.data[order(match(merged.obj@meta.data$tcr_cdr3s_aa,
orderby)),]
ggplotColours <− function(n = 6, h = c(0, 360) + 15){
  if ((diff(h) %% 360) < 1) h[2] <− h[2] − 360/n
  hcl(h=(seq(h[1], h[2], length = n)), c = 100, 1 = 65)
}
mycol <− ggplotColours(length(top3.clones))
mycol <− c(mycol, ″grey80″)
mylabel <− c(top3.clones, ″non-TCR-T-cells″)
top3_tcrt_clones_umap <− DimPlot(merged.obj, reduction = ″umap″, pt.size = 4, order = mylabel, label =
F, group.by = ″tcr_cdr3s_aa″) + theme(plot.title = element_blank( )) +
scale_colour_manual(values=mycol, limits = mylabel)
name <− paste(prefix, ″_top3.TCRT.UMAP.pdf″, sep = ″″)
ggsave(top3_tcrt_clones_umap, file=file.path(figurepath, name), width = 20, height = 10)
‘‘‘
## Activated T cells
‘‘‘{r, message=FALSE, echo=FALSE, warning=FALSE, fig.width=10, fig.height=8}
act_scores_umap + ggtitle(″Activation scores″)
‘‘‘
## Top 3 TCR-T clones
‘‘‘{r, message=FALSE, echo=FALSE, warning=FALSE, fig.width=20, fig.height=10}
top3_tcrt_clones_umap
‘‘‘
‘‘‘{r, message=FALSE, include=FALSE, warning=FALSE}
# Report TCR sequences
consensus_seqs = readDNAStringSet(file.path(rawdatapath, tcr_ids[1], ″consensus.fasta″))
consensus_seqs <− as.data.frame(consensus_seqs)
consensus_seqs$raw_consensus_id <− rownames(consensus_seqs)
colnames(consensus_seqs) <− c(″consensus″, ″raw_consensus_id″)
meta <− read.table(file.path(rawdatapath, tcr_ids[1], ″filtered_contig_annotations.csv″), sep = ″,″, header
= T)
meta1 <− merge(meta, consensus_seqs, by = ″raw_consensus_id″, all.x = TRUE, sort = FALSE)
clonotypes <− read.table(file.path(rawdatapath, tcr_ids[1], ″clonotypes.csv″), sep = ″,″, header = T)
meta1 <− merge(meta1, clonotypes, by.x = ″raw_clonotype_id″, by.y = ″clonotype_id″, all.x = TRUE,
sort = FALSE)
meta$barcode1 <− paste(meta$barcode, meta$chain, sep = ″_″)
meta$barcode1 <− paste(meta$barcode1, meta$raw_consensus_id, sep = ″_″)
meta1$barcode1 <− paste(meta1$barcode, meta1$chain, sep = ″_″)
meta1$barcode1 <− paste(meta1$barcode1, meta1$raw_consensus_id, sep = ″_″)
index <− match(meta$barcode1, meta1$barcode1)
meta1 <− meta1[index, ]
meta1.top3 <− meta1[meta1$cdr3s_aa %in% top3.clones, ]
meta1.top3 <− meta1.top3[!is.na(meta1.top3$consensus), ]
meta1.top3 <− meta1.top3[order(match(meta1.top3$cdr3s_aa, top3.clones)),]
meta1.top3$chain_cdr3 <− paste(meta1.top3$chain, meta1.top3$cdr3s_aa, sep = ″_″)
meta1.top3 <− meta1.top3[, c(″chain″, ″cdr3s_aa″, ″consensus″, ″chain_cdr3″)]
meta1.top3 = meta1.top3[!duplicated(meta1.top3$chain_cdr3),]
meta1.top3 <− meta1.top3[, c(″chain″, ″cdr3s_aa″, ″consensus″)]
name <− paste(prefix, ″_top3.tcrt.clones.txt″, sep = ″″)
write.table(meta1.top3, file.path(datapath, name), quote = F, sep = ″\t″, row.names = F)
‘‘‘
‘‘‘{r, message=FALSE, include=FALSE, warning=FALSE}
# Session information
sessionInfo( )
‘‘‘
```

### Example 8. Multiclonal TCR-T Manufactured with Single Genome Insertion Using Lentiviral Vectors

In certain situations, lentiviral vector can be used to manufacture multiclonal TCR-T cells. The challenge is that, unlike CRISPR/Cas9-guided site-specific genome knock-in (which allows knock-in into a gene locus with monoallelic expression such as TRAC or loci on sex chromosomes), genome insertion mediated by lentiviral vector may be random. After lentiviral transduction, 5 to 10 insertions in the same cell can be routinely observed. If a mixture of lentiviral particle containing multiple TCR genes (each lentiviral particle containing one TCR gene) is used to transduce a population of T cells, lentiviral vector DNA of multiple TCRs may be inserted to one T cells. This may result in the expression of multiple TRA chains and multiple TRB chains in the same cells, and these TRA and TRB chains may assemble into TCR heterodimers in a non-native fashion.

Methods provided herein can be used to solve the above mentioned problems. For example, a single TCR gene can be used to make a batch of lentiviral particles. This batch of lentiviral particle can be used to transduce one fraction of the patient's peripheral T cells, resulting in one batch of TCR-T cells. This can be repeated for all TCR genes. At the end, the batches of TCR-T cells, each expressing one TCR, can be mixed and then infused to the patient. For another example, a mixture of lentiviral particles can be used to transduce the patient's peripheral T cells at low multiplicity of infection (MOI). A pilot experiment can be conducted first to determine the dilution factor of the lentiviral particles so that the functional MOI (e.g., the fraction of T cells that expresses one or more exogenous TCRs) can be from about 5% to 15%. Here we show that (1) when this functional MOI is maintained, only one copy of lentiviral vector DNA can be inserted into the recipient T cell's genome, and (2) the single insertion result in sufficient level of exogenous TCR expression. Four constructs of lentiviral particles were prepared:
LentiA: CMVgzTCR-mCherry
LentiB: CMVgzTCR-YFP
LentiC: LibBg-mCherry
LentiD: LibBg-YFP

In each of these constructs there is a TCR expression cassette, driven by an EF1A promoter, and a fluorescent reporter expression cassette, driven by a hPGK promoter. The TCR in LentiA and LentiB (called CMVgzTCR) is specific to an HLA-A*02:01-presented CMV-derived peptide. LentiC and LentiD are mixtures of lentiviral particles, where the TCR was a library of >100 TCRs (called LibBg) cloned from a healthy donor. None of these TCRs was CMV-specific. LentiA and LentiC contain the mCherry fluorescent reporter. LentiB and LentiD contain the YFP fluorescent reporter.

The LentiA and LentiB viral particles were mixed at 1:1 molar ratio according to their viral titrations. The mixture was named CMVgzTCR-mChY. Similarly, LentiC and Lenti viral particles were mixed at 1:1 molar ratio. The mixture was named LibBg-mChY.

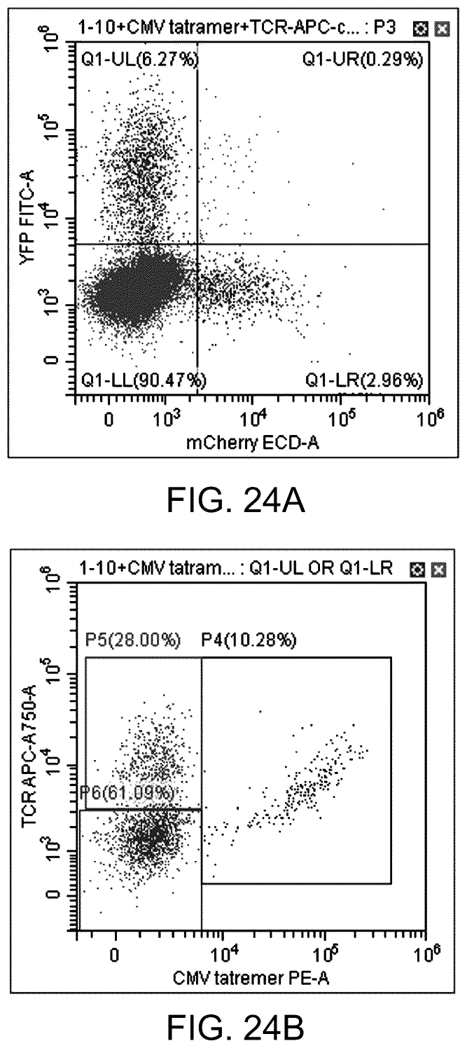

The CMVgzTCR-mChY virus was mixed with LibBg-mChY at the ratios of 1:9. And this mixture was used to infect an engineered Jurkat single clone JB4P2D5, with endogenous TCR knocked out at a functional MOI of ˜10%. The dilution factor to achieve this functional MOI was determined before the experiment. The infected Jurkat cells were then examined by flowcytometry. As shown in FIG. 24A, about 10% of Jurkat cells express a fluorescent reporter. Specifically, 6.27% expressed YFP and 2.96% expressed mCherry. Since the mCherry-carrying lentiviral DNA and YFP-carrying lentiviral DNA were inserted independently into the Jurkat genome, (6.27%*2.96%=) 0.18% of Jurkat cells expressed both mCherry and YFP. Indeed, about 0.29% of Jurkat cells were found to express both reporters (within experimental error from the expected value).

FIG. 24B shows the staining of the anti-TCR antibody (Y-axis) and CMV-derived pMHC tetramer (X-axis) is bound by CMVgzTCR of all the Jurkat cells in the gates Q1-UL, Q1-UR, Q1-LR of FIG. 24A combined (e.g., all cells that express a fluorescent reporter). CMVgzTCR-mChY was mixed with LibBg-mChY at 1:9 ratio, and about 10% of Jurkat cells bound the CMV-derived pMHC tetramer. Indeed, 10.28% of the Jurkat cells showed tetramer staining (see P4 of FIG. 24B). This means virtually all Jurkat cells that are inserted with lentiviral DNA encoding the CMVgzTCR express the CMVgzTCR at sufficiently high level, even if only one copy of lentiviral DNA is inserted. Although 61.9% of cells (P6) appear to be TCR negative, this is due to the fact that many of the TCRs in the LibBg library do not express well. Nevertheless, when the TCR is known to be functional (such as CMVgzTCR), the expression level is sufficiently high.

TABLE 1
Example sequences used in the present disclosure

| Name | Sequence |
|---|---|
| Seq ID | GAGGGCAGGGGTAGTCTCCTCACCTGTGGTGATGTGGAAGAAAATCCCGGACCCA |
| E5.1NT | TGAGCATCGGCCTCCTGTGCTGTGCAGCCTTGTCTCTCCTGTGGGCAGGTCCAGTG |
| | AATGCTGGTGTCACTCAGACCCCAAAATTCCAGGTCCTGAAGACAGGACAGAGCA |
| | TGACACTGCAGTGTGCCCAGGATATGAACCATGAATACATGTCCTGGTATCGACA |
| | AGACCCAGGCATGGGGCTGAGGCTGATTCATTACTCAGTTGGTGCTGGTATCACTG |
| | ACCAAGGAGAAGTCCCCAATGGCTACAATGTCTCCAGATCAACCACAGAGGATTT |
| | CCCGCTCAGGCTGCTGTCGGCTGCTCCCTCCCAGACATCTGTGTACTTCTGTGCCA |
| | GCAGTTACGTCGGGAACACCGGGGAGCTGTTTTTTGGAGAAGGCTCTAGGCTGAC |
| | CGTACTGGaggacctgaacaaggtgttcccacccgaggtcgctgtgtttgagccatcagaagcagagatctcccacacccaaaa |
| | ggccacactggtgtgcctggccacaggcttcttccccgaccacgtggagctgagctggtgggtgaatgggaaggaggtgcacagtgg |
| | ggtcagcacggacccgcagcccctcaaggagcagcccgccctcaatgactccagatactgcctgagcagccgcctgaggGTGtcg |
| | gccaccttctggcagaacccccgcaaccacttccgctgtcaagtccagttctacgggctctoggagaatgacgagtggacccaggatag |
| | ggccaaacccgtcacccagatcgtcagegccgaggcctggggtagagcagactgtggctttacctcggtgtcctaccagcaaggggtc |
| | ctgtctgccaccatcctctatgagatcctgctagggaaggccaccctgtatgctgtgctggtcagcgcccttgtgttgatggccatggtcaa |
| | gagaaaggatttcCGGGCTAAACGATCAGGTTCCGGAGCCACCAATTTCAGTCTTCTCAAA |
| | CAAGCTGGTGATGTTGAAGAGAATCCCGGcCCaATGGAGACACTCTTGGGCCTGCT |
| | TATCCTTTGGCTGCAGCTGCAATGGGTGAGCAGCAAACAGGAGGTGACGCAGATT |
| | CCTGCAGCTCTGAGTGTCCCAGAAGGAGAAAACTTGGTTCTCAACTGCAGTTTCAC |
| | TGATAGCGCTATTTACAACCTCCAGTGGTTTAGGCAGGACCCTGGGAAAGGTCTG |
| | ACATCTCTGTTGCTTATTCAGTCAAGTCAGAGAGAGCAAACAAGTGGAAGACTTA |
| | ATGCCTCGCTGGATAAATCATCAGGACGTAGTACTTTATACATTGCAGCTTCTCAG |
| | CCTGGTGACTCAGCCACCTACCTCTGTGCTGTGAGGCCCCTGTACGGAGGAAGCTA |
| | CATACCTACATTTGGAAGAGGAACCAGCCTTATTGTTCATCCGTAT |
| Seq ID | EGRGSLLTCGDVEENPGPMSIGLLCCAALSLLWAGPVNAGVTQTPKFQVLKTGQSMT |
| E5.1AA | LQCAQDMNHEYMSWYRQDPGMGLRLIHYSVGAGITDQGEVPNGYNVSRSTTEDFPL |
| | RLLSAAPSQTSVYFCASSYVGNTGELFFGEGSRLTVLEDLNKVFPPEVAVFEPSEAEISH |
| | TOKATLVCLATGFFPDHVELSWWVNGKEVHSGVSTDPQPLKEQPALNDSRYCLSSRL |
| | RVSATFWQNPRNHFRCQVQFYGLSENDEWTQDRAKPVTQIVSAEAWGRADCGFTSV |
| | SYQQGVLSATILYEILLGKATLYAVLVSALVLMAMVKRKDFRAKRSGSGATNFSLLK |
| | QAGDVEENPGPMETLLGLLILWLQLQWVSSKQEVTQIPAALSVPEGENLVLNCSFTDS |
| | AIYNLQWFRQDPGKGLTSLLLIQSSQREQTSGRLNASLDKSSGRSTLYIAASQPGDSAT |
| | YLCAVRPLYGGSYIPTFGRGTSLIVHPY |
| Seq ID | GAGGGCAGGGGTAGTCTCCTCACCTGTGGCGATGTGGAAGAGAATCCCGGACCCA |
| E5.2NT | TGTCCAACCAGGTGCTGTGTTGTGTAGTGCTTTGTTTTTTGGGTGCCAACACAGTTG |
| | ACGGAGGAATAACCCAATCTCCCAAGTACCTCTTCAGGAAGGAAGGACAGAATGT |
| | CACACTTAGTTGCGAACAGAACCTTAATCACGACGCAATGTACTGGTACCGCCAG |
| | GACCCTGGGCAAGGCCTTAGGCTCATTTATTATAGTCAGATAGTGAATGACTTTCA |
| | GAAGGGGGATATAGCCGAAGGCTATTCAGTCTCCCGCGAGAAAAAGGAATCTTTT |
| | CCACTTACCGTAACCTCTGCACAGAAAAATCCCACTGCTTTCTATCTGTGTGCATC |
| | ATCTCCAGGGGCGCTTTATGAGCAGTATTTTGGTCCCGGAACTCGATTGACGGTGA |
| | CAGAAGACCTCAAAAACGTCTTTCCCCCGGAGGTAGCCGTCTTTGAGCCCTCCGAA |
| | GCGGAGATCAGTCACACTCAGAAAGCAACGCTGGTCTGTCTGGCGACCGGTTTTT |
| | ACCCAGACCATGTGGAACTGTCTTGGTGGGTCAATGGCAAGGAAGTTCATTCAGG |
| | CGTTAGTACGGATCCTCAACCGCTGAAAGAGCAACCCGCTTTGAACGACTCAAGG |
| | TACTGCTTGTCATCACGGTTGCGGGTATCAGCAACTTTTTGGCAGAATCCCCGCAA |
| | CCATTTCCGCTGCCAGGTTCAATTCTATGGTCTTAGTGAGAACGACGAATGGACTC |
| | AAGACAGGGCCAAACCGGTGACTCAAATTGTATCAGCAGAGGCGTGGGGGAGGG |
| | CAGATTGCGGCTTTACAAGTGAAAGCTATCAGCAGGGGGTCTTGTCCGCTACAATC |
| | CTTTACGAAATCCTTTTGGGGAAGGCGACACTCTATGCGGTTCTGGTGTCAGCTCT |
| | TGTTCTTATGGCCATGGTCAAGCGGAAAGACAGCAGGGGCCGGGCTAAACGATCA |
| | GGTTCCGGAGCCACCAATTTCAGTCTTCTCAAACAAGCTGGTGATGTTGAAGAGA |
| | ATCCCGGCCCAATGACCAGTATACGAGCCGTGTTCATCTTTCTGTGGCTTCAACTC |
| | GACCTTGTCAACGGTGAAAACGTTGAACAGCACCCTTCTACTCTGAGCGTACAAG |
| | AGGGTGACTCCGCCGTAATAAAATGCACCTACTCAGACAGCGCCTCCAACTATTTC |
| | CCGTGGTACAAGCAGGAACTTGGTAAACGCCCGCAGTTGATCATTGATATCCGAA |
| | GCAATGTCGGCGAGAAGAAGGATCAGAGAATAGCCGTGACCTTGAATAAAACAG |
| | CCAAGCACTTCTCCCTGCACATCACTGAAACTCAGCCTGAGGATTCTGCTGTATAC |
| | TTTTGCGCGGCTACGGAAGATCTTACTCTCATTTGGGGTGCCGGCACCAAGCTGAT |
| | CATTAAGCCCGAc |
| Seq ID | EGRGSLLTCGDVEENPGPMSNQVLCCVVLCFLGANTVDGGITQSPKYLFRKEGQNVT |
| E5.2AA | LSCEQNLNHDAMYWYRQDPGQGLRLIYYSQIVNDFQKGDIAEGYSVSREKKESFPLT |
| | VTSAQKNPTAFYLCASSPGALYEQYFGPGTRLTVTEDLKNVFPPEVAVFEPSEAEISHT |
| | QKATLVCLATGFYPDHVELSWWVNGKEVHSGVSTDPQPLKEQPALNDSRYCLSSRLR |
| | VSATFWQNPRNHFRCQVQFYGLSENDEWTQDRAKPVTQIVSAEAWGRADCGFTSES |
| | YQQGVLSATILYEILLGKATLYAVLVSALVLMAMVKRKDSRGRAKRSGSGATNFSLL |
| | KQAGDVEENPGPMTSIRAVFIFLWLQLDLVNGENVEQHPSTLSVQEGDSAVIKCTYSD |
| | SASNYFPWYKQELGKRPQLIIDIRSNVGEKKDQRIAVTLNKTAKHFSLHITETQPEDSA |
| | VYFCAATEDLTLIWGAGTKLIIKPD |
| Seq ID | aaaggcaggaggtcggaAAGAATAAACAATGAGAGTCACATTAAAAACACAAAATCCTAC |
| E5.3NT | GGAAATACTGAAGAATGAGTCTCAGCACTAAGGAAAAGCCTCCAGCAGCTCCTGC |
| | TTTCTGAGGGTGAAGGATAGACGCTGTGGCTCTGCATGACTCACTAGCACTCTATC |
| | ACGGCCATATTCTGGCAGGGTCAGTGGCTCCAActaacatttgtttggtactttacagtttattaaatagatgt |
| | ttatatggagaagctctcatttctttctcagaagagcctggctaggaaggtggatgaggcaccatattcattttgcaggtgaaattcctgaga |
| | tgtaaggagctgctgtgacttgctcaaggccttatatcgagtaaacggtagcgctggggcttagacgcaggtgttctgatttatagttcaA |
| | AACCTCTATCAATGAGAGAGCAATCTCCTGGTAATGTGATAGATTTCCCAACTTAA |
| | TGCCAACATACCATAAACCTCCCATTCTGCTAATGCCCAGCCTAAGTTGGGGAGAC |
| | CACTCCAGATTCCAAGATGTACAGTTTGCTTTGCTGGGCCTTTTTCCCATGCCTGCC |
| | TTTACTCTGCCAGAGTTATATTGCTGGGGTTTTGAAGAAGATCCTATTAAATAAAA |
| | GAATAAGCAGTATTATTAAGTAGCCCTGCATTTCAGGTTTCCTTGAGTGGCAGGCC |
| | AGGCCTGGCCGTGAACGTTCACTGAAATCATGGCCTCTTGGCCAAGATTGATAGCT |
| | TGTGCCTGTCCCTGAGTCCCAGTCCATCACGAGCAGCTGGTTTCTAAGATGCTATT |
| | TCCCGTATAAAGCATGAGACCGTGACTTGCCAGCCCCACAGAGCCCCGCCCTTGTC |
| | CATCACTGGCATCTGGACTCCAGCCTGGGTTGGGGCAAAGAGGGAAATGAGATCA |
| | TGTCCTAACCCTGATCCTCTTGTCCCACAGATTCCGGATCCGGA |
| Seq ID | ATCCAGAACCCTGACCCTGCCGTGTACCAGCTGAGAGACTCTAAATCCAGTGACA |
| E5.4NT | AGTCTGTCTGCCTATTCACCGATTTTGATTCTCAAACAAATGTGTCACAAAGTAAG |
| | GATTCTGATGTGTATATCACAGACAAAACTGTGCTAGACATGAGGTCTATGGACTT |
| | CAAGAGCAACAGTGCTGTGGCCTGGAGCAACAAATCTGACTTTGCATGTGCAAAC |
| | GCCTTCAACAACAGCATTATTCCAGAAGACACCTTCTTCCCCAGCCCAGGTAAGGG |
| | CAGCTTTGGTGCCTTCGCAGGCTGTTTCCTTGCTTCAGGAATGGCCAGGTTCTGCC |
| | CAGAGCTCTGGTCAATGATGTCTAAAACTCCTCTGATTGGTGGTCTCGGCCTTATC |
| | CATTGCCACCAAAACCCTCTTTTTACTAAGAAACAGTGAGCCTTGTTCTGGCAGTC |
| | CAGAGAATGACACGGGAAAAAAGCAGATGAAGAGAAGGTGGCAGGAGAGGGCA |
| | CGTGGCCCAGCCTCAGTCTCTCCAACTGAGTTCCTGCCTGCCTGCCTTTGCTCAGA |
| | CTGTTTGCCCCTTACTGCTCTTCTAGGCCTCATTCTAAGCCCCTTCTCCAAGTTGCC |
| | TCTCCTTATTTCTCCCTGTCTGCCAAAAAATCTTTCCCAGCTCACTAAGTCAGTCTC |
| | ACGCAGTCACTC |

TABLE 2
Sequences of TCR1, TCR2 and TCR3 from Patient 1.

| TCR | chain | cdr3s_aa | consensus |
|---|---|---|---|
| 1 | TRA | CAVHR | ACAGAAGTGGCGCCTCTGAGAAAAGAAGGTTGGAATTATCGTAATTTG |
| | | TDSWG | TTTCTAGGCTGAGATACCAGCATGGAGAAAATGTTGGAGTGTGCATTC |
| | | KLQF | ATAGTCTTGTGGCTTCAGCTTGGCTGGTTGAGTGGAGAAGACCAGGTG |
| | | | ACGCAGAGTCCCGAGGCCCTGAGACTCCAGGAGGGAGAGAGTAGCAG |
| | | | TCTCAACTGCAGTTACACAGTCAGCGGTTTAAGAGGGCTGTTCTGGTAT |
| | | | AGGCAAGATCCTGGGAAAGGCCCTGAATTCCTCTTCACCCTGTATTCAG |
| | | | CTGGGGAAGAAAAGGAGAAAGAAAGGCTAAAAGCCACATTAACAAAG |
| | | | AAGGAAAGCTTTCTGCACATCACAGCCCCTAAACCTGAAGACTCAGCC |
| | | | ACTTATCTCTGTGCTGTGCACAGGACTGACAGCTGGGGGAAATTGCAG |
| | | | TTTGGATCAGGGACCCAGGTTGTGGTCACCCCAGATATCCAGAACCCT |
| | | | GACCCTGCCGTGTACCAGCTGAGAGACT |
| 1 | TRB | CASSLG | CTCAGAGGACCAGTATCCCTCACAGGGTGACACCTGACCAGCTCTGTC |
| | | ARELA | CCACCTGGCCATGGGCTCCAGGTACCTCTGATGGGAAGACCTTTGTCTC |
| | | KNIQYF | TTGGGAACAAGTGAATCCTTGGCACAGGCCCAGTGGATTCTGCTGTGC |
| | | | AGAACAGAGAGCAGTGGACCTCAGGAGGCCTGCAAGGGGAGGACATA |
| | | | GGACAGTGACATCACAGTATGCCCCTCCCACCAGGAAAAGCAAGGCTG |
| | | | AGAATTTAGCTCTTTCCCAGGAGGACCAAGCCCTGAGCACAGACACAG |
| | | | TGCTGCCTGCCCCTTTGTGCCATGGGCTCCAGGCTGCTCTGTTGGGTGC |
| | | | TGCTTTGTCTCCTGGGAGCAGGCCCAGTAAAGGCTGGAGTCACTCAAA |
| | | | CTCCAAGATATCTGATCAAAACGAGAGGACAGCAAGTGACACTGAGCT |
| | | | GCTCCCCTATCTCTGGGCATAGGAGTGTATCCTGGTACCAACAGACCCC |
| | | | AGGACAGGGCCTTCAGTTCCTCTTTGAATACTTCAGTGAGACACAGAG |
| | | | AAACAAAGGAAACTTCCCTGGTCGATTCTCAGGGCGCCAGTTCTCTAA |
| | | | CTCTCGCTCTGAGATGAATGTGAGCACCTTGGAGCTGGGGGACTCGGC |
| | | | CCTTTATCTTTGCGCCAGCAGCTTGGGGGCTCGGGAACTAGCCAAAAA |
| | | | CATTCAGTACTTCGGCGCCGGGACCCGGCTCTCAGTGCTGGAGGACCT |
| | | | GAAAAACGTGTTCCCACCCGAGGTCGCTGTGTTTGAGCCATCAGA |
| 2 | TRA | CAVIGE | AAAGCAGATTCTTTTTATGATTTTTAAAGTAGAAATATCCATTCTAGGT |
| | | YGNKL | GCATTTTTTAAGGGTTTAAAATTTGAATCCTCAGTGAACCAGGGCAGAG |
| | | VF | AAGAATGATGAAATCCTTGAGAGTTTTACTAGTGATCCTGTGGCTTCAG |
| | | | TTGAGCTGGGTTTGGAGCCAACAGAAGGAGGTGGAGCAGAATTCTGGA |
| | | | CCCCTCAGTGTTCCAGAGGGAGCCATTGCCTCTCTCAACTGCACTTACA |
| | | | GTGACCGAGGTTCCCAGTCCTTCTTCTGGTACAGACAATATTCTGGGAA |
| | | | AAGCCCTGAGTTGATAATGTTCATATACTCCAATGGTGACAAAGAAGA |
| | | | TGGAAGGTTTACAGCACAGCTCAATAAAGCCAGCCAGTATGTTTCTCTG |
| | | | CTCATCAGAGACTCCCAGCCCAGTGATTCAGCCACCTACCTCTGTGCCG |
| | | | TGATTGGAGAATATGGAAACAAGCTGGTCTTTGGCGCAGGAACCATTC |
| | | | TGAGAGTCAAGTCCTATATCCAGAACCCTGACCCTGCCGTGTACCAGCT |
| | | | GAGAGACT |
| 2 | TRB | CASSYG | TTATATGGGGATGCTCACAGAGGGCCTGGTCTAGAATATTCCACATCTG |
| | | GYEQY | CTCTCACTCTGCCATGGACTCCTGGACCTTCTGCTGTGTGTCCCTTTGCA |
| | | F | TCCTGGTAGCGAAGCATACAGATGCTGGAGTTATCCAGTCACCCCGCC |
| | | | ATGAGGTGACAGAGATGGGACAAGAAGTGACTCTGAGATGTAAACCA |
| | | | ATTTCAGGCCACAACTCCCTTTTCTGGTACAGACAGACCATGATGCGGG |
| | | | GACTGGAGTTGCTCATTTACTTTAACAACAACGTTCCGATAGATGATTC |
| | | | AGGGATGCCCGAGGATCGATTCTCAGCTAAGATGCCTAATGCATCATT |
| | | | CTCCACTCTGAAGATCCAGCCCTCAGAACCCAGGGACTCAGCTGTGTA |
| | | | CTTCTGTGCCAGCAGTTATGGGGGCTACGAGCAGTACTTCGGGCCGGG |
| | | | CACCAGGCTCACGGTCACAGAGGACCTGAAAAACGTGTTCCCACCCGA |
| | | | GGTCGCTGTGTTTGAGCCATCAGA |
| 3 | TRA | CADSSG | GATCTTAATTGGGAAGAACAAGGATGACATCCATTCGAGCTGTATTTAT |
| | | TYKYIF | ATTCCTGTGGCTGCAGCTGGACTTGGTGAATGGAGAGAATGTGGAGCA |
| | | | GCATCCTTCAACCCTGAGTGTCCAGGAGGGAGACAGCGCTGTTATCAA |
| | | | GTGTACTTATTCAGACAGTGCCTCAAACTACTTCCCTTGGTATAAGCAA |
| | | | GAACTTGGAAAAAGACCTCAGCTTATTATAGACATTCGTTCAAATGTG |
| | | | GGCGAAAAGAAAGACCAACGAATTGCTGTTACATTGAACAAGACAGCC |
| | | | AAACATTTCTCCCTGCACATCACAGAGACCCAACCTGAAGACTCGGCT |
| | | | GTCTACTTCTGTGCAGATTCCTCAGGAACCTACAAATACATCTTTGGAA |
| | | | CAGGCACCAGGCTGAAGGTTTTAGCAAATATCCAGAACCCTGACCCTG |
| | | | CCGTGTACCAGCTGAGAGACT |
| 3 | TRB | CASSW | ATGGTCTCATGGCTTTTTTCGTAGATGGGGGCTGTTGCTCACAGTGACC |
| | | GRDLNS | CTGATTGGGCAAAGCTCCCATCCTTCCCTGACCCTGCCATGGGCACCAG |
| | | PLHF | GCTCCTCTGCTGGGCGGCCCTCTGTCTCCTGGGAGCAGAACTCACAGAA |
| | | | GCTGGAGTTGCCCAGTCTCCCAGATATAAGATTATAGAGAAAAGGCAG |
| | | | AGTGTGGCTTTTTGGTGCAATCCTATATCTGGCCATGCTACCCTTTACTG |
| | | | GTACCAGCAGATCCTGGGACAGGGCCCAAAGCTTCTGATTCAGTTTCA |
| | | | GAATAACGGTGTAGTGGATGATTCACAGTTGCCTAAGGATCGATTTTCT |
| | | | GCAGAGAGGCTCAAAGGAGTAGACTCCACTCTCAAGATCCAGCCTGCA |
| | | | AAGCTTGAGGACTCGGCCGTGTATCTCTGTGCCAGCAGCTGGGGGCGG |
| | | | GATTTGAATTCACCCCTCCACTTTGGGAATGGGACCAGGCTCACTGTGA |
| | | | CAGAGGACCTGAACAAGGTGTTCCCACCCGAGGTCGCTGTGTTTGAGC |
| | | | CATCAGA |
| NS | TRA | CAASN | GATCTTAATTGGGAAGAACAAGGATGACATCCATTCGAGCTGTATTTAT |
| | | GTGNQ | ATTCCTGTGGCTGCAGCTGGACTTGGTGAATGGAGAGAATGTGGAGCA |
| | | FYF | GCATCCTTCAACCCTGAGTGTCCAGGAGGGAGACAGCGCTGTTATCAA |
| | | | GTGTACTTATTCAGACAGTGCCTCAAACTACTTCCCTTGGTATAAGCAA |
| | | | GAACTTGGAAAAAGACCTCAGCTTATTATAGACATTCGTTCAAATGTG |
| | | | GGCGAAAAGAAAGACCAACGAATTGCTGTTACATTGAACAAGACAGCC |
| | | | AAACATTTCTCCCTGCACATCACAGAGACCCAACCTGAAGACTCGGCT |
| | | | GTCTACTTCTGTGCAGCAAGTAATGGGACCGGTAACCAGTTCTATTTTG |
| | | | GGACAGGGACAAGTTTGACGGTCATTCCAAATATCCAGAACCCTGACC |
| | | | CTGCCGTGTACCAGCTGAGAGACT |
| NS | TRB | CASSYT | GCTTTCCTGCTGTCTCGTGATACTAAATGAGTCTCTTTTAGATCTGATGG |
| | | AGRSYE | TTTTAAAATGGGATTTTCCCTGCACAAGCTCTCTCTTTTATGTTGCCATC |
| | | QYF | CAAGTAAGACATGACTTGCCCCTCCTTGCCTTCTGCCATGATTGTGAGG |
| | | | CCTCCCCAGCCATGTGGAACTAGGAAAAGTGAATCAAAACCAAGGGAC |
| | | | ATGCTGAGACAACTGGAGAAATTTGAATGTGAAGCATTTGTGGAGGCA |
| | | | ATGATGTCACTGTGGGAACTGCCATGAGAGGACAGGGACGTCCCTCCT |
| | | | CCTCTGCTTTTGCTCACAGTGACCCTGATTGGGCAAAGCTCCCATCCTT |
| | | | CCCTGACCCTGCCATGGGCACCAGGCTCCTCTGCTGGGCGGCCCTCTGT |
| | | | CTCCTGGGAGCAGAACTCACAGAAGCTGGAGTTGCCCAGTCTCCCAGA |
| | | | TATAAGATTATAGAGAAAAGGCAGAGTGTGGCTTTTTGGTGCAATCCT |
| | | | ATATCTGGCCATGCTACCCTTTACTGGTACCAGCAGATCCTGGGACAGG |
| | | | GCCCAAAGCTTCTGATTCAGTTTCAGAATAACGGTGTAGTGGATGATTC |
| | | | ACAGTTGCCTAAGGATCGATTTTCTGCAGAGAGGCTCAAAGGAGTAGA |
| | | | CTCCACTCTCAAGATCCAGCCTGCAAAGCTTGAGGACTCGGCCGTGTAT |
| | | | CTCTGTGCCAGCAGCTATACAGCGGGACGATCCTACGAGCAGTACTTC |
| | | | GGGCCGGGCACCAGGCTCACGGTCACAGAGGACCTGAAAAACGTGTTC |
| | | | CCACCCGAGGTCGCTGTGTTTGAGCCATCAGA |

TABLE 3
Example preconditioning/lymphodepletion regimen

| Drug | Time (I.V.) | Dose | |
|---|---|---|---|
| cyclophosphamide | Day −6 | 0.5 | g/m²/day |
| cyclophosphamide | Day −5 | 0.5 | g/m²/day |
| fludarabine | Day −4 | 30 | mg/m²/day |
| fludarabine | Day −3 | 30 | mg/m²/day |
| fludarabine | Day −2 | 30 | mg/m²/day |

TABLE 4
The UMI counts from different type of KI event

| A | B | C | D | E | F |
|---|---|---|---|---|---|
| 1 | 21 | 0 | 8 | 0 | 29 |
| 2 | 22 | 6 | 0 | 0 | 28 |
| 3 | 26 | 0 | 0 | 0 | 26 |
| 4 | 19 | 5 | 0 | 0 | 24 |
| 5 | 24 | 0 | 0 | 0 | 24 |
| 6 | 17 | 6 | 0 | 0 | 23 |
| 7 | 23 | 0 | 0 | 0 | 23 |
| 8 | 23 | 0 | 0 | 0 | 23 |
| 9 | 22 | 0 | 0 | 0 | 22 |
| 10 | 21 | 0 | 0 | 0 | 21 |
| 11 | 19 | 0 | 2 | 0 | 21 |
| 12 | 21 | 0 | 0 | 0 | 21 |
| 13 | 21 | 0 | 0 | 0 | 21 |
| 14 | 15 | 0 | 5 | 0 | 20 |
| 15 | 17 | 0 | 3 | 0 | 20 |
| 16 | 13 | 0 | 7 | 0 | 20 |
| 17 | 20 | 0 | 0 | 0 | 20 |
| 18 | 14 | 5 | 0 | 0 | 19 |
| 19 | 19 | 0 | 0 | 0 | 19 |
| 20 | 19 | 0 | 0 | 0 | 19 |
| 21 | 16 | 0 | 3 | 0 | 19 |
| 22 | 19 | 0 | 0 | 0 | 19 |
| 23 | 17 | 0 | 1 | 0 | 18 |
| 24 | 18 | 0 | 0 | 0 | 18 |
| 25 | 17 | 1 | 0 | 0 | 18 |
| 26 | 18 | 0 | 0 | 0 | 18 |
| 27 | 13 | 5 | 0 | 0 | 18 |
| 28 | 17 | 0 | 1 | 0 | 18 |
| 29 | 18 | 0 | 0 | 0 | 18 |
| 30 | 16 | 0 | 2 | 0 | 18 |
| 31 | 18 | 0 | 0 | 0 | 18 |
| 32 | 17 | 0 | 0 | 0 | 17 |
| 33 | 17 | 0 | 0 | 0 | 17 |
| 34 | 15 | 0 | 2 | 0 | 17 |
| 35 | 17 | 0 | 0 | 0 | 17 |
| 36 | 15 | 0 | 1 | 0 | 16 |
| 37 | 13 | 3 | 0 | 0 | 16 |
| 38 | 16 | 0 | 0 | 0 | 16 |
| 39 | 16 | 0 | 0 | 0 | 16 |
| 40 | 16 | 0 | 0 | 0 | 16 |
| 41 | 16 | 0 | 0 | 0 | 16 |
| 42 | 8 | 0 | 8 | 0 | 16 |
| 43 | 15 | 0 | 1 | 0 | 16 |
| 44 | 13 | 3 | 0 | 0 | 16 |
| 45 | 12 | 0 | 4 | 0 | 16 |
| 46 | 14 | 0 | 2 | 0 | 16 |
| 47 | 15 | 0 | 0 | 0 | 15 |
| 48 | 15 | 0 | 0 | 0 | 15 |
| 49 | 15 | 0 | 0 | 0 | 15 |
| 50 | 10 | 5 | 0 | 0 | 15 |
| 51 | 15 | 0 | 0 | 0 | 15 |
| 52 | 15 | 0 | 0 | 0 | 15 |
| 53 | 0 | 0 | 15 | 0 | 15 |
| 54 | 15 | 0 | 0 | 0 | 15 |
| 55 | 14 | 0 | 0 | 0 | 14 |
| 56 | 9 | 5 | 0 | 0 | 14 |
| 57 | 14 | 0 | 0 | 0 | 14 |
| 58 | 14 | 0 | 0 | 0 | 14 |
| 59 | 14 | 0 | 0 | 0 | 14 |
| 60 | 9 | 0 | 5 | 0 | 14 |
| 61 | 14 | 0 | 0 | 0 | 14 |
| 62 | 0 | 0 | 14 | 0 | 14 |
| 63 | 14 | 0 | 0 | 0 | 14 |
| 64 | 11 | 0 | 2 | 0 | 13 |
| 65 | 11 | 0 | 2 | 0 | 13 |
| 66 | 13 | 0 | 0 | 0 | 13 |
| 67 | 13 | 0 | 0 | 0 | 13 |
| 68 | 9 | 0 | 4 | 0 | 13 |
| 69 | 0 | 0 | 13 | 0 | 13 |
| 70 | 13 | 0 | 0 | 0 | 13 |
| 71 | 7 | 6 | 0 | 0 | 13 |
| 72 | 13 | 0 | 0 | 0 | 13 |
| 73 | 13 | 0 | 0 | 0 | 13 |
| 74 | 13 | 0 | 0 | 0 | 13 |
| 75 | 13 | 0 | 0 | 0 | 13 |
| 76 | 1 | 0 | 12 | 0 | 13 |
| 77 | 13 | 0 | 0 | 0 | 13 |
| 78 | 12 | 0 | 1 | 0 | 13 |
| 79 | 13 | 0 | 0 | 0 | 13 |
| 80 | 13 | 0 | 0 | 0 | 13 |
| 81 | 13 | 0 | 0 | 0 | 13 |
| 82 | 13 | 0 | 0 | 0 | 13 |
| 83 | 11 | 0 | 1 | 0 | 12 |
| 84 | 0 | 0 | 11 | 1 | 12 |
| 85 | 12 | 0 | 0 | 0 | 12 |
| 86 | 0 | 0 | 12 | 0 | 12 |
| 87 | 12 | 0 | 0 | 0 | 12 |
| 88 | 9 | 3 | 0 | 0 | 12 |
| 89 | 12 | 0 | 0 | 0 | 12 |
| 90 | 6 | 6 | 0 | 0 | 12 |
| 91 | 9 | 0 | 3 | 0 | 12 |
| 92 | 10 | 0 | 2 | 0 | 12 |
| 93 | 12 | 0 | 0 | 0 | 12 |
| 94 | 7 | 5 | 0 | 0 | 12 |
| 95 | 12 | 0 | 0 | 0 | 12 |
| 96 | 12 | 0 | 0 | 0 | 12 |
| 97 | 7 | 0 | 5 | 0 | 12 |
| 98 | 9 | 0 | 3 | 0 | 12 |
| 99 | 0 | 0 | 12 | 0 | 12 |
| 100 | 12 | 0 | 0 | 0 | 12 |
| 101 | 12 | 0 | 0 | 0 | 12 |
| 102 | 9 | 0 | 2 | 0 | 11 |
| 103 | 11 | 0 | 0 | 0 | 11 |
| 104 | 11 | 0 | 0 | 0 | 11 |
| 105 | 11 | 0 | 0 | 0 | 11 |
| 106 | 8 | 0 | 3 | 0 | 11 |
| 107 | 11 | 0 | 0 | 0 | 11 |
| 108 | 11 | 0 | 0 | 0 | 11 |
| 109 | 6 | 5 | 0 | 0 | 11 |
| 110 | 11 | 0 | 0 | 0 | 11 |
| 111 | 11 | 0 | 0 | 0 | 11 |
| 112 | 11 | 0 | 0 | 0 | 11 |
| 113 | 11 | 0 | 0 | 0 | 11 |
| 114 | 11 | 0 | 0 | 0 | 11 |
| 115 | 11 | 0 | 0 | 0 | 11 |
| 116 | 11 | 0 | 0 | 0 | 11 |
| 117 | 11 | 0 | 0 | 0 | 11 |
| 118 | 11 | 0 | 0 | 0 | 11 |
| 119 | 7 | 4 | 0 | 0 | 11 |
| 120 | 11 | 0 | 0 | 0 | 11 |
| 121 | 11 | 0 | 0 | 0 | 11 |
| 122 | 11 | 0 | 0 | 0 | 11 |
| 123 | 11 | 0 | 0 | 0 | 11 |
| 124 | 11 | 0 | 0 | 0 | 11 |
| 125 | 11 | 0 | 0 | 0 | 11 |
| 126 | 7 | 4 | 0 | 0 | 11 |
| 127 | 11 | 0 | 0 | 0 | 11 |
| 128 | 11 | 0 | 0 | 0 | 11 |
| 129 | 10 | 0 | 1 | 0 | 11 |
| 130 | 9 | 0 | 2 | 0 | 11 |
| 131 | 10 | 0 | 1 | 0 | 11 |
| 132 | 11 | 0 | 0 | 0 | 11 |
| 133 | 11 | 0 | 0 | 0 | 11 |
| 134 | 11 | 0 | 0 | 0 | 11 |
| 135 | 11 | 0 | 0 | 0 | 11 |
| 136 | 11 | 0 | 0 | 0 | 11 |
| 137 | 11 | 0 | 0 | 0 | 11 |
| 138 | 10 | 0 | 0 | 0 | 10 |
| 139 | 0 | 0 | 9 | 1 | 10 |
| 140 | 7 | 0 | 3 | 0 | 10 |
| 141 | 7 | 0 | 3 | 0 | 10 |
| 142 | 10 | 0 | 0 | 0 | 10 |
| 143 | 10 | 0 | 0 | 0 | 10 |
| 144 | 10 | 0 | 0 | 0 | 10 |
| 145 | 7 | 3 | 0 | 0 | 10 |
| 146 | 10 | 0 | 0 | 0 | 10 |
| 147 | 10 | 0 | 0 | 0 | 10 |
| 148 | 8 | 2 | 0 | 0 | 10 |
| 149 | 10 | 0 | 0 | 0 | 10 |
| 150 | 10 | 0 | 0 | 0 | 10 |
| 151 | 8 | 2 | 0 | 0 | 10 |
| 152 | 0 | 0 | 10 | 0 | 10 |
| 153 | 10 | 0 | 0 | 0 | 10 |
| 154 | 6 | 4 | 0 | 0 | 10 |
| 155 | 10 | 0 | 0 | 0 | 10 |
| 156 | 8 | 0 | 2 | 0 | 10 |
| 157 | 10 | 0 | 0 | 0 | 10 |
| 158 | 10 | 0 | 0 | 0 | 10 |
| 159 | 10 | 0 | 0 | 0 | 10 |
| 160 | 10 | 0 | 0 | 0 | 10 |
| 161 | 10 | 0 | 0 | 0 | 10 |
| 162 | 10 | 0 | 0 | 0 | 10 |
| 163 | 10 | 0 | 0 | 0 | 10 |
| 164 | 8 | 0 | 2 | 0 | 10 |
| 165 | 10 | 0 | 0 | 0 | 10 |
| 166 | 10 | 0 | 0 | 0 | 10 |
| 167 | 10 | 0 | 0 | 0 | 10 |
| 168 | 10 | 0 | 0 | 0 | 10 |
| 169 | 10 | 0 | 0 | 0 | 10 |
| 170 | 10 | 0 | 0 | 0 | 10 |
| 171 | 10 | 0 | 0 | 0 | 10 |
| 172 | 10 | 0 | 0 | 0 | 10 |
| 173 | 10 | 0 | 0 | 0 | 10 |
| 174 | 10 | 0 | 0 | 0 | 10 |
| 175 | 10 | 0 | 0 | 0 | 10 |
| 176 | 5 | 5 | 0 | 0 | 10 |
| 177 | 10 | 0 | 0 | 0 | 10 |
| 178 | 10 | 0 | 0 | 0 | 10 |
| 179 | 10 | 0 | 0 | 0 | 10 |
| 180 | 10 | 0 | 0 | 0 | 10 |
| 181 | 10 | 0 | 0 | 0 | 10 |
| 182 | 5 | 5 | 0 | 0 | 10 |
| 183 | 10 | 0 | 0 | 0 | 10 |
| 184 | 10 | 0 | 0 | 0 | 10 |
| 185 | 10 | 0 | 0 | 0 | 10 |
| 186 | 6 | 4 | 0 | 0 | 10 |
| 187 | 10 | 0 | 0 | 0 | 10 |
| 188 | 10 | 0 | 0 | 0 | 10 |
| 189 | 0 | 0 | 5 | 5 | 10 |
| 190 | 10 | 0 | 0 | 0 | 10 |
| 191 | 10 | 0 | 0 | 0 | 10 |
| 192 | 9 | 0 | 0 | 0 | 9 |
| 193 | 7 | 2 | 0 | 0 | 9 |
| 194 | 9 | 0 | 0 | 0 | 9 |
| 195 | 8 | 0 | 1 | 0 | 9 |
| 196 | 7 | 0 | 2 | 0 | 9 |
| 197 | 9 | 0 | 0 | 0 | 9 |
| 198 | 9 | 0 | 0 | 0 | 9 |
| 199 | 9 | 0 | 0 | 0 | 9 |
| 200 | 9 | 0 | 0 | 0 | 9 |
| 201 | 9 | 0 | 0 | 0 | 9 |
| 202 | 9 | 0 | 0 | 0 | 9 |
| 203 | 9 | 0 | 0 | 0 | 9 |
| 204 | 9 | 0 | 0 | 0 | 9 |
| 205 | 9 | 0 | 0 | 0 | 9 |
| 206 | 9 | 0 | 0 | 0 | 9 |
| 207 | 9 | 0 | 0 | 0 | 9 |
| 208 | 7 | 2 | 0 | 0 | 9 |
| 209 | 9 | 0 | 0 | 0 | 9 |
| 210 | 9 | 0 | 0 | 0 | 9 |
| 211 | 9 | 0 | 0 | 0 | 9 |
| 212 | 9 | 0 | 0 | 0 | 9 |
| 213 | 9 | 0 | 0 | 0 | 9 |
| 214 | 9 | 0 | 0 | 0 | 9 |
| 215 | 9 | 0 | 0 | 0 | 9 |
| 216 | 9 | 0 | 0 | 0 | 9 |
| 217 | 9 | 0 | 0 | 0 | 9 |
| 218 | 9 | 0 | 0 | 0 | 9 |
| 219 | 9 | 0 | 0 | 0 | 9 |
| 220 | 9 | 0 | 0 | 0 | 9 |
| 221 | 9 | 0 | 0 | 0 | 9 |
| 222 | 9 | 0 | 0 | 0 | 9 |
| 223 | 9 | 0 | 0 | 0 | 9 |
| 224 | 9 | 0 | 0 | 0 | 9 |
| 225 | 9 | 0 | 0 | 0 | 9 |
| 226 | 9 | 0 | 0 | 0 | 9 |
| 227 | 9 | 0 | 0 | 0 | 9 |
| 228 | 9 | 0 | 0 | 0 | 9 |
| 229 | 9 | 0 | 0 | 0 | 9 |
| 230 | 9 | 0 | 0 | 0 | 9 |
| 231 | 5 | 4 | 0 | 0 | 9 |
| 232 | 9 | 0 | 0 | 0 | 9 |
| 233 | 9 | 0 | 0 | 0 | 9 |
| 234 | 9 | 0 | 0 | 0 | 9 |
| 235 | 9 | 0 | 0 | 0 | 9 |
| 236 | 9 | 0 | 0 | 0 | 9 |
| 237 | 9 | 0 | 0 | 0 | 9 |
| 238 | 9 | 0 | 0 | 0 | 9 |
| 239 | 9 | 0 | 0 | 0 | 9 |
| 240 | 9 | 0 | 0 | 0 | 9 |
| 241 | 9 | 0 | 0 | 0 | 9 |
| 242 | 9 | 0 | 0 | 0 | 9 |
| 243 | 9 | 0 | 0 | 0 | 9 |
| 244 | 9 | 0 | 0 | 0 | 9 |
| 245 | 9 | 0 | 0 | 0 | 9 |
| 246 | 9 | 0 | 0 | 0 | 9 |
| 247 | 9 | 0 | 0 | 0 | 9 |
| 248 | 9 | 0 | 0 | 0 | 9 |
| 249 | 8 | 0 | 0 | 0 | 8 |
| 250 | 8 | 0 | 0 | 0 | 8 |
| 251 | 7 | 0 | 1 | 0 | 8 |
| 252 | 7 | 0 | 1 | 0 | 8 |
| 253 | 7 | 0 | 1 | 0 | 8 |
| 254 | 0 | 0 | 8 | 0 | 8 |
| 255 | 5 | 3 | 0 | 0 | 8 |
| 256 | 8 | 0 | 0 | 0 | 8 |
| 257 | 8 | 0 | 0 | 0 | 8 |
| 258 | 8 | 0 | 0 | 0 | 8 |
| 259 | 8 | 0 | 0 | 0 | 8 |
| 260 | 8 | 0 | 0 | 0 | 8 |
| 261 | 8 | 0 | 0 | 0 | 8 |
| 262 | 8 | 0 | 0 | 0 | 8 |
| 263 | 8 | 0 | 0 | 0 | 8 |
| 264 | 5 | 3 | 0 | 0 | 8 |
| 265 | 8 | 0 | 0 | 0 | 8 |
| 266 | 8 | 0 | 0 | 0 | 8 |
| 267 | 4 | 0 | 4 | 0 | 8 |
| 268 | 8 | 0 | 0 | 0 | 8 |
| 269 | 8 | 0 | 0 | 0 | 8 |
| 270 | 8 | 0 | 0 | 0 | 8 |
| 271 | 8 | 0 | 0 | 0 | 8 |
| 272 | 8 | 0 | 0 | 0 | 8 |
| 273 | 8 | 0 | 0 | 0 | 8 |
| 274 | 8 | 0 | 0 | 0 | 8 |
| 275 | 8 | 0 | 0 | 0 | 8 |
| 276 | 8 | 0 | 0 | 0 | 8 |
| 277 | 8 | 0 | 0 | 0 | 8 |
| 278 | 8 | 0 | 0 | 0 | 8 |
| 279 | 8 | 0 | 0 | 0 | 8 |
| 280 | 0 | 0 | 8 | 0 | 8 |
| 281 | 8 | 0 | 0 | 0 | 8 |
| 282 | 8 | 0 | 0 | 0 | 8 |
| 283 | 8 | 0 | 0 | 0 | 8 |
| 284 | 5 | 3 | 0 | 0 | 8 |
| 285 | 8 | 0 | 0 | 0 | 8 |
| 286 | 8 | 0 | 0 | 0 | 8 |
| 287 | 8 | 0 | 0 | 0 | 8 |
| 288 | 8 | 0 | 0 | 0 | 8 |
| 289 | 6 | 2 | 0 | 0 | 8 |
| 290 | 5 | 3 | 0 | 0 | 8 |
| 291 | 8 | 0 | 0 | 0 | 8 |
| 292 | 8 | 0 | 0 | 0 | 8 |
| 293 | 0 | 0 | 8 | 0 | 8 |
| 294 | 0 | 0 | 8 | 0 | 8 |
| 295 | 8 | 0 | 0 | 0 | 8 |
| 296 | 6 | 0 | 2 | 0 | 8 |
| 297 | 8 | 0 | 0 | 0 | 8 |
| 298 | 6 | 2 | 0 | 0 | 8 |
| 299 | 8 | 0 | 0 | 0 | 8 |
| 300 | 0 | 0 | 8 | 0 | 8 |
| 301 | 8 | 0 | 0 | 0 | 8 |
| 302 | 8 | 0 | 0 | 0 | 8 |
| 303 | 8 | 0 | 0 | 0 | 8 |
| 304 | 6 | 0 | 2 | 0 | 8 |
| 305 | 8 | 0 | 0 | 0 | 8 |
| 306 | 8 | 0 | 0 | 0 | 8 |
| 307 | 8 | 0 | 0 | 0 | 8 |
| 308 | 8 | 0 | 0 | 0 | 8 |
| 309 | 0 | 0 | 8 | 0 | 8 |
| 310 | 8 | 0 | 0 | 0 | 8 |
| 311 | 8 | 0 | 0 | 0 | 8 |
| 312 | 8 | 0 | 0 | 0 | 8 |
| 313 | 8 | 0 | 0 | 0 | 8 |
| 314 | 8 | 0 | 0 | 0 | 8 |
| 315 | 8 | 0 | 0 | 0 | 8 |
| 316 | 8 | 0 | 0 | 0 | 8 |
| 317 | 8 | 0 | 0 | 0 | 8 |
| 318 | 7 | 0 | 0 | 0 | 7 |
| 319 | 7 | 0 | 0 | 0 | 7 |
| 320 | 7 | 0 | 0 | 0 | 7 |
| 321 | 3 | 0 | 4 | 0 | 7 |
| 322 | 5 | 0 | 2 | 0 | 7 |
| 323 | 3 | 0 | 4 | 0 | 7 |
| 324 | 7 | 0 | 0 | 0 | 7 |
| 325 | 7 | 0 | 0 | 0 | 7 |
| 326 | 7 | 0 | 0 | 0 | 7 |
| 327 | 4 | 3 | 0 | 0 | 7 |
| 328 | 7 | 0 | 0 | 0 | 7 |
| 329 | 0 | 0 | 7 | 0 | 7 |
| 330 | 7 | 0 | 0 | 0 | 7 |
| 331 | 7 | 0 | 0 | 0 | 7 |
| 332 | 6 | 1 | 0 | 0 | 7 |
| 333 | 7 | 0 | 0 | 0 | 7 |
| 334 | 7 | 0 | 0 | 0 | 7 |
| 335 | 7 | 0 | 0 | 0 | 7 |
| 336 | 7 | 0 | 0 | 0 | 7 |
| 337 | 7 | 0 | 0 | 0 | 7 |
| 338 | 7 | 0 | 0 | 0 | 7 |
| 339 | 7 | 0 | 0 | 0 | 7 |
| 340 | 7 | 0 | 0 | 0 | 7 |
| 341 | 7 | 0 | 0 | 0 | 7 |
| 342 | 7 | 0 | 0 | 0 | 7 |
| 343 | 7 | 0 | 0 | 0 | 7 |
| 344 | 5 | 2 | 0 | 0 | 7 |
| 345 | 7 | 0 | 0 | 0 | 7 |
| 346 | 7 | 0 | 0 | 0 | 7 |
| 347 | 7 | 0 | 0 | 0 | 7 |
| 348 | 7 | 0 | 0 | 0 | 7 |
| 349 | 7 | 0 | 0 | 0 | 7 |
| 350 | 7 | 0 | 0 | 0 | 7 |
| 351 | 7 | 0 | 0 | 0 | 7 |
| 352 | 7 | 0 | 0 | 0 | 7 |
| 353 | 6 | 0 | 1 | 0 | 7 |
| 354 | 7 | 0 | 0 | 0 | 7 |
| 355 | 4 | 3 | 0 | 0 | 7 |
| 356 | 7 | 0 | 0 | 0 | 7 |
| 357 | 7 | 0 | 0 | 0 | 7 |
| 358 | 7 | 0 | 0 | 0 | 7 |
| 359 | 7 | 0 | 0 | 0 | 7 |
| 360 | 7 | 0 | 0 | 0 | 7 |
| 361 | 7 | 0 | 0 | 0 | 7 |
| 362 | 7 | 0 | 0 | 0 | 7 |
| 363 | 7 | 0 | 0 | 0 | 7 |
| 364 | 7 | 0 | 0 | 0 | 7 |
| 365 | 7 | 0 | 0 | 0 | 7 |
| 366 | 7 | 0 | 0 | 0 | 7 |
| 367 | 7 | 0 | 0 | 0 | 7 |
| 368 | 7 | 0 | 0 | 0 | 7 |
| 369 | 7 | 0 | 0 | 0 | 7 |
| 370 | 7 | 0 | 0 | 0 | 7 |
| 371 | 7 | 0 | 0 | 0 | 7 |
| 372 | 7 | 0 | 0 | 0 | 7 |
| 373 | 7 | 0 | 0 | 0 | 7 |
| 374 | 7 | 0 | 0 | 0 | 7 |
| 375 | 7 | 0 | 0 | 0 | 7 |
| 376 | 7 | 0 | 0 | 0 | 7 |
| 377 | 5 | 2 | 0 | 0 | 7 |
| 378 | 7 | 0 | 0 | 0 | 7 |
| 379 | 7 | 0 | 0 | 0 | 7 |
| 380 | 7 | 0 | 0 | 0 | 7 |
| 381 | 7 | 0 | 0 | 0 | 7 |
| 382 | 7 | 0 | 0 | 0 | 7 |
| 383 | 4 | 3 | 0 | 0 | 7 |
| 384 | 7 | 0 | 0 | 0 | 7 |
| 385 | 7 | 0 | 0 | 0 | 7 |
| 386 | 5 | 0 | 2 | 0 | 7 |
| 387 | 7 | 0 | 0 | 0 | 7 |
| 388 | 7 | 0 | 0 | 0 | 7 |
| 389 | 7 | 0 | 0 | 0 | 7 |
| 390 | 7 | 0 | 0 | 0 | 7 |
| 391 | 7 | 0 | 0 | 0 | 7 |
| 392 | 7 | 0 | 0 | 0 | 7 |
| 393 | 7 | 0 | 0 | 0 | 7 |
| 394 | 7 | 0 | 0 | 0 | 7 |
| 395 | 7 | 0 | 0 | 0 | 7 |
| 396 | 7 | 0 | 0 | 0 | 7 |
| 397 | 7 | 0 | 0 | 0 | 7 |
| 398 | 7 | 0 | 0 | 0 | 7 |
| 399 | 7 | 0 | 0 | 0 | 7 |
| 400 | 7 | 0 | 0 | 0 | 7 |
| 401 | 7 | 0 | 0 | 0 | 7 |
| 402 | 7 | 0 | 0 | 0 | 7 |
| 403 | 7 | 0 | 0 | 0 | 7 |
| 404 | 7 | 0 | 0 | 0 | 7 |
| 405 | 0 | 0 | 6 | 0 | 6 |
| 406 | 5 | 0 | 1 | 0 | 6 |
| 407 | 4 | 0 | 2 | 0 | 6 |
| 408 | 5 | 0 | 1 | 0 | 6 |
| 409 | 5 | 0 | 1 | 0 | 6 |
| 410 | 4 | 0 | 2 | 0 | 6 |
| 411 | 6 | 0 | 0 | 0 | 6 |
| 412 | 6 | 0 | 0 | 0 | 6 |
| 413 | 6 | 0 | 0 | 0 | 6 |
| 414 | 4 | 0 | 2 | 0 | 6 |
| 415 | 0 | 0 | 6 | 0 | 6 |
| 416 | 6 | 0 | 0 | 0 | 6 |
| 417 | 6 | 0 | 0 | 0 | 6 |
| 418 | 6 | 0 | 0 | 0 | 6 |
| 419 | 6 | 0 | 0 | 0 | 6 |
| 420 | 6 | 0 | 0 | 0 | 6 |
| 421 | 6 | 0 | 0 | 0 | 6 |
| 422 | 4 | 2 | 0 | 0 | 6 |
| 423 | 6 | 0 | 0 | 0 | 6 |
| 424 | 3 | 3 | 0 | 0 | 6 |
| 425 | 6 | 0 | 0 | 0 | 6 |
| 426 | 6 | 0 | 0 | 0 | 6 |
| 427 | 6 | 0 | 0 | 0 | 6 |
| 428 | 5 | 0 | 1 | 0 | 6 |
| 429 | 3 | 3 | 0 | 0 | 6 |
| 430 | 4 | 2 | 0 | 0 | 6 |
| 431 | 6 | 0 | 0 | 0 | 6 |
| 432 | 6 | 0 | 0 | 0 | 6 |
| 433 | 6 | 0 | 0 | 0 | 6 |
| 434 | 6 | 0 | 0 | 0 | 6 |
| 435 | 6 | 0 | 0 | 0 | 6 |
| 436 | 6 | 0 | 0 | 0 | 6 |
| 437 | 3 | 3 | 0 | 0 | 6 |
| 438 | 6 | 0 | 0 | 0 | 6 |
| 439 | 6 | 0 | 0 | 0 | 6 |
| 440 | 6 | 0 | 0 | 0 | 6 |
| 441 | 6 | 0 | 0 | 0 | 6 |
| 442 | 6 | 0 | 0 | 0 | 6 |
| 443 | 6 | 0 | 0 | 0 | 6 |
| 444 | 3 | 3 | 0 | 0 | 6 |
| 445 | 6 | 0 | 0 | 0 | 6 |
| 446 | 6 | 0 | 0 | 0 | 6 |
| 447 | 6 | 0 | 0 | 0 | 6 |
| 448 | 3 | 0 | 3 | 0 | 6 |
| 449 | 4 | 0 | 2 | 0 | 6 |
| 450 | 6 | 0 | 0 | 0 | 6 |
| 451 | 6 | 0 | 0 | 0 | 6 |
| 452 | 4 | 2 | 0 | 0 | 6 |
| 453 | 6 | 0 | 0 | 0 | 6 |
| 454 | 6 | 0 | 0 | 0 | 6 |
| 455 | 6 | 0 | 0 | 0 | 6 |
| 456 | 6 | 0 | 0 | 0 | 6 |
| 457 | 6 | 0 | 0 | 0 | 6 |
| 458 | 4 | 2 | 0 | 0 | 6 |
| 459 | 6 | 0 | 0 | 0 | 6 |
| 460 | 6 | 0 | 0 | 0 | 6 |
| 461 | 6 | 0 | 0 | 0 | 6 |
| 462 | 6 | 0 | 0 | 0 | 6 |
| 463 | 6 | 0 | 0 | 0 | 6 |
| 464 | 6 | 0 | 0 | 0 | 6 |
| 465 | 6 | 0 | 0 | 0 | 6 |
| 466 | 6 | 0 | 0 | 0 | 6 |
| 467 | 6 | 0 | 0 | 0 | 6 |
| 468 | 6 | 0 | 0 | 0 | 6 |
| 469 | 6 | 0 | 0 | 0 | 6 |
| 470 | 6 | 0 | 0 | 0 | 6 |
| 471 | 4 | 0 | 2 | 0 | 6 |
| 472 | 6 | 0 | 0 | 0 | 6 |
| 473 | 6 | 0 | 0 | 0 | 6 |
| 474 | 4 | 2 | 0 | 0 | 6 |
| 475 | 6 | 0 | 0 | 0 | 6 |
| 476 | 6 | 0 | 0 | 0 | 6 |
| 477 | 6 | 0 | 0 | 0 | 6 |
| 478 | 6 | 0 | 0 | 0 | 6 |
| 479 | 6 | 0 | 0 | 0 | 6 |
| 480 | 6 | 0 | 0 | 0 | 6 |
| 481 | 6 | 0 | 0 | 0 | 6 |
| 482 | 6 | 0 | 0 | 0 | 6 |
| 483 | 3 | 3 | 0 | 0 | 6 |
| 484 | 6 | 0 | 0 | 0 | 6 |
| 485 | 6 | 0 | 0 | 0 | 6 |
| 486 | 6 | 0 | 0 | 0 | 6 |
| 487 | 6 | 0 | 0 | 0 | 6 |
| 488 | 5 | 0 | 1 | 0 | 6 |
| 489 | 6 | 0 | 0 | 0 | 6 |
| 490 | 6 | 0 | 0 | 0 | 6 |
| 491 | 4 | 2 | 0 | 0 | 6 |
| 492 | 6 | 0 | 0 | 0 | 6 |
| 493 | 6 | 0 | 0 | 0 | 6 |
| 494 | 6 | 0 | 0 | 0 | 6 |
| 495 | 6 | 0 | 0 | 0 | 6 |
| 496 | 6 | 0 | 0 | 0 | 6 |
| 497 | 0 | 0 | 6 | 0 | 6 |
| 498 | 4 | 2 | 0 | 0 | 6 |
| 499 | 6 | 0 | 0 | 0 | 6 |
| 500 | 6 | 0 | 0 | 0 | 6 |
| 501 | 6 | 0 | 0 | 0 | 6 |
| 502 | 6 | 0 | 0 | 0 | 6 |
| 503 | 6 | 0 | 0 | 0 | 6 |
| 504 | 6 | 0 | 0 | 0 | 6 |
| 505 | 6 | 0 | 0 | 0 | 6 |
| 506 | 2 | 0 | 4 | 0 | 6 |
| 507 | 6 | 0 | 0 | 0 | 6 |
| 508 | 5 | 1 | 0 | 0 | 6 |
| 509 | 6 | 0 | 0 | 0 | 6 |
| 510 | 6 | 0 | 0 | 0 | 6 |
| 511 | 4 | 2 | 0 | 0 | 6 |
| 512 | 6 | 0 | 0 | 0 | 6 |
| 513 | 6 | 0 | 0 | 0 | 6 |
| 514 | 6 | 0 | 0 | 0 | 6 |
| 515 | 6 | 0 | 0 | 0 | 6 |
| 516 | 6 | 0 | 0 | 0 | 6 |
| 517 | 6 | 0 | 0 | 0 | 6 |
| 518 | 6 | 0 | 0 | 0 | 6 |
| 519 | 6 | 0 | 0 | 0 | 6 |
| 520 | 6 | 0 | 0 | 0 | 6 |
| 521 | 6 | 0 | 0 | 0 | 6 |
| 522 | 6 | 0 | 0 | 0 | 6 |
| 523 | 6 | 0 | 0 | 0 | 6 |
| 524 | 6 | 0 | 0 | 0 | 6 |
| 525 | 6 | 0 | 0 | 0 | 6 |
| 526 | 6 | 0 | 0 | 0 | 6 |
| 527 | 6 | 0 | 0 | 0 | 6 |
| 528 | 6 | 0 | 0 | 0 | 6 |
| 529 | 6 | 0 | 0 | 0 | 6 |
| 530 | 6 | 0 | 0 | 0 | 6 |
| 531 | 6 | 0 | 0 | 0 | 6 |
| 532 | 6 | 0 | 0 | 0 | 6 |
| 533 | 6 | 0 | 0 | 0 | 6 |
| 534 | 6 | 0 | 0 | 0 | 6 |
| 535 | 4 | 0 | 1 | 0 | 5 |
| 536 | 0 | 0 | 5 | 0 | 5 |
| 537 | 5 | 0 | 0 | 0 | 5 |
| 538 | 5 | 0 | 0 | 0 | 5 |
| 539 | 5 | 0 | 0 | 0 | 5 |
| 540 | 0 | 0 | 4 | 1 | 5 |
| 541 | 5 | 0 | 0 | 0 | 5 |
| 542 | 3 | 0 | 2 | 0 | 5 |
| 543 | 0 | 0 | 3 | 2 | 5 |
| 544 | 4 | 0 | 1 | 0 | 5 |
| 545 | 5 | 0 | 0 | 0 | 5 |
| 546 | 5 | 0 | 0 | 0 | 5 |
| 547 | 5 | 0 | 0 | 0 | 5 |
| 548 | 5 | 0 | 0 | 0 | 5 |
| 549 | 5 | 0 | 0 | 0 | 5 |
| 550 | 5 | 0 | 0 | 0 | 5 |
| 551 | 0 | 0 | 5 | 0 | 5 |
| 552 | 5 | 0 | 0 | 0 | 5 |
| 553 | 3 | 2 | 0 | 0 | 5 |
| 554 | 5 | 0 | 0 | 0 | 5 |
| 555 | 5 | 0 | 0 | 0 | 5 |
| 556 | 5 | 0 | 0 | 0 | 5 |
| 557 | 5 | 0 | 0 | 0 | 5 |
| 558 | 5 | 0 | 0 | 0 | 5 |
| 559 | 5 | 0 | 0 | 0 | 5 |
| 560 | 5 | 0 | 0 | 0 | 5 |
| 561 | 5 | 0 | 0 | 0 | 5 |
| 562 | 5 | 0 | 0 | 0 | 5 |
| 563 | 5 | 0 | 0 | 0 | 5 |
| 564 | 2 | 0 | 3 | 0 | 5 |
| 565 | 5 | 0 | 0 | 0 | 5 |
| 566 | 5 | 0 | 0 | 0 | 5 |
| 567 | 5 | 0 | 0 | 0 | 5 |
| 568 | 5 | 0 | 0 | 0 | 5 |
| 569 | 5 | 0 | 0 | 0 | 5 |
| 570 | 5 | 0 | 0 | 0 | 5 |
| 571 | 3 | 2 | 0 | 0 | 5 |
| 572 | 5 | 0 | 0 | 0 | 5 |
| 573 | 5 | 0 | 0 | 0 | 5 |
| 574 | 5 | 0 | 0 | 0 | 5 |
| 575 | 5 | 0 | 0 | 0 | 5 |
| 576 | 5 | 0 | 0 | 0 | 5 |
| 577 | 5 | 0 | 0 | 0 | 5 |
| 578 | 5 | 0 | 0 | 0 | 5 |
| 579 | 5 | 0 | 0 | 0 | 5 |
| 580 | 5 | 0 | 0 | 0 | 5 |
| 581 | 5 | 0 | 0 | 0 | 5 |
| 582 | 3 | 2 | 0 | 0 | 5 |
| 583 | 3 | 2 | 0 | 0 | 5 |
| 584 | 0 | 0 | 5 | 0 | 5 |
| 585 | 5 | 0 | 0 | 0 | 5 |
| 586 | 5 | 0 | 0 | 0 | 5 |
| 587 | 5 | 0 | 0 | 0 | 5 |
| 588 | 5 | 0 | 0 | 0 | 5 |
| 589 | 5 | 0 | 0 | 0 | 5 |
| 590 | 5 | 0 | 0 | 0 | 5 |
| 591 | 5 | 0 | 0 | 0 | 5 |
| 592 | 0 | 0 | 5 | 0 | 5 |
| 593 | 5 | 0 | 0 | 0 | 5 |
| 594 | 3 | 2 | 0 | 0 | 5 |
| 595 | 5 | 0 | 0 | 0 | 5 |
| 596 | 5 | 0 | 0 | 0 | 5 |
| 597 | 5 | 0 | 0 | 0 | 5 |
| 598 | 5 | 0 | 0 | 0 | 5 |
| 599 | 5 | 0 | 0 | 0 | 5 |
| 600 | 5 | 0 | 0 | 0 | 5 |
| 601 | 0 | 0 | 5 | 0 | 5 |
| 602 | 3 | 2 | 0 | 0 | 5 |
| 603 | 5 | 0 | 0 | 0 | 5 |
| 604 | 5 | 0 | 0 | 0 | 5 |
| 605 | 5 | 0 | 0 | 0 | 5 |
| 606 | 5 | 0 | 0 | 0 | 5 |
| 607 | 5 | 0 | 0 | 0 | 5 |
| 608 | 5 | 0 | 0 | 0 | 5 |
| 609 | 5 | 0 | 0 | 0 | 5 |
| 610 | 5 | 0 | 0 | 0 | 5 |
| 611 | 5 | 0 | 0 | 0 | 5 |
| 612 | 3 | 0 | 2 | 0 | 5 |
| 613 | 5 | 0 | 0 | 0 | 5 |
| 614 | 5 | 0 | 0 | 0 | 5 |
| 615 | 5 | 0 | 0 | 0 | 5 |
| 616 | 5 | 0 | 0 | 0 | 5 |
| 617 | 5 | 0 | 0 | 0 | 5 |
| 618 | 5 | 0 | 0 | 0 | 5 |
| 619 | 5 | 0 | 0 | 0 | 5 |
| 620 | 5 | 0 | 0 | 0 | 5 |
| 621 | 5 | 0 | 0 | 0 | 5 |
| 622 | 5 | 0 | 0 | 0 | 5 |
| 623 | 5 | 0 | 0 | 0 | 5 |
| 624 | 5 | 0 | 0 | 0 | 5 |
| 625 | 5 | 0 | 0 | 0 | 5 |
| 626 | 5 | 0 | 0 | 0 | 5 |
| 627 | 5 | 0 | 0 | 0 | 5 |
| 628 | 5 | 0 | 0 | 0 | 5 |
| 629 | 5 | 0 | 0 | 0 | 5 |
| 630 | 5 | 0 | 0 | 0 | 5 |
| 631 | 5 | 0 | 0 | 0 | 5 |
| 632 | 5 | 0 | 0 | 0 | 5 |
| 633 | 5 | 0 | 0 | 0 | 5 |
| 634 | 5 | 0 | 0 | 0 | 5 |
| 635 | 5 | 0 | 0 | 0 | 5 |
| 636 | 5 | 0 | 0 | 0 | 5 |
| 637 | 5 | 0 | 0 | 0 | 5 |
| 638 | 5 | 0 | 0 | 0 | 5 |
| 639 | 5 | 0 | 0 | 0 | 5 |
| 640 | 5 | 0 | 0 | 0 | 5 |
| 641 | 4 | 1 | 0 | 0 | 5 |
| 642 | 5 | 0 | 0 | 0 | 5 |
| 643 | 5 | 0 | 0 | 0 | 5 |
| 644 | 5 | 0 | 0 | 0 | 5 |
| 645 | 3 | 2 | 0 | 0 | 5 |
| 646 | 5 | 0 | 0 | 0 | 5 |
| 647 | 5 | 0 | 0 | 0 | 5 |
| 648 | 5 | 0 | 0 | 0 | 5 |
| 649 | 1 | 0 | 4 | 0 | 5 |
| 650 | 5 | 0 | 0 | 0 | 5 |
| 651 | 5 | 0 | 0 | 0 | 5 |
| 652 | 3 | 2 | 0 | 0 | 5 |
| 653 | 5 | 0 | 0 | 0 | 5 |
| 654 | 5 | 0 | 0 | 0 | 5 |
| 655 | 5 | 0 | 0 | 0 | 5 |
| 656 | 5 | 0 | 0 | 0 | 5 |
| 657 | 5 | 0 | 0 | 0 | 5 |
| 658 | 5 | 0 | 0 | 0 | 5 |
| 659 | 5 | 0 | 0 | 0 | 5 |
| 660 | 5 | 0 | 0 | 0 | 5 |
| 661 | 5 | 0 | 0 | 0 | 5 |
| 662 | 5 | 0 | 0 | 0 | 5 |
| 663 | 5 | 0 | 0 | 0 | 5 |
| 664 | 5 | 0 | 0 | 0 | 5 |
| 665 | 5 | 0 | 0 | 0 | 5 |
| 666 | 5 | 0 | 0 | 0 | 5 |
| 667 | 5 | 0 | 0 | 0 | 5 |
| 668 | 5 | 0 | 0 | 0 | 5 |
| 669 | 5 | 0 | 0 | 0 | 5 |
| 670 | 0 | 0 | 5 | 0 | 5 |
| 671 | 5 | 0 | 0 | 0 | 5 |
| 672 | 5 | 0 | 0 | 0 | 5 |
| 673 | 5 | 0 | 0 | 0 | 5 |
| 674 | 5 | 0 | 0 | 0 | 5 |
| 675 | 0 | 0 | 5 | 0 | 5 |
| 676 | 5 | 0 | 0 | 0 | 5 |
| 677 | 5 | 0 | 0 | 0 | 5 |
| 678 | 5 | 0 | 0 | 0 | 5 |
| 679 | 5 | 0 | 0 | 0 | 5 |
| 680 | 5 | 0 | 0 | 0 | 5 |
| 681 | 5 | 0 | 0 | 0 | 5 |
| 682 | 5 | 0 | 0 | 0 | 5 |
| 683 | 5 | 0 | 0 | 0 | 5 |
| 684 | 5 | 0 | 0 | 0 | 5 |
| 685 | 5 | 0 | 0 | 0 | 5 |
| 686 | 5 | 0 | 0 | 0 | 5 |
| 687 | 5 | 0 | 0 | 0 | 5 |
| 688 | 5 | 0 | 0 | 0 | 5 |
| 689 | 5 | 0 | 0 | 0 | 5 |
| 690 | 5 | 0 | 0 | 0 | 5 |
| 691 | 5 | 0 | 0 | 0 | 5 |
| 692 | 5 | 0 | 0 | 0 | 5 |
| 693 | 5 | 0 | 0 | 0 | 5 |
| 694 | 5 | 0 | 0 | 0 | 5 |
| 695 | 5 | 0 | 0 | 0 | 5 |
| 696 | 5 | 0 | 0 | 0 | 5 |
| 697 | 5 | 0 | 0 | 0 | 5 |
| 698 | 5 | 0 | 0 | 0 | 5 |
| 699 | 5 | 0 | 0 | 0 | 5 |
| 700 | 5 | 0 | 0 | 0 | 5 |
| 701 | 5 | 0 | 0 | 0 | 5 |
| 702 | 5 | 0 | 0 | 0 | 5 |
| 703 | 4 | 0 | 0 | 0 | 4 |
| 704 | 3 | 0 | 1 | 0 | 4 |
| 705 | 4 | 0 | 0 | 0 | 4 |
| 706 | 0 | 0 | 4 | 0 | 4 |
| 707 | 4 | 0 | 0 | 0 | 4 |
| 708 | 4 | 0 | 0 | 0 | 4 |
| 709 | 4 | 0 | 0 | 0 | 4 |
| 710 | 4 | 0 | 0 | 0 | 4 |
| 711 | 4 | 0 | 0 | 0 | 4 |
| 712 | 4 | 0 | 0 | 0 | 4 |
| 713 | 0 | 0 | 4 | 0 | 4 |
| 714 | 4 | 0 | 0 | 0 | 4 |
| 715 | 3 | 0 | 1 | 0 | 4 |
| 716 | 4 | 0 | 0 | 0 | 4 |
| 717 | 4 | 0 | 0 | 0 | 4 |
| 718 | 0 | 0 | 4 | 0 | 4 |
| 719 | 4 | 0 | 0 | 0 | 4 |
| 720 | 4 | 0 | 0 | 0 | 4 |
| 721 | 4 | 0 | 0 | 0 | 4 |
| 722 | 4 | 0 | 0 | 0 | 4 |
| 723 | 4 | 0 | 0 | 0 | 4 |
| 724 | 4 | 0 | 0 | 0 | 4 |
| 725 | 4 | 0 | 0 | 0 | 4 |
| 726 | 4 | 0 | 0 | 0 | 4 |
| 727 | 4 | 0 | 0 | 0 | 4 |
| 728 | 4 | 0 | 0 | 0 | 4 |
| 729 | 4 | 0 | 0 | 0 | 4 |
| 730 | 2 | 2 | 0 | 0 | 4 |
| 731 | 4 | 0 | 0 | 0 | 4 |
| 732 | 4 | 0 | 0 | 0 | 4 |
| 733 | 4 | 0 | 0 | 0 | 4 |
| 734 | 4 | 0 | 0 | 0 | 4 |
| 735 | 0 | 0 | 4 | 0 | 4 |
| 736 | 4 | 0 | 0 | 0 | 4 |
| 737 | 4 | 0 | 0 | 0 | 4 |
| 738 | 4 | 0 | 0 | 0 | 4 |
| 739 | 4 | 0 | 0 | 0 | 4 |
| 740 | 0 | 0 | 4 | 0 | 4 |
| 741 | 4 | 0 | 0 | 0 | 4 |
| 742 | 4 | 0 | 0 | 0 | 4 |
| 743 | 4 | 0 | 0 | 0 | 4 |
| 744 | 4 | 0 | 0 | 0 | 4 |
| 745 | 4 | 0 | 0 | 0 | 4 |
| 746 | 4 | 0 | 0 | 0 | 4 |
| 747 | 4 | 0 | 0 | 0 | 4 |
| 748 | 4 | 0 | 0 | 0 | 4 |
| 749 | 4 | 0 | 0 | 0 | 4 |
| 750 | 4 | 0 | 0 | 0 | 4 |
| 751 | 4 | 0 | 0 | 0 | 4 |
| 752 | 4 | 0 | 0 | 0 | 4 |
| 753 | 4 | 0 | 0 | 0 | 4 |
| 754 | 4 | 0 | 0 | 0 | 4 |
| 755 | 4 | 0 | 0 | 0 | 4 |
| 756 | 4 | 0 | 0 | 0 | 4 |
| 757 | 4 | 0 | 0 | 0 | 4 |
| 758 | 4 | 0 | 0 | 0 | 4 |
| 759 | 4 | 0 | 0 | 0 | 4 |
| 760 | 0 | 0 | 4 | 0 | 4 |
| 761 | 3 | 0 | 1 | 0 | 4 |
| 762 | 4 | 0 | 0 | 0 | 4 |
| 763 | 4 | 0 | 0 | 0 | 4 |
| 764 | 4 | 0 | 0 | 0 | 4 |
| 765 | 4 | 0 | 0 | 0 | 4 |
| 766 | 4 | 0 | 0 | 0 | 4 |
| 767 | 2 | 0 | 2 | 0 | 4 |
| 768 | 2 | 0 | 2 | 0 | 4 |
| 769 | 4 | 0 | 0 | 0 | 4 |
| 770 | 4 | 0 | 0 | 0 | 4 |
| 771 | 4 | 0 | 0 | 0 | 4 |
| 772 | 0 | 0 | 4 | 0 | 4 |
| 773 | 4 | 0 | 0 | 0 | 4 |
| 774 | 4 | 0 | 0 | 0 | 4 |
| 775 | 3 | 0 | 1 | 0 | 4 |
| 776 | 4 | 0 | 0 | 0 | 4 |
| 777 | 4 | 0 | 0 | 0 | 4 |
| 778 | 2 | 2 | 0 | 0 | 4 |
| 779 | 4 | 0 | 0 | 0 | 4 |
| 780 | 4 | 0 | 0 | 0 | 4 |
| 781 | 4 | 0 | 0 | 0 | 4 |
| 782 | 4 | 0 | 0 | 0 | 4 |
| 783 | 4 | 0 | 0 | 0 | 4 |
| 784 | 4 | 0 | 0 | 0 | 4 |
| 785 | 4 | 0 | 0 | 0 | 4 |
| 786 | 4 | 0 | 0 | 0 | 4 |
| 787 | 0 | 0 | 4 | 0 | 4 |
| 788 | 2 | 2 | 0 | 0 | 4 |
| 789 | 4 | 0 | 0 | 0 | 4 |
| 790 | 4 | 0 | 0 | 0 | 4 |
| 791 | 4 | 0 | 0 | 0 | 4 |
| 792 | 4 | 0 | 0 | 0 | 4 |
| 793 | 4 | 0 | 0 | 0 | 4 |
| 794 | 4 | 0 | 0 | 0 | 4 |
| 795 | 2 | 2 | 0 | 0 | 4 |
| 796 | 4 | 0 | 0 | 0 | 4 |
| 797 | 4 | 0 | 0 | 0 | 4 |
| 798 | 4 | 0 | 0 | 0 | 4 |
| 799 | 2 | 0 | 2 | 0 | 4 |
| 800 | 4 | 0 | 0 | 0 | 4 |
| 801 | 4 | 0 | 0 | 0 | 4 |
| 802 | 4 | 0 | 0 | 0 | 4 |
| 803 | 4 | 0 | 0 | 0 | 4 |
| 804 | 4 | 0 | 0 | 0 | 4 |
| 805 | 4 | 0 | 0 | 0 | 4 |
| 806 | 4 | 0 | 0 | 0 | 4 |
| 807 | 4 | 0 | 0 | 0 | 4 |
| 808 | 4 | 0 | 0 | 0 | 4 |
| 809 | 4 | 0 | 0 | 0 | 4 |
| 810 | 4 | 0 | 0 | 0 | 4 |
| 811 | 4 | 0 | 0 | 0 | 4 |
| 812 | 0 | 0 | 4 | 0 | 4 |
| 813 | 4 | 0 | 0 | 0 | 4 |
| 814 | 0 | 0 | 4 | 0 | 4 |
| 815 | 4 | 0 | 0 | 0 | 4 |
| 816 | 4 | 0 | 0 | 0 | 4 |
| 817 | 4 | 0 | 0 | 0 | 4 |
| 818 | 4 | 0 | 0 | 0 | 4 |
| 819 | 4 | 0 | 0 | 0 | 4 |
| 820 | 4 | 0 | 0 | 0 | 4 |
| 821 | 4 | 0 | 0 | 0 | 4 |
| 822 | 4 | 0 | 0 | 0 | 4 |
| 823 | 0 | 0 | 4 | 0 | 4 |
| 824 | 4 | 0 | 0 | 0 | 4 |
| 825 | 4 | 0 | 0 | 0 | 4 |
| 826 | 4 | 0 | 0 | 0 | 4 |
| 827 | 0 | 0 | 4 | 0 | 4 |
| 828 | 4 | 0 | 0 | 0 | 4 |
| 829 | 4 | 0 | 0 | 0 | 4 |
| 830 | 4 | 0 | 0 | 0 | 4 |
| 831 | 4 | 0 | 0 | 0 | 4 |
| 832 | 4 | 0 | 0 | 0 | 4 |
| 833 | 4 | 0 | 0 | 0 | 4 |
| 834 | 4 | 0 | 0 | 0 | 4 |
| 835 | 4 | 0 | 0 | 0 | 4 |
| 836 | 0 | 0 | 4 | 0 | 4 |
| 837 | 4 | 0 | 0 | 0 | 4 |
| 838 | 4 | 0 | 0 | 0 | 4 |
| 839 | 4 | 0 | 0 | 0 | 4 |
| 840 | 4 | 0 | 0 | 0 | 4 |
| 841 | 4 | 0 | 0 | 0 | 4 |
| 842 | 4 | 0 | 0 | 0 | 4 |
| 843 | 4 | 0 | 0 | 0 | 4 |
| 844 | 4 | 0 | 0 | 0 | 4 |
| 845 | 4 | 0 | 0 | 0 | 4 |
| 846 | 4 | 0 | 0 | 0 | 4 |
| 847 | 4 | 0 | 0 | 0 | 4 |
| 848 | 4 | 0 | 0 | 0 | 4 |
| 849 | 4 | 0 | 0 | 0 | 4 |
| 850 | 0 | 0 | 4 | 0 | 4 |
| 851 | 4 | 0 | 0 | 0 | 4 |
| 852 | 4 | 0 | 0 | 0 | 4 |
| 853 | 0 | 0 | 4 | 0 | 4 |
| 854 | 4 | 0 | 0 | 0 | 4 |
| 855 | 4 | 0 | 0 | 0 | 4 |
| 856 | 4 | 0 | 0 | 0 | 4 |
| 857 | 3 | 0 | 0 | 0 | 3 |
| 858 | 3 | 0 | 0 | 0 | 3 |
| 859 | 2 | 0 | 1 | 0 | 3 |
| 860 | 3 | 0 | 0 | 0 | 3 |
| 861 | 2 | 0 | 1 | 0 | 3 |
| 862 | 3 | 0 | 0 | 0 | 3 |
| 863 | 3 | 0 | 0 | 0 | 3 |
| 864 | 2 | 0 | 1 | 0 | 3 |
| 865 | 0 | 0 | 2 | 1 | 3 |
| 866 | 3 | 0 | 0 | 0 | 3 |
| 867 | 3 | 0 | 0 | 0 | 3 |
| 868 | 3 | 0 | 0 | 0 | 3 |
| 869 | 3 | 0 | 0 | 0 | 3 |
| 870 | 1 | 0 | 2 | 0 | 3 |
| 871 | 3 | 0 | 0 | 0 | 3 |
| 872 | 3 | 0 | 0 | 0 | 3 |
| 873 | 3 | 0 | 0 | 0 | 3 |
| 874 | 3 | 0 | 0 | 0 | 3 |
| 875 | 3 | 0 | 0 | 0 | 3 |
| 876 | 3 | 0 | 0 | 0 | 3 |
| 877 | 3 | 0 | 0 | 0 | 3 |
| 878 | 3 | 0 | 0 | 0 | 3 |
| 879 | 3 | 0 | 0 | 0 | 3 |
| 880 | 3 | 0 | 0 | 0 | 3 |
| 881 | 3 | 0 | 0 | 0 | 3 |
| 882 | 3 | 0 | 0 | 0 | 3 |
| 883 | 3 | 0 | 0 | 0 | 3 |
| 884 | 3 | 0 | 0 | 0 | 3 |
| 885 | 3 | 0 | 0 | 0 | 3 |
| 886 | 3 | 0 | 0 | 0 | 3 |
| 887 | 3 | 0 | 0 | 0 | 3 |
| 888 | 3 | 0 | 0 | 0 | 3 |
| 889 | 0 | 0 | 3 | 0 | 3 |
| 890 | 3 | 0 | 0 | 0 | 3 |
| 891 | 3 | 0 | 0 | 0 | 3 |
| 892 | 0 | 0 | 3 | 0 | 3 |
| 893 | 3 | 0 | 0 | 0 | 3 |
| 894 | 3 | 0 | 0 | 0 | 3 |
| 895 | 3 | 0 | 0 | 0 | 3 |
| 896 | 3 | 0 | 0 | 0 | 3 |
| 897 | 3 | 0 | 0 | 0 | 3 |
| 898 | 0 | 0 | 3 | 0 | 3 |
| 899 | 3 | 0 | 0 | 0 | 3 |
| 900 | 3 | 0 | 0 | 0 | 3 |
| 901 | 3 | 0 | 0 | 0 | 3 |
| 902 | 3 | 0 | 0 | 0 | 3 |
| 903 | 3 | 0 | 0 | 0 | 3 |
| 904 | 3 | 0 | 0 | 0 | 3 |
| 905 | 0 | 0 | 3 | 0 | 3 |
| 906 | 3 | 0 | 0 | 0 | 3 |
| 907 | 3 | 0 | 0 | 0 | 3 |
| 908 | 3 | 0 | 0 | 0 | 3 |
| 909 | 3 | 0 | 0 | 0 | 3 |
| 910 | 3 | 0 | 0 | 0 | 3 |
| 911 | 3 | 0 | 0 | 0 | 3 |
| 912 | 3 | 0 | 0 | 0 | 3 |
| 913 | 3 | 0 | 0 | 0 | 3 |
| 914 | 3 | 0 | 0 | 0 | 3 |
| 915 | 3 | 0 | 0 | 0 | 3 |
| 916 | 3 | 0 | 0 | 0 | 3 |
| 917 | 3 | 0 | 0 | 0 | 3 |
| 918 | 3 | 0 | 0 | 0 | 3 |
| 919 | 3 | 0 | 0 | 0 | 3 |
| 920 | 3 | 0 | 0 | 0 | 3 |
| 921 | 3 | 0 | 0 | 0 | 3 |
| 922 | 0 | 0 | 3 | 0 | 3 |
| 923 | 3 | 0 | 0 | 0 | 3 |
| 924 | 3 | 0 | 0 | 0 | 3 |
| 925 | 3 | 0 | 0 | 0 | 3 |
| 926 | 3 | 0 | 0 | 0 | 3 |
| 927 | 3 | 0 | 0 | 0 | 3 |
| 928 | 3 | 0 | 0 | 0 | 3 |
| 929 | 0 | 0 | 3 | 0 | 3 |
| 930 | 3 | 0 | 0 | 0 | 3 |
| 931 | 3 | 0 | 0 | 0 | 3 |
| 932 | 0 | 0 | 3 | 0 | 3 |
| 933 | 3 | 0 | 0 | 0 | 3 |
| 934 | 3 | 0 | 0 | 0 | 3 |
| 935 | 3 | 0 | 0 | 0 | 3 |
| 936 | 0 | 0 | 3 | 0 | 3 |
| 937 | 3 | 0 | 0 | 0 | 3 |
| 938 | 3 | 0 | 0 | 0 | 3 |
| 939 | 3 | 0 | 0 | 0 | 3 |
| 940 | 0 | 0 | 3 | 0 | 3 |
| 941 | 3 | 0 | 0 | 0 | 3 |
| 942 | 3 | 0 | 0 | 0 | 3 |
| 943 | 3 | 0 | 0 | 0 | 3 |
| 944 | 3 | 0 | 0 | 0 | 3 |
| 945 | 3 | 0 | 0 | 0 | 3 |
| 946 | 3 | 0 | 0 | 0 | 3 |
| 947 | 3 | 0 | 0 | 0 | 3 |
| 948 | 3 | 0 | 0 | 0 | 3 |
| 949 | 3 | 0 | 0 | 0 | 3 |
| 950 | 3 | 0 | 0 | 0 | 3 |
| 951 | 3 | 0 | 0 | 0 | 3 |
| 952 | 3 | 0 | 0 | 0 | 3 |
| 953 | 3 | 0 | 0 | 0 | 3 |
| 954 | 1 | 0 | 2 | 0 | 3 |
| 955 | 3 | 0 | 0 | 0 | 3 |
| 956 | 3 | 0 | 0 | 0 | 3 |
| 957 | 3 | 0 | 0 | 0 | 3 |
| 958 | 3 | 0 | 0 | 0 | 3 |
| 959 | 3 | 0 | 0 | 0 | 3 |
| 960 | 0 | 0 | 3 | 0 | 3 |
| 961 | 3 | 0 | 0 | 0 | 3 |
| 962 | 3 | 0 | 0 | 0 | 3 |
| 963 | 3 | 0 | 0 | 0 | 3 |
| 964 | 3 | 0 | 0 | 0 | 3 |
| 965 | 3 | 0 | 0 | 0 | 3 |
| 966 | 3 | 0 | 0 | 0 | 3 |
| 967 | 3 | 0 | 0 | 0 | 3 |
| 968 | 3 | 0 | 0 | 0 | 3 |
| 969 | 3 | 0 | 0 | 0 | 3 |
| 970 | 3 | 0 | 0 | 0 | 3 |
| 971 | 3 | 0 | 0 | 0 | 3 |
| 972 | 3 | 0 | 0 | 0 | 3 |
| 973 | 3 | 0 | 0 | 0 | 3 |
| 974 | 3 | 0 | 0 | 0 | 3 |
| 975 | 3 | 0 | 0 | 0 | 3 |
| 976 | 3 | 0 | 0 | 0 | 3 |
| 977 | 0 | 0 | 3 | 0 | 3 |
| 978 | 3 | 0 | 0 | 0 | 3 |
| 979 | 3 | 0 | 0 | 0 | 3 |
| 980 | 3 | 0 | 0 | 0 | 3 |
| 981 | 3 | 0 | 0 | 0 | 3 |
| 982 | 3 | 0 | 0 | 0 | 3 |
| 983 | 3 | 0 | 0 | 0 | 3 |
| 984 | 3 | 0 | 0 | 0 | 3 |
| 985 | 3 | 0 | 0 | 0 | 3 |
| 986 | 3 | 0 | 0 | 0 | 3 |
| 987 | 3 | 0 | 0 | 0 | 3 |
| 988 | 3 | 0 | 0 | 0 | 3 |
| 989 | 3 | 0 | 0 | 0 | 3 |
| 990 | 3 | 0 | 0 | 0 | 3 |
| 991 | 3 | 0 | 0 | 0 | 3 |
| 992 | 3 | 0 | 0 | 0 | 3 |
| 993 | 3 | 0 | 0 | 0 | 3 |
| 994 | 3 | 0 | 0 | 0 | 3 |
| 995 | 3 | 0 | 0 | 0 | 3 |
| 996 | 0 | 0 | 3 | 0 | 3 |
| 997 | 3 | 0 | 0 | 0 | 3 |
| 998 | 3 | 0 | 0 | 0 | 3 |
| 999 | 3 | 0 | 0 | 0 | 3 |
| 1000 | 3 | 0 | 0 | 0 | 3 |
| 1001 | 3 | 0 | 0 | 0 | 3 |
| 1002 | 3 | 0 | 0 | 0 | 3 |
| 1003 | 3 | 0 | 0 | 0 | 3 |
| 1004 | 3 | 0 | 0 | 0 | 3 |
| 1005 | 3 | 0 | 0 | 0 | 3 |
| 1006 | 3 | 0 | 0 | 0 | 3 |
| 1007 | 3 | 0 | 0 | 0 | 3 |
| 1008 | 3 | 0 | 0 | 0 | 3 |
| 1009 | 3 | 0 | 0 | 0 | 3 |
| 1010 | 0 | 0 | 3 | 0 | 3 |
| 1011 | 3 | 0 | 0 | 0 | 3 |
| 1012 | 3 | 0 | 0 | 0 | 3 |
| 1013 | 3 | 0 | 0 | 0 | 3 |
| 1014 | 3 | 0 | 0 | 0 | 3 |
| 1015 | 3 | 0 | 0 | 0 | 3 |
| 1016 | 3 | 0 | 0 | 0 | 3 |
| 1017 | 3 | 0 | 0 | 0 | 3 |
| 1018 | 3 | 0 | 0 | 0 | 3 |
| 1019 | 3 | 0 | 0 | 0 | 3 |
| 1020 | 3 | 0 | 0 | 0 | 3 |
| 1021 | 3 | 0 | 0 | 0 | 3 |
| 1022 | 3 | 0 | 0 | 0 | 3 |
| 1023 | 3 | 0 | 0 | 0 | 3 |
| 1024 | 3 | 0 | 0 | 0 | 3 |
| 1025 | 0 | 0 | 3 | 0 | 3 |
| 1026 | 3 | 0 | 0 | 0 | 3 |
| 1027 | 2 | 0 | 1 | 0 | 3 |
| 1028 | 0 | 0 | 3 | 0 | 3 |
| 1029 | 3 | 0 | 0 | 0 | 3 |
| 1030 | 3 | 0 | 0 | 0 | 3 |
| 1031 | 0 | 0 | 3 | 0 | 3 |
| 1032 | 3 | 0 | 0 | 0 | 3 |
| 1033 | 3 | 0 | 0 | 0 | 3 |
| 1034 | 3 | 0 | 0 | 0 | 3 |
| 1035 | 3 | 0 | 0 | 0 | 3 |
| 1036 | 3 | 0 | 0 | 0 | 3 |
| 1037 | 3 | 0 | 0 | 0 | 3 |
| 1038 | 3 | 0 | 0 | 0 | 3 |
| 1039 | 0 | 0 | 3 | 0 | 3 |
| 1040 | 3 | 0 | 0 | 0 | 3 |
| 1041 | 3 | 0 | 0 | 0 | 3 |
| 1042 | 3 | 0 | 0 | 0 | 3 |
| 1043 | 3 | 0 | 0 | 0 | 3 |
| 1044 | 3 | 0 | 0 | 0 | 3 |
| 1045 | 3 | 0 | 0 | 0 | 3 |
| 1046 | 3 | 0 | 0 | 0 | 3 |
| 1047 | 3 | 0 | 0 | 0 | 3 |
| 1048 | 3 | 0 | 0 | 0 | 3 |
| 1049 | 3 | 0 | 0 | 0 | 3 |
| 1050 | 3 | 0 | 0 | 0 | 3 |
| 1051 | 3 | 0 | 0 | 0 | 3 |
| 1052 | 3 | 0 | 0 | 0 | 3 |
| 1053 | 3 | 0 | 0 | 0 | 3 |
| 1054 | 3 | 0 | 0 | 0 | 3 |
| 1055 | 3 | 0 | 0 | 0 | 3 |
| 1056 | 3 | 0 | 0 | 0 | 3 |
| 1057 | 3 | 0 | 0 | 0 | 3 |
| 1058 | 3 | 0 | 0 | 0 | 3 |
| 1059 | 3 | 0 | 0 | 0 | 3 |
| 1060 | 0 | 0 | 3 | 0 | 3 |
| 1061 | 3 | 0 | 0 | 0 | 3 |
| 1062 | 3 | 0 | 0 | 0 | 3 |
| 1063 | 3 | 0 | 0 | 0 | 3 |
| 1064 | 3 | 0 | 0 | 0 | 3 |
| 1065 | 3 | 0 | 0 | 0 | 3 |
| 1066 | 3 | 0 | 0 | 0 | 3 |
| 1067 | 3 | 0 | 0 | 0 | 3 |
| 1068 | 3 | 0 | 0 | 0 | 3 |
| 1069 | 3 | 0 | 0 | 0 | 3 |
| 1070 | 3 | 0 | 0 | 0 | 3 |
| 1071 | 3 | 0 | 0 | 0 | 3 |
| 1072 | 3 | 0 | 0 | 0 | 3 |
| 1073 | 3 | 0 | 0 | 0 | 3 |
| 1074 | 0 | 0 | 3 | 0 | 3 |
| 1075 | 3 | 0 | 0 | 0 | 3 |
| 1076 | 3 | 0 | 0 | 0 | 3 |
| 1077 | 3 | 0 | 0 | 0 | 3 |
| 1078 | 3 | 0 | 0 | 0 | 3 |
| 1079 | 3 | 0 | 0 | 0 | 3 |
| 1080 | 3 | 0 | 0 | 0 | 3 |
| 1081 | 3 | 0 | 0 | 0 | 3 |
| 1082 | 0 | 0 | 2 | 1 | 3 |
| 1083 | 3 | 0 | 0 | 0 | 3 |
| 1084 | 3 | 0 | 0 | 0 | 3 |
| 1085 | 3 | 0 | 0 | 0 | 3 |
| 1086 | 3 | 0 | 0 | 0 | 3 |
| 1087 | 3 | 0 | 0 | 0 | 3 |
| 1088 | 3 | 0 | 0 | 0 | 3 |
| 1089 | 3 | 0 | 0 | 0 | 3 |
| 1090 | 3 | 0 | 0 | 0 | 3 |
| 1091 | 3 | 0 | 0 | 0 | 3 |
| 1092 | 0 | 0 | 3 | 0 | 3 |
| 1093 | 3 | 0 | 0 | 0 | 3 |
| 1094 | 0 | 0 | 3 | 0 | 3 |
| 1095 | 0 | 0 | 3 | 0 | 3 |
| 1096 | 3 | 0 | 0 | 0 | 3 |
| 1097 | 3 | 0 | 0 | 0 | 3 |
| 1098 | 3 | 0 | 0 | 0 | 3 |
| 1099 | 3 | 0 | 0 | 0 | 3 |
| 1100 | 0 | 0 | 3 | 0 | 3 |
| 1101 | 3 | 0 | 0 | 0 | 3 |
| 1102 | 3 | 0 | 0 | 0 | 3 |
| 1103 | 3 | 0 | 0 | 0 | 3 |
| 1104 | 3 | 0 | 0 | 0 | 3 |
| 1105 | 3 | 0 | 0 | 0 | 3 |
| 1106 | 3 | 0 | 0 | 0 | 3 |
| 1107 | 3 | 0 | 0 | 0 | 3 |
| 1108 | 3 | 0 | 0 | 0 | 3 |
| 1109 | 0 | 0 | 3 | 0 | 3 |
| 1110 | 0 | 0 | 3 | 0 | 3 |
| 1111 | 3 | 0 | 0 | 0 | 3 |
| 1112 | 3 | 0 | 0 | 0 | 3 |
| 1113 | 3 | 0 | 0 | 0 | 3 |
| 1114 | 3 | 0 | 0 | 0 | 3 |
| 1115 | 2 | 0 | 0 | 0 | 2 |
| 1116 | 2 | 0 | 0 | 0 | 2 |
| 1117 | 2 | 0 | 0 | 0 | 2 |
| 1118 | 2 | 0 | 0 | 0 | 2 |
| 1119 | 2 | 0 | 0 | 0 | 2 |
| 1120 | 0 | 0 | 2 | 0 | 2 |
| 1121 | 2 | 0 | 0 | 0 | 2 |
| 1122 | 2 | 0 | 0 | 0 | 2 |
| 1123 | 2 | 0 | 0 | 0 | 2 |
| 1124 | 2 | 0 | 0 | 0 | 2 |
| 1125 | 2 | 0 | 0 | 0 | 2 |
| 1126 | 1 | 0 | 1 | 0 | 2 |
| 1127 | 0 | 0 | 2 | 0 | 2 |
| 1128 | 2 | 0 | 0 | 0 | 2 |
| 1129 | 0 | 0 | 2 | 0 | 2 |
| 1130 | 2 | 0 | 0 | 0 | 2 |
| 1131 | 0 | 0 | 2 | 0 | 2 |
| 1132 | 0 | 0 | 2 | 0 | 2 |
| 1133 | 0 | 0 | 2 | 0 | 2 |
| 1134 | 2 | 0 | 0 | 0 | 2 |
| 1135 | 0 | 0 | 2 | 0 | 2 |
| 1136 | 2 | 0 | 0 | 0 | 2 |
| 1137 | 2 | 0 | 0 | 0 | 2 |
| 1138 | 2 | 0 | 0 | 0 | 2 |
| 1139 | 2 | 0 | 0 | 0 | 2 |
| 1140 | 2 | 0 | 0 | 0 | 2 |
| 1141 | 2 | 0 | 0 | 0 | 2 |
| 1142 | 2 | 0 | 0 | 0 | 2 |
| 1143 | 2 | 0 | 0 | 0 | 2 |
| 1144 | 2 | 0 | 0 | 0 | 2 |
| 1145 | 2 | 0 | 0 | 0 | 2 |
| 1146 | 2 | 0 | 0 | 0 | 2 |
| 1147 | 0 | 0 | 2 | 0 | 2 |
| 1148 | 2 | 0 | 0 | 0 | 2 |
| 1149 | 2 | 0 | 0 | 0 | 2 |
| 1150 | 2 | 0 | 0 | 0 | 2 |
| 1151 | 2 | 0 | 0 | 0 | 2 |
| 1152 | 2 | 0 | 0 | 0 | 2 |
| 1153 | 2 | 0 | 0 | 0 | 2 |
| 1154 | 2 | 0 | 0 | 0 | 2 |
| 1155 | 0 | 0 | 2 | 0 | 2 |
| 1156 | 2 | 0 | 0 | 0 | 2 |
| 1157 | 2 | 0 | 0 | 0 | 2 |
| 1158 | 2 | 0 | 0 | 0 | 2 |
| 1159 | 2 | 0 | 0 | 0 | 2 |
| 1160 | 2 | 0 | 0 | 0 | 2 |
| 1161 | 2 | 0 | 0 | 0 | 2 |
| 1162 | 2 | 0 | 0 | 0 | 2 |
| 1163 | 2 | 0 | 0 | 0 | 2 |
| 1164 | 2 | 0 | 0 | 0 | 2 |
| 1165 | 2 | 0 | 0 | 0 | 2 |
| 1166 | 2 | 0 | 0 | 0 | 2 |
| 1167 | 2 | 0 | 0 | 0 | 2 |
| 1168 | 0 | 0 | 2 | 0 | 2 |
| 1169 | 2 | 0 | 0 | 0 | 2 |
| 1170 | 2 | 0 | 0 | 0 | 2 |
| 1171 | 2 | 0 | 0 | 0 | 2 |
| 1172 | 2 | 0 | 0 | 0 | 2 |
| 1173 | 2 | 0 | 0 | 0 | 2 |
| 1174 | 2 | 0 | 0 | 0 | 2 |
| 1175 | 2 | 0 | 0 | 0 | 2 |
| 1176 | 2 | 0 | 0 | 0 | 2 |
| 1177 | 2 | 0 | 0 | 0 | 2 |
| 1178 | 0 | 0 | 2 | 0 | 2 |
| 1179 | 2 | 0 | 0 | 0 | 2 |
| 1180 | 0 | 0 | 2 | 0 | 2 |
| 1181 | 2 | 0 | 0 | 0 | 2 |
| 1182 | 2 | 0 | 0 | 0 | 2 |
| 1183 | 0 | 0 | 2 | 0 | 2 |
| 1184 | 2 | 0 | 0 | 0 | 2 |
| 1185 | 2 | 0 | 0 | 0 | 2 |
| 1186 | 2 | 0 | 0 | 0 | 2 |
| 1187 | 2 | 0 | 0 | 0 | 2 |
| 1188 | 2 | 0 | 0 | 0 | 2 |
| 1189 | 0 | 0 | 2 | 0 | 2 |
| 1190 | 0 | 0 | 2 | 0 | 2 |
| 1191 | 0 | 0 | 2 | 0 | 2 |
| 1192 | 2 | 0 | 0 | 0 | 2 |
| 1193 | 2 | 0 | 0 | 0 | 2 |
| 1194 | 2 | 0 | 0 | 0 | 2 |
| 1195 | 2 | 0 | 0 | 0 | 2 |
| 1196 | 2 | 0 | 0 | 0 | 2 |
| 1197 | 2 | 0 | 0 | 0 | 2 |
| 1198 | 2 | 0 | 0 | 0 | 2 |
| 1199 | 2 | 0 | 0 | 0 | 2 |
| 1200 | 2 | 0 | 0 | 0 | 2 |
| 1201 | 2 | 0 | 0 | 0 | 2 |
| 1202 | 2 | 0 | 0 | 0 | 2 |
| 1203 | 2 | 0 | 0 | 0 | 2 |
| 1204 | 0 | 0 | 2 | 0 | 2 |
| 1205 | 2 | 0 | 0 | 0 | 2 |
| 1206 | 2 | 0 | 0 | 0 | 2 |
| 1207 | 2 | 0 | 0 | 0 | 2 |
| 1208 | 2 | 0 | 0 | 0 | 2 |
| 1209 | 2 | 0 | 0 | 0 | 2 |
| 1210 | 2 | 0 | 0 | 0 | 2 |
| 1211 | 2 | 0 | 0 | 0 | 2 |
| 1212 | 2 | 0 | 0 | 0 | 2 |
| 1213 | 0 | 0 | 2 | 0 | 2 |
| 1214 | 2 | 0 | 0 | 0 | 2 |
| 1215 | 2 | 0 | 0 | 0 | 2 |
| 1216 | 2 | 0 | 0 | 0 | 2 |
| 1217 | 2 | 0 | 0 | 0 | 2 |
| 1218 | 2 | 0 | 0 | 0 | 2 |
| 1219 | 2 | 0 | 0 | 0 | 2 |
| 1220 | 2 | 0 | 0 | 0 | 2 |
| 1221 | 2 | 0 | 0 | 0 | 2 |
| 1222 | 2 | 0 | 0 | 0 | 2 |
| 1223 | 2 | 0 | 0 | 0 | 2 |
| 1224 | 2 | 0 | 0 | 0 | 2 |
| 1225 | 0 | 0 | 2 | 0 | 2 |
| 1226 | 2 | 0 | 0 | 0 | 2 |
| 1227 | 0 | 0 | 2 | 0 | 2 |
| 1228 | 0 | 0 | 2 | 0 | 2 |
| 1229 | 2 | 0 | 0 | 0 | 2 |
| 1230 | 2 | 0 | 0 | 0 | 2 |
| 1231 | 2 | 0 | 0 | 0 | 2 |
| 1232 | 2 | 0 | 0 | 0 | 2 |
| 1233 | 2 | 0 | 0 | 0 | 2 |
| 1234 | 2 | 0 | 0 | 0 | 2 |
| 1235 | 2 | 0 | 0 | 0 | 2 |
| 1236 | 2 | 0 | 0 | 0 | 2 |
| 1237 | 2 | 0 | 0 | 0 | 2 |
| 1238 | 2 | 0 | 0 | 0 | 2 |
| 1239 | 2 | 0 | 0 | 0 | 2 |
| 1240 | 2 | 0 | 0 | 0 | 2 |
| 1241 | 2 | 0 | 0 | 0 | 2 |
| 1242 | 0 | 0 | 2 | 0 | 2 |
| 1243 | 2 | 0 | 0 | 0 | 2 |
| 1244 | 2 | 0 | 0 | 0 | 2 |
| 1245 | 2 | 0 | 0 | 0 | 2 |
| 1246 | 2 | 0 | 0 | 0 | 2 |
| 1247 | 2 | 0 | 0 | 0 | 2 |
| 1248 | 0 | 0 | 2 | 0 | 2 |
| 1249 | 2 | 0 | 0 | 0 | 2 |
| 1250 | 0 | 0 | 2 | 0 | 2 |
| 1251 | 2 | 0 | 0 | 0 | 2 |
| 1252 | 2 | 0 | 0 | 0 | 2 |
| 1253 | 2 | 0 | 0 | 0 | 2 |
| 1254 | 0 | 0 | 2 | 0 | 2 |
| 1255 | 2 | 0 | 0 | 0 | 2 |
| 1256 | 2 | 0 | 0 | 0 | 2 |
| 1257 | 2 | 0 | 0 | 0 | 2 |
| 1258 | 2 | 0 | 0 | 0 | 2 |
| 1259 | 0 | 0 | 2 | 0 | 2 |
| 1260 | 2 | 0 | 0 | 0 | 2 |
| 1261 | 2 | 0 | 0 | 0 | 2 |
| 1262 | 2 | 0 | 0 | 0 | 2 |
| 1263 | 0 | 0 | 2 | 0 | 2 |
| 1264 | 2 | 0 | 0 | 0 | 2 |
| 1265 | 0 | 0 | 2 | 0 | 2 |
| 1266 | 2 | 0 | 0 | 0 | 2 |
| 1267 | 2 | 0 | 0 | 0 | 2 |
| 1268 | 2 | 0 | 0 | 0 | 2 |
| 1269 | 2 | 0 | 0 | 0 | 2 |
| 1270 | 0 | 0 | 2 | 0 | 2 |
| 1271 | 2 | 0 | 0 | 0 | 2 |
| 1272 | 2 | 0 | 0 | 0 | 2 |
| 1273 | 2 | 0 | 0 | 0 | 2 |
| 1274 | 0 | 0 | 2 | 0 | 2 |
| 1275 | 2 | 0 | 0 | 0 | 2 |
| 1276 | 2 | 0 | 0 | 0 | 2 |
| 1277 | 2 | 0 | 0 | 0 | 2 |
| 1278 | 2 | 0 | 0 | 0 | 2 |
| 1279 | 2 | 0 | 0 | 0 | 2 |
| 1280 | 2 | 0 | 0 | 0 | 2 |
| 1281 | 2 | 0 | 0 | 0 | 2 |
| 1282 | 2 | 0 | 0 | 0 | 2 |
| 1283 | 2 | 0 | 0 | 0 | 2 |
| 1284 | 2 | 0 | 0 | 0 | 2 |
| 1285 | 2 | 0 | 0 | 0 | 2 |
| 1286 | 2 | 0 | 0 | 0 | 2 |
| 1287 | 0 | 0 | 2 | 0 | 2 |
| 1288 | 2 | 0 | 0 | 0 | 2 |
| 1289 | 2 | 0 | 0 | 0 | 2 |
| 1290 | 2 | 0 | 0 | 0 | 2 |
| 1291 | 2 | 0 | 0 | 0 | 2 |
| 1292 | 2 | 0 | 0 | 0 | 2 |
| 1293 | 2 | 0 | 0 | 0 | 2 |
| 1294 | 2 | 0 | 0 | 0 | 2 |
| 1295 | 2 | 0 | 0 | 0 | 2 |
| 1296 | 2 | 0 | 0 | 0 | 2 |
| 1297 | 2 | 0 | 0 | 0 | 2 |
| 1298 | 2 | 0 | 0 | 0 | 2 |
| 1299 | 2 | 0 | 0 | 0 | 2 |
| 1300 | 2 | 0 | 0 | 0 | 2 |
| 1301 | 0 | 0 | 2 | 0 | 2 |
| 1302 | 2 | 0 | 0 | 0 | 2 |
| 1303 | 2 | 0 | 0 | 0 | 2 |
| 1304 | 2 | 0 | 0 | 0 | 2 |
| 1305 | 2 | 0 | 0 | 0 | 2 |
| 1306 | 2 | 0 | 0 | 0 | 2 |
| 1307 | 2 | 0 | 0 | 0 | 2 |
| 1308 | 2 | 0 | 0 | 0 | 2 |
| 1309 | 0 | 0 | 2 | 0 | 2 |
| 1310 | 0 | 0 | 2 | 0 | 2 |
| 1311 | 2 | 0 | 0 | 0 | 2 |
| 1312 | 2 | 0 | 0 | 0 | 2 |
| 1313 | 2 | 0 | 0 | 0 | 2 |
| 1314 | 2 | 0 | 0 | 0 | 2 |
| 1315 | 2 | 0 | 0 | 0 | 2 |
| 1316 | 0 | 0 | 2 | 0 | 2 |
| 1317 | 2 | 0 | 0 | 0 | 2 |
| 1318 | 2 | 0 | 0 | 0 | 2 |
| 1319 | 2 | 0 | 0 | 0 | 2 |
| 1320 | 2 | 0 | 0 | 0 | 2 |
| 1321 | 2 | 0 | 0 | 0 | 2 |
| 1322 | 2 | 0 | 0 | 0 | 2 |
| 1323 | 2 | 0 | 0 | 0 | 2 |
| 1324 | 2 | 0 | 0 | 0 | 2 |
| 1325 | 2 | 0 | 0 | 0 | 2 |
| 1326 | 0 | 0 | 2 | 0 | 2 |
| 1327 | 0 | 0 | 2 | 0 | 2 |
| 1328 | 2 | 0 | 0 | 0 | 2 |
| 1329 | 2 | 0 | 0 | 0 | 2 |
| 1330 | 2 | 0 | 0 | 0 | 2 |
| 1331 | 2 | 0 | 0 | 0 | 2 |
| 1332 | 0 | 0 | 2 | 0 | 2 |
| 1333 | 2 | 0 | 0 | 0 | 2 |
| 1334 | 0 | 0 | 2 | 0 | 2 |
| 1335 | 2 | 0 | 0 | 0 | 2 |
| 1336 | 2 | 0 | 0 | 0 | 2 |
| 1337 | 2 | 0 | 0 | 0 | 2 |
| 1338 | 2 | 0 | 0 | 0 | 2 |
| 1339 | 2 | 0 | 0 | 0 | 2 |
| 1340 | 2 | 0 | 0 | 0 | 2 |
| 1341 | 0 | 0 | 2 | 0 | 2 |
| 1342 | 2 | 0 | 0 | 0 | 2 |
| 1343 | 2 | 0 | 0 | 0 | 2 |
| 1344 | 2 | 0 | 0 | 0 | 2 |
| 1345 | 2 | 0 | 0 | 0 | 2 |
| 1346 | 2 | 0 | 0 | 0 | 2 |
| 1347 | 0 | 0 | 2 | 0 | 2 |
| 1348 | 2 | 0 | 0 | 0 | 2 |
| 1349 | 2 | 0 | 0 | 0 | 2 |
| 1350 | 2 | 0 | 0 | 0 | 2 |
| 1351 | 2 | 0 | 0 | 0 | 2 |
| 1352 | 2 | 0 | 0 | 0 | 2 |
| 1353 | 2 | 0 | 0 | 0 | 2 |
| 1354 | 2 | 0 | 0 | 0 | 2 |
| 1355 | 2 | 0 | 0 | 0 | 2 |
| 1356 | 0 | 0 | 2 | 0 | 2 |
| 1357 | 0 | 0 | 2 | 0 | 2 |
| 1358 | 2 | 0 | 0 | 0 | 2 |
| 1359 | 0 | 0 | 2 | 0 | 2 |
| 1360 | 2 | 0 | 0 | 0 | 2 |
| 1361 | 2 | 0 | 0 | 0 | 2 |
| 1362 | 2 | 0 | 0 | 0 | 2 |
| 1363 | 2 | 0 | 0 | 0 | 2 |
| 1364 | 2 | 0 | 0 | 0 | 2 |
| 1365 | 2 | 0 | 0 | 0 | 2 |
| 1366 | 2 | 0 | 0 | 0 | 2 |
| 1367 | 2 | 0 | 0 | 0 | 2 |
| 1368 | 2 | 0 | 0 | 0 | 2 |
| 1369 | 2 | 0 | 0 | 0 | 2 |
| 1370 | 2 | 0 | 0 | 0 | 2 |
| 1371 | 2 | 0 | 0 | 0 | 2 |
| 1372 | 2 | 0 | 0 | 0 | 2 |
| 1373 | 2 | 0 | 0 | 0 | 2 |
| 1374 | 2 | 0 | 0 | 0 | 2 |
| 1375 | 2 | 0 | 0 | 0 | 2 |
| 1376 | 0 | 0 | 2 | 0 | 2 |
| 1377 | 2 | 0 | 0 | 0 | 2 |
| 1378 | 2 | 0 | 0 | 0 | 2 |
| 1379 | 2 | 0 | 0 | 0 | 2 |
| 1380 | 0 | 0 | 2 | 0 | 2 |
| 1381 | 2 | 0 | 0 | 0 | 2 |
| 1382 | 2 | 0 | 0 | 0 | 2 |
| 1383 | 0 | 0 | 2 | 0 | 2 |
| 1384 | 2 | 0 | 0 | 0 | 2 |
| 1385 | 2 | 0 | 0 | 0 | 2 |
| 1386 | 2 | 0 | 0 | 0 | 2 |
| 1387 | 2 | 0 | 0 | 0 | 2 |
| 1388 | 2 | 0 | 0 | 0 | 2 |
| 1389 | 2 | 0 | 0 | 0 | 2 |
| 1390 | 2 | 0 | 0 | 0 | 2 |
| 1391 | 2 | 0 | 0 | 0 | 2 |
| 1392 | 2 | 0 | 0 | 0 | 2 |
| 1393 | 2 | 0 | 0 | 0 | 2 |
| 1394 | 2 | 0 | 0 | 0 | 2 |
| 1395 | 0 | 0 | 2 | 0 | 2 |
| 1396 | 1 | 0 | 1 | 0 | 2 |
| 1397 | 0 | 0 | 2 | 0 | 2 |
| 1398 | 2 | 0 | 0 | 0 | 2 |
| 1399 | 2 | 0 | 0 | 0 | 2 |
| 1400 | 2 | 0 | 0 | 0 | 2 |
| 1401 | 2 | 0 | 0 | 0 | 2 |
| 1402 | 2 | 0 | 0 | 0 | 2 |
| 1403 | 2 | 0 | 0 | 0 | 2 |
| 1404 | 2 | 0 | 0 | 0 | 2 |
| 1405 | 2 | 0 | 0 | 0 | 2 |
| 1406 | 2 | 0 | 0 | 0 | 2 |
| 1407 | 2 | 0 | 0 | 0 | 2 |
| 1408 | 0 | 0 | 2 | 0 | 2 |
| 1409 | 2 | 0 | 0 | 0 | 2 |
| 1410 | 2 | 0 | 0 | 0 | 2 |
| 1411 | 0 | 0 | 2 | 0 | 2 |
| 1412 | 0 | 0 | 2 | 0 | 2 |
| 1413 | 2 | 0 | 0 | 0 | 2 |
| 1414 | 2 | 0 | 0 | 0 | 2 |
| 1415 | 2 | 0 | 0 | 0 | 2 |
| 1416 | 2 | 0 | 0 | 0 | 2 |
| 1417 | 2 | 0 | 0 | 0 | 2 |
| 1418 | 2 | 0 | 0 | 0 | 2 |
| 1419 | 2 | 0 | 0 | 0 | 2 |
| 1420 | 2 | 0 | 0 | 0 | 2 |
| 1421 | 2 | 0 | 0 | 0 | 2 |
| 1422 | 2 | 0 | 0 | 0 | 2 |
| 1423 | 2 | 0 | 0 | 0 | 2 |
| 1424 | 2 | 0 | 0 | 0 | 2 |
| 1425 | 2 | 0 | 0 | 0 | 2 |
| 1426 | 2 | 0 | 0 | 0 | 2 |
| 1427 | 2 | 0 | 0 | 0 | 2 |
| 1428 | 2 | 0 | 0 | 0 | 2 |
| 1429 | 2 | 0 | 0 | 0 | 2 |
| 1430 | 2 | 0 | 0 | 0 | 2 |
| 1431 | 0 | 0 | 2 | 0 | 2 |
| 1432 | 2 | 0 | 0 | 0 | 2 |
| 1433 | 0 | 0 | 2 | 0 | 2 |
| 1434 | 2 | 0 | 0 | 0 | 2 |
| 1435 | 2 | 0 | 0 | 0 | 2 |
| 1436 | 2 | 0 | 0 | 0 | 2 |
| 1437 | 2 | 0 | 0 | 0 | 2 |
| 1438 | 2 | 0 | 0 | 0 | 2 |
| 1439 | 2 | 0 | 0 | 0 | 2 |
| 1440 | 2 | 0 | 0 | 0 | 2 |
| 1441 | 2 | 0 | 0 | 0 | 2 |
| 1442 | 0 | 0 | 2 | 0 | 2 |
| 1443 | 2 | 0 | 0 | 0 | 2 |
| 1444 | 2 | 0 | 0 | 0 | 2 |
| 1445 | 2 | 0 | 0 | 0 | 2 |
| 1446 | 0 | 0 | 2 | 0 | 2 |
| 1447 | 2 | 0 | 0 | 0 | 2 |
| 1448 | 2 | 0 | 0 | 0 | 2 |
| 1449 | 2 | 0 | 0 | 0 | 2 |
| 1450 | 2 | 0 | 0 | 0 | 2 |
| 1451 | 2 | 0 | 0 | 0 | 2 |
| 1452 | 0 | 0 | 2 | 0 | 2 |
| 1453 | 2 | 0 | 0 | 0 | 2 |
| 1454 | 2 | 0 | 0 | 0 | 2 |
| 1455 | 2 | 0 | 0 | 0 | 2 |
| 1456 | 2 | 0 | 0 | 0 | 2 |
| 1457 | 2 | 0 | 0 | 0 | 2 |
| 1458 | 2 | 0 | 0 | 0 | 2 |
| 1459 | 2 | 0 | 0 | 0 | 2 |
| 1460 | 2 | 0 | 0 | 0 | 2 |
| 1461 | 2 | 0 | 0 | 0 | 2 |
| 1462 | 2 | 0 | 0 | 0 | 2 |
| 1463 | 2 | 0 | 0 | 0 | 2 |
| 1464 | 2 | 0 | 0 | 0 | 2 |
| 1465 | 0 | 0 | 2 | 0 | 2 |
| 1466 | 2 | 0 | 0 | 0 | 2 |
| 1467 | 0 | 0 | 2 | 0 | 2 |
| 1468 | 0 | 0 | 2 | 0 | 2 |
| 1469 | 2 | 0 | 0 | 0 | 2 |
| 1470 | 0 | 0 | 2 | 0 | 2 |
| 1471 | 0 | 0 | 2 | 0 | 2 |
| 1472 | 2 | 0 | 0 | 0 | 2 |
| 1473 | 0 | 0 | 2 | 0 | 2 |
| 1474 | 0 | 0 | 2 | 0 | 2 |
| 1475 | 2 | 0 | 0 | 0 | 2 |
| 1476 | 0 | 0 | 2 | 0 | 2 |
| 1477 | 2 | 0 | 0 | 0 | 2 |
| 1478 | 2 | 0 | 0 | 0 | 2 |
| 1479 | 2 | 0 | 0 | 0 | 2 |
| 1480 | 0 | 0 | 2 | 0 | 2 |
| 1481 | 0 | 0 | 2 | 0 | 2 |
| 1482 | 0 | 0 | 2 | 0 | 2 |
| 1483 | 2 | 0 | 0 | 0 | 2 |
| 1484 | 2 | 0 | 0 | 0 | 2 |
| 1485 | 2 | 0 | 0 | 0 | 2 |
| 1486 | 2 | 0 | 0 | 0 | 2 |
| 1487 | 2 | 0 | 0 | 0 | 2 |
| 1488 | 2 | 0 | 0 | 0 | 2 |
| 1489 | 2 | 0 | 0 | 0 | 2 |
| 1490 | 2 | 0 | 0 | 0 | 2 |
| 1491 | 0 | 0 | 2 | 0 | 2 |
| 1492 | 2 | 0 | 0 | 0 | 2 |
| 1493 | 2 | 0 | 0 | 0 | 2 |
| 1494 | 2 | 0 | 0 | 0 | 2 |
| 1495 | 2 | 0 | 0 | 0 | 2 |
| 1496 | 2 | 0 | 0 | 0 | 2 |
| 1497 | 2 | 0 | 0 | 0 | 2 |
| 1498 | 2 | 0 | 0 | 0 | 2 |
| 1499 | 2 | 0 | 0 | 0 | 2 |
| 1500 | 0 | 0 | 2 | 0 | 2 |
| 1501 | 2 | 0 | 0 | 0 | 2 |
| 1502 | 2 | 0 | 0 | 0 | 2 |
| 1503 | 2 | 0 | 0 | 0 | 2 |
| 1504 | 2 | 0 | 0 | 0 | 2 |
| 1505 | 2 | 0 | 0 | 0 | 2 |
| 1506 | 0 | 0 | 2 | 0 | 2 |
| 1507 | 2 | 0 | 0 | 0 | 2 |
| 1508 | 2 | 0 | 0 | 0 | 2 |
| 1509 | 2 | 0 | 0 | 0 | 2 |
| 1510 | 0 | 0 | 2 | 0 | 2 |
| 1511 | 2 | 0 | 0 | 0 | 2 |
| 1512 | 2 | 0 | 0 | 0 | 2 |
| 1513 | 0 | 0 | 2 | 0 | 2 |
| 1514 | 2 | 0 | 0 | 0 | 2 |
| 1515 | 2 | 0 | 0 | 0 | 2 |
| 1516 | 2 | 0 | 0 | 0 | 2 |
| 1517 | 0 | 0 | 2 | 0 | 2 |
| 1518 | 0 | 0 | 2 | 0 | 2 |
| 1519 | 2 | 0 | 0 | 0 | 2 |
| 1520 | 0 | 0 | 2 | 0 | 2 |
| 1521 | 2 | 0 | 0 | 0 | 2 |
| 1522 | 0 | 0 | 2 | 0 | 2 |
| 1523 | 2 | 0 | 0 | 0 | 2 |
| 1524 | 2 | 0 | 0 | 0 | 2 |
| 1525 | 2 | 0 | 0 | 0 | 2 |
| 1526 | 2 | 0 | 0 | 0 | 2 |
| 1527 | 0 | 0 | 2 | 0 | 2 |
| 1528 | 2 | 0 | 0 | 0 | 2 |
| 1529 | 2 | 0 | 0 | 0 | 2 |
| 1530 | 0 | 0 | 2 | 0 | 2 |
| 1531 | 2 | 0 | 0 | 0 | 2 |
| 1532 | 0 | 0 | 2 | 0 | 2 |
| 1533 | 0 | 0 | 2 | 0 | 2 |
| 1534 | 2 | 0 | 0 | 0 | 2 |
| 1535 | 0 | 0 | 2 | 0 | 2 |
| 1536 | 2 | 0 | 0 | 0 | 2 |
| 1537 | 2 | 0 | 0 | 0 | 2 |
| 1538 | 2 | 0 | 0 | 0 | 2 |
| 1539 | 2 | 0 | 0 | 0 | 2 |
| 1540 | 0 | 0 | 1 | 0 | 1 |
| 1541 | 1 | 0 | 0 | 0 | 1 |
| 1542 | 0 | 0 | 1 | 0 | 1 |
| 1543 | 1 | 0 | 0 | 0 | 1 |
| 1544 | 0 | 0 | 1 | 0 | 1 |
| 1545 | 0 | 0 | 1 | 0 | 1 |
| 1546 | 0 | 0 | 1 | 0 | 1 |
| 1547 | 1 | 0 | 0 | 0 | 1 |
| 1548 | 0 | 0 | 1 | 0 | 1 |
| 1549 | 1 | 0 | 0 | 0 | 1 |
| 1550 | 1 | 0 | 0 | 0 | 1 |
| 1551 | 1 | 0 | 0 | 0 | 1 |
| 1552 | 0 | 0 | 1 | 0 | 1 |
| 1553 | 0 | 0 | 1 | 0 | 1 |
| 1554 | 0 | 0 | 1 | 0 | 1 |
| 1555 | 0 | 0 | 1 | 0 | 1 |
| 1556 | 0 | 0 | 1 | 0 | 1 |
| 1557 | 1 | 0 | 0 | 0 | 1 |
| 1558 | 0 | 0 | 1 | 0 | 1 |
| 1559 | 1 | 0 | 0 | 0 | 1 |
| 1560 | 0 | 0 | 1 | 0 | 1 |
| 1561 | 1 | 0 | 0 | 0 | 1 |
| 1562 | 1 | 0 | 0 | 0 | 1 |
| 1563 | 0 | 0 | 1 | 0 | 1 |
| 1564 | 1 | 0 | 0 | 0 | 1 |
| 1565 | 1 | 0 | 0 | 0 | 1 |
| 1566 | 1 | 0 | 0 | 0 | 1 |
| 1567 | 1 | 0 | 0 | 0 | 1 |
| 1568 | 0 | 0 | 1 | 0 | 1 |
| 1569 | 1 | 0 | 0 | 0 | 1 |
| 1570 | 1 | 0 | 0 | 0 | 1 |
| 1571 | 1 | 0 | 0 | 0 | 1 |
| 1572 | 0 | 0 | 1 | 0 | 1 |
| 1573 | 0 | 0 | 1 | 0 | 1 |
| 1574 | 0 | 0 | 1 | 0 | 1 |
| 1575 | 0 | 0 | 1 | 0 | 1 |
| 1576 | 0 | 0 | 1 | 0 | 1 |
| 1577 | 1 | 0 | 0 | 0 | 1 |
| 1578 | 1 | 0 | 0 | 0 | 1 |
| 1579 | 0 | 0 | 1 | 0 | 1 |
| 1580 | 1 | 0 | 0 | 0 | 1 |
| 1581 | 1 | 0 | 0 | 0 | 1 |
| 1582 | 0 | 0 | 1 | 0 | 1 |
| 1583 | 0 | 0 | 1 | 0 | 1 |
| 1584 | 0 | 0 | 1 | 0 | 1 |
| 1585 | 1 | 0 | 0 | 0 | 1 |
| 1586 | 1 | 0 | 0 | 0 | 1 |
| 1587 | 0 | 0 | 1 | 0 | 1 |
| 1588 | 0 | 0 | 1 | 0 | 1 |
| 1589 | 1 | 0 | 0 | 0 | 1 |
| 1590 | 0 | 0 | 1 | 0 | 1 |
| 1591 | 0 | 0 | 1 | 0 | 1 |
| 1592 | 1 | 0 | 0 | 0 | 1 |
| 1593 | 1 | 0 | 0 | 0 | 1 |
| 1594 | 0 | 0 | 1 | 0 | 1 |
| 1595 | 1 | 0 | 0 | 0 | 1 |
| 1596 | 1 | 0 | 0 | 0 | 1 |
| 1597 | 0 | 0 | 1 | 0 | 1 |
| 1598 | 0 | 0 | 1 | 0 | 1 |
| 1599 | 1 | 0 | 0 | 0 | 1 |
| 1600 | 0 | 0 | 1 | 0 | 1 |
| 1601 | 1 | 0 | 0 | 0 | 1 |
| 1602 | 1 | 0 | 0 | 0 | 1 |
| 1603 | 1 | 0 | 0 | 0 | 1 |
| 1604 | 1 | 0 | 0 | 0 | 1 |
| 1605 | 0 | 0 | 1 | 0 | 1 |
| 1606 | 1 | 0 | 0 | 0 | 1 |
| 1607 | 0 | 0 | 1 | 0 | 1 |
| 1608 | 1 | 0 | 0 | 0 | 1 |
| 1609 | 0 | 0 | 1 | 0 | 1 |
| 1610 | 1 | 0 | 0 | 0 | 1 |
| 1611 | 1 | 0 | 0 | 0 | 1 |
| 1612 | 0 | 0 | 1 | 0 | 1 |
| 1613 | 0 | 0 | 1 | 0 | 1 |
| 1614 | 0 | 0 | 1 | 0 | 1 |
| 1615 | 1 | 0 | 0 | 0 | 1 |
| 1616 | 0 | 0 | 1 | 0 | 1 |
| 1617 | 1 | 0 | 0 | 0 | 1 |
| 1618 | 0 | 0 | 1 | 0 | 1 |
| 1619 | 1 | 0 | 0 | 0 | 1 |
| 1620 | 0 | 0 | 1 | 0 | 1 |
| 1621 | 0 | 0 | 1 | 0 | 1 |
| 1622 | 0 | 0 | 1 | 0 | 1 |
| 1623 | 1 | 0 | 0 | 0 | 1 |
| 1624 | 0 | 0 | 1 | 0 | 1 |
| 1625 | 1 | 0 | 0 | 0 | 1 |
| 1626 | 0 | 0 | 1 | 0 | 1 |
| 1627 | 0 | 0 | 1 | 0 | 1 |
| 1628 | 1 | 0 | 0 | 0 | 1 |
| 1629 | 1 | 0 | 0 | 0 | 1 |
| 1630 | 1 | 0 | 0 | 0 | 1 |
| 1631 | 1 | 0 | 0 | 0 | 1 |
| 1632 | 1 | 0 | 0 | 0 | 1 |
| 1633 | 0 | 0 | 1 | 0 | 1 |
| 1634 | 1 | 0 | 0 | 0 | 1 |
| 1635 | 1 | 0 | 0 | 0 | 1 |
| 1636 | 1 | 0 | 0 | 0 | 1 |
| 1637 | 1 | 0 | 0 | 0 | 1 |
| 1638 | 1 | 0 | 0 | 0 | 1 |
| 1639 | 1 | 0 | 0 | 0 | 1 |
| 1640 | 0 | 0 | 1 | 0 | 1 |
| 1641 | 0 | 0 | 1 | 0 | 1 |
| 1642 | 0 | 0 | 1 | 0 | 1 |
| 1643 | 1 | 0 | 0 | 0 | 1 |
| 1644 | 1 | 0 | 0 | 0 | 1 |
| 1645 | 0 | 0 | 1 | 0 | 1 |
| 1646 | 1 | 0 | 0 | 0 | 1 |
| 1647 | 0 | 0 | 1 | 0 | 1 |
| 1648 | 1 | 0 | 0 | 0 | 1 |
| 1649 | 1 | 0 | 0 | 0 | 1 |
| 1650 | 1 | 0 | 0 | 0 | 1 |
| 1651 | 1 | 0 | 0 | 0 | 1 |
| 1652 | 1 | 0 | 0 | 0 | 1 |
| 1653 | 1 | 0 | 0 | 0 | 1 |
| 1654 | 1 | 0 | 0 | 0 | 1 |
| 1655 | 1 | 0 | 0 | 0 | 1 |
| 1656 | 1 | 0 | 0 | 0 | 1 |
| 1657 | 1 | 0 | 0 | 0 | 1 |
| 1658 | 1 | 0 | 0 | 0 | 1 |
| 1659 | 0 | 0 | 1 | 0 | 1 |
| 1660 | 0 | 0 | 1 | 0 | 1 |
| 1661 | 1 | 0 | 0 | 0 | 1 |
| 1662 | 0 | 0 | 1 | 0 | 1 |
| 1663 | 0 | 0 | 1 | 0 | 1 |
| 1664 | 1 | 0 | 0 | 0 | 1 |
| 1665 | 1 | 0 | 0 | 0 | 1 |
| 1666 | 0 | 0 | 1 | 0 | 1 |
| 1667 | 1 | 0 | 0 | 0 | 1 |
| 1668 | 0 | 0 | 1 | 0 | 1 |
| 1669 | 1 | 0 | 0 | 0 | 1 |
| 1670 | 1 | 0 | 0 | 0 | 1 |
| 1671 | 0 | 0 | 1 | 0 | 1 |
| 1672 | 0 | 0 | 1 | 0 | 1 |
| 1673 | 0 | 0 | 1 | 0 | 1 |
| 1674 | 1 | 0 | 0 | 0 | 1 |
| 1675 | 1 | 0 | 0 | 0 | 1 |
| 1676 | 1 | 0 | 0 | 0 | 1 |
| 1677 | 0 | 0 | 1 | 0 | 1 |
| 1678 | 1 | 0 | 0 | 0 | 1 |
| 1679 | 1 | 0 | 0 | 0 | 1 |
| 1680 | 1 | 0 | 0 | 0 | 1 |
| 1681 | 1 | 0 | 0 | 0 | 1 |
| 1682 | 1 | 0 | 0 | 0 | 1 |
| 1683 | 1 | 0 | 0 | 0 | 1 |
| 1684 | 1 | 0 | 0 | 0 | 1 |
| 1685 | 1 | 0 | 0 | 0 | 1 |
| 1686 | 1 | 0 | 0 | 0 | 1 |
| 1687 | 1 | 0 | 0 | 0 | 1 |
| 1688 | 0 | 0 | 1 | 0 | 1 |
| 1689 | 1 | 0 | 0 | 0 | 1 |
| 1690 | 1 | 0 | 0 | 0 | 1 |
| 1691 | 1 | 0 | 0 | 0 | 1 |

Column A: Cell (Rank);
Column B: UMIs from most abundant productive KI event;
Column C: UMIs from 2nd most abundant productive KI event;
Column D: UMIs from most abundant unproductive KI event;
Column E: UMIs from 2nd most abundant unproductive KI event;
Column F: Total evaluable UMI

### Example 9. Treatment of a Colorectal Cancer Patient with Liver Metastasis (Patient ID CMI-0102)

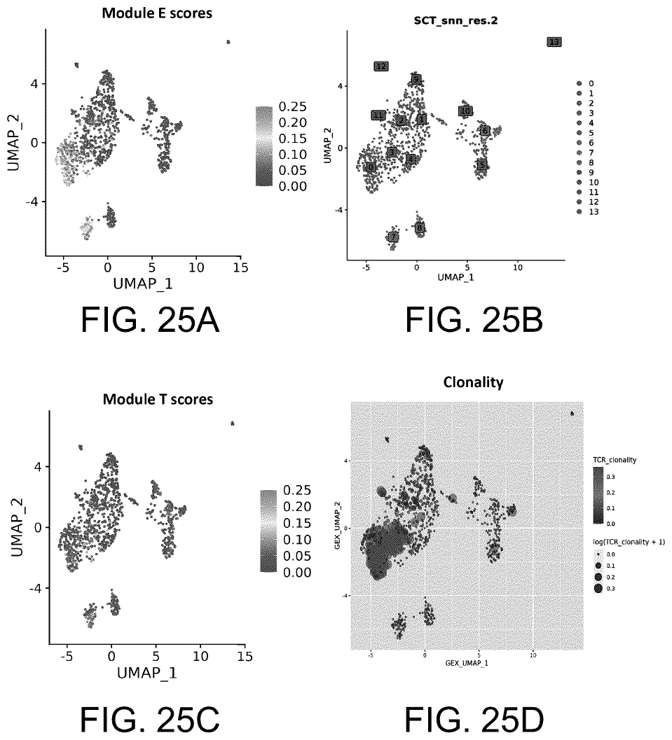

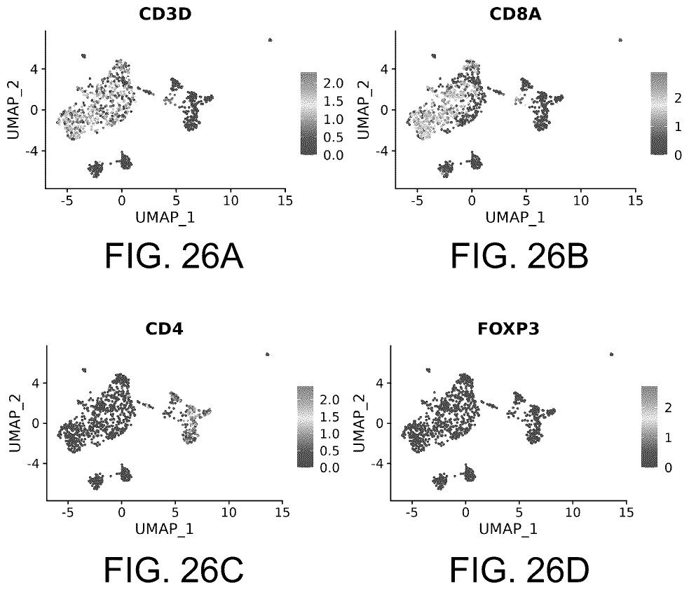

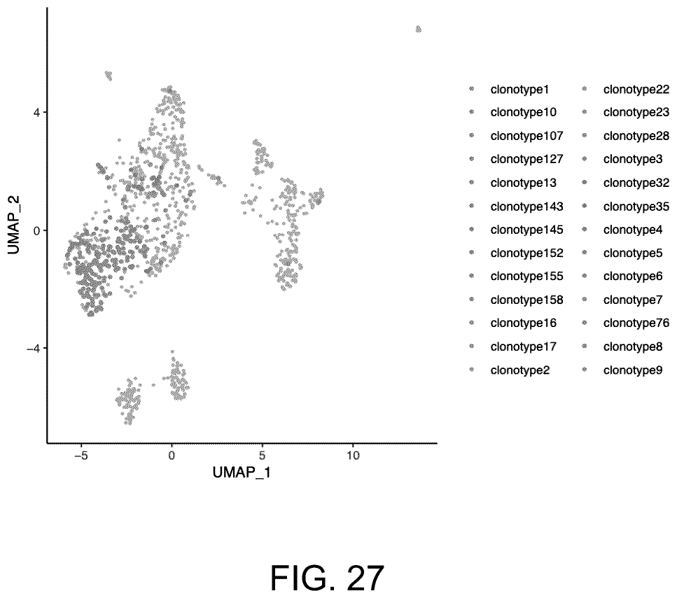

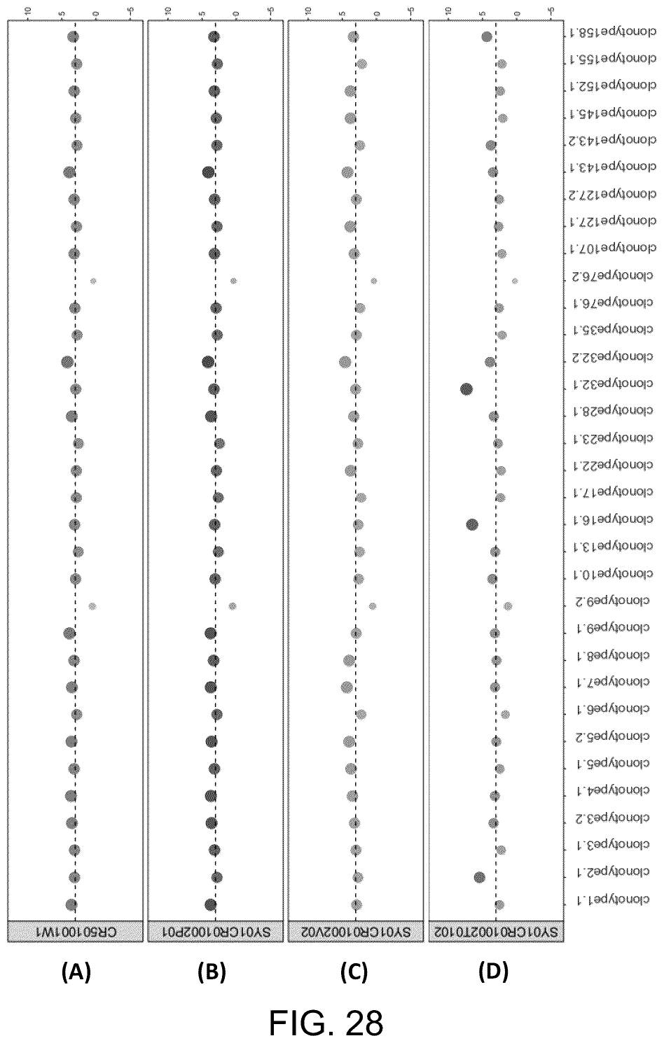

A patient having colorectal cancer with liver metastasis was enrolled in the clinical trial. The clinical protocol used here was similar to the protocol in Example 11. The patient has failed 2 previous lines of therapy. Six (6) fine needle aspirates on liver tumor metastasis (S4, S9, right lobe) was taken. These aspirates were dissociated into single cell suspension and subjected to single-cell RNA-Seq using a 10× Genomics instrument and kits (similar to Example 4). The single-cell gene expression profile data is summarized in the UMAPs of FIGS. 25A-D and FIGS. 26A-D. Each T cell was then given a “Module E” score based on expression levels of a set of about 30 genes implicated T cell exhaustion, and “Module T” score based on expression levels of a set of about 30 genes implicated in T cell activation and secondary lymphoid structure formation. Similarly, “Module N” and “Module X” scores were obtained using other gene sets that have been associated with tumor-reactivity. Each TCR is then given a Module E score, which is defined as the highest Module E score of all T cells expressing this TCR. Each TCR is given a Module T score, Module N score and Module X score using the same definition. If a TCR is ranked in the top 10% with any Module scoring, the TCR is chosen to produce synthetic TIL cells. As shown in Table 7, a total of 33 TCRs were chosen. The T cells carrying these TCRs in the biopsy sample are shown on the UMAP of FIG. 27. These 33 TCRs were individually synthesized, sequence verified, quantified, and mixed at equimolar proportion to form R&D-grade TCR plasmid mixture. The proportion of each TCR in this mixture was confirmed using an NGS assay. The results (FIG. 28, panel A) show that the vast majority of these TCRs have a proportion of about 1/33 (i.e., ˜3%). This R&D-grade TCR plasmid mixture was then used to transform an E. coli cell suspension, which was then expanded to about 40 L culture for plasmid amplification in a GMP-compliant process, which yielded about 40 mg of plasmid mixture. This plasmid mixture was then used to package GMP-grade AAV6 viral particles. Finally, the AAV6 viral particles was used to knock-in the 33 autologous TCRs into the TRAC locus of about 3 billion T cells obtained from the patient's periphery via leukapheresis, generating about 1.6×10¹⁰ synthetic TIL cells (T cells exogenously expressing an autologous TCR). Surprisingly, in the GMP grade plasmid mixture (FIG. 28, panel B), AAV6 viral particle mixture (FIG. 28, panel C), and synthetic TIL cell mixture (FIG. 28, panel D), the proportion of the vast majority (more than 80%) of the TCRs were maintained within 2-fold of 1/33 (i.e., 1.5% to 6%). More than 95% of the cells in the infusion product were confirmed to be T cells. Using an RNA-FISH method detecting the exogenous TCR transcript within the synthetic TIL cells, it was observed that as high as 54% of the infusion product is synthetic TIL cells (e.g., cells exogenously expressing an autologous TCR).

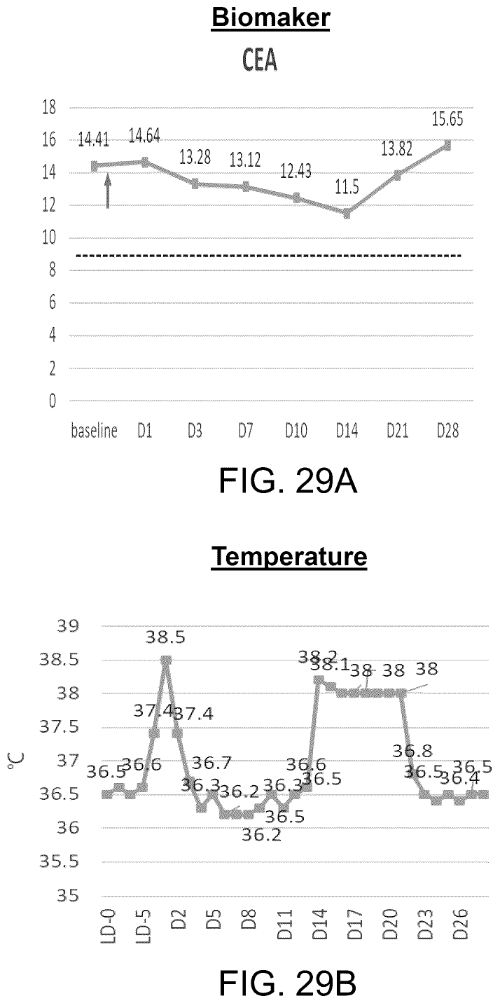

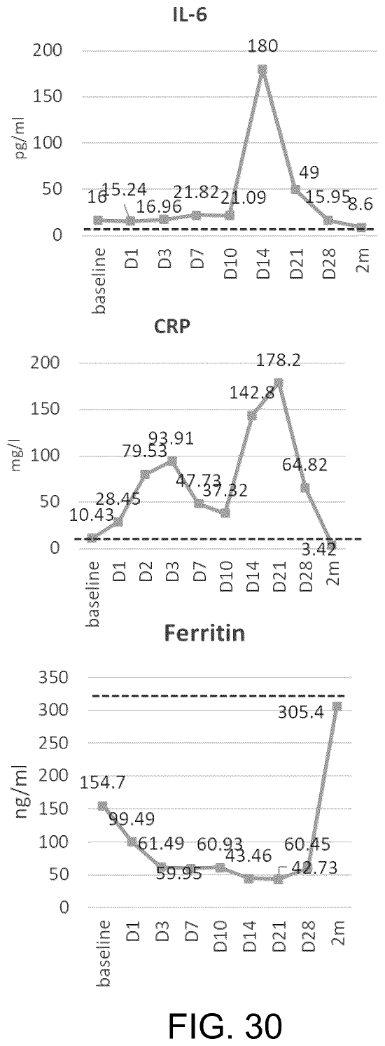

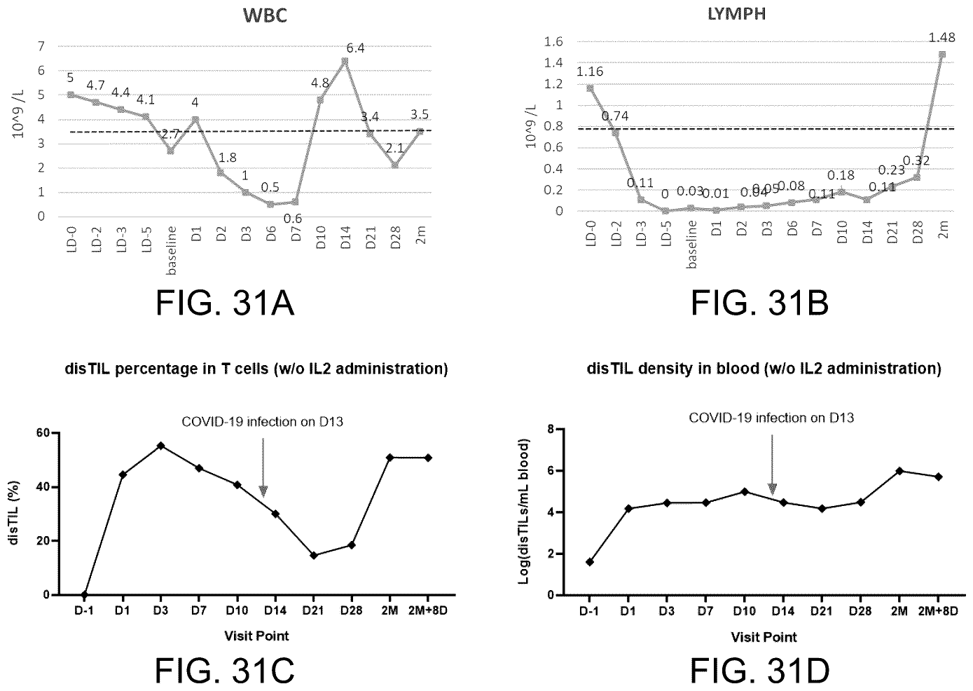

The patient underwent successful lymphodepletion (FIG. 31B) before infusion of about 1×10⁹ synthetic TIL product. Although the targets of the 33 autologous TCRs were not explicitly known, the synthetic TIL product was well tolerated with only Grade 1 and 2 adverse events (e.g., Grade 1 fever, Grade 2 CRS, and Grade 1 hypotension). The CEA level decreased by about 20% at Day 14 post infusion. The elevated level of temperature, IL-6, CRP and white blood cell coincided with a COVID infection (FIGS. 29A, 29B, 30, and 31A).

The synthetic TIL showed remarkable level of persistence and expansion. At Day 3 of infusion, the percentage of synthetic TIL cells among the patient's peripheral T cells rose to about 50% and only gradually decreased over the next month. Surprisingly, at 2 months after infusion (2M time point of FIGS. 31A-D), as the total lymphocyte count recovered to pre-lymphodepletion level, the percentage of synthetic TIL cells among peripheral T cells rose back to about 50% (FIG. 31C). The absolute number of cells stayed even in blood prior to 2M time point, and after recovery of the blood compartment, about 0.6×10⁶ to 1×10⁶ synthetic TIL cells (in some time point, over 1 million synthetic TIL cells) per mL of peripheral blood (FIG. 31D) were present, which is significantly higher than what is typically achieved by conventional TIL therapy.

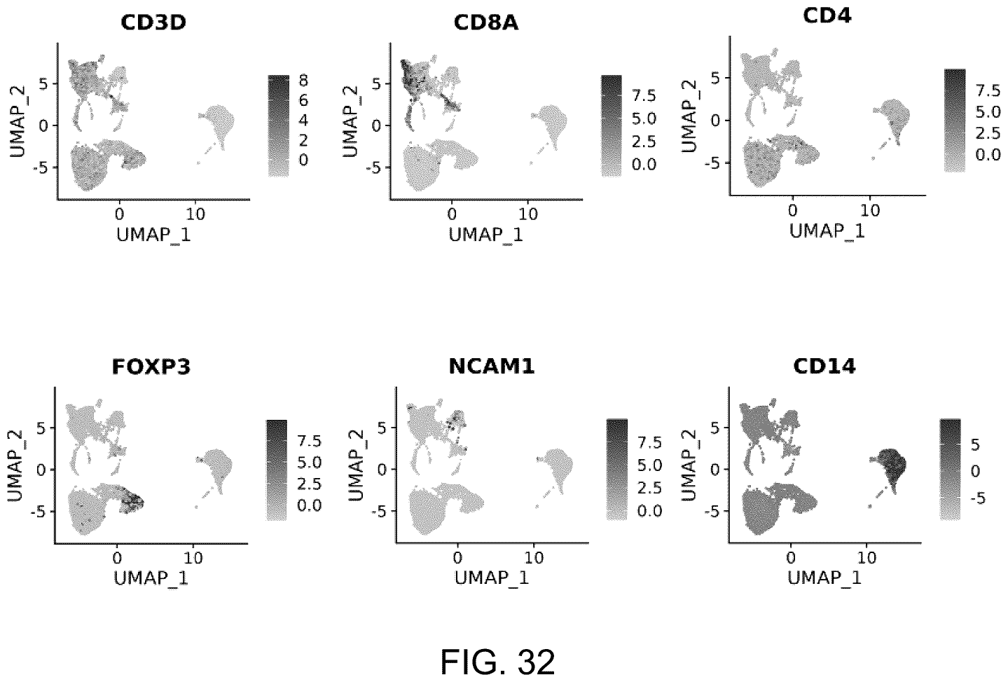

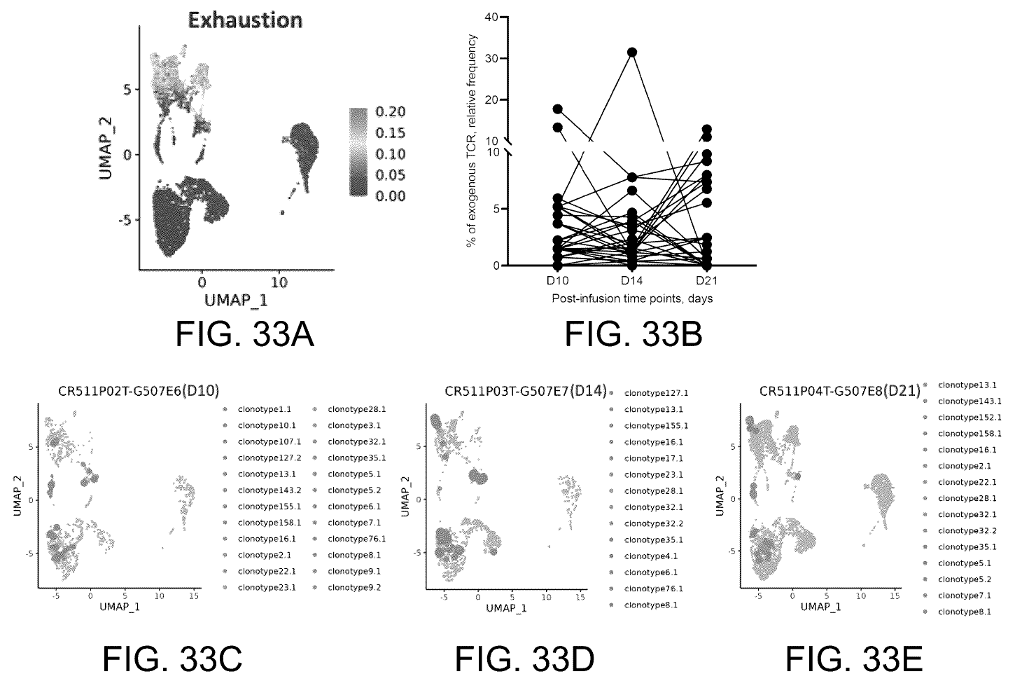

The patient's peripheral T cells at day 10, day 14 and day 21 after infusion were analyzed using the method described in Example 3. This scRNA-Seq-based method also allowed us to track the transcriptional profile of all peripheral T cells, including synthetic TIL cells. Data from all 3 samples (on 3 different days) were merged to produce the UMAPs shown on FIG. 32 and FIG. 33A. The grayscale of FIG. 33A shows the Module E score of each T cell, highlighting the ones exhibiting high level of signature associated with antigen experience and T cell exhaustion. It was observed that several clones had their frequency rise on day 21 (FIG. 33B). Interestingly, although most synthetic TIL cells appear in cells with low Module E score (FIG. 33C), at later days synthetic TIL cells with high Module E scores were readily detectable (FIGS. 33D and 33E).

### Example 10. Treatment of a Cervical Cancer Patient (Patient ID CMI-0104)

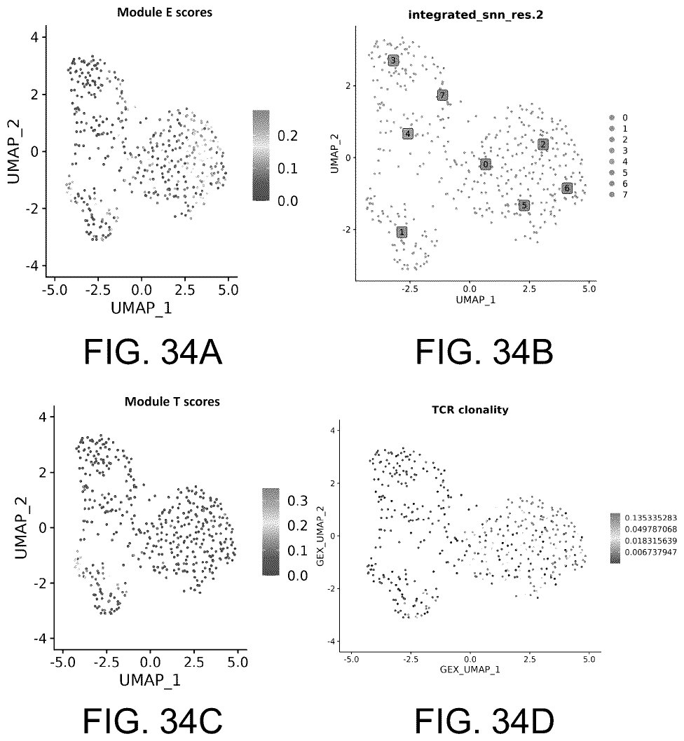

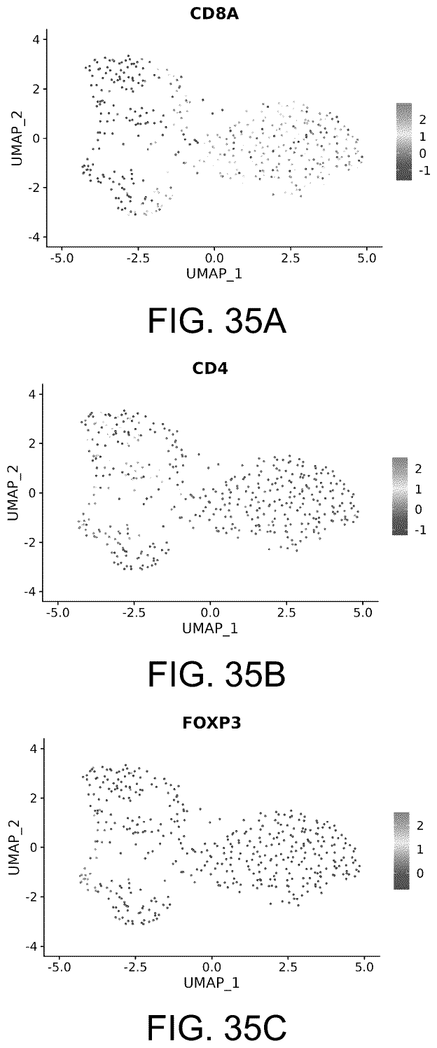

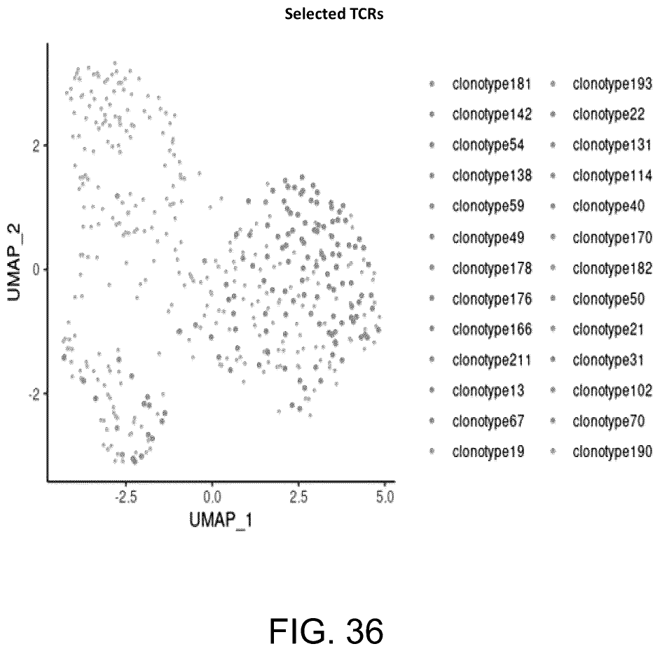

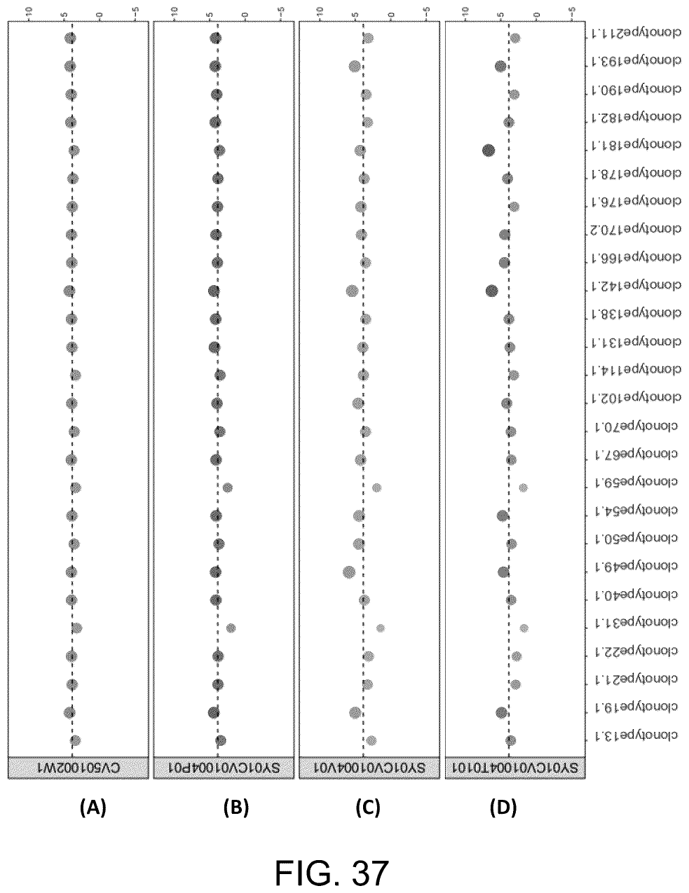

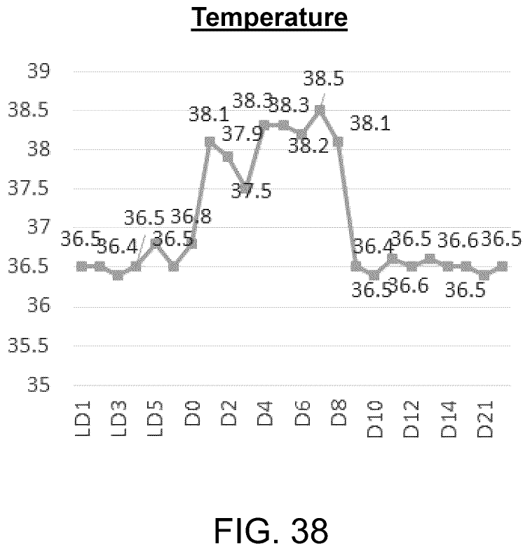

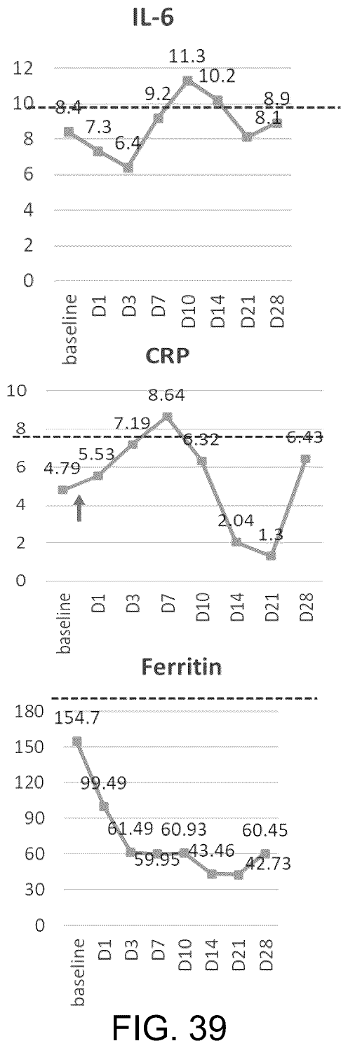

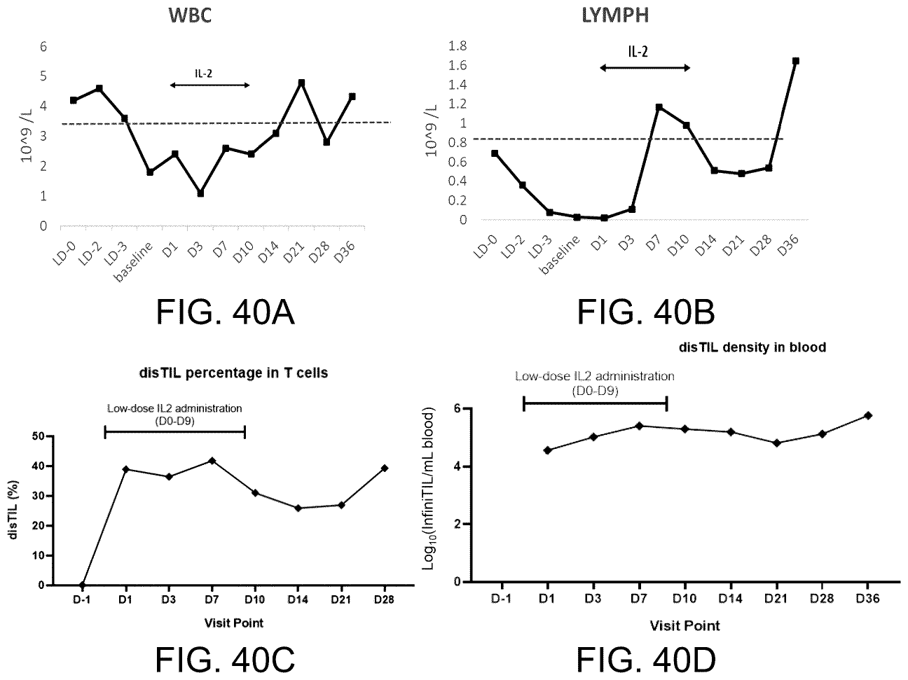

A patient having HPV16+ cervical cancer (Patient ID CMI-0104) was enrolled in the clinical trial. The clinical protocol used was similar to the protocol in Example 11. The patient has failed 1 previous line of therapy. Six (6) fine needle aspirates through vaginal fornix were taken. Similar to the Example 9, scRNA-Seq analysis was conducted (FIGS. 34A-D and 35A-C), and a total of 26 TCRs (Table 11, FIG. 36) were chosen to make synthetic TIL cells. The TCR-encoding plasmids were also mixed at equimolar proportion, and these proportions were well maintained at GMP plasmid mixture, AAV6 viral particle mixture and synthetic T cell mixture (FIG. 37). Vital signs and CRS markers showed high tolerability (FIGS. 38 and 39), with only Grade 1 adverse events (e.g., Grade 1 Fever, Grade 1 CRS, and Grade 1 IL-6 elevation). The patient received low dose IL-2 infusion on days indicated on FIGS. 40A-D. Similar to patient CMI-0102, synthetic TIL cells also showed remarkable persistence in patient CMI-0104 (FIGS. 40C and 40D). After 4-6 weeks into recovery of endogenous levels, the synthetic TIL product accounts for >50% total cell counts. The absolute number of cells steadily increased in counts with IL-2 infusion for the first month before tapering and then increasing at 5 weeks. After recovery of the blood compartment at day 36, more than 0.45×10⁶ synthetic TIL cells per mL of peripheral blood were present.

TABLE 5
Patient history summary of patient CMI-0102
Patient History Summary

| | |
|---|---|
| Age | 64 |
| Gender | Male |
| Diagnosis | July 2019 - Primary Diagnosis colon cancer |
| | September 2020 - Hepatic metastases |
| Molecular Analysis | MSS |
| | KRAS Exon 2/3/4 WT, NRAS V600E WT |
| Prior Therapies | Failed 2 prior lines (2 cycle total) |
| Medical History | July 2019 - sigmoidectomy |
| | September 2020 - Chemotherapy 6 cycles (specific drug is unknown) 2020 September |
| | Jun. 22, 2022-Aug. 17, 2022 - mFOLFOX (oxaliplatin 85 mg/m², Leucovorin |
| | Calcium 400 mg/m², 5-FU 400 mg/m²) + cetuximab 500 mg/m² 4 cycles |

TABLE 6
Study timeline of patient CMI-0102
Patient Timeline on Study

| | |
|---|---|
| Screen | Aug. 25, 2022 |
| Biopsy | Sep. 9, 2022 - 6 fine needles aspirate on liver |
| | tumor metastasis(S4, S9, right lobe) |
| TCR Selection | Oct. 19, 2022 - 33 TCRs selected |
| PBMC Collection | Nov. 3, 2022 |
| Lymphodepletion | Dec. 7, 2022 |
| Infusion | Dec. 13, 2022 - 1 × 10⁹ Cells |
| IL-2 | NA |
| Combinations | NA |
| 28 Day Follow Up | PD (baseline Dec. 6, 2022: 42.50 mm vs D 28 |
| | Jan. 10, 2023: 52.69 mm 24%) |
| Second Lymphodepletion | Pending |
| 2^{nd} Infusion | NA |
| 2^{nd} Dose IL-2 | NA |
| 2^{nd} Dose Combination | NA |
| Current Status | PD - (2 m Feb. 7, 2023: 58.31 mm 37.2%) |

TABLE 7
TCRs selected for manufacturing of synthetic TILs to be administered
into patient CMI-0102.

| Clonotype | | CD4/ | | Module | Module | Module | Module |
|---|---|---|---|---|---|---|---|
| ID | CDR3s | CD8 | Clonality | E | X | T | N |
| clonotype | CATVSYSGAGSYQLTF; | CD8 | 1 | 0.15573 | 0.16630 | 0.26111 | 0.12737 |
| 155.1 | CASSGPTSASSSYNEQFF | | | | | | |
| clonotype | CATFGTASKLTF; | CD8 | 1 | 0.13067 | 0.21142 | 0.18982 | 0.06126 |
| 76.1 | CASSEAGVGGYTF | | | | | | |
| clonotype | CATFGTASKLTF; | CD8 | 1 | 0.13067 | 0.21142 | 0.18982 | 0.06126 |
| 76.2 | CASSEAGVGGYTF | | | | | | |
| clonotype | CAMRDPTGNQFYF; | CD8 | 4 | 0.06074 | 0.05506 | 0.17247 | 0.06963 |
| 13.1 | CASSLAGTSSGANVLTF | | | | | | |
| clonotype | CALSEALDYGGATNKLIF; | CD8 | 150 | 0.08599 | 0.10113 | 0.13953 | 0.11281 |
| 1.1 | CASSFLAGGEQFF | | | | | | |
| clonotype | CAVGLNTDKLIF; | CD8 | 1 | 0.02862 | 0.12637 | 0.13915 | 0.07883 |
| 127.1 | CASSQEEGIYYGELFF | | | | | | |
| clonotype | CAVRDAHTGGFKTIF; | CD8 | 1 | 0.02862 | 0.12637 | 0.13915 | 0.07883 |
| 127.2 | CASSQEEGIYYGELFF | | | | | | |
| clonotype | CAENGGRNDYKLSF; | CD8 | 2 | 0.07147 | 0.07180 | 0.12102 | 0.10140 |
| 32.1 | CASSYDSGGSDTQYF | | | | | | |
| clonotype | CAVNPNNDMRF; | CD8 | 2 | 0.07147 | 0.07180 | 0.12102 | 0.10140 |
| 32.2 | CASSYDSGGSDTQYF | | | | | | |
| clonotype | CASLDGGSQGNLIF; | CD8 | 5 | 0.12984 | 0.11116 | 0.10759 | 0.11095 |
| 10.1 | CASSPFDSANEQFF | | | | | | |
| clonotype | CAVQAMNRDDKIIF; | CD8 | 3 | 0.14305 | 0.10798 | 0.10091 | 0.11838 |
| 17.1 | CASSLQGVNTEAFF | | | | | | |
| clonotype | CAPAGYGQNFVF; | CD8 | 1 | 0.02566 | 0.06726 | 0.09908 | 0.08439 |
| 143.1 | CASSQSGSSPSYEQYF | | | | | | |
| clonotype | CAVVDSNYQLIW; | CD8 | 1 | 0.02566 | 0.06726 | 0.09908 | 0.08439 |
| 143.2 | CASSQSGSSPSYEQYF | | | | | | |
| clonotype | CAVANYGQNFVF; | CD8 | 1 | 0.05084 | 0.14028 | 0.09317 | 0.09971 |
| 145.1 | CASAGWMMDTILNLQYF | | | | | | |
| clonotype | CAVPYSGTYKYIF; | CD8 | 2 | 0.08978 | 0.14180 | 0.08372 | 0.09953 |
| 35.1 | CASGLVGSGGPNRPQHF | | | | | | |
| clonotype | CVVSPSLSGNTPLVF; | CD8 | 1 | 0.02584 | 0.13514 | 0.05979 | 0.11179 |
| 152.1 | CASSERWLAGGGADTQYF | | | | | | |
| clonotype | CAASMGTGRRALTF; | CD8 | 6 | 0.12421 | 0.07956 | 0.05521 | 0.10202 |
| 9.1 | CASSIDRVQQYF | | | | | | |
| clonotype | CALTHSSNTGKLIF; | CD8 | 6 | 0.12421 | 0.07956 | 0.05521 | 0.10202 |
| 9.2 | CASSIDRVQQYF | | | | | | |
| clonotype | CAVIVDKIIF; | CD8 | 2 | 0.12047 | 0.05636 | 0.04482 | 0.08014 |
| 23.1 | CASSLTGDFYEQYF | | | | | | |
| clonotype | CALPGYSSASKIIF; | CD8 | 10 | 0.09458 | 0.09024 | 0.04183 | 0.12783 |
| 5.1 | CASSQPGQGVEQYF | | | | | | |
| clonotype | CAVDGAGSYQLTF; | CD8 | 10 | 0.09458 | 0.09024 | 0.04183 | 0.12783 |
| 5.2 | CASSQPGQGVEQYF | | | | | | |
| clonotype | CAGAPHSNFGNEKLTF; | CD8 | 11 | 0.06951 | 0.10397 | 0.03912 | 0.10919 |
| 4.1 | CASSTSGALRSYNEQFF | | | | | | |
| clonotype | CAVEAEAGANSKLTF; | CD8 | 1 | 0.04089 | 0.09512 | 0.03824 | 0.11950 |
| 107.1 | CASSLSLGGDTQYF | | | | | | |
| clonotype | CAVNMVGQKLLF; | CD8 | 2 | 0.10335 | 0.12358 | 0.03614 | 0.13090 |
| 28.1 | CASRQGTSGSYEQYF | | | | | | |
| clonotype | CALNPQMANFNKFYF; | CD8 | 3 | 0.12498 | 0.08008 | 0.03421 | 0.08153 |
| 16.1 | CASSWGGDRKTTYEQYF | | | | | | |
| clonotype | CVVSEGSNYQLIW; | CD8 | 1 | 0.13884 | 0.08867 | 0.03410 | 0.11868 |
| 158.1 | CASSEGSYEQYF | | | | | | |
| clonotype | CAEDNYGQNFVF; | CD8 | 11 | 0.12427 | 0.05806 | 0.02758 | 0.08946 |
| 3.1 | CASSQDNLGGAVNTEAFF | | | | | | |
| clonotype | CAVPSGGSYIPTF; | CD8 | 11 | 0.12427 | 0.05806 | 0.02758 | 0.08946 |
| 3.2 | CASSQDNLGGAVNTEAFF | | | | | | |
| clonotype | CAVTFYGGSQGNLIF; | CD8 | 10 | 0.02987 | 0.15924 | 0.02713 | 0.10509 |
| 6.1 | CASSLGREVQETQYF | | | | | | |
| clonotype | CALMRDSSYKLIF; | CD8 | 2 | 0.00433 | 0.14303 | 0.02470 | 0.07763 |
| 22.1 | CASSYLGENYGYTF | | | | | | |
| clonotype | CAVRDPSGGSYIPTF; | CD8 | 8 | 0.00196 | 0.05357 | 0.01580 | 0.12207 |
| 7.1 | CATSRAEEAEAFF | | | | | | |
| clonotype | CAAENLNSGYSTLTF; | CD8 | 12 | 0.04101 | 0.06517 | 0.00967 | 0.09192 |
| 2.1 | CASSLDNSPLHF | | | | | | |
| clonotype | CASSRPQGAQKLVF; | CD8 | 6 | 0.02347 | 0.14019 | 0.00000 | 0.10391 |
| 8.1 | CASSDVIEQFF | | | | | | |

TABLE 8A
Summary of manufacturing for patient CMI-0102
Plasmid and AAV Production

| Measurement | Result | | Release Criteria | |
|---|---|---|---|---|
| Plasmid Content | 0.98 | mg/mL | 0.80-1.20 | mg/mL |
| Plasmid Purity | 1.87 | | 1.80-2.00 | |
| Supercoiled Content | 89% | | ≥85% | |
| Plasmid Other QC | Appearance, pH, Identity and Integrity, | | | |
| Passed | DNA Sequence, HCD, HCP, Antibiotic | | | |
| | Residue, Endotoxin, Sterility | | | |
| AAV Titer | 8.03 × 10¹¹ | vg/mL | ≥1.00 × 10⁹ | vg/mL |

| | | |
|---|---|---|
| Empty capsid content | 7% | ≤15% |

| | |
|---|---|
| AAV Other QC Passed | Appearance, pH, Osmolality, Capsid |
| | protein, Sequence, HCD, HCP, BSA |
| | Residue, E1A Residue, SV40LTA |
| | Residue, Nuclease Residue, |
| | Mycoplasma, Endotoxin, Sterility |

TABLE 8B
Summary of manufacturing for patient CMI-0102

| Measurement | Result |
|---|---|
| Total Leukopak | 7.88E9 |
| Number of T Cells Recovered | 3.17E9 |
| Fold Expansion | 12 |
| Knock out Efficiency | 91% |
| Knock In Efficiency | 35% |
| Total Cell Number Produced | 1.64E10 |
| % Exogenous TCR | 54.02 |
| Final Product Phenotype | 96.21 |
| (CD62L⁺CD3⁺) | |
| Number of Present TCRs (>0.1 * 1/N) | 32 |

TABLE 9
Patient history summary of patient CMI-0104
Patient History Summary

| | |
|---|---|
| Age | 43 |
| Gender | Female |
| Diagnosis | May 2022 |
| Molecular Analysis | rIIIC1 cervical cancer-squamous carcinoma, |
| | HPV16+ |
| Prior Therapies | Failed 1 prior line |
| Medical History | Jul. 27, 2022-Sep. 23, 2022 Pelvic irradiation |
| | (IMRT) (33 cycles) |
| | Jul. 28, 2022-Aug. 20, 2022 Cisplatin 40 mg D1 |
| | concurrent radiochemotherapy (4 cycles) |
| | Aug. 14, 2022, Sep. 25, 2022 Terelizumab (2 |
| | cycles) |

TABLE 10
Study timeline of patient CMI-0104
Patient Timeline on Study

| | |
|---|---|
| Screen | Oct. 31, 2022 |
| Biopsy | Nov. 2, 2022 - 6 fine needles aspirate through |
| | Vaginal fornix |
| TCR Selection | Nov. 24, 2022 - 26 TCRs selected |
| PBMC Collection | Dec. 8, 2022 - Concentrated cell capacity 117 |
| | mL |
| Lymphodepletion | Jan. 4, 2023 - Cyclophosphamide 500 mg/m²/d, |
| | Fludarabine 25 mg/m²/d 3 days |
| Infusion | Jan. 10, 2023 - 3 × 10⁹ Cells |
| IL-2 | Jan. 10, 2023-Jan. 19, 2023 500,000 IU bid |
| Combinations | None |
| 28 Day Follow Up | Feb. 7, 2023 - SD (D −7 Jan. 3, 2023: 61.50 |
| | mm vs D 28 Feb. 7, 2023: 60.88 mm) |

TABLE 11
TCRs selected for manufacturing of synthetic TILs to be administered
into patient CMI-0104

| Clonotype | | CD4/ | | Module | Module | Module | Module |
|---|---|---|---|---|---|---|---|
| ID | CDR3s | CD8 | Clonality | E | X | T | N |
| clonotype | CASSSIDSREQYF; | CD8 | 1 | 0.22811 | 0.26430 | 0.34982 | 0.17126 |
| 181 | CAVRVPSFGNVLHC | | | | | | |
| clonotype | CASSDGNNQPQHF; | CD8 | 3 | 0.24361 | 0.31376 | 0.33361 | 0.14874 |
| 142 | CAVINDYKLSF | | | | | | |
| clonotype | CASSSANYGYTF; | CD8 | 4 | 0.20759 | 0.24809 | 0.21104 | 0.10589 |
| 54 | CAGPMKTSYDKVIF | | | | | | |
| clonotype | CAVRPGNTGKLIF; | CD8 | 1 | 0.12374 | 0.20313 | 0.14964 | 0.12658 |
| 178 | CASRRTGEFQEQYF | | | | | | |
| clonotype | CASSLGGVGGYTF; | CD8 | 1 | 0.20196 | 0.22451 | 0.14844 | 0.15512 |
| 176 | CAVRDTGNQFYF | | | | | | |
| clonotype | CAVQVGGNKLTF; | CD8 | 1 | 0.13721 | 0.15530 | 0.14036 | 0.11045 |
| 166 | CASRRIGGGGNEKLFF | | | | | | |
| clonotype | CASSDGQLAPGELFF; | CD8 | 2 | 0.08460 | 0.19015 | 0.13691 | 0.14210 |
| 211 | CVVNGRDSSYKLIF | | | | | | |
| clonotype | CASSLSGGNGANVLTF; | CD8 | 2 | 0.14300 | 0.20181 | 0.12714 | 0.17550 |
| 13 | CAASHNYGQNFVF | | | | | | |
| clonotype | CASSSSGRGNEQFF; | CD8 | 2 | 0.18579 | 0.21008 | 0.12366 | 0.17710 |
| 67 | CALKYNYGQNFVF | | | | | | |
| clonotype | CASRGGPQRETQYF; | CD8 | 3 | 0.00622 | 0.08428 | 0.11631 | 0.08644 |
| 19 | CAASNSGNTPLVF | | | | | | |
| clonotype | CATEGNFGNEKLTF; | CD8 | 81 | 0.14958 | 0.13105 | 0.10380 | 0.14034 |
| 114 | CATSRLAGEEQYF | | | | | | |
| clonotype | CAETLLSSGGSYIPTF; | CD8 | 11 | 0.10115 | 0.16208 | 0.10373 | 0.12542 |
| 40 | CASRPGTSQEDEQYF | | | | | | |
| clonotype | CASSVGLTTGDTQYF; | CD8 | 2 | 0.13600 | 0.11931 | 0.09645 | 0.13839 |
| 70 | CALQNAASGTYKYIF | | | | | | |
| clonotype | CAASREGGGADGLTF; | CD8 | 1 | 0.15522 | 0.21294 | 0.08499 | 0.16020 |
| 21 | CASSSNYEQYF | | | | | | |
| clonotype | CASSPGGASQATYEQYF; | CD8 | 1 | 0.16038 | 0.11431 | 0.05157 | 0.16029 |
| 182 | CAVRVPWGGYNKLIF | | | | | | |
| clonotype | CAVRDMIQAAGNKLTF; | CD8 | 15 | 0.18094 | 0.19918 | 0.04070 | 0.16090 |
| 170 | CATSSVGAPDEKLFF | | | | | | |
| clonotype | CASFTSGTYKYIF; | CD8 | 1 | 0.13130 | 0.09781 | 0.03817 | 0.17682 |
| 102 | CASSQDQYRGNYGYTF | | | | | | |
| clonotype | CAGGGSSGGGADGLTF; | CD8 | 2 | 0.15749 | 0.11858 | 0.02243 | 0.15265 |
| 50 | CASSHPLGEVTREQYF | | | | | | |
| clonotype | CASSLAGRLGETQYF; | CD8 | 2 | 0.09470 | 0.07438 | 0.00330 | 0.13353 |
| 190 | CAVSYYGQNFVF | | | | | | |
| clonotype | CASSIQGLYSNQPQHF; | CD4 | 1 | 0.09149 | 0.30546 | 0.20440 | 0.10786 |
| 138 | CAVGMEYGNKLVF | | | | | | |
| clonotype | CALDNNAGNMLTF; | CD4 | 1 | 0.09638 | 0.20508 | 0.18296 | 0.10285 |
| 59 | CASSVRGLAGAGADTQYF | | | | | | |
| clonotype | CASSYGYGYTF; | CD4 | 1 | 0.14206 | 0.17295 | 0.16820 | 0.08645 |
| 49 | CAGGADGLTF | | | | | | |
| clonotype | CASTYTGELFF; | CD4 | 1 | 0.02361 | 0.20710 | 0.11586 | 0.08951 |
| 193 | CAVVIWFGNEKLTF | | | | | | |
| clonotype | CASSSSTLGNTIYF; | CD4 | 1 | 0.08632 | 0.19077 | 0.11568 | 0.06508 |
| 22 | CAASRNGNNAGNMLTF | | | | | | |
| clonotype | CASSLQGANTEAFF; | CD4 | 1 | 0.05430 | 0.12428 | 0.11097 | 0.06373 |
| 131 | CAVEDRDGGATNKLIF | | | | | | |
| clonotype | CASSLAGTGQETQYF; | CD4 | 1 | 0.03038 | 0.20619 | 0.03582 | 0.09276 |
| 31 | CAEKHGGSQGNLIF | | | | | | |

TABLE 12A
Summary of manufacturing for patient CMI-0104
Plasmid and AAV Production

| Measurement | Result | | Release Criteria | |
|---|---|---|---|---|
| Plasmid Content | 1.02 | mg/mL | 0.80-1.20 | mg/mL |
| Plasmid Purity | 1.91 | | 1.80-2.00 | |
| Supercoiled Content | 89% | | ≥85% | |
| Plasmid Other QC | Appearance, pH, Identity and Integrity, | | | |
| Passed | DNA Sequence, HCD, HCP, Antibiotic | | | |
| | Residue, Endotoxin, Sterility | | | |
| AAV Titer | 5.37 × 10¹¹ | vg/mL | ≥1.00 × 10⁹ | vg/mL |

| | | |
|---|---|---|
| Empty capsid content | 10% | ≤15% |

| | |
|---|---|
| AAV Other QC Passed | Appearance, pH, Osmolality, Capsid |
| | protein, Sequence, HCD, HCP, BSA |
| | Residue, E1A Residue, SV40LTA |
| | Residue, Nuclease Residue, |
| | Mycoplasma, Endotoxin, Sterility |

TABLE 12B
Summary of manufacturing for patient CMI-0104

| Measurement | Result |
|---|---|
| Total Leukopak | 4.88E9 |
| Number of T Cells Recovered | 1.42E9 |
| Fold Expansion | 34 |
| Knock out Efficiency | 90% |
| Knock In Efficiency | 56% |
| Total Cell Number Produced | 2.67E10 |
| % Exogenous TCR | 63.64% |
| Final Product Phenotype | 87.28% |
| (CD62L⁺/CD3⁺) | |
| Number of Present TCRs (>0.1 * 1/N) | 26 |

### Example 11. Example Clinical Protocol of Using Synthetic TILs for Treatment

This example provides an example clinical protocol of using the polyclonal synthetic TILs (e.g., disTIL) described herein to treat a patient with cancer. Any criteria or numbers provided in this example may be adjusted.

Study title: An open, single-arm early clinical study of disTIL injection in patients with advanced malignant solid tumors

Primary objectives: To evaluate the safety and tolerability of TIL administered in subjects with advanced solid tumor patients; To characterize the RP2D (recommended phase II dose) and MTD (Maximal Tolerable Dose) in patients with advanced solid tumor; To characterize PK; To characterize PD; Preliminarily evaluate the effectiveness of disTIL injection in the treatment of advanced malignant solid tumor; Screen reactive TCR from PBMC compared with tissue to evaluate the possibility of noninvasive treatment

Study design: This study is conducted according to the requirements of Chinese regulatory guidelines such as GCP, Technical Guidelines for Research and Evaluation of Cell Therapy Products (Trial), And Technical Guidelines for Clinical Trials of Immunotherapy Products (Trial). The design of this study was based on “Some Thoughts on Non-registered Clinical Trials for Drug Registration Review” published by Gao Jianchao, Clinical Biological Products department of CDE.

This study was a single-arm, open-label, dose-increasing study, to explore the safety, tolerability and cytokinetic characteristics of the drug, and to preliminarily observe the efficacy of the drug in adult patients with advanced malignant solid tumors, so as to explore the appropriate dose for phase II clinical practice. Due to the cell drugs are long survived, the activity and toxic are not obviously dose dependent, and the dose increase may be accompanied by the increase of toxicity instead of a necessary remedial effect. Thus, it's not appropriate for MTD to recommend effective dose. In this study, the RP2D will be determined by safety, preliminary efficacy, the endpoints of pharmacodynamics and pharmacokinetics endpoints (ORR, cytokine secretion levels, disTIL cell amplification and duration).

a. Dose Escalation Study

One research center will be selected in this clinical trial. The initial plan is to divide into 4 dose groups. 1 subject will be concluded in dose 1 and 2 groups respectively. If the investigator and partner deem that further evaluation is needed based on the safety event of the patient, the “3+3” dose escalation method will be adopted. The MTD of the third dose group and subsequent dose groups will be determined according to the “3+3” dose escalation method. For the planned dose group of 3 subjects, if 1 DLT occurs among 3 subjects in a dose group, 3 additional subjects should be added to the same dose group (up to 6 subjects in the dose group to complete DLT evaluation): if no DLT occurs in the additional 3 subjects, the dose increase will be continued; If 1 of the 3 additional subjects develops DLT, the dose increase is stopped; If DLT occurs in >1 of the 3 additional subjects, the dose increment will be stopped and a further 3 subjects will be enrolled for DLT evaluation by lowering one dose. If MTD is not observed at the end of the ascent of the maximum dose group, the investigator and partners will determine whether to increase the dose group based on the preclinical data of disTIL, the feasibility of disTIL CMC, human safety tolerance, pharmacokinetics, and pharmacopotency, while ensuring the safety of the subjects. The dose groups during the study period can be increased, decreased or adjusted by the investigator and the partner based on the pharmacokinetic results obtained.

b. Dose Extension Study

In order to further evaluate the efficacy and safety of disTIL in patients with advanced solid tumors, the investigator and partners may select one or two dose groups for a total of 3-6 subjects after preliminary data on the safety of the dose groups are obtained. This is an early exploratory study, and it is planned to enroll 11-30 patients with advanced malignant solid tumors (including 8-24 patients with dose escalation and 3-6 patients with dose extension).

Sample size: Considering the failure of TCR identification, cell preparation, or the inability of cell transfusion due to rapid disease progression during cell preparation, the number of subjects involved in cell preparation may be more than the planned number of cases in this study.

Safety: Cytokine release syndrome (CRS) and neurotoxicity will be graded using ASTCT 2019 criteria, while the remaining adverse events (AE) will be graded using CTCAE version 5.0 criteria. Unless otherwise specified in the protocol, AE occurring throughout the study period will be assessed. All SAE only related to the study procedure from the time the patient signed the ICF to lymphodepletion should be collected and reported. All SAE meeting the reporting criteria for SAE from the beginning of lymphodepleting chemotherapy should be reported, except SAE determined by the investigator to be due to disease progression. All adverse events will be collected from lymphodepletion until disease progression, withdrawal from the study, death, or 28 days after the last cell transfusion, whichever occurred first. For patients who signed the ICF but did not undergo any disTIL cell transfusion for any reason, only all AE and SAE related procedure or treatment were collected within 28 days of a study (e.g., mononuclear cell collection, tumor tissue biopsy, lymphocyte clearance chemotherapy) or the initiation of new antitumor therapy, whichever occurred first. Adverse events were classified by vital signs, physical examination, 12-lead electrocardiogram, clinical laboratory test, ECOG score, AE and serious adverse events (SAE), and adverse events of special concern (AESI).

Efficacy: Efficacy evaluation criteria: Efficacy is evaluated according to solid tumor evaluation criteria (RECIST 1.1). (1) ORR: Objective Response Rate (ORR) BOR=# with CR+# with PR. (2) OS: The length of time from the date of the start of disTIL treatment to the time of death (from any cause). (3) DCR: Percentage of patients that meet CR, PR and SD criteria set in this study according to RECIST v1.1: DCR (proportion of patients)=# with CR+# with PR+# with SD/# with CR+# with PR+# with SD+# with PD. (4) The time length between recruitment and confirmed subsequent disease progression according to RECIST 1.1. (5) the time length between the first confirmed objective response per RECIST 1.1 to the treatment and the subsequent disease progression per RECIST 1.1.

Time limit: about 2 years.

Study population: Patients (male/female) must fulfill all of the following screening criteria to be eligible for the study:

Inclusion Criteria: (1) diagnosis of advanced malignant solid tumors must be histologically or cytologically confirmed; (2) Age: 18 years to 75 years; (3) Test subjects have failed standard treatment regimens (received one systemic therapy at least), or there are no standard treatment regimens available, and can't be excised completely, including primary hepatocellular carcinoma, cervical cancer, malignant melanoma, renal cancer, colorectal cancer, lung cancer, bladder cancer, head and neck cancer, esophageal cancer, sarcoma, stomach cancer; (4) At least 1 evaluable tumor lesion; (5) Test subjects must have tumor regions eligible for biopsy or resection to separate TILs, tumor cells and normal cells. In patients with surgical resection, at least ≥1.5 cm³ tumor mass and >0.5 g paracancer tissue could be isolated. For patients undergoing biopsy, a minimum of 16G biopsy needle is required to obtain at least 4 pieces of puncture tissue (single source or multiple lesions combined); (6) ECOG: 0-1 and expected life-span more than 3 months; (7) Hematology and Chemistry: (creatinine clearance rate)≥50 mL/min; absolute neutrophil count (ANC)≥1.5×10⁹/L; platelet count (PLT)≥75×10⁹/L; (Hb)≥90 g/L; (oxyhemoglobin saturation)≥91%; Total bilirubin≤2×ULN; ALT and AST≤2.5×ULN; The investigators determined ALT and AST abnormalities as a result of disease (e.g., primary hepatocellular carcinoma, nonhepatocellular metastasis, or bile duct obstruction) or Gilbert syndrome, The index can be relaxed to ≤5×ULN; (8) Subject will return to an acceptable baseline state from all toxicity associated with prior treatment, or return to normal or level 1 on the NCI CTCAE 5.0 scale, as determined by the investigator; Except for toxicity, such as alopecia and vitiligo, which the investigator judged would not increase the safety risk of drug infusion in subsequent studies; (9) Be able to understand and sign the informed consent document; Be able to stick to follow-up visit plan and other requirements in the agreement.

Exclusion criteria: (1) Hepatitis B surface antigen (HBsAg) or hepatitis B core antibody (HBcAb) positive and peripheral blood hepatitis B virus (HBV) DNA titer detection is not within the normal reference range; Hepatitis C virus (HCV) antibody positive and peripheral blood HCV RNA positive; Persons with positive antibodies to human immunodeficiency virus (HIV); Treponema pallidum specific antibody positive; (2) Patients with CNS metastasis of malignant tumors and/or other unstable CNS diseases (hemorrhage, active infarction, infection, etc.); (3) A live attenuated vaccine was administered within 4 weeks prior to cell infusion; (4) History of allergy to chemical compound consisting of chemical and biologic substances resembling cell therapy; (5) Poorly controlled hypertension (systolic blood pressure >160 mmHg and/or diastolic blood pressure >90 mmHg) or cardiovascular and cerebrovascular diseases with clinical significance (e.g. active), Such as cerebrovascular accident (within 6 months prior to signing the informed consent), myocardial infarction (within 6 months prior to signing the informed consent), unstable angina, New York heart association (NYHA) class for II or above of congestive heart failure, or severe arrhythmia can't use drugs to control or have potential impact on research and treatment; Ecg results showed clinically significant abnormalities or mean QTcB≥450 ms in 3 consecutive sessions (with an interval of at least 5 minutes); (6) Severe physical or mental diseases; (7) Patients with pulmonary hypertension; (8) Have a systemic active infection requiring treatment, or have positive blood cultures (or imaging evidence of infection); (9) Autoimmune diseases: A history of autoimmune diseases deemed unsuitable for the study by the investigator, such as systemic lupus erythematosus, vasculitis, and invasive lung disease, should be excluded (except vitiligo subjects); (10) Within 2 weeks prior to screening and during the study period planning to use (if long-term) of systemic cortisone steroids (external and Inhalation preparation are permitted), hydroxyurea, immunomodulators (e.g., a or γ interferon, GM-CSF, mTOR inhibitors, cyclocytase, thymosin, etc.); (11) Women who are pregnant or lactating, and female subjects who plan to have a pregnancy within 1 year after cell transfusion or male subjects whose partners plan to have a pregnancy within 1 year after their cell transfusion; (12) Subjects who have any co-existing medical condition or disease that the investigator determines may affect the conduct of the study; (13) Patients who received other cellular gene therapy products within 6 months prior to cell transfusion and were considered unsuitable for inclusion by the investigator; (14) Patients judged by the investigator to have difficulty completing all visits or procedures required by the study protocol (including the follow-up period), or have insufficient compliance to participate in the study; Or patients that the investigator deemed unsuitable for inclusion.

Subjects are required to meet the following criteria prior to single sampling: ANC≥1.5×10⁹/L; PLT≥50×10⁹/L; No other conditions were determined by the investigator to be unsuitable for monotherapy.

Subjects will meet the following criteria before beginning lymphodepletion: (1) The investigators determined that there was no significant active infection; (2) Subjects' body temperature ≤38° C. within 24 hours prior to dosing (excluding tumor fever as determined by investigator); (3) Subjects should complete relevant examinations according to the visiting schedule for 3 days prior to lymphodepletion. The results were evaluated by the researcher to be able to carry out lymphodepletion; (4) No other conditions were determined by the investigator to be unsuitable for lymphodepletion.

Subjects will meet the following criteria before cell infusion: (1) The investigators determined that there was no significant active infection; (2) Neurotoxicity ≤class 1 (refer to 14.4 definitions); (3) Body temperature ≤38° C. within 24 hours prior to cell infusion (excluding tumor fever as determined by the investigator); (4) Subjects will not receive therapeutic doses of corticosteroids (≥5 mg/day of prednisone or equivalent corticosteroids) or other immunosuppressive drugs (other than topical and inhaled therapy) for 5 days prior to cell infusion; (5) The investigator determined that there was no significant change in subjects' baseline or clinical presentation that would increase the risk of cell infusion compared to the inclusion/exclusion criteria; (6) Prior to cell infusion (rest assessment), subjects will complete relevant examinations according to the visit schedule. It was evaluated by researchers to be able to carry out cell transfusion; (7) No other conditions were determined by the investigator to be unsuitable for cell infusion.

disTIL preparation: About 100-150 mL of peripheral blood mononuclear cells were collected by blood cell mononuclear apparatus for disTIL cell production. The number of peripheral blood mononuclear cells collected should be 5.0×10⁹-1×10¹⁰. If the minimum number of peripheral blood mononuclear cells collected cannot be 5.0×10⁹, the corresponding milliliter number should be adjusted.

Treatment: Lymphodepletion: 7 days (D-7) before disTIL cell transfusion, the subjects received pre-treatment of lymphocyte elimination, and the chemotherapy regimen for lymphocyte elimination was FC regimen: fludarabine 25 mg/m²/d was intravenously injected from D-7 to D-3 for a total of 5 days. From the 7^{th} day to the 6^{th} day, cyclophosphamide was injected intravenously at 60 mg/kg/d for 2 days.

Cell infusion: DO was intravenously injected according to dose groups, and was administered according to dose groups (Table 13). The administration plan was one time.

TABLE 13
Dose escalation phase return plan

| | Dosage | Case |
|---|---|---|
| Dose 1 | 1 × 10⁹ cells | 1-6 |
| Dose2 | 1 × 10¹⁰ cells | 1-6 |
| Dose3 | 3 × 10¹⁰ cells | 3-6 |
| Dose4 | 1 × 10¹¹ cells or the maximum | 3-6 |
| | productive capacity | |

IL-2: The use of IL-2 was limited to a subset of subjects discussed by investigators and collaborators to explore the potential of IL-2 to assist in the amplification of disTIL. For subjects receiving IL-2 in combination: After the first infusion of disTIL cells, patients will be given a small dose of IL-2 subcutaneously, 500000 U/time, twice a day (10-12 h interval) (If patients develop AE, the investigator will decide to stop the administration or continue the administration after AE remission according to the clinical situation of patients, the total number of days of injection is recommended to be 14 days). The first IL-2 injection was performed within 30 minutes after cell infusion. The investigator and collaborators may adjust the IL-2 dose based on the safety/PK/efficacy data of subjects already treated with combined IL-2, and may adjust the IL-2 dose to 1000000 U/dose, 200000 U/dose, or 400000 U/dose while ensuring the safety of the subjects.

DisTIL repeated infusion: Subjects are allowed to receive cell infusion therapy again if the following two conditions are met:
(1) The best outcome of the subjects' last cell infusion was partial response (PR) or above, or disease stabilization (SD) with reduced tumor focus, or if the investigator and collaborators determined that the subjects would benefit from a second infusion;
(2) The copy number of exogenous TCR in peripheral blood was lower than the detection line, or the peak concentration was more than 10 times lower.

Repeated infusion may require another biopsy, another PBMC collection, and another lymphodepletion. Specific execution and infusion dose will be determined by the investigator and the partner.

Dose limiting toxicity (DLT): CRS and neurotoxicity were based on ASTCT2019 standards, and the severity of other adverse events was graded according to NCI-CTCAE version 5.0, and DLT was determined by the investigator. DLT is defined as the presence of any of the following conditions that may be/likely to be/definitely be related to cell transfusion despite treatment: (1) Grade 4 or 5 cytokine release syndrome associated with disTIL injection treatment was present; (2) Grade 3 cytokine release syndrome due to disTIL injection treatment was present and did not resolve below grade 2 within 7 days; (3) Grade 3-5 anaphylaxis associated with disTIL injection; (4) Grade 3 and above organ toxicity related to disTIL injection treatment; (5) Grade 3 neurotoxicity caused by disTIL injection treatment and not relieved to grade 2 and below or grade 4 neurotoxicity within 3 days; (6) Any unanticipated toxicity that the investigator or collaborators determine is necessary to terminate the study. Except for: (1) Grade 3 to 4 tumor lysis syndrome lasted <7 days; (2) Non-blood related toxicity: (a) Any grade of fever, including neutropenic fever, (b) Grade 3 diarrhea lasts <72 hours, (c) Grade 3 nausea and/or vomiting <72 hours, (d) Grade 3 fatigue lasts <7 days, (e) Grade 3 to 4 elevation of transaminase, bilirubin, creatine kinase, urea, or creatinine lasted for <7 days, (f) Any non-blood-related grade 3 adverse event that is rapidly reversible (remission to baseline or grade 2 or below within 7 days); (3) Aphasia/language impairment or confusion/cognitive impairment resolved to grade 1 or below within 2 weeks and to baseline within 4 weeks; (4) Rapid allergic reactions (fever, chills, rash, urticaria, dyspnea, hypotension, and/or nausea associated with cell infusion, etc.) that occur within 2 hours of cell infusion have resolved to grade 2 or below after standard treatment; (5) Hematological toxicity is expected for AE and is not considered as DLT.

Maximum tolerated dose (MTD): The maximum tolerated dose is defined as the maximum dose when DLT was present in no more than two of the six patients.

### Study Endpoints:

### Primary Outcome Measures:

DLT, adverse events, ECOG score, laboratory tests, vital signs, physical examination, etc.; RP2D and MTD

### Secondary Outcome Measures:

PK endpoints: the maximum concentration (Cmax) of disTIL cells (different exogenous TCR and total exogenous TCR copy number) amplified in peripheral blood after drug administration, the time to reach the maximum concentration (Tmax), and the area under the curve auc0-28d at 28 days and auc0-90d at 90 days;
PD endpoints: cytokine secretion levels in peripheral blood

### Endpoint of Validity:

Objective response rate (ORR): The ratio of the best overall response (BOR) to the total number of patients with PR or CR (RECIST 1.1 criteria)
Overall survival (OS): the time from the beginning of disTIL cell therapy to death from any cause;
Disease control rate (DCR): The percentage of evaluable patients who received disTIL cell therapy who achieved remission (CR+PR) and disease stabilization
Disease progression-free survival (PFS): the time from disTIL cell therapy to first disease progression or death from any cause;
Duration of remission (DOR): the time from the first assessment of CR or PR to the first assessment of disease recurrence or death from any cause, whichever came first;

### Exploratory Study Endpoints:

The possibility that specific TCR screened from tumor tissue could be found in peripheral blood.

### Statistical Methods:

1. Analysis sets: For statistical analysis, the following analysis set is defined.

TABLE 14
Analysis set and description

| Analysis set | description |
|---|---|
| Safety Set | Patients who received a transfusion of disTIL cells |
| PK Set | Patients who received disTIL cell infusion therapy |
| | did not or did not have an event that affected |
| | pharmacokinetic analysis. |
| PD Set | Patients who received disTIL cell infusion therapy |
| | and had baseline and at least one post-baseline |
| | pharmacodynamic parameter that could be evaluated |
| Efficacy | Patients who received disTIL cell transfusion, had |
| assessable | baseline tumor evaluation, and at least one post- |
| set | baseline tumor evaluation |

2. General analysis: After the research plan is determined, statistical professionals are responsible for making statistical analysis plan (SAP), and detailed statistical analysis methods will be detailed in SAP. The measurement data were statistically described with the number of cases, mean, standard deviation, median, maximum, minimum, etc. Counting data or grade data are expressed by frequency and frequency. Data from the last time of lymphocytic clearance chemotherapy and the day before cell infusion will be used as baseline data.
3. Safety and tolerability analysis: The security analysis will be performed in the security analysis set. Safety was assessed by a summary of AE/SAE/AESI (CRS, ICANS, and uncontrolled T cell proliferation), changes in laboratory results, abnormalities in electrocardiograms, vital signs, and changes in ECOG scores. CRS and neurotoxicity will be graded using ASTCT 2019 criteria, while the remaining AE will be graded using CTCAE version 5.0 criteria. Adverse events were coded using the International Dictionary of Medical Terms (MedDRA). According to the classification of human organ system, the number and frequency of AE after the collection of patients' mononuclear cells and the first infusion of cells until the end of the study were summarized with corresponding terms. All AESI (CRS, ICANS), SAE (including death), and AE resulting in permanent cessation of cell transfusion during cell transfusion should be summarized in a separate list. Changes in laboratory results will be summarized according to CTCAE version 5.0 classification. For laboratory indicators, the highest levels that occurred during the trial are summarized in terms of counts and percentages. Changes in ECG abnormalities, vital signs, and ECOG scores were compared with baseline and pre-infusion levels for descriptive statistics. Partial vital signs and laboratory results will be diagonally summarized for each subject based on time.
4. Curative effect analysis: Efficacy analysis will be performed in a efficacy assessable cluster. Tumor assessment will be based on RECIST1.1 criteria. Tumor evaluation is performed by a center investigator or a designated investigator. Objective response rate (ORR), disease control rate (DCR) and 95% confidence interval (CI) were calculated. Kaplan-Meier method was used to estimate the median PFS/OS/DOR with a 95% confidence interval. Subjects who did not develop disease progression or death at the analysis date, or who did not develop disease progression at the time of any further antineoplastic treatment, will be culled at the last adequate tumor evaluation prior to the cut-off date or the antineoplastic treatment date. If disease progression or death is recorded after a single missing tumor evaluation, the actual event date of disease progression/death is used for the PFS event date. If disease progression or death is recorded after ≥2 missing tumor evaluations, the date of the last adequate tumor evaluation without disease progression is taken as the PFS deletion time for these subjects. Kaplan-Meier method and appropriate summary statistics were used to describe the PFS assessed by the investigator.
5. Pharmacokinetic (PK) analysis: The PK analysis will be in PK concentration. Descriptive statistical analysis was performed on PK parameters at each visit time point, and the mean, standard deviation, median, minimum, maximum, geometric mean and geometric mean variation coefficient were reported.
6. Pharmacodynamic (PD) analysis: The PD analysis will take place in PD concentration. Descriptive statistical analysis was conducted on PD parameters at each visit time point, and the mean, standard deviation, median, minimum, maximum, geometric mean and variation coefficient of geometric mean were reported.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. (canceled)

2. (canceled)

3. (canceled)

4. (canceled)

5. (canceled)

6. (canceled)

7. (canceled)

8. (canceled)

9. A method of producing a plurality of synthetic tumor-infiltrating lymphocytes (TILs), the method comprising:
(a) obtaining a resection or biopsy sample comprising a plurality of T cells from a solid tumor lesion of a subject;
(b) sequencing the plurality of T cells of the resection or biopsy sample to obtain (i) expression level of at least 10 genes and (ii) natively paired T-cell receptor (TCR) alpha chain and beta chain sequences of TCRs of the plurality of T cells;
(c) synthesizing a plurality of polynucleotide sequences encoding the TCRs or a subset thereof from the natively paired TCR alpha chain and beta chain sequences obtained in (b) comprising a first polynucleotide sequence encoding a first natively paired TCR of a first T cell of the plurality of T cells and a second polynucleotide sequence encoding a second natively paired TCR of a second T cell of the plurality of T cells, and wherein the first polynucleotide sequence is different from the second polynucleotide sequence; and
(d) delivering the plurality of polynucleotide sequences or derivative thereof into a plurality of recipient cells by contacting a mixture comprising the plurality of polynucleotide sequences or derivative thereof with a plurality of recipient cells, thereby producing the plurality of synthetic TILs.

10. (canceled)

11. (canceled)

12. (canceled)

13. (canceled)

14. (canceled)

15. (canceled)

16. (canceled)

17. The method of claim 9, wherein delivering the plurality of polynucleotide sequences or derivative thereof into the plurality of recipient cells in (d) comprises site-specific knocking in a polynucleotide sequence into a genomic locus of a recipient cell of the plurality of recipient cells.

18. (canceled)

19. The method of claim 17, wherein the polynucleotide sequence is site-specifically knocked in in-frame into the genomic locus of the recipient cell of the plurality of recipient cells.

20. (canceled)

21. (canceled)

22. The method of claim 17, wherein the site-specific knocking in the polynucleotide sequence into the genomic locus comprises using a gene editing method selected from the group consisting of clustered regularly interspaced short palindromic repeats (CRISPR), transcription activator-like effector (TALE) nucleases (TALENs), meganucleases, zinc finger nuclease (ZFN), or megaTAL nucleases method.

23. The method of claim 22, wherein the site-specific knocking in the polynucleotide sequence into the genomic locus comprises using CRISPR method.

24. The method of claim 23, wherein the CRISPR method comprises delivering a ribonucleoprotein (RNP) complex comprising a Cas9 protein and a single guide RNA (sgRNA) into the recipient cell.

25. The method of claim 24, wherein the sgRNA targets the TRAC locus.

26. The method of claim 22, wherein the site-specific knocking in the polynucleotide sequence into the genomic locus comprises cutting the genomic locus using the gene editing method, and inserting the polynucleotide sequence into the genomic locus via homologous recombination.

27. The method of claim 9, wherein an endogenous TCR is inactivated in a recipient cell of the plurality of recipient cells.

28. The method of claim 27, wherein the endogenous TCR is knocked down or knocked out in the recipient cell of the plurality of recipient cells.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. A method of producing a plurality of synthetic tumor-infiltrating lymphocytes (TILs), the method comprising:
(a) delivering a plurality of polynucleotide sequences or derivative thereof into a plurality of recipient cells by contacting a mixture comprising the plurality of polynucleotide sequences or derivative thereof with the plurality of recipient cells, wherein each polynucleotide sequence of the plurality encodes a cognate pair of TCR alpha chain and a TCR beta chain, and wherein each polynucleotide sequence is knocked into a genomic locus for monoallelic expression of the polynucleotide sequence; and
(b) inactivating an endogenous TCR gene of each recipient cell of the plurality of recipient cells or an endogenous TCR gene of each recipient cell of the plurality of recipient cells has been inactivated.

79. (canceled)

80. The method of claim 78, wherein the genomic locus is TRAC locus.

81. The method of claim 80, wherein the endogenous TCR gene is inactivated by knocking down or knocking out TRBC locus.

82. The method of claim 80, wherein the mixture comprises at least about 2, 3, 4, 5, 10, 20, 30, 40, 50, 100, 500, 1,000 or more different polynucleotide sequences encoding at least about 2, 3, 4, 5, 10, 20, 30, 40, 50, 100, 500, 1,000 or more different natively paired TCRs.

83. (canceled)

84. (canceled)

85. (canceled)

86. The method of claim 78, wherein an endogenous TCR gene of each recipient cell of the plurality of recipient cells has been inactivated prior to delivering in (a).

87. (canceled)

88. (canceled)

89. (canceled)

90. The method of claim 78, wherein inactivating in (b) is done concurrently, prior to or after delivering in (a).

91. The method of claim 78, further comprising expanding the plurality of synthetic TILs.

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. A method of treating cancer in a subject in need thereof, the method comprising administering to the subject a therapeutically effective amount of a pharmaceutical composition, wherein the pharmaceutical composition comprises one or more synthetic TILs of claim 9, and a pharmaceutically acceptable carrier, or a mixture comprising at least 2, 5, 10, 15, 20, 25, 30, 50, 100, 200, 500, or 1000 modified cells, each modified cell of the mixture exogenously expressing a polynucleotide sequence encoding a distinct cognate pair of a TCR alpha chain and a TCR beta chain, wherein the polynucleotide sequence is knocked into a genomic locus for monoallelic expression of the polynucleotide sequence, and wherein an endogenous TCR gene of the modified cell is inactivated.

125. (canceled)

126. (canceled)

127. (canceled)

128. (canceled)

129. (canceled)

130. (canceled)

131. (canceled)

132. (canceled)

133. The method of claim 124, wherein the modified cell is a T cell.

134. (canceled)

135. The method of claim 133, wherein the T cell is an allogenic T cell or an autologous T cell.

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)
